(19) United States
(12) Patent Application Publication
(10) Pub. No.: US 2021/0038607 A1
(43) Pub. Date: Feb. 11, 2021

(54) OXADIAZOLYLTHIOPHENE DERIVATIVES USEFUL AS HISTONE DEACETYLASE INHIBITORS

(71) Applicant: Karus Therapeutics Limited, Oxfordshire (GB)

(72) Inventors: Stephen Joseph Shuttleworth, Oxfordshire (GB); Alice Elizabeth Gatland, Oxfordshire (GB); Daniel John Finnemore, Oxfordshire (GB); Rikki Peter Alexander, Oxfordshire (GB); Franck Alexandre Silva, Oxfordshire (GB); Alexander Richard Liam Cecil, Oxfordshire (GB)

(21) Appl. No.: 16/977,258

(22) PCT Filed: Mar. 1, 2019
(86) PCT No.: PCT/GB2019/050580

§ 371 Date: Sep. 1, 2020

(30) Foreign Application Priority Data
Mar. 1, 2018 (GB) 1803361.3

(51) Int. Cl. A61K 31/5377; A61K 45/06; C07D 417/14; A61K 31/497; A61K 31/444; C07D 413/14; C07D 471/04; A61K 31/4375; A61K 31/4985; A61K 31/501

(52) U.S. Cl. CPC A61K 31/5377; A61K 45/06; C07D 417/14; A61K 31/497; A61K 31/501; C07D 413/14; C07D 471/04; A61K 31/4375; A61K 31/4985; A61K 31/444

(57) ABSTRACT

A compound of Formula I: (I) or a pharmaceutically acceptable salt thereof, wherein: each R′ is QR¹; each Q is independently selected from a bond, —C₁-C₁₀ alkylene, —C₂-C₁₀ alkenylene, —C(O)—, —C(O)O—, —C(O)N(R¹)—, —C(O)N(R¹)SO₂—N(R¹)C(O)—, —N(R¹)—, —N(SO₂(R¹)), —N(R¹)SO₂—C(O)NR⁴R⁵—, —N(R⁴R⁵)C(O)—, —N(R⁴R⁵)—S—, —SO—, —SO₂—, —S(O)O—, —SO₂N(R¹)— and —O—; each R¹ is independently selected from H, C₁-C₁₀ alkyl, C₂-C₁₀ alkenyl, C₂-C₁₀ alkynyl, C₁-C₁₀ haloalkyl, C₁-C₁₀ heteroalkyl, aryl, heteroaryl, C₃-C₁₀ cycloalkyl, —(C₁-C₁₀ alkylene)-C₃-C₁₀ cycloalkyl, halogen, cyano, C₁-C₁₀ alkylene-aryl, C₁-C₁₀ alkylene heteroaryl, C₁-C₁₀ heterocycloalkyl and —(C₁-C₁₀ alkylene)-C₁-C₁₀ heterocycloalkyl. The compounds are inhibitors of HDAC and therefore have potential utility in the therapy of a number of conditions including cancer and inflammation.

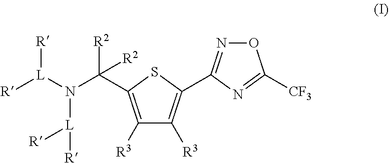

### FIELD OF THE INVENTION

The present invention relates to novel compounds which are inhibitors of histone deacetylase (HDAC) and therefore have therapeutic utility.

### BACKGROUND OF THE INVENTION

HDACs are zinc metalloenzymes that catalyse the hydrolysis of acetylated lysine residues. In histones, this returns lysines to their protonated state and is a global mechanism of eukaryotic transcriptional control, resulting in tight packaging of DNA in the nucleosome. Additionally, reversible lysine acetylation is an important regulatory process for non-histone proteins. HDAC6, for example, is principally a non-histone protein deacetylase, regulating the acetylation status of several substrates including tubulin, Hsp90, cortactin and β-catenin. Thus, compounds which are able to modulate HDAC have important therapeutic potential.

WO2016/067038, WO2014/181137, WO2017/222950, WO2017/222951, and WO2017/222952 all disclose compounds reported to act as inhibitors of HDAC.

### SUMMARY OF THE INVENTION

A compound of Formula I

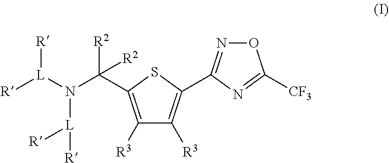

or a pharmaceutically acceptable salt thereof, wherein:

each R′ is QR¹;

each Q is independently selected from a bond, —C₁-C₁₀ alkylene, —C₂-C₁₀ alkenylene, —C(O)—, —C(O)O—, —C(O)N(R¹)—, —C(O)N(R¹)SO₂—, —N(R¹)C(O)—, —N(R¹)—, —N(SO₂(R¹)), —N(R¹)SO₂—, —C(O)NR⁴R⁵—, —N(R⁴R⁵)C(O)—, —N(R⁴R⁵)—, —S—, —SO—, —SO₂—, —S(O)O—, —SO₂N(R¹)— and —O—;

each R¹ is independently selected from H, C₁-C₁₀ alkyl, C₂-C₁₀ alkenyl, C₂-C₁₀ alkynyl, C₁-C₁₀ haloalkyl, C₁-C₁₀ heteroalkyl, aryl, heteroaryl, C₃-C₁₀ cycloalkyl, —(C₁-C₁₀ alkylene)-C₃-C₁₀ cycloalkyl, halogen, cyano, C₁-C₁₀ alkylene-aryl, C₁-C₁₀ alkylene heteroaryl, C₁-C₁₀ heterocycloalkyl and —(C₁-C₁₀ alkylene)-C₁-C₁₀ heterocycloalkyl;

each R² is independently selected from H, halogen and C₁-C₄ alkyl;

each R³ is independently selected from H, halogen, C₁-C₄ alkyl and C₁-C₁₀ haloalkyl;

each R⁴ and R⁵, when taken together with the nitrogen to which they are attached, form a 4- to 10-membered heteroarylene or heterocycloalkylene linker; and

each L is independently selected from a 5- to 10-membered nitrogen-containing heteroaryl;

wherein each alkyl, heteroalkyl, cycloalkyl, heterocycloalkyl, aryl or heteroaryl may be substituted by up to three substituents selected from C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl) amino C₁-C₃ alkyl, bis(C₁-C₃ alkyl) amino C₁-C₃ alkyl, C₁-C₃ alkyl sulfonylamino, halo, nitro, cyano, C₁-C₃ haloalkyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl.

The compounds of the invention may be useful as an inhibitor of HDAC, i.e. they may be used in a method of treating a disease associated with an over-expression of HDAC.

### DESCRIPTION OF THE INVENTION

### Definitions

As used herein “alkyl” means a C₁-C₁₀ alkyl group, which can be linear or branched. Preferably, it is a C₁-C₆ alkyl moiety. More preferably it is a C₁-C₄ alkyl moiety. Examples include methyl, ethyl, n-propyl and t-butyl. It may be divalent, e.g. propylene.

As used herein “heteroalkyl” means an alkyl as defined above containing up to 4 heteroatoms selected from oxygen, nitrogen and sulfur.

As used herein “cycloalkyl” contains from 3 to 10 carbon atoms. It may be monovalent or divalent. It may be an alkyl group, as defined above, containing a cyclic portion (or completely cyclic).

As used herein “alkenyl” means a C₂-C₁₀ alkenyl group. Preferably, it is a C₂-C₆ alkenyl group. More preferably, it is a C₂-C₄ alkenyl group. The alkenyl radicals may be mono- or di-saturated, more preferably monosaturated. Examples include vinyl, allyl, 1-propenyl, isopropenyl and 1-butenyl. It may be divalent, e.g. propenylene.

As used herein “alkynyl” means a C₂-C₁₀ alkynyl group which can be linear or branched. Preferably, it is a C₂-C₄ alkynyl group or moiety. It may be divalent.

As used herein “haloalkyl” means a C₁-C₁₀ alkyl group as defined above, which may be substituted with up to 10 halogen atoms or more preferably up to 5 halogens. For example, they may be substituted by 1, 2, 3, 4 or 5 halogen atoms. Preferably, the halogen is fluorine. Preferably the haloalkyl is selected from —CF₃, —CHF₂, and —CH₂F. In some embodiments the haloalkyl is —CF₃. In other embodiments the haloalkyl is —CHF₂.

As used herein “aryl” means a monocyclic, bicyclic or tricyclic monovalent, divalent, trivalent or tetravalent (as appropriate) aromatic radical, such as phenyl, biphenyl, naphthyl, anthracenyl, which can be optionally substituted by preferably up to three substituents selected from C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃alkyl) amino C₁-C₃ alkyl, bis(C₁-C₃ alkyl) amino C₁-C₃ alkyl, C₁-C₃ alkyl sulfonylamino, halo, nitro, cyano, trifluoromethyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl.

As used herein “heteroaryl” means a monocyclic, bicyclic or tricyclic monovalent, divalent, trivalent, tetravalent (as appropriate) aromatic radical containing at least one and up to four heteroatoms selected from oxygen, nitrogen and sulfur, such as furanyl, pyrrolyl, thiazolyl, isothiazolyl, tetrazolyl, imidazolyl, oxazolyl, isoxazolyl, thienyl, pyrazolyl, pyridinyl, pyrazinyl, pyrimidinyl, indolyl, azaindolyl, isoindolyl, quinolyl, isoquinolyl, triazolyl, thiadiazolyl, oxadiazolyl, said radical being optionally substituted by preferably up to three substituents selected from C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl) amino C₁-C₃ alkyl, bis(C₁-C₃ alkyl) amino C₁-C₃ alkyl, C₁-C₃ alkyl sulfonylamino, halo, nitro, cyano, trifluoromethyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl.

As used herein “heterocycloalkyl” is a monovalent or divalent carbocyclic radical containing up to 4 heteroatoms selected from oxygen, nitrogen and sulfur. It may be monocyclic or bicyclic. It may contain up to 10 carbon atoms in the ring. It is preferably saturated. At least one of the carbons in the ring may be attached via a double bond to an oxygen atom, i.e. there may be a carboxy functional group within the ring.

As used herein, the above groups can be followed by the suffix -ene. This means that the group is divalent, i.e. a linker group.

### Preferred Groups of the Invention

The linker groups listed in the claims are not ‘direction specific’. They can be reversed. Therefore, the group —C(O)N(R¹)— can also be —N(R¹)C(O)—.

Each R′ is independently QR¹. In some embodiments, at least one R′ is H. Preferably, at least one R′ on each L is H, i.e. preferably at least one L is monosubstituted. In some embodiments, both L groups are monosubstituted.

In some embodiments, Q is independently selected from a bond, —C(O)N(R¹)—, —C(O)O— and —C(O)—, and R is independently selected from H, C₁-C₁₀ alkyl, C₁-C₁₀ haloalkyl, C₁-C₁₀ heterocycloalkyl (e.g. morpholine or piperazine) and heteroaryl. Preferably, each R¹ is independently selected from H, C₁-C₁₀ haloalkyl and C₁-C₁₀ heterocycloalkyl.

R⁴ and R, when taken together with the nitrogen to which they are attached, form a 4- to 10-membered heteroarylene or heterocycloalkylene linker. For example, the group —C(O)NR⁴R⁵— covers such groups as:

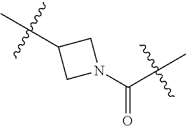 [SMILES: *C(=O)N1CC(*)C1]

In some embodiments, at least one L is a 6-membered nitrogen-containing heteroaryl. In some embodiments, at least one L is a 5-membered nitrogen-containing heteroaryl. In some embodiments, each L is independently selected from a 5- or 6-membered nitrogen-containing heteroaryl.

In some embodiments, each L is a 6-membered nitrogen-containing heteroaryl.

In some embodiments, at least one L, preferably each L, contains two nitrogen atoms.

In some embodiments, at least one L, preferably each L, is independently selected from the group consisting of pyridyl, pyrazolyl, pyrazinyl, pyrimidinyl, pyridazinyl, thiadiazolyl, oxadiazolyl, imidazolyl, pyrazolopyrimidinyl, imidazo(1,2-b)pyridazinyl, and (1,2,4)triazolo(4,3-b)pyridazinyl.

Preferably, at least one L, preferably each L, is pyrazinyl or pyridazinyl.

L may be selected from the group consisting of:

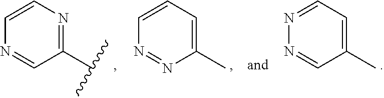

In some embodiments, at least one R² is H. In some embodiments, each R² is H.

In some embodiments, at least one R³, preferably each R³, is H or halogen. In some embodiments, halogen is fluorine. In some embodiments each R³ is H. In some embodiments, one R³ is H and one R³ is halogen, for example fluorine.

In some embodiments, one L group is monosubstituted and the other L group is unsubstituted (i.e. R′ is H on that group).

In some embodiments, at least one R′ is independently selected from H, C₁-C₁₀ haloalkyl (for example CF₃), heterocycloalkyl, heteroaryl, cyano, —C(O)OR¹ and —C(O)R¹, wherein R¹ is heteroaryl or heterocycloalkyl (e.g. morpholine or piperazine).

For example, provided here in an embodiment, are compounds of Formula II and pharmaceutically acceptable salts thereof:

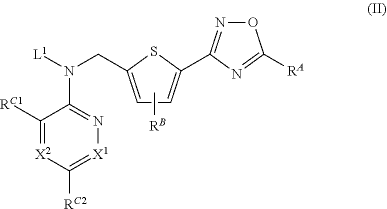

wherein

R^{A} is selected from the group consisting of CF₃, CF₂H, CFH₂ and cyano;

R^{B} is H or F;

X¹ is CR^{C3} or N;

X² is CR^{C3} or N; wherein only one of X¹ or X² is N;

L¹ is a 6-membered heteroaryl having two nitrogens, wherein L¹ is optionally substituted on one or more free carbons with a substituent independently selected for each occurrence from the group consisting of halogen, cyano, C₁₋₃alkyl (optionally substituted with one, two or three halogens), C₁₋₃alkoxy (optionally substituted with one, two or three halogens), C(O)OR^{c}, NR^{a}R^{b}, C(O)NR^{a}R^{b}, S(O)_{w}—C₁₋₃alkyl (where w is 0, 1, or 2), C₃₋₄cycloalkyl, and heteroaryl;

R^{C1}, R^{C2}, and R^{C3} are each independently selected from the group consisting of hydrogen, halogen, cyano, C₁₋₃alkyl (optionally substituted with one, two or three halogens), C₁₋₃alkoxy (optionally substituted with one, two or three halogens), C(O)OR^{c}, NR^{a}R^{b}, C(O)NR^{a}R^{b}, S(O)_{w}—C₁₋₃alkyl (where w is 0, 1, or 2), C₃₋₄cycloalkyl, and heteroaryl;

R^{c} is H or C₁₋₄alkyl; and

R^{a} and R^{b} are for each occurrence independently selected from H and C₁₋₃ alkyl, or R^{a} and R^{b} taken together with the nitrogen to which they are attached form a 4-6 membered monocyclic heterocyclic ring, optionally substituted with a substituent selected from the group consisting of halogen, C₁₋₃alkyl (optionally substituted with one, two or three halogens), C(O)OR^{c}, and C₁₋₃alkoxy (optionally substituted with one, two or three halogens).

For example, L¹ may be selected from the group consisting of:

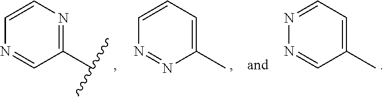

Exemplary compounds of Formulas IIIa, IIIb and IIIc (and pharmaceutically acceptable salts thereof) are also provided:

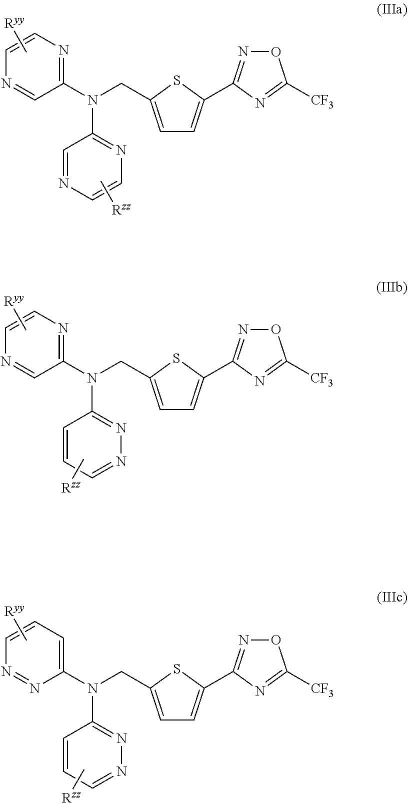

wherein R^{yy} is H or CF₃; and R^{zz} is selected from the group consisting of hydrogen, halogen, cyano, C₁₋₃alkyl (optionally substituted with one, two or three halogens), C₁₋₃alkoxy (optionally substituted with one, two or three halogens), C(O)OR^{c}, NR^{a}R^{b}, C(O)NR^{a}R^{b}; S(O)_{w}—C₁₋₃alkyl (where w is 0, 1, or 2), C₃₋₄cycloalkyl, and heteroaryl; and

wherein R^{a}, R^{b} and R^{c} are as defined above.

A pharmaceutical composition of the invention comprises a compound as defined above, and a pharmaceutically acceptable carrier or diluent. A pharmaceutical composition of the invention typically contains up to 85 wt % of a compound of the invention, based upon the total weight of the composition. More typically, it contains up to 50 wt % of a compound of the invention, based upon the total weight of the composition. Preferred pharmaceutical compositions are sterile and pyrogen-free. Further, the pharmaceutical compositions provided by the invention typically contain a compound of the invention which is a substantially pure optical isomer. Preferably, the pharmaceutical composition comprises a pharmaceutically acceptable salt form of a compound of the invention. For example, contemplated herein is a pharmaceutically acceptable composition comprising a disclosed compound and a pharmaceutically acceptable excipient.

As used herein, a pharmaceutically acceptable salt is a salt with a pharmaceutically acceptable acid or base. Pharmaceutically acceptable acids include both inorganic acids such as hydrochloric, sulfuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, ethanedisulfonic, salicylic, stearic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aryl amines or heterocyclic amines.

For the avoidance of doubt, the present invention also embraces prodrugs which react in vivo to give a compound of the present invention.

The compounds of the present invention are found to be inhibitors of HDAC. The compounds of the present invention are therefore therapeutically useful in the treatment of conditions affected by HDAC activity.

The compounds of the invention may be prepared by synthetic routes that will be apparent to those skilled in the art, e.g. based on the Examples.

The compounds of the present invention are found to be inhibitors of HDAC. The compounds of the present invention are therefore therapeutically useful.

The compounds of the invention and compositions comprising them may be administered in a variety of dosage forms. In one embodiment, a pharmaceutical composition comprising a compound of the invention may be formulated in a format suitable for oral, rectal, parenteral, intranasal or transdermal administration or administration by inhalation or by suppository. Typical routes of administration are parenteral, intranasal or transdermal administration or administration by inhalation.

The compounds of the invention can be administered orally, for example as tablets, troches, lozenges, aqueous or oily suspensions, dispersible powders or granules. Preferred pharmaceutical compositions of the invention are compositions suitable for oral administration, for example tablets and capsules.

The compounds of the invention may also be administered parenterally, whether subcutaneously, intravenously, intramuscularly, intrasternally, transdermally or by infusion techniques. The compounds may also be administered as suppositories.

The compounds of the invention may also be administered by inhalation. An advantage of inhaled medications is their direct delivery to the area of rich blood supply in comparison to many medications taken by oral route. Thus, the absorption is very rapid as the alveoli have an enormous surface area and rich blood supply and first pass metabolism is bypassed. A further advantage may be to treat diseases of the pulmonary system, such that delivering drugs by inhalation delivers them to the proximity of the cells which are required to be treated.

The present invention also provides an inhalation device containing such a pharmaceutical composition. Typically said device is a metered dose inhaler (MDI), which contains a pharmaceutically acceptable chemical propellant to push the medication out of the inhaler.

The compounds of the invention may also be administered by intranasal administration. The nasal cavity's highly permeable tissue is very receptive to medication and absorbs it quickly and efficiently, more so than drugs in tablet form. Nasal drug delivery is less painful and invasive than injections, generating less anxiety among patients. By this method absorption is very rapid and first pass metabolism is usually bypassed, thus reducing inter-patient variability. Further, the present invention also provides an intranasal device containing such a pharmaceutical composition.

The compounds of the invention may also be administered by transdermal administration. The present invention therefore also provides a transdermal patch containing a compound of the invention.

The compounds of the invention may also be administered by sublingual administration. The present invention therefore also provides a sub-lingual tablet comprising a compound of the invention.

A compound of the invention may also be formulated with an agent which reduces degradation of the substance by processes other than the normal metabolism of the patient, such as anti-bacterial agents, or inhibitors of protease enzymes which might be the present in the patient or in commensural or parasite organisms living on or within the patient, and which are capable of degrading the compound.

Liquid dispersions for oral administration may be syrups, emulsions and suspensions.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

In one embodiment the compounds of the present invention may be used in combination with other therapeutic agents. In this embodiment, the combination product may be formulated such that it comprises each of the medicaments for simultaneous, separate or sequential use.

The compounds of the present invention can be used in both the treatment and prevention of cancer and can be used in a monotherapy or in a combination therapy. When used in a combination therapy, the compounds of the present invention are typically used together with small chemical compounds such as signal transduction pathway inhibitors (including but not restricted to PI3K-AKT-mTOR pathway inhibitors, Ras-Raf-MEK-ERK inhibitors, HSP90 inhibitors, Hedgehog pathway inhibitors, growth factor receptor inhibitors), bromodomain inhibitors, immunotherapeutics (including but not restricted to anti-PD-1, anti-CD20 and anti-CD38 agents), platinum complexes, anti-metabolites, DNA topoisomerase inhibitors, but also radiation, antibody-based therapies (for example herceptin and rituximab), anti-cancer vaccination, gene therapy, cellular therapies, hormone therapies or cytokine therapy.

In one embodiment of the invention a compound of the invention is used in combination with another chemotherapeutic or antineoplastic agent in the treatment of a cancer. Examples of such other chemotherapeutic or antineoplastic agents include signal transduction pathway inhibitors, immunotherapeutics, immunotherapeutics, platinum complexes such as cisplatin and carboplatin, mitoxantrone, vinca alkaloids for example vincristine and vinblastine, anthracycline antibiotics for example daunorubicin and doxorubicin, alkylating agents for example chlorambucil and melphalan, taxanes for example paclitaxel, antifolates for example methotrexate and tomudex, epipodophyllotoxins for example etoposide, camptothecins for example irinotecan and its active metabolite SN38 and DNA methylation inhibitors for example the DNA methylation inhibitors disclosed in WO02/085400.

According to the invention, therefore, products are provided which contain a compound of the invention and another chemotherapeutic or antineoplastic agent as a combined preparation for simultaneous, separate or sequential use in alleviating a cancer. Also provided according to the invention is the use of compound of the invention in the manufacture of a medicament for use in the alleviation of cancer by co-administration with another chemotherapeutic or antineoplastic agent. The compound of the invention and the said other agent may be administrated in any order. In both these cases the compound of the invention and the other agent may be administered together or, if separately, in any order as determined by a physician.

HDAC is believed to contribute to the pathology and/or symptomology of several different diseases such that reduction of the activity of HDAC in a subject through inhibition of HDAC may be used to therapeutically address these disease states. Examples of various diseases that may be treated using the HDAC inhibitors of the present invention are described herein.

One set of indications that HDAC inhibitors of the present invention may be used to treat are those involving undesirable or uncontrolled cell proliferation. Such indications include benign tumors, various types of cancers such as primary tumors and tumor metastasis, restenosis (e.g. coronary, carotid, and cerebral lesions), abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants. More specific indications for HDAC inhibitors include, but are not limited to prostate cancer, endometrial cancer, ovarian cancer, cholangiocarcinoma, liver cancer, pancreatic cancer, lung cancer, leukemia, lymphoma, multiple myeloma, uterine cancer, bladder cancer, renal cancer, oesophageal cancer, breast cancer, gastric cancer, colorectal cancer, neuroblastoma, medulloblastoma, glioma, and melanoma.

In one embodiment, a method is provided for treating diseases associated with undesired and uncontrolled cell proliferation. The method comprises administering to a subject suffering from uncontrolled cell proliferation a therapeutically effective amount of a HDAC inhibitor according to the present invention, such that said uncontrolled cell proliferation is reduced. The particular dosage of the inhibitor to be used will depend on the severity of the disease state, the route of administration, and related factors that can be determined by the attending physician. Generally, acceptable and effective daily doses are amounts sufficient to effectively slow or eliminate uncontrolled cell proliferation.

HDAC inhibitors according to the present invention may also be used in conjunction with other agents to inhibit undesirable and uncontrolled cell proliferation. Examples of other anti-cell proliferation agents that may be used in conjunction with the HDAC inhibitors of the present invention include, but are not limited to, retinoid acid and derivatives thereof, 2-methoxyestradiol, Angiostatin™ protein, Endostatin™ protein, suramin, squalamine, tissue inhibitor of metalloproteinase-1, tissue inhibitor of metalloproteinase-2, plasminogen activator inhibitor-1, plasminogen activator inhibitor-2, cartilage-derived inhibitor, paclitaxel, platelet factor 4, protamine sulfate (clupeine), sulfated chitin derivatives (prepared from queen crab shells), sulfated polysaccharide peptidoglycan complex (sp-pg), staurosporine, modulators of matrix metabolism, including for example, proline analogs ((1-azetidine-2-carboxylic acid (LACA), cishydroxyproline, d,l-3,4-dehydroproline, thiaproline), beta-aminopropionitrile fumarate, 4-propyl-5-(4-pyridinyl)-2(3H)-oxazolone; methotrexate, mitoxantrone, heparin, interferons, 2 macroglobulin-serum, chimp-3, chymostatin, beta-cyclodextrin tetradecasulfate, eponemycin; fumagillin, gold sodium thiomalate, d-penicillamine (CDPT), beta-1-anticollagenase-serum, alpha-2-antiplasmin, bisantrene, lobenzarit disodium, n-(2-carboxyphenyl-4-chloroanthronilic acid disodium or “CCA”, thalidomide; angiostatic steroid, carboxyaminoimidazole; metaloproteinase inhibitors such as BB94. Other anti-angiogenesis agents that may be used include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

Generally, cells in benign tumors retain their differentiated features and do not divide in a completely uncontrolled manner. A benign tumor is usually localized and nonmetastatic. Specific types of benign tumors that can be treated using HDAC inhibitors of the present invention include hemangiomas, hepatocellular adenoma, cavernous hemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In the case of malignant tumors, cells become undifferentiated, do not respond to the body's growth control signals, and multiply in an uncontrolled manner. Malignant tumors are invasive and capable of spreading to distant sites (metastasizing). Malignant tumors are generally divided into two categories: primary and secondary. Primary tumors arise directly from the tissue in which they are found. Secondary tumors, or metastases, are tumors that originated elsewhere in the body but have now spread to distant organs. Common routes for metastasis are direct growth into adjacent structures, spread through the vascular or lymphatic systems, and tracking along tissue planes and body spaces (peritoneal fluid, cerebrospinal fluid, etc.).

Specific types of cancers or malignant tumors, either primary or secondary, that can be treated using the HDAC inhibitors of the present invention include, but are not limited to, leukemia, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglloneuromas, hyperplastic corneal nerve tumor, marfanoid habitus tumor, Wilms' tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforme, leukemias, lymphomas, malignant melanomas, epidermoid carcinomas, and other carcinomas and sarcomas.

The HDAC inhibitors of the present invention may also be used to treat abnormal cell proliferation due to insults to body tissue during surgery. These insults may arise as a result of a variety of surgical procedures such as joint surgery, bowel surgery, and cheloid scarring. Diseases that produce fibrotic tissue that may be treated using the HDAC inhibitors of the present invention include emphysema. Repetitive motion disorders that may be treated using the present invention include carpal tunnel syndrome. An example of a cell proliferative disorder that may be treated using the invention is a bone tumor.

Proliferative responses associated with organ transplantation that may be treated using HDAC inhibitors of the invention include proliferative responses contributing to potential organ rejections or associated complications. Specifically, these proliferative responses may occur during transplantation of the heart, lung, liver, kidney, and other body organs or organ systems.

Abnormal angiogenesis that may be treated using this invention include those abnormal angiogenesis accompanying rheumatoid arthritis, ischemic-reperfusion related brain edema and injury, cortical ischemia, ovarian hyperplasia and hypervascularity, polycystic ovary syndrome, endometriosis, psoriasis, diabetic retinopathy, and other ocular angiogenic diseases such as retinopathy of prematurity (retrolental fibroplastic), macular degeneration, corneal graft rejection, neuroscular glaucoma and Oster Webber syndrome.

Examples of diseases associated with uncontrolled angiogenesis that may be treated according to the present invention include, but are not limited to retinal/choroidal neovascularization and corneal neovascularization. Examples of diseases which include some component of retinal/choroidal neovascularization include, but are not limited to, Best's diseases, myopia, optic pits, Stargart's diseases, Paget's disease, vein occlusion, artery occlusion, sickle cell anemia, sarcoid, syphilis, pseudoxanthoma elasticum carotid apo structive diseases, chronic uveitis/vitritis, mycobacterial infections, Lyme's disease, systemic lupus erythematosus, retinopathy of prematurity, Eale's disease, diabetic retinopathy, macular degeneration, Bechet's diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy. Examples of corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

Chronic inflammatory diseases associated with uncontrolled angiogenesis may also be treated using HDAC inhibitors of the present invention. Chronic inflammation depends on continuous formation of capillary sprouts to maintain an influx of inflammatory cells. The influx and presence of the inflammatory cells produce granulomas and thus maintains the chronic inflammatory state. Inhibition of angiogenesis using a HDAC inhibitor alone or in conjunction with other anti-inflammatory agents may prevent the formation of the granulomas and thus alleviate the disease. Examples of chronic inflammatory diseases include, but are not limited to, inflammatory bowel diseases such as Crohn's disease and ulcerative colitis, psoriasis, sarcoidosis, and rheumatoid arthritis.

Inflammatory bowel diseases such as Crohn's disease and ulcerative colitis are characterized by chronic inflammation and angiogenesis at various sites in the gastrointestinal tract. For example, Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhoea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhoea. These inflammatory bowel diseases are generally caused by chronic granulomatous inflammation throughout the gastrointestinal tract, involving new capillary sprouts surrounded by a cylinder of inflammatory cells. Inhibition of angiogenesis by these inhibitors should inhibit the formation of the sprouts and prevent the formation of granulomas. Inflammatory bowel diseases also exhibit extra intestinal manifestations, such as skin lesions. Such lesions are characterized by inflammation and angiogenesis and can occur at many sites other the gastrointestinal tract. Inhibition of angiogenesis by HDAC inhibitors according to the present invention can reduce the influx of inflammatory cells and prevent lesion formation.

Sarcoidosis, another chronic inflammatory disease, is characterized as a multisystem granulomatous disorder. The granulomas of this disease can form anywhere in the body. Thus, the symptoms depend on the site of the granulomas and whether the disease is active. The granulomas are created by the angiogenic capillary sprouts providing a constant supply of inflammatory cells. By using HDAC inhibitors according to the present invention to inhibit angiogenesis, such granulomas formation can be inhibited. Psoriasis, also a chronic and recurrent inflammatory disease, is characterized by papules and plaques of various sizes. Treatment using these inhibitors alone or in conjunction with other anti-inflammatory agents should prevent the formation of new blood vessels necessary to maintain the characteristic lesions and provide the patient relief from the symptoms.

Rheumatoid arthritis (RA) is also a chronic inflammatory disease characterized by non-specific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Treatment using HDAC inhibitors according to the present invention alone or in conjunction with other anti-RA agents may prevent the formation of new blood vessels necessary to maintain the chronic inflammation.

The compounds of the present invention can further be used in the treatment of cardiac/vasculature diseases such as hypertrophy, hypertension, myocardial infarction, reperfusion, ischemic heart disease, angina, arrhythmias, hypercholesterolemia, and atherosclerosis. The compounds can further be used to treat neurodegenerative disorders and CNS disorders such as acute and chronic neurological diseases, including a neuromuscular disorder (e.g. spinal muscular atrophy), stroke, Huntington's disease, Parkinson's disease, Amyotrophic lateral sclerosis and Alzheimer's disease. The compounds can be used in the treatment of Charcot-Marie-Tooth disease, polycystic liver disease and rhabdomyolysis.

The compounds of the present invention can also be used as antimicrobial agents, for example antibacterial agents. The invention therefore also provides a compound for use in the treatment of a bacterial infection. The compounds of the present invention can be used as anti-infectious compounds against viral, bacterial, fungal and parasitic infections. Examples of infections include protozoal parasitic infections (including plasmodium, Cryptosporidium parvum, Toxoplasma gondii, sarcocystis neurona and Eimeria sp.)

The compounds of the present invention are particularly suitable for the treatment of undesirable or uncontrolled cell proliferation, preferably for the treatment of benign tumors/hyperplasias and malignant tumors, more preferably for the treatment of malignant tumors and most preferably for the treatment of chronic lymphocytic leukemia (CLL), breast cancer, prostate cancer, ovarian cancer, mesothelioma, T-cell lymphoma.

In a preferred embodiment of the invention, the compounds of the invention are used to alleviate cancer, cardiac hypertrophy, chronic heart failure, an inflammatory condition, a cardiovascular disease, a hemoglobinopathy, a thalassemia, a sickle cell disease, a CNS disorder, an autoimmune disease, organ transplant rejection, diabetes, osteoporosis, MDS, benign prostatic hyperplasia, oral leukoplakia, a genetically related metabolic disorder, an infection, Rubens-Taybi, fragile X syndrome, or alpha-1 antitrypsin deficiency, or to accelerate wound healing, to protect hair follicles or as an immunosuppressant.

Typically, said inflammatory condition is a skin inflammatory condition (for example psoriasis, epidermolysis bullosa, epidermolysis bullosa acquisita, acne or eczema), a musculoskeletal inflammatory condition (in particular rheumatoid arthritis (RA), juvenile rheumatoid arthritis, ankylosing spondylitis or osteoarthritis), asthma, chronic obstructive pulmonary disease (COPD), an inflammatory condition of the gastrointestinal tract, (for example inflammatory bowel disease (IBD) including Crohn's disease and ulcerative colitis).

Typically, said cancer is chronic lymphocytic leukemia, breast cancer, prostate cancer, ovarian cancer, mesothelioma or T-cell lymphoma.

Typically, said cardiovascular disease is hypertension, myocardial infarction (MI), ischemic heart disease (IHD) (reperfusion), angina pectoris, arrhythmia, hypercholesterolemia, hyperlipidemia, atherosclerosis, stroke, myocarditis, congestive heart failure, primary and secondary i.e. dilated (congestive) cardiomyopathy, hypertrophic cardiomyopathy, restrictive cardiomyopathy, peripheral vascular disease, tachycardia, high blood pressure or thrombosis.

Typically, said genetically related metabolic disorder is cystic fibrosis (CF), peroxisome biogenesis disorder or adrenoleukodystrophy.

Typically, the compounds of the invention are used as an immunosuppressant following organ transplant.

Typically, said infection is a viral, bacterial, fungal or parasitic infection, in particular an infection by S aureus, P acne, candida or aspergillus.

Typically, said CNS disorder is Huntingdon's disease, Alzheimer's disease, Parkinson's disease, stroke, spinal cord injury, multiple sclerosis, amyotrophic lateral sclerosis.

In this embodiment, the compounds of the invention may be used to alleviate cancer, cardiac hypertrophy, chronic heart failure, an inflammatory condition, a cardiovascular disease, a hemoglobinopathy, a thalassemia, a sickle cell disease, a CNS disorder, an autoimmune disease, diabetes or osteoporosis, or are used as an immunosuppressant.

The compounds of the invention may also be used to alleviate chronic lymphocytic leukemia (CLL), breast cancer, prostate cancer, ovarian cancer, mesothelioma, T-cell lymphoma, cardiac hypertrophy, chronic heart failure or a skin inflammatory condition, in particular psoriasis, acne or eczema.

The compounds of the present invention can be used in the treatment of animals, preferably in the treatment of mammals and more preferably in the treatment of humans.

The compounds of the invention may, where appropriate, be used prophylactically to reduce the incidence of such conditions.

In use, a therapeutically effective amount of a compound of the invention is administered to a patient. A typical dose is from about 0.001 to 50 mg per kg of body weight, according to the activity of the specific compound, the age, weight and conditions of the subject to be treated, the type and severity of the disease and the frequency and route of administration.

The present invention will now be illustrated by the following examples.

### EXAMPLES

### Example S

### N-(1,2,5-Thiadiazol-3-yl)-N-({5-[5-(trifluoromethyl)-1,2,4-oxadiazol-3-yl]thiophen-2-yl}methyl)pyrazin-2-amine

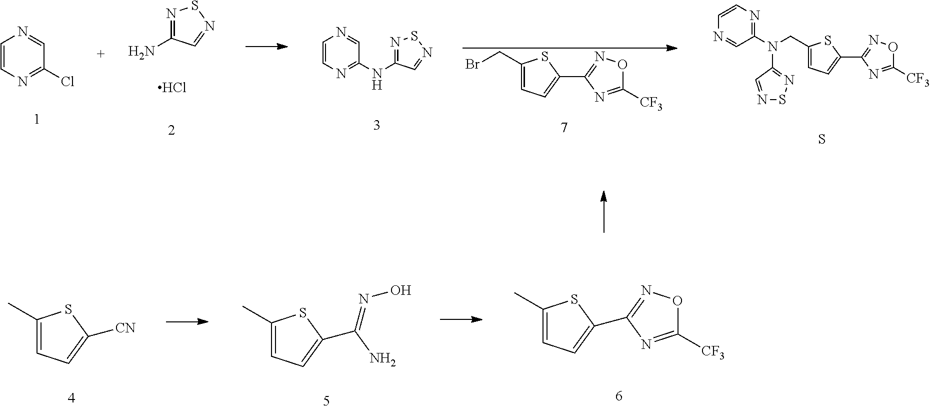

Under Ar(g), to a mixture of 2-chloropyrazine (1) (229 mg, 2.0 mmol), 1,2,5-thiadiazol-3-amine hydrochloride (2) (250 mg, 1.8 mmol), Cs₂CO₃ (1.18 g, 3.6 mmol) was added degassed dry 1,4-dioxane (13 mL). The reaction mixture was then flushed with Ar(g) for 1 min before Pd₂(dba)₃ (83 mg, 0.09 mmol) and Xantphos (116 mg, 0.20 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (63 mg, 19%).

To a solution of (4) (9.94 g, 80.7 mmol) in EtOH (200 mL) was added NH₂OH (50% in H₂O, 5.44 mL, 88.7 mmol). The reaction mixture was heated up to 80° C. for 2.5 h. Once cooled down to rt, it was concentrated in vacuo to yield (5) as a yellow solid (11.95 g, 95%).

To a solution of (5) (11.95 g, 76.5 mmol) in THF (190 mL) was added Et₃N (32 mL, 230 mmol) followed by trifluoroacetic anhydride (21.3 mL, 152.3 mmol) at 0° C. The reaction mixture was allowed to warm up to rt overnight. It was then basified with NaOH solution (5%, 50 mL) and concentrated in vacuo to remove THF. The reaction mixture was diluted with brine (50 mL) and extracted with EtOAc (4×50 mL). The combined organics were dried over MgSO₄, filtered and concentrated in vacuo. Purification by silica gel column chromatography with hexane/EtOAc (1:0-9:1) yielded (6) as a colourless oil (12.3 g, 69%).

To a solution of (6) (5.0 g, 21.4 mmol) in MeCN (100 mL) was added dibenzoyl peroxide (75%, 345 mg, 1.07 mmol) and N-bromosuccinimide (4.18 g, 23.5 mmol). The reaction mixture was heated up to 70° C. for 3 h. Once cooled down, it was concentrated in vacuo. The residue was dissolved in EtOAc (150 mL) and washed sequentially with NaOH solution (5%, 3×50 mL) and brine (50 mL). The organic phase was dried over MgSO₄, filtered and concentrated in vacuo. Purification by silica gel column chromatography with hexane/EtOAc (1:0-9:1) yielded (7) as a light yellow solid (2.06 g, 31%).

¹H NMR (300 MHz, DMSO-d₆), δ: 7.81 (d, J=3.8 Hz, 1H), 7.41 (d, J=4.0 Hz, 1H), 5.10 (s, 2H). ¹⁹F NMR (282 MHz, DMSO-d₆), δ: −64.75 (s, 3F).

Under Ar(g), to a solution of (3) (63 mg, 0.35 mmol) in dry DMF (1.3 mL) was added NaH (60%, 17 mg, 0.43 mmol) in one portion at 0° C. After 5 min, a solution of (7) (132 mg, 0.42 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (S) as a solid (87 mg, 60%).

LCMS (ES): Found 412.3 [M+H]⁺.

¹H NMR (400 MHz, DMSO-d₆), δ: 9.04 (s, 1H), 9.00 (d, J=1.5 Hz, 1H), 8.44 (dd, J=2.5, 1.5 Hz, 1H), 8.33 (d, J=2.7 Hz, 1H), 7.78 (d, J=3.8 Hz, 1H), 7.39 (d, J=3.8 Hz, 1H), 5.72 (s, 2H).

### Example T

### N-(1,3-Thiazol-4-yl)-N-({5-[5-(trifluoromethyl)-1,2,4-oxadiazol-3-yl]thiophen-2-yl}methyl)pyrazin-2-amine

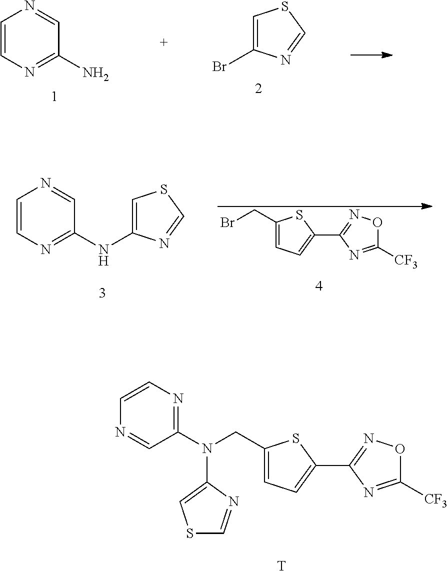

Under Ar(g), to a mixture of pyrazin-2-amine (1) (209 mg, 2.2 mmol), 4-bromothiazole (2) (328 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (15 mg, 4%).

Under Ar(g), to a solution of (3) (15 mg, 0.08 mmol) in dry DMF (0.7 mL) was added NaH (60%, 4.1 mg, 0.10 mmol) in one portion at 0° C. After 5 min, a solution of (4) (32 mg, 0.10 mmol) in dry DMF (0.6 mL) was added dropwise. The reaction mixture was allowed to warm up to rt overnight. Water (0.01 mL) was then added to quench the remaining NaH. The reaction mixture was purified by acidic prep LCMS to yield (T) as a solid (15 mg, 44%).

LCMS (ES): Found 411.2 [M+H]⁺.

¹H NMR (400 MHz, DMSO-d₆), δ: 9.13 (d, J=2.1 Hz, 1H), 8.37 (d, J=1.5 Hz, 1H), 8.32 (dd, J=2.7, 1.5 Hz, 1H), 8.08 (d, J=2.7 Hz, 1H), 7.75 (d, J=3.8 Hz, 1H), 7.63 (d, J=2.1 Hz, 1H), 7.23 (d, J=3.8 Hz, 1H), 5.50 (s, 2H).

### Example U

### N-(Pyridin-2-yl)-N-({5-[5-(trifluoromethyl)-1,2,4-oxadiazol-3-yl]thiophen-2-yl}methyl)pyridin-2-amine

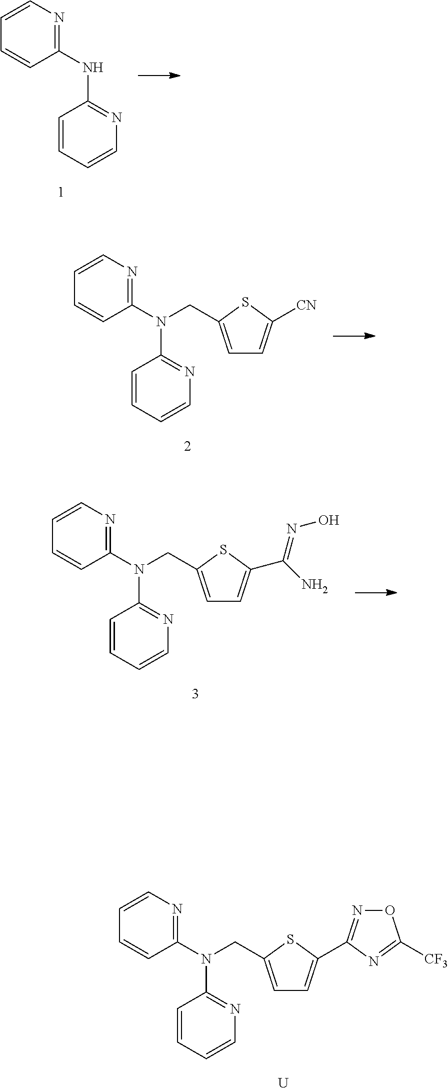

NaH (60%, 26 mg, 0.64 mmol) was added to a suspension of (1) (100 mg, 0.58 mmol) in DMF (1 mL) at 0° C. under Ar(g). The reaction mixture was stirred for 50 min then 5-(bromomethyl)thiophene-2-carbonitrile (130 mg, 0.64 mmol) was added dropwise as a solution in DMF (1 mL). It was allowed to warm up to rt overnight, poured into a mixture of H₂O (10 mL) and brine (10 mL), then extracted with EtOAc (4×10 mL). The combined organics were dried over MgSO₄, filtered and concentrated in vacuo. Purification by silica gel column chromatography with hexane/EtOAc (1:0-3:2) yielded (2) as a colourless oil (86 mg, 50%).

To a solution of (2) (85 mg, 0.29 mmol) in EtOH (50 mL) was added NH₂OH (50% in H₂O. 0.02 mL, 0.32 mmol) at rt. The reaction mixture was heated up to 80° C. for 5 h and NH₂OH (50% in H₂O, 0.02 mL, 0.032 mmol) was re-charged. Heating was continued for a further 1.5 h and the reaction mixture was concentrated in vacuo to yield (3) as a pale yellow foam (95 mg, 100%), which was carried as such into the next step.

To a solution of (3) (95 mg, 0.29 mmol) in dry THF (3 mL) was added Et₃N (0.12 mL, 0.91 mmol) followed by trifluoroacetic anhydride (0.085 mL, 0.61 mmol) at 0° C. under Ar(g). The reaction mixture was allowed to warm up to rt for 3 h. Reagents were then re-charged: Et₃N (0.12 mL, 0.91 mmol) followed by trifluoroacetic anhydride (0.085 mL, 0.61 mmol). The reaction mixture was stirred at rt for a further 16 h before being quenched with NaOH solution (5%, 1 mL). it was then partitioned between brine (10 mL) and EtOAc (10 mL). The aqueous phase was re-extracted with EtOAc (2×10 mL) and the combined organics were dried over MgSO₄, filtered and concentrated in vacuo. Purification by silica gel column chromatography with hexane/EtOAc (1:0-7:3) yielded (U) as a pale yellow solid (76 mg, 64%).

LCMS (ES): Found 404.0 [M+H]⁺.

¹H NMR (300 MHz, DMSO-d₆), δ: 8.37 (ddd, J=5.3, 1.9, 0.8 Hz, 2H), 7.64-7.73 (m, 3H), 7.25-7.28 (m, 1H), 7.21-7.25 (m, 1H), 7.18 (d, J=3.8 Hz, 1H), 7.01 (ddd, J=7.2, 4.9, 0.8 Hz, 2H), 5.61 (s, 2H).

¹⁹F NMR (282 MHz, DMSO-d₆), δ: −64.79 (s, 3F).

### Example V

### N-(Pyridin-2-yl)-N-({5-[5-(trifluoromethyl)-1,2,4-oxadiazol-3-yl]thiophen-2-yl}methyl)pyrazin-2-amine

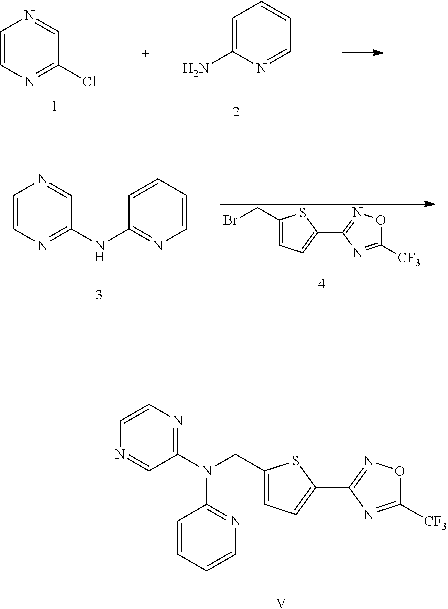

Under Ar(g), to a solution of (3) (50 mg, 0.29 mmol) in DMF (1.5 mL) was added NaH (60%, 14 mg, 0.35 mmol) in one portion at 0° C. After 5 min, a solution of (4) (109 mg, 0.35 mmol) in DMF (0.5 mL) was added dropwise over 1 min. The reaction mixture was stirred for 1 h, then allowed to warm up to rt overnight. Water (5 mL), brine (5 mL) and EtOAc (25 mL) were added. The organic phase was separated, extracting the aqueous phase with EtOAc (2×10 mL). The combined organics extracts were dried over MgSO₄ and concentrated in vacuo. Purification by silica gel column chromatography with hexane/EtOAc (1:0-2:3) yielded (V) as a yellow oil (6 mg, 5%).

LCMS (ES): Found 405.0 [M+H]⁺.

¹H NMR (300 MHz, Methanol-d₄), δ: 8.47 (d, J=1.5 Hz, 1H), 8.40 (ddd, J=4.9, 1.9, 0.8 Hz, 1H), 8.36 (dd, J=2.6, 1.5 Hz, 1H), 8.07 (d, J=2.6 Hz, 1H), 7.79 (ddd, J=8.3, 7.3, 1.9 Hz, 1H), 7.67 (d, J=3.8 Hz, 1H), 7.37 (d, J=8.3 Hz, 1H), 7.11-7.18 (m, 2H), 5.63 (s, 2H).

¹⁹F NMR (282 MHz, Methanol-d₄), δ: −67.44 (s, 3F).

### Example W

### N-[6-(Morpholin-4-yl)pyridin-2-yl]-N-({5-[5-(trifluoromethyl)-1,2,4-oxadiazol-3-yl]thiophen-2-yl}methyl)pyrazin-2-amine

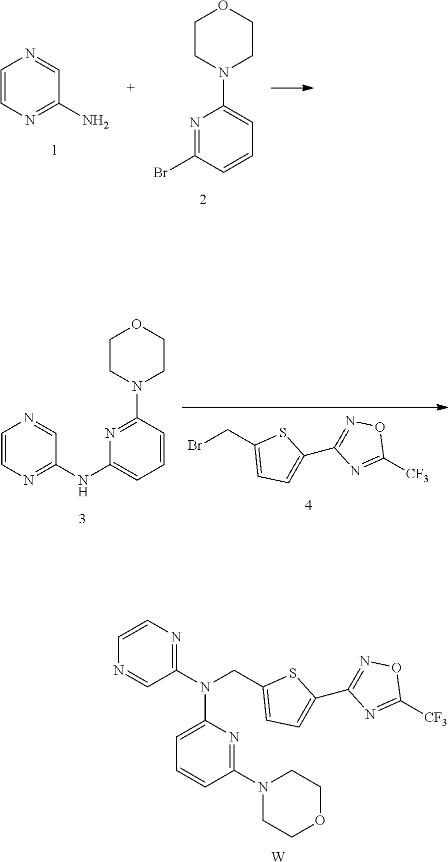

Under Ar(g), to a mixture of pyrazin-2-amine (1) (209 mg, 2.2 mmol), 2-bromo-6-morpholinopyridine (2) (486 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. Once cooled down to rt, EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (124 mg, 24%).

LCMS (ES): Found 258.1 [M+H]⁺.

Under Ar(g), to a solution of (3) (77 mg, 0.30 mmol) in dry DMF (1.3 mL) was added NaH (60%, 14 mg, 0.36 mmol) in one portion at 0° C. After 5 min, a solution of (4) (112 mg, 0.36 mmol) in dry DMF (1.2 mL) was added dropwise. The reaction mixture was stirred for 1 h then allowed to warm up to rt overnight. Water (0.01 mL) was then added to quench the remaining NaH. The reaction mixture was purified by acidic prep LCMS to yield (W) as a solid (42 mg, 29%).

LCMS (ES): Found 490.4 [M+H]⁺.

¹H NMR (400 MHz, DMSO-d₆), δ: 8.66 (d, J=1.6 Hz, 1H), 8.36 (dd, J=2.8, 1.5 Hz, 1H), 8.12 (d, J=2.7 Hz, 1H), 7.73 (d, J=3.8 Hz, 1H), 7.55 (t, J=8.1 Hz, 1H), 7.23 (d, J=3.8 Hz, 1H), 6.64 (d, J=7.8 Hz, 1H), 6.52 (d, J=8.3 Hz, 1H), 5.55 (s, 2H), 3.67 (m, 4H), 3.41 (m, 4H).

### Example X

### N-(Pyrazin-2-yl)-N-({5-[5-(trifluoromethyl)-1,2,4-oxadiazol-3-yl]thiophen-2-yl}methyl)-1,5-naphthyridin-2-amine

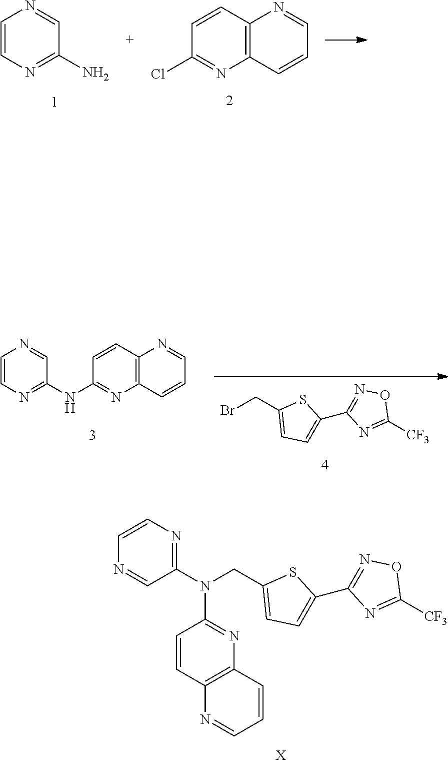

Under Ar(g), to a mixture of pyrazin-2-amine (1) (209 mg, 2.2 mmol), 2-chloro-1,5-naphthyridine (2) (329 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (121 mg, 27%).

LCMS (ES): Found 224.3 [M+H]⁺.

Under Ar(g), to a solution of (3) (67 mg, 0.30 mmol) in dry DMF (1.3 mL) was added NaH (60%, 14.4 mg, 0.36 mmol) in one portion at 0° C. After 5 min, a solution of (4) (112 mg, 0.36 mmol) in dry DMF (1.2 mL) was added dropwise. The reaction mixture was allowed to warm up to rt overnight. The reaction mixture was purified by acidic prep LCMS to yield (X) as a solid (38 mg, 28%).

LCMS (ES): Found 456.4 [M+H]⁺.

¹H NMR (400 MHz, DMSO-d₆), δ: 8.89 (d, J=1.5 Hz, 1H), 8.83 (dd, J=4.3, 1.6 Hz, 1H), 8.48 (dd, J=2.7, 1.5 Hz, 1H), 8.35 (d, J=2.6 Hz, 1H), 8.28 (d, J=9.3 Hz, 1H), 8.18-8.24 (m, 1H), 7.76 (d, J=9.3 Hz, 1H), 7.70-7.74 (m, 2H), 7.34 (d, J=3.8 Hz, 1H), 5.78 (s, 2H).

### Example Y

### N-(Pyrazin-2-yl)-N-({5-[5-(trifluoromethyl)-1,2,4-oxadiazol-3-yl]thiophen-2-yl}methyl)-1,7-naphthyridin-2-amine

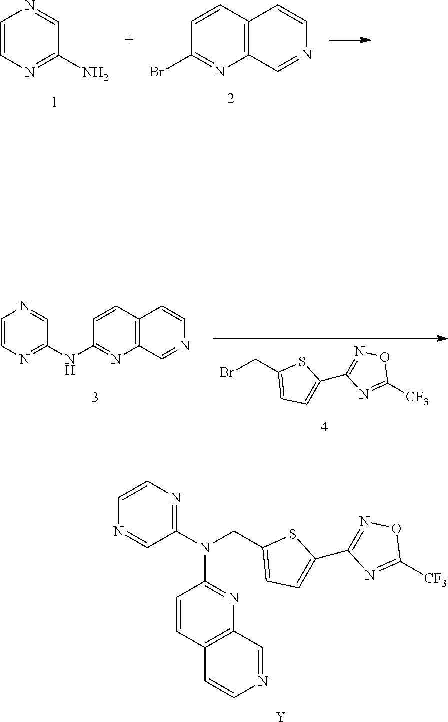

Under Ar(g), to a mixture of pyrazin-2-amine (1) (125 mg, 1.3 mmol), 2-bromo-1,7-naphthyridine (2) (250 mg, 1.2 mmol), Cs₂CO₃ (0.78 g, 2.4 mmol) was added degassed dry 1,4-dioxane (13 mL). The reaction mixture was then flushed with Ar(g) for 1 min before Pd₂(dba)₃ (55 mg, 0.06 mmol) and Xantphos (76 mg, 0.13 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (115 mg, 43%).

LCMS (ES): Found 224.2 [M+H]⁺.

Under Ar(g), to a solution of (3) (67 mg, 0.30 mmol) in dry DMF (1.3 mL) was added NaH (60%, 14.4 mg, 0.36 mmol) in one portion at 0° C. After 5 min, a solution of (4) (112 mg, 0.36 mmol) in dry DMF (1 mL) was added dropwise. The reaction mixture was allowed to warm up to rt overnight. The reaction mixture was purified by acidic prep LCMS to yield (Y) as a solid (58 mg, 43%).

LCMS (ES): Found 456.4 [M+H]⁺.

¹H NMR (400 MHz, DMSO-d₆), δ: 9.18 (s, 1H), 8.89 (d, J=1.5 Hz, 1H), 8.51 (d, J=5.4 Hz, 1H), 8.49 (dd, J=2.6, 1.5 Hz, 1H), 8.37 (d, J=2.7 Hz, 1H), 8.30 (d, J=9.1 Hz, 1H), 7.83 (d, J=5.3 Hz, 1H), 7.76 (d, J=9.1 Hz, 1H), 7.73 (d, J=3.8 Hz, 1H), 7.35 (d, J=3.8 Hz, 1H), 5.79 (s, 2H).

### Example Z

### N-{Pyrido[2,3-b]pyrazin-6-yl}-N-({5-[5-(trifluoromethyl)-1,2,4-oxadiazol-3-yl]thiophen-2-yl}methyl)pyrazin-2-amine

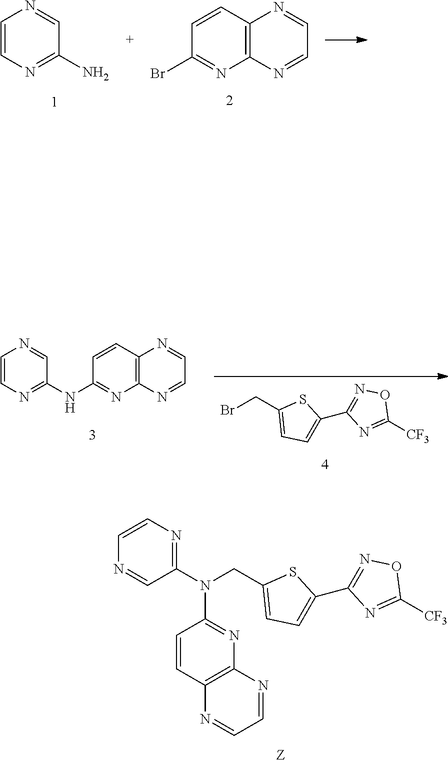

Under Ar(g), to a mixture of pyrazin-2-amine (1) (125 mg, 1.3 mmol), 6-bromopyrido[2,3-b]pyrazine (2) (250 mg, 1.2 mmol), Cs₂CO₃ (0.78 g, 2.4 mmol) was added degassed dry 1,4-dioxane (13 mL). The reaction mixture was then flushed with Ar(g) for 1 min before Pd₂(dba)₃ (55 mg, 0.06 mmol) and Xantphos (76 mg, 0.13 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (24 mg, 9%).

NaH (60%, 5.2 mg, 0.13 mmol) was added to a solution of (3) (24 mg, 0.11 mmol) in dry DMF (0.7 mL). After 5 min, a solution of (4) (41 mg, 0.13 mmol) in dry DMF (0.6 mL) was added. The reaction mixture was stirred overnight. Water (0.01 mL) was then added to quench the remaining NaH. The reaction mixture was purified by acidic prep LCMS to yield (Z) as a solid (12 mg, 23%).

LCMS (ES): Found 457.2 [M+H]⁺.

¹H NMR (400 MHz, DMSO-d₆), δ: 8.98 (dd, J=3.2, 1.7 Hz, 2H), 8.85 (d, J=2.0 Hz, 1H), 8.54 (dd, J=2.7, 1.5 Hz, 1H), 8.45 (d, J=2.6 Hz, 1H), 8.34 (d, J=9.2 Hz, 1H), 7.80 (d, J=9.1 Hz, 1H), 7.74 (d, J=3.8 Hz, 1H), 7.34 (d, J=3.8 Hz, 1H), 5.81 (s, 2H).

### Example AA

### N-(5-Cyclopropylpyridin-2-yl)-N-({5-[5-(trifluoromethyl)-1,2,4-oxadiazol-3-yl]thiophen-2-yl}methyl)pyrazin-2-amine

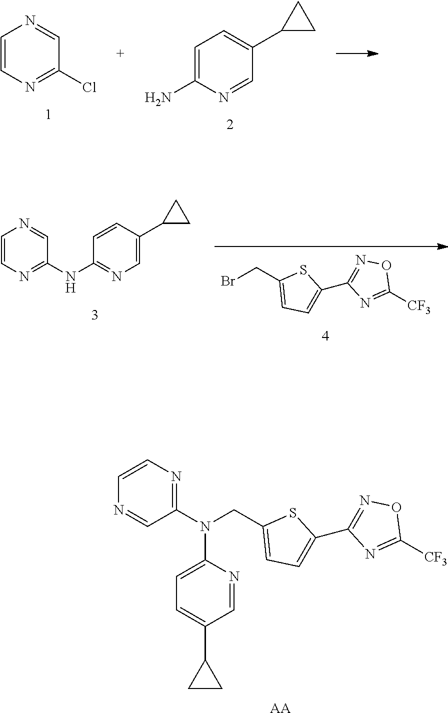

Under Ar(g), to a mixture of 2-chloropyrazine (1) (235 mg, 2.1 mmol), 5-cyclopropylpyridin-2-amine (2) (250 mg, 1.9 mmol), Cs₂CO₃ (1.21 g, 3.7 mmol) was added degassed dry 1,4-dioxane (13 mL). The reaction mixture was then flushed with Ar(g) for 1 min before Pd₂(dba)₃ (85 g, 0.1 mmol) and Xantphos (119 mg, 0.2 mmol) were added. The reaction mixture was heated up to 90° C. for 40 h. Once cooled down to rt, it was concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (237 mg, 60%).

LCMS (ES): Found 213.4 [M+H]⁺.

NaH (60%, 14 mg, 0.36 mmol) was added to a solution of (3) (63 mg, 0.3 mmol) in dry DMF (1.25 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (AA) as a solid (46 mg, 35%).

LCMS (ES): Found 445.4 [M+H]⁺.

¹H NMR (400 MHz, DMSO-d₆), δ: 8.52 (d, J=1.5 Hz, 1H), 8.33 (dd, J=2.6, 1.5 Hz, 1H), 8.24 (d, J=2.5 Hz, 1H), 8.11 (d, J=2.7 Hz, 1H), 7.72 (d, J=3.7 Hz, 1H), 7.44 (dd, J=8.6, 2.5 Hz, 1H), 7.33 (d, J=8.6 Hz, 1H), 7.22 (d, J=3.8 Hz, 1H), 5.56 (s, 2H), 1.89-1.99 (m, 1H), 0.94-1.01 (m, 2H), 0.68-0.76 (m, 2H).

### Example AB

### N-({5-[5-(Trifluoromethyl)-1,2,4-oxadiazol-3-yl]thiophen-2-yl}methyl)-N-[5-(trifluoromethyl)pyridin-2-yl]pyrazin-2-amine

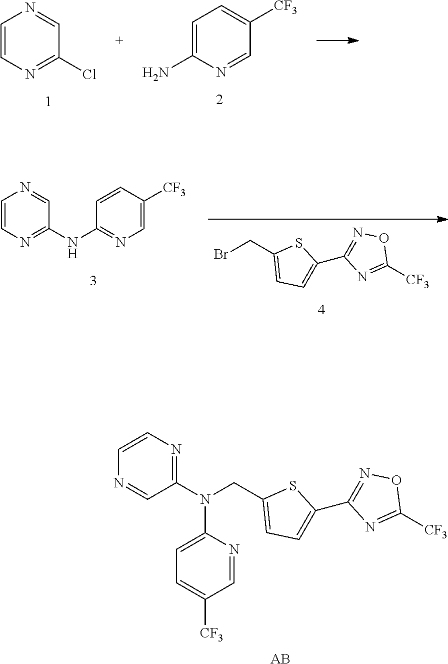 [SMILES: Clc1cnccn1 ; Nc1ccc(C(F)(F)F)cn1 ; FC(F)(F)c1ccc(Nc2cnccn2)nc1 ; FC(F)(F)c1nc(-c2ccc(CBr)s2)no1 ; FC(F)(F)c1ccc(N(Cc2ccc(-c3noc(C(F)(F)F)n3)s2)c2cnccn2)nc1 ; "AB"]

Under Ar(g), to a mixture of 2-chloropyrazine (1) (252 mg, 2.2 mmol), 2-amino-5-(trifluoromethyl)pyridine (2) (324 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (354 mg, 74%).

LCMS (ES): Found 241.2 [M+H]⁺.

NaH (60%, 24 mg, 0.6 mmol) was added to a solution of (3) (72 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (AB) as a solid (48 mg, 8%).

LCMS (ES): Found 473.3 [M+H]⁺.

¹H NMR (400 MHz, DMSO-d₆), δ: 8.87 (d, J=1.5 Hz, 1H), 8.68-8.74 (m, 1H), 8.50 (dd, J=2.7, 1.5 Hz, 1H), 8.35 (d, J=2.7 Hz, 1H), 8.06 (dd, J=8.9, 2.6 Hz, 1H), 7.75 (d, J=3.8 Hz, 1H), 7.49 (d, J=8.9 Hz, 1H), 7.30 (d, J=3.8 Hz, 1H), 5.67 (s, 2H).

### Example AC

### N,N-Dimethyl-6-[pyrazin-2-yl({5-[5-(trifluoromethyl)-1,2,4-oxadiazol-3-yl]thiophen-2-yl}methyl)amino]pyridine-3-carboxamide

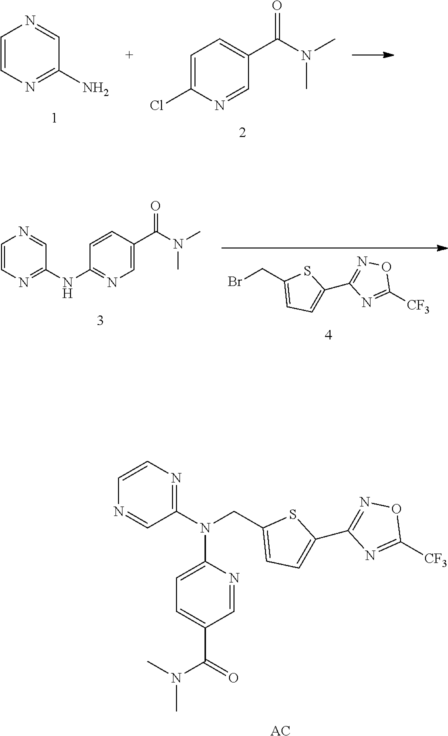 [SMILES: Nc1cnccn1 ; CN(C)C(=O)c1ccc(Cl)nc1 ; CN(C)C(=O)c1ccc(Nc2cnccn2)nc1 ; FC(F)(F)c1nc(-c2ccc(CBr)s2)no1 ; CN(C)C(=O)c1ccc(N(Cc2ccc(-c3noc(C(F)(F)F)n3)s2)c2cnccn2)nc1 ; "AC"]

Under Ar(g), to a mixture of pyrazin-2-amine (1) (209 mg, 2.2 mmol), 6-chloro-N,N-dimethylpyridine-3-carboxamide (2) (369 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (200 mg, 41%).

LCMS (ES): Found 244.1 [M+H]⁺.

NaH (60%, 24 mg, 0.6 mmol) was added to a solution of (3) (73 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (AC) as a solid (49 mg, 34%).

LCMS (ES): Found 476.1 [M+H]⁺.

¹H NMR (400 MHz, DMSO-d₆), δ: 8.79 (d, J=1.5 Hz, 1H), 8.40-8.46 (m, 2H), 8.25 (d, J=2.7 Hz, 1H), 7.82 (dd, J=8.6, 2.3 Hz, 1H), 7.74 (d, J=3.8 Hz, 1H), 7.42 (d, J=8.4 Hz, 1H), 7.28 (d, J=3.8 Hz, 1H), 5.64 (s, 2H), 3.00 (s, 6H).

### Example AD

### N-[5-(Morpholin-4-yl)pyridin-2-yl]-N-({5-[5-(trifluoromethyl)-1,2,4-oxadiazol-3-yl]thiophen-2-yl}methyl)pyrazin-2-amine

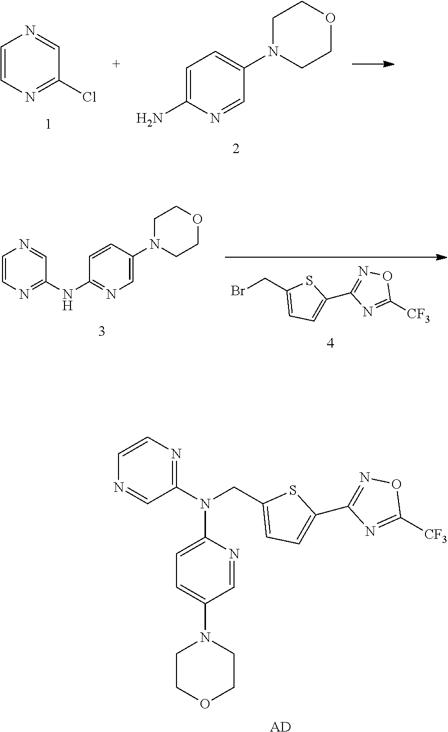 [SMILES: Clc1cnccn1 ; Nc1ccc(N2CCOCC2)cn1 ; c1cnc(Nc2ccc(N3CCOCC3)cn2)cn1 ; FC(F)(F)c1nc(-c2ccc(CBr)s2)no1 ; FC(F)(F)c1nc(-c2ccc(CN(c3ccc(N4CCOCC4)cn3)c3cnccn3)s2)no1 ; "AD"]

Under Ar(g), to a mixture of 2-chloropyrazine (1) (252 mg, 2.2 mmol), 5-morpholinopyridin-2-amine (2) (358 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (256 mg, 50%).

LCMS (ES): Found 258.2 [M+H]⁺.

NaH (60%, 24 mg, 0.6 mmol) was added to a solution of (3) (77 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (AD) as a solid (20 mg, 14%).

LCMS (ES): Found 490.4 [M+H]⁺.

¹H NMR (400 MHz, DMSO-d₆), δ: 8.30 (d, J=1.5 Hz, 1H), 8.27 (dd, J=2.7, 1.5 Hz, 1H), 8.14 (d, J=3.0 Hz, 1H), 8.02 (d, J=2.8 Hz, 1H), 7.73 (d, J=3.7 Hz, 1H), 7.45 (dd, J=9.0, 3.1 Hz, 1H), 7.34 (d, J=8.9 Hz, 1H), 7.19 (d, J=3.8 Hz, 1H), 5.49 (s, 2H), 3.71-3.79 (m, 4H), 3.13-3.20 (m, 4H).

### Example AE

### N-{3-Methyl-3H-imidazo[4,5-c]pyridin-6-yl}-N-({5-[5-(trifluoromethyl)-1,2,4-oxadiazol-3-yl]thiophen-2-yl}methyl)pyrazin-2-amine

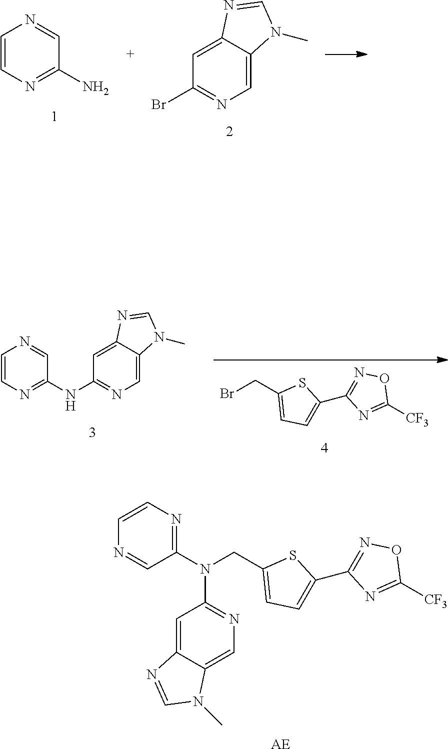 [SMILES: Nc1cnccn1 ; Cn1cnc2cc(Br)ncc21 ; Cn1cnc2cc(Nc3cnccn3)ncc21 ; FC(F)(F)c1nc(-c2ccc(CBr)s2)no1 ; Cn1cnc2cc(N(Cc3ccc(-c4noc(C(F)(F)F)n4)s3)c3cnccn3)ncc21 ; "AE"]

Under Ar(g), to a mixture of pyrazin-2-amine (1) (123 mg, 1.3 mmol), 6-bromo-3-methylimidazo[4,5-c]pyridine (2) (250 mg, 1.2 mmol), Cs₂CO₃ (0.77 g, 2.4 mmol) was added degassed dry 1,4-dioxane (13 mL). The reaction mixture was then flushed with Ar(g) for 1 min before Pd₂(dba)₃ (54 mg, 0.06 mmol) and Xantphos (75 mg, 0.13 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (58 mg, 22%).

NaH (60%, 12 mg, 0.3 mmol) was added to a solution of (3) (58 mg, 0.26 mmol) in dry DMF (1.2 mL). After 5 min, a solution of (4) (97 mg, 0.31 mmol) in dry DMF (1.0 mL) was added. The reaction mixture was stirred overnight. Water (0.01 mL) was then added to quench the remaining NaH. The reaction mixture was purified by acidic prep LCMS to yield (AE) as a solid (9.5 mg, 8%).

LCMS (ES): Found 459.3 [M+H]⁺.

¹H NMR (400 MHz, DMSO-d₆), δ: 8.83 (d, J=0.9 Hz, 1H), 8.42 (s, 1H), 8.29 (dd, J=2.7, 1.6 Hz, 1H), 8.21 (d, J=1.5 Hz, 1H), 8.02 (d, J=2.6 Hz, 1H), 7.67-7.73 (m, 2H), 7.18 (d, J=3.8 Hz, 1H), 5.57 (s, 2H), 3.94 (s, 3H).

### Example AF

### N-{Pyrido[3,4-b]pyrazin-7-yl}-N-({5-[5-(trifluoromethyl)-1,2,4-oxadiazol-3-yl]thiophen-2-yl}methyl)pyrazin-2-amine

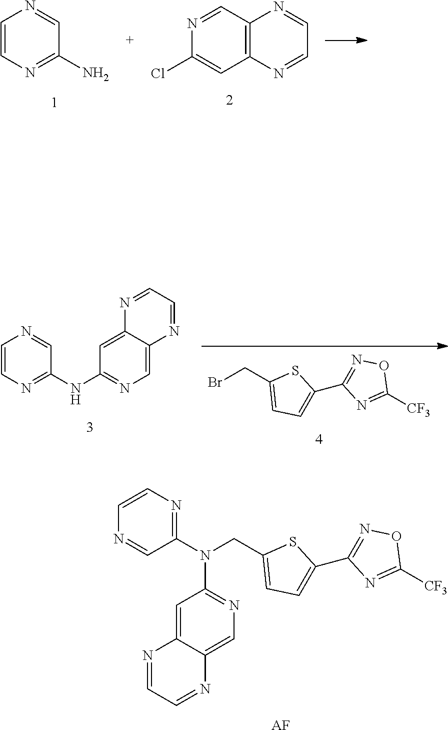 [SMILES: Nc1cnccn1 ; Clc1cc2nccnc2cn1 ; c1cnc(Nc2cc3nccnc3cn2)cn1 ; FC(F)(F)c1nc(-c2ccc(CBr)s2)no1 ; FC(F)(F)c1nc(-c2ccc(CN(c3cnccn3)c3cc4nccnc4cn3)s2)no1 ; "AF"]

Under Ar(g), to a mixture of pyrazin-2-amine (1) (158 mg, 1.7 mmol), 7-chloro-pyrido[3,4-b]pyrazine (2) (250 mg, 1.5 mmol), Cs₂CO₃ (0.99 g, 3.0 mmol) was added degassed dry 1,4-dioxane (13 mL). The reaction mixture was then flushed with Ar(g) for 1 min before Pd₂(dba)₃ (69 mg, 0.08 mmol) and Xantphos (96 mg, 0.17 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (61 mg, 18%).

NaH (60%, 13.1 mg, 0.33 mmol) was added to a solution of (3) (61 mg, 0.27 mmol) in dry DMF (1.2 mL). After 5 min, a solution of (4) (102 mg, 0.33 mmol) in dry DMF (1.0 mL) was added. The reaction mixture was stirred overnight. Water (0.01 mL) was then added to quench the remaining NaH. The reaction mixture was purified by acidic prep LCMS to yield (AF) as a solid (31 mg, 25%).

LCMS (ES): Found 457.3 [M+H]⁺.

¹H NMR (400 MHz, DMSO-d₆), δ: 9.33 (s, 1H), 9.06 (d, J=1.8 Hz, 1H), 8.90 (d, J=1.8 Hz, 1H), 8.81 (d, J=1.5 Hz, 1H), 8.47 (dd, J=2.6, 1.5 Hz, 1H), 8.27 (d, J=2.7 Hz, 1H), 7.89 (s, 1H), 7.74 (d, J=3.8 Hz, 1H), 7.35 (d, J=3.8 Hz, 1H), 5.76 (s, 2H).

### Example AG

### N-(4-Fluoropyridin-2-yl)-N-({5-[5-(trifluoromethyl)-1,2,4-oxadiazol-3-yl]thiophen-2-yl}methyl)pyrazin-2-amine

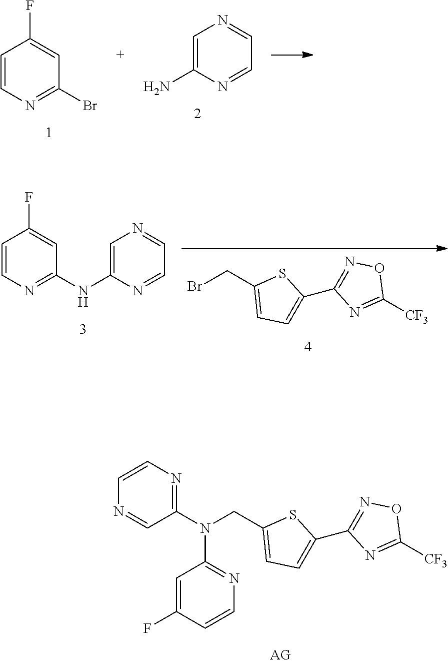

NaH (60%, 13 mg, 0.32 mmol) was added to a suspension of (3) (51 mg, 0.27 mmol) in DMF (2 mL) at 0° C. The reaction mixture was stirred for 20 min then (4) (100 mg, 0.32 mmol) was added as a solid. It was allowed to warm up to rt over 2 h, poured into a mixture of H₂O (5 mL) and brine (5 mL), then extracted with EtOAc (3×15 mL). The combined organics were washed with brine (3×5 mL), dried over MgSO₄, filtered and concentrated in vacuo. Purification by silica gel column chromatography with hexane/EtOAc (9:1-1:1) yielded (AG) as a viscous yellow oil (34 mg, 30%).

LCMS (ES): Found 422.9 [M+H]⁺.

¹H NMR (300 MHz, DMSO-d₆), δ: 8.76 (d, J=1.5 Hz, 1H), 8.42 (dd, J=2.6, 1.5 Hz, 1H), 8.36 (dd, J=9.3, 5.7 Hz, 1H), 8.25 (d, J=2.6 Hz, 1H), 7.74 (d, J=3.8 Hz, 1H), 7.33 (dd, J=11.9, 2.1 Hz, 1H), 7.28 (d, J=3.8 Hz, 1H), 7.00 (ddd, J=8.2, 5.9, 2.3 Hz, 1H), 5.62 (s, 2H).

¹⁹F NMR (282 MHz, DMSO-d₆), δ: −64.80 (s, 3F), −101.80 (m, 1F).

### Example AH

### N-(4-Cyclopropylpyridin-2-yl)-N-({5-[5-(trifluoromethyl)-1,2,4-oxadiazol-3-yl]thiophen-2-yl}methyl)pyrazin-2-amine

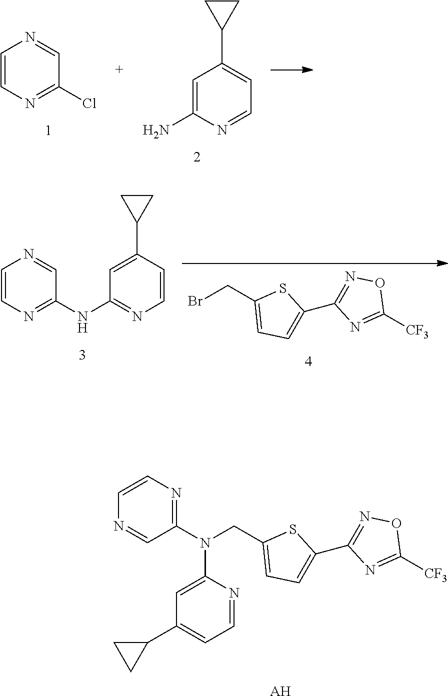

Under Ar(g), to a mixture of 2-chloropyrazine (1) (235 mg, 2.1 mmol), 4-cyclopropylpyridin-2-amine (2) (250 mg, 1.9 mmol), Cs₂CO₃ (1.2 g, 3.7 mmol) was added degassed dry 1,4-dioxane (13 mL). The reaction mixture was then flushed with Ar(g) for 1 min before Pd₂(dba)₃ (85 mg, 0.09 mmol) and Xantphos (119 mg, 0.2 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (165 mg, 42%).

LCMS (ES): Found 213.4 [M+H]⁺.

NaH (60%, 24 mg, 0.6 mmol) was added to a solution of (3) (64 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (AH) as a solid (33 mg, 25%).

LCMS (ES): Found 445.5 [M+H]⁺.

¹H NMR (400 MHz, DMSO-d₆), δ: 8.58 (d, J=1.5 Hz, 1H), 8.36 (dd, J=2.8, 1.5 Hz, 1H), 8.17 (d, J=5.3 Hz, 1H), 8.14 (d, J=2.7 Hz, 1H), 7.73 (d, J=3.9 Hz, 1H), 7.23 (d, J=3.8 Hz, 1H), 7.16 (d, J=1.4 Hz, 1H), 6.78 (dd, J=5.3, 1.5 Hz, 1H), 5.58 (s, 2H), 1.89-1.98 (m, 1H), 1.01-1.09 (m, 2H), 0.77-0.85 (m, 2H).

### Example AI

### N-({5-[5-(Trifluoromethyl)-1,2,4-oxadiazol-3-yl]thiophen-2-yl}methyl)-N-[4-(trifluoromethyl)pyridin-2-yl]pyrazin-2-amine

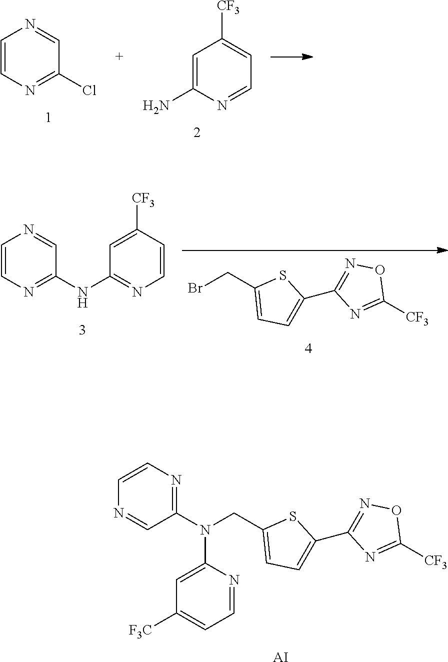

Under Ar(g), to a mixture of 2-chloropyrazine (1) (252 mg, 2.2 mmol), 2-amino-4-(trifluoromethyl)pyridine (2) (324 mg, 2.0 mmol), Cs₂CO₃ (1.3 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (115 mg, 24%).

LCMS (ES): Found 241.1 [M+H]⁺.

Under Ar(g), to a solution of (3) (72 mg, 0.30 mmol) in dry DMF (1.3 mL) was added NaH (60%, 14 mg, 0.36 mmol) in one portion at 0° C. After 5 min, a solution of (4) (112 mg, 0.36 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (AI) as a solid (61 mg, 43%).

LCMS (ES): Found 473.3 [M+H]⁺.

¹H NMR (400 MHz, DMSO-d₆), δ: 8.81 (d, J=1.5 Hz, 1H), 8.59 (d, J=5.2 Hz, 1H), 8.44 (dd, J=2.7, 1.5 Hz, 1H), 8.29 (d, J=2.6 Hz, 1H), 7.71-7.77 (m, 2H), 7.35-7.41 (m, 1H), 7.30 (d, J=3.8 Hz, 1H), 5.69 (s, 2H).

### Example AJ

### N-(Pyrazin-2-yl)-N-({5-[5-(trifluoromethyl)-1,2,4-oxadiazol-3-yl]thiophen-2-yl}methyl)-1,6-naphthyridin-5-amine

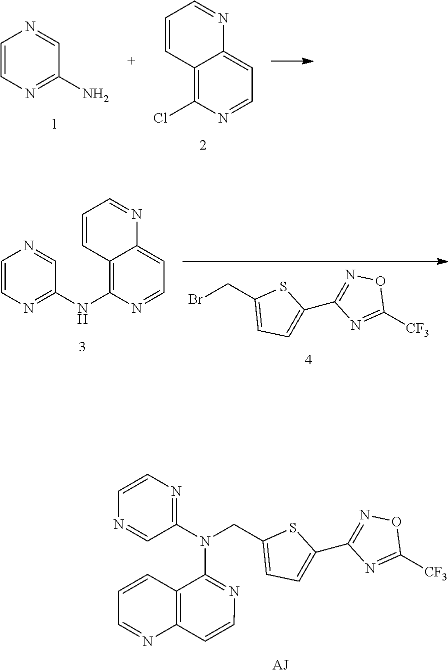

Under Ar(g), to a mixture of pyrazin-2-amine (1) (159 mg, 1.7 mmol), 5-chloro-1,6-naphthyridine (2) (250 mg, 1.5 mmol), Cs₂CO₃ (0.99 g, 3.0 mmol) was added degassed dry 1,4-dioxane (13 mL). The reaction mixture was then flushed with Ar(g) for 1 min before Pd₂(dba)₃ (70 mg, 0.08 mmol) and Xantphos (97 mg, 0.17 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (161 mg, 47%).

LCMS (ES): Found 224.1 [M+H]⁺.

NaH (60%, 24 mg, 0.6 mmol) was added to a solution of (3) (67 mg, 0.30 mmol) in dry DMF (1.3 mL). After 5 min, a solution of (4) (112 mg, 0.36 mmol) in dry DMF (1.0 mL) was added. The reaction mixture was stirred overnight. Water (0.01 mL) was then added to quench the remaining NaH. The reaction mixture was purified by acidic prep LCMS to yield (AJ) as a solid (36 mg, 26%).

LCMS (ES): Found 456.4 [M+H]⁺.

¹H NMR (400 MHz, DMSO-d₆), δ: 8.77 (dd, J=4.1, 1.7 Hz, 1H), 8.42-8.50 (m, 2H), 8.25 (dd, J=2.7, 1.5 Hz, 1H), 8.06 (d, J=2.7 Hz, 1H), 8.04 (d, J=1.4 Hz, 1H), 7.71-7.79 (m, 2H), 7.68 (d, J=3.8 Hz, 1H), 7.21 (d, J=3.8 Hz, 1H), 5.79 (s, 2H).

### Example AK

### N-(Pyrazin-2-yl)-N-({5-[5-(trifluoromethyl)-1,2,4-oxadiazol-3-yl]thiophen-2-yl}methyl)-2,6-naphthyridin-1-amine

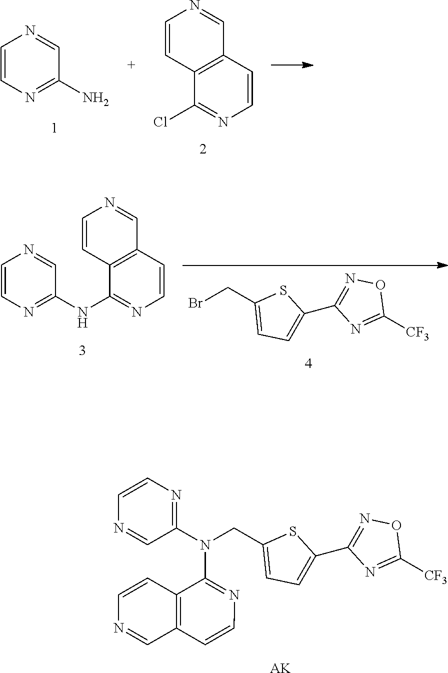

Under Ar(g), to a mixture of pyrazin-2-amine (1) (159 mg, 1.7 mmol), 1-chloro-[2,6]naphthyridine (2) (250 mg, 1.5 mmol), Cs₂CO₃ (0.99 g, 3.0 mmol) was added degassed dry 1,4-dioxane (13 mL). The reaction mixture was then flushed with Ar(g) for 1 min before Pd₂(dba)₃ (70 mg, 0.08 mmol) and Xantphos (97 mg, 0.17 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (158 mg, 46%).

LCMS (ES): Found 224.2 [M+H]⁺.

NaH (60%, 14.4 mg, 0.36 mmol) was added to a solution of (3) (67 mg, 0.3 mmol) in dry DMF (1.25 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (AK) as a solid (57 mg, 42%).

LCMS (ES): Found 456.3 [M+H]⁺.

¹H NMR (400 MHz, DMSO-d₆), δ: 9.49 (d, J=1.0 Hz, 1H), 8.65 (d, J=5.6 Hz, 1H), 8.55 (d, J=5.9 Hz, 1H), 8.25 (d, J=1.5 Hz, 1H), 8.21 (dd, J=2.7, 1.5 Hz, 1H), 8.14 (d, J=2.7 Hz, 1H), 7.95-8.00 (m, 1H), 7.71 (d, J=3.8 Hz, 1H), 7.34-7.41 (m, 1H), 7.27 (d, J=3.8 Hz, 1H), 5.69 (s, 2H).

### Example AL

### N-{Pyrido[3,4-b]pyrazin-5-yl}-N-({5-[5-(trifluoromethyl)-1,2,4-oxadiazol-3-yl]thiophen-2-yl}methyl)pyrazin-2-amine

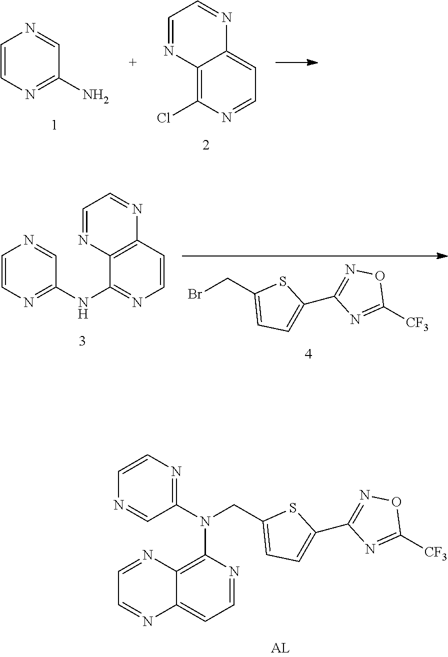

Under Ar(g), to a mixture of pyrazin-2-amine (1) (158 mg, 1.7 mmol), 5-chloropyrido[4,3-b]pyrazine (2) (250 mg, 1.5 mmol), Cs₂CO₃ (0.99 g, 3.0 mmol) was added degassed dry 1,4-dioxane (13 mL). The reaction mixture was then flushed with Ar(g) for 1 min before Pd₂(dba)₃ (69 mg, 0.08 mmol) and Xantphos (96 mg, 0.17 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (112 mg, 33%).

Under Ar(g), to a solution of (3) (66 mg, 0.3 mmol) in dry DMF (1.3 mL) was added NaH (60%, 14 mg, 0.36 mmol) in one portion at 0° C. After 5 min, a solution of (4) (110 mg, 0.35 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (AL) as a solid (64 mg, 48%).

LCMS (ES): Found 457.3 [M+H]⁺.

¹H NMR (400 MHz, DMSO-d₆), δ: 9.10 (d, J=1.8 Hz, 1H), 8.75 (d, J=1.8 Hz, 1H), 8.67 (d, J=5.8 Hz, 1H), 8.29 (d, J=1.5 Hz, 1H), 8.25 (dd, J=2.7, 1.4 Hz, 1H), 8.14 (d, J=2.7 Hz, 1H), 7.80 (d, J=5.8 Hz, 1H), 7.70 (d, J=3.8 Hz, 1H), 7.24 (d, J=3.8 Hz, 1H), 5.80 (s, 2H).

### Example AM

### N-(3-Cyclopropylpyridin-2-yl)-N-({5-[5-(trifluoromethyl)-1,2,4-oxadiazol-3-yl]thiophen-2-yl}methyl)pyrazin-2-amine

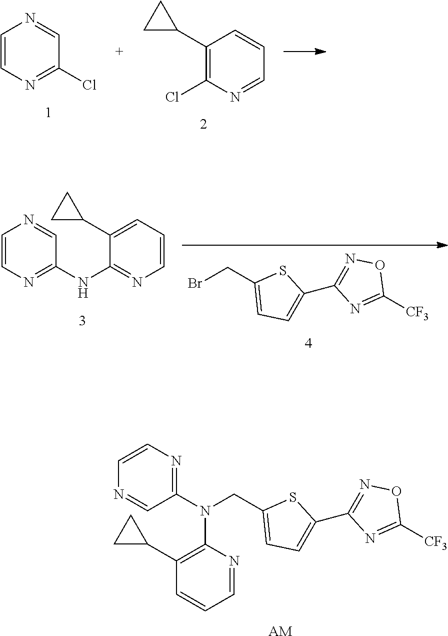

Under Ar(g), to a mixture of 2-chloropyrazine (1) (235 mg, 2.1 mmol), 3-cyclopropylpyridin-2-amine (2) (250 mg, 1.9 mmol), Cs₂CO₃ (1.2 g, 3.7 mmol) was added degassed dry 1,4-dioxane (13 mL). The reaction mixture was then flushed with Ar(g) for 1 min before Pd₂(dba)₃ (85 mg, 0.09 mmol) and Xantphos (119 mg, 0.20 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (78 mg, 20%).

LCMS (ES): Found 213.1 [M+H]⁺.

Under Ar(g), to a solution of (3) (64 mg, 0.30 mmol) in dry DMF (1.3 mL) was added NaH (60%, 14 mg, 0.36 mmol) in one portion at 0° C. After 5 min, a solution of (4) (112 mg, 0.36 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (AM) as a solid (71 mg, 53%).

LCMS (ES): Found 445.4 [M+H]⁺.

¹H NMR (400 MHz, DMSO-d₆), δ: 8.38 (dd, J=4.6, 1.8 Hz, 1H), 8.28 (dd, J=2.7, 1.5 Hz, 1H), 8.01 (d, J=2.7 Hz, 1H), 7.77 (d, J=1.5 Hz, 1H), 7.72 (d, J=3.8 Hz, 1H), 7.48 (dd, J=7.8, 1.8 Hz, 1H), 7.32 (dd, J=7.8, 4.7 Hz, 1H), 7.18 (d, J=3.8 Hz, 1H), 5.47 (s, 2H), 1.52-1.61 (m, 1H), 0.63-0.77 (m, 4H).

### Example AN

### N-[3-(Morpholin-4-yl)pyridin-2-yl]-N-({5-[5-(trifluoromethyl)-1,2,4-oxadiazol-3-yl]thiophen-2-yl}methyl)pyrazin-2-amine

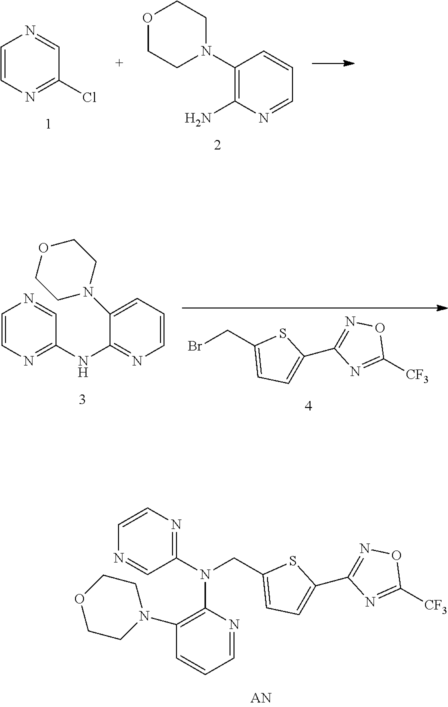

Under Ar(g), to a mixture of 2-chloropyrazine (1) (176 mg, 1.5 mmol), 3-morpholinopyridin-2-amine (2) (250 mg, 1.4 mmol), Cs₂CO₃ (0.9 g, 2.8 mmol) was added degassed dry 1,4-dioxane (13 mL). The reaction mixture was then flushed with Ar(g) for 1 min before Pd₂(dba)₃ (64 mg, 0.07 mmol) and Xantphos (89 mg, 0.15 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (197 mg, 55%).

LCMS (ES): Found 258.1 [M+H]⁺.

NaH (60%, 24 mg, 0.6 mmol) was added to a solution of (3) (77 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (AN) as a solid (47 mg, 32%).

LCMS (ES): Found 490.5 [M+H]⁺.

¹H NMR (400 MHz, DMSO-d₆), δ: 8.31 (dd, J=2.8, 1.5 Hz, 1H), 8.20 (dd, J=4.6, 1.5 Hz, 1H), 8.05 (d, J=2.8 Hz, 1H), 7.88 (d, J=1.5 Hz, 1H), 7.70 (d, J=3.8 Hz, 1H), 7.52 (dd, J=7.9, 1.6 Hz, 1H), 7.28 (dd, J=8.0, 4.7 Hz, 1H), 7.17 (d, J=3.8 Hz, 1H), 5.57 (s, 2H), 3.17 (m, 4H), 2.73 (m, 4H).

### Example AO

### N-(Pyridin-3-yl)-N-({5-[5-(trifluoromethyl)-1,2,4-oxadiazol-3-yl]thiophen-2-yl}methyl)pyrazin-2-amine

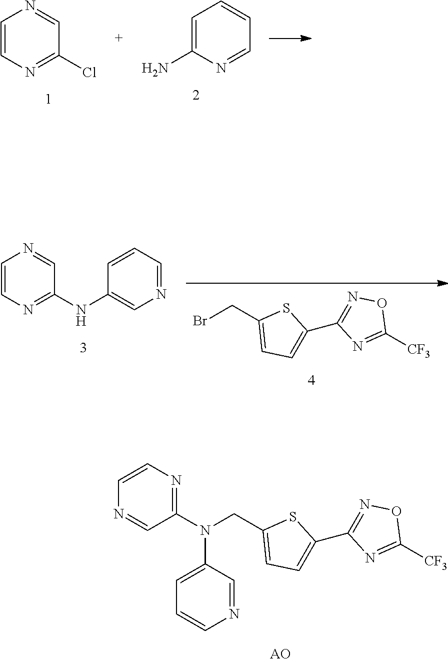 [SMILES: Clc1cnccn1 ; Nc1ccccn1 ; c1cncc(Nc2cnccn2)c1 ; FC(F)(F)c1nc(-c2ccc(CBr)s2)no1 ; FC(F)(F)c1nc(-c2ccc(CN(c3cccnc3)c3cnccn3)s2)no1]

Under Ar(g), to a solution of pyridin-3-amine (2) (94 mg, 1 mmol) in DMF (5 mL) was added NaH (60%, 44 mg, 1.1 mmol) as a single portion. The reaction mixture was stirred for 0.5 h, then 2-chloropyrazine (1) (113 mg, 1 mmol) was added and the reaction mixture was heated up to 80° C. for 1 h. Once cooled down to rt, it was poured into Na₂CO₃ solution (20 mL) and extracted with CH₂Cl₂ (2×20 mL). The combined organic fractions were dried over MgSO₄, filtered and concentrated in vacuo. Purification by silica gel column chromatography with CH₂Cl₂/MeOH (1:0-9:1) yielded (3) as a solid (48 mg, 28%) LCMS (ES): Found 173.1 [M+H]⁺.

Under Ar(g), to a solution of (3) (86 mg, 0.5 mmol) in DMF (5 mL) was added NaH (60%, 24 mg, 0.6 mmol) as a single portion. The reaction mixture was stirred for 0.5 h, cooled down to −5° C., then a solution of (4) (157 mg, 0.5 mmol) in DMF (1 mL) was added dropwise. The reaction mixture was allowed to warm up to rt over 2 h, then poured into saturated NH₄Cl solution (10 mL) and extracted with CH₂Cl₂ (2×10 mL). The combined organic fractions were dried over MgSO₄, filtered and concentrated in vacuo. Purifications by silica gel column chromatography with CH₂Cl₂/MeOH (1:0-19:1) then neat EtOAc yielded (AO) as a solid (63 mg, 31%).

LCMS (ES): Found 404.8 [M+H]⁺.

¹H NMR (300 MHz, DMSO-d₆), δ: 8.58 (br s, 1H), 8.50 (br d, J=4.1 Hz, 1H), 8.29 (dd, J=2.6, 1.5 Hz, 1H), 8.04 (d, J=2.6 Hz, 1H), 7.98 (d, J=1.3 Hz, 1H), 7.77-7.83 (m, 1H), 7.74 (d, J=3.8 Hz, 1H), 7.49 (dd, J=8.1, 4.7 Hz, 1H), 7.16 (d, J=3.8 Hz, 1H), 5.43 (s, 2H).

¹⁹F NMR (282 MHz, DMSO-d₆), δ: −64.78 (s, 3F).

### Example AP

### N-[6-(Pyrrolidin-1-yl)pyridin-3-yl]-N-({5-[5-(trifluoromethyl)-1,2,4-oxadiazol-3-yl]thiophen-2-yl}methyl)pyrazin-2-amine

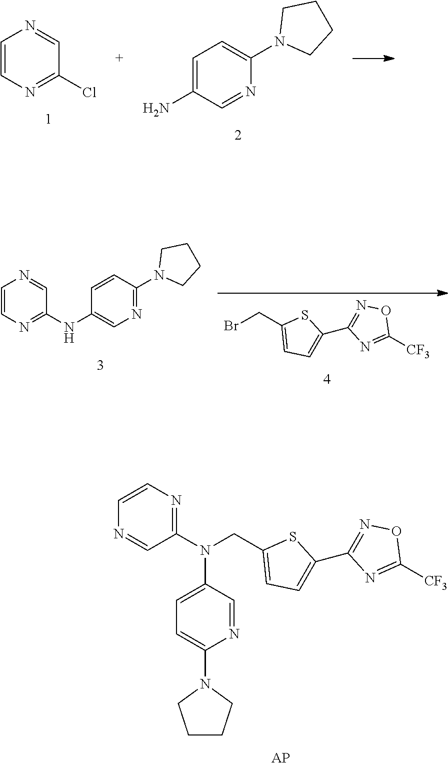 [SMILES: Clc1cnccn1 ; Nc1ccc(N2CCCC2)nc1 ; c1cnc(Nc2ccc(N3CCCC3)nc2)cn1 ; FC(F)(F)c1nc(-c2ccc(CBr)s2)no1 ; FC(F)(F)c1nc(-c2ccc(CN(c3ccc(N4CCCC4)nc3)c3cnccn3)s2)no1]

To degassed dioxane (10 mL) were added sequentially 6-pyrrolidinyl-pyridin-3-amine (2) (301 mg, 1.8 mmol), 2-chloropyrazine (1) (0.20 mL, 2.20 mmol), Pd₂(dba)₃ (51 mg, 0.06 mmol), XantPhos (64 mg, 0.11 mmol) and Cs₂CO₃ (1.20 g, 3.7 mmol) with continued degassing. The reaction mixture was heated up to 100° C. for 2 d. Once cooled down to rt, it was poured into a brine solution (50%, 20 mL) and extracted with EtOAc (3×20 mL). The combined organic extracts were dried over MgSO₄, filtered and concentrated in vacuo. Purification by silica gel column chromatography with hexane/EtOAc (1:0-0:1) gave a residue which was re-dissolved in CH₂Cl₂/MeOH (4:1, 50 mL) and swirled with MP-TMT resin (424 mg, 1.3 mmol/g) at rt overnight. The solution was filtered, the resin washed with CH₂Cl₂/MeOH (4:1, 100 mL) and the filtrate concentrated in vacuo. Purification by silica gel column chromatography with CH₂Cl₂/EtOAc (1:0-48:1) yielded (3) as an orange solid (54 mg, 12%).

LCMS (ES): Found 242.0 [M+H]⁺.

¹H NMR (300 MHz, DMSO-d₆), δ: 9.04 (s, 1H), 8.25 (d, J=2.6 Hz, 1H), 8.07 (d, J=1.5 Hz, 1H), 7.98 (dd, J=2.8, 1.5 Hz, 1H), 7.79 (d, J=2.6 Hz, 1H), 7.75 (dd, J=8.9, 2.7 Hz, 1H), 6.44 (d, J=8.9 Hz, 1H), 3.33-3.41 (m, 4H), 1.88-1.98 (m, 4H).

To a solution of (3) (41 mg, 0.17 mmol) in anhydrous DMF (2 mL) was added KO^{t}Bu (1M in THF, 0.20 mL) at 0° C. The solution was stirred for 10 min before the addition of (4) (58 mg, 0.19 mmol). The reaction mixture was allowed to warm up to rt for 1 h, poured into a brine solution (50%, 10 mL) and extracted with EtOAc (3×10 mL). The combined organic extracts were dried over MgSO₄, filtered and concentrated in vacuo. Purification by silica gel column chromatography with hexane/EtOAc (1:0-2:1) yielded (AP) as an orange oil (59 mg, 74%).

LCMS (ES): Found 473.9 [M+H]⁺.

¹H NMR (300 MHz, DMSO-d₆), δ: 8.17-8.25 (m, 1H), 7.93 (dd, J=6.9, 2.6 Hz, 2H), 7.69-7.78 (m, 2H), 7.37 (dd, J=8.9, 2.7 Hz, 1H), 7.12 (d, J=3.8 Hz, 1H), 6.51 (d, J=8.9 Hz, 1H), 5.25 (s, 2H), 3.34-3.44 (m, 4H), 1.87-1.98 (m, 4H).

¹⁹F NMR (282 MHz, DMSO-d₆), −64.78 (s, 3F).

### Example AQ

### N-({5-[5-(Trifluoromethyl)-1,2,4-oxadiazol-3-yl]thiophen-2-yl}methyl)-N-[5-(trifluoromethyl)pyridin-3-yl]pyrazin-2-amine

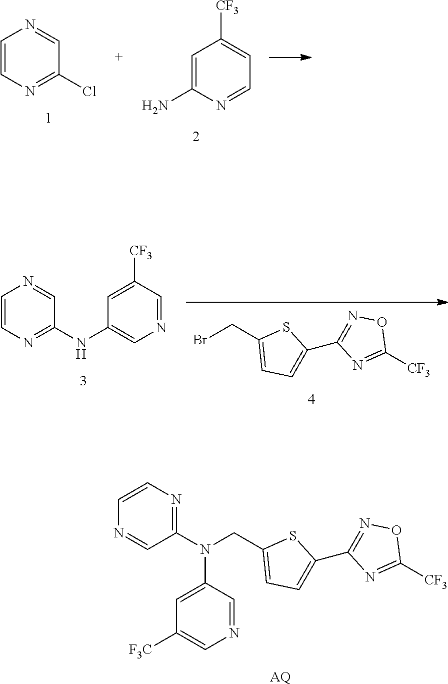

To degassed dioxane (10 mL) were added sequentially 5-trifluoromethylpyridin-3-amine (2) (309 mg, 1.9 mmol), 2-chloropyrazine (1) (0.26 mL, 2.86 mmol), Pd₂(dba)₃ (52 mg, 0.06 mmol), XantPhos (66 mg, 0.11 mmol) and Cs₂CO₃ (1.24 g, 3.8 mmol) with continued degassing. The reaction mixture was heated up to 90° C. overnight. An additional portion of 2-chloropyrazine (0.17 mL, 1.91 mmol) was then added and the reaction mixture was heated up to 90° C. overnight. Once cooled down to rt, it was poured into H₂O (20 mL) and extracted with EtOAc (3×20 mL). The combined organic extracts were dried over MgSO₄, filtered and concentrated in vacuo. Purification by silica gel column chromatography with hexane/EtOAc (1:0-1:2) gave a residue which was re-dissolved in CH₂Cl₂/MeOH (4:1, 50 mL) and swirled with MP-TMT resin (440 mg, 1.3 mmol/g) at rt overnight. The solution was filtered, the resin washed with CH₂Cl₂/MeOH (4:1, 100 mL) and the filtrate concentrated in vacuo to yield (3) as a yellow solid (202 mg, 44%).

LCMS (ES): Found 241.0 [M+H]⁺.

¹H NMR (300 MHz, DMSO-d₆), δ: 10.14 (s, 1H), 9.00 (d, J=2.4 Hz, 1H), 8.69 (t, J=2.0 Hz, 1H), 8.52 (d, J=0.9 Hz, 1H), 8.32 (d, J=1.5 Hz, 1H), 8.25 (dd, J=2.8, 1.5 Hz, 1H), 8.08 (d, J=2.8 Hz, 1H).

¹⁹F NMR (282 MHz, DMSO-d₆), δ: −61.09 (s, 3F).

To a solution of (3) (42 mg, 0.17 mmol) in anhydrous DMF (2 mL) was added NaH (60%, 7.6 mg, 0.19 mmol) at 0° C. The reaction mixture was stirred for 20 min then (4) (65 mg, 0.21 mmol) was added as a solid. It was then allowed to warm up to rt overnight. The reaction mixture was re-charged with NaH (60%, 3.5 mg, 0.09 mmol) and (4) (3.5 mg, 0.09 mmol) then stirred at rt overnight. It was then poured into a brine solution (50%, 10 mL) and extracted with EtOAc (3×10 mL). The combined organic extracts were dried over MgSO₄, filtered and concentrated in vacuo. Purification by silica gel column chromatography with hexane/EtOAc (1:0-2:1) yielded (AQ) as an orange oil (21 mg, 26%).

LCMS (ES): Found 472.8 [M+H]⁺.

¹H NMR (300 MHz, DMSO-d₆), δ: 8.87 (d, J=2.3 Hz, 1H), 8.82 (d, J=0.9 Hz, 1H), 8.30 (t, J=2.0 Hz, 2H), 8.25 (d, J=1.5 Hz, 1H), 8.13 (d, J=2.6 Hz, 1H), 7.76 (d, J=3.8 Hz, 1H), 7.22 (d, J=3.8 Hz, 1H), 5.53 (s, 2H).

¹⁹F NMR (282 MHz, DMSO-d₆), δ: −64.79 (s, 3F), −60.74 (s, 3F).

### Example AR

### N-(Pyridin-4-yl)-N-({5-[5-(trifluoromethyl)-1,2,4-oxadiazol-3-yl]thiophen-2-yl}methyl)pyrazin-2-amine

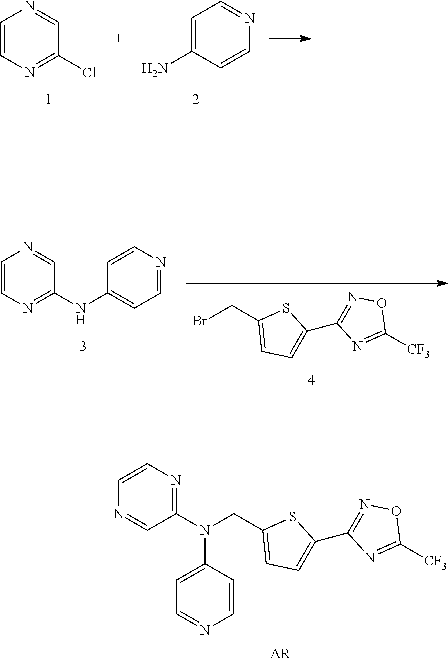 [SMILES: Clc1cnccn1 ; Nc1ccncc1 ; c1cc(Nc2cnccn2)ccn1 ; FC(F)(F)c1nc(-c2ccc(CBr)s2)no1 ; FC(F)(F)c1nc(-c2ccc(CN(c3ccncc3)c3cnccn3)s2)no1 ; "AR"]

To degassed dioxane (10 mL) were added sequentially 4-aminopyridine (2) (308 mg, 3.3 mmol), 2-chloropyrazine (1) (0.35 mL, 3.9 mmol), Pd₂(dba)₃ (90 mg, 0.1 mmol), XantPhos (113 mg, 0.2 mmol) and Cs₂CO₃ (2.1 g, 6.6 mmol) with continued degassing. The reaction mixture was heated up to 90° C. for 2 d. Once cooled down to rt, it was poured into a brine solution (50%, 20 mL) and extracted with EtOAc (3×20 mL). The combined organic extracts were dried over MgSO₄, filtered and concentrated in vacuo. The residue was re-dissolved in CH₂Cl₂/MeOH (4:1, 50 mL) and swirled with MP-TMT resin (770 mg, 1.3 mmol/g) at rt overnight. The solution was filtered, the resin washed with CH₂Cl₂/MeOH (4:1, 100 mL) and the filtrate concentrated in vacuo. Purification by silica gel column chromatography with EtOAc/MeOH (1:0-48:1) yielded (3) as a white solid (255 mg, 45%).

LCMS (ES): Found 173.0 [M+H]⁺.

¹H NMR (300 MHz, DMSO-d₆), δ: 9.95 (s, 1H), 8.36 (dd, J=4.7, 1.5 Hz, 2H), 8.31 (d, J=1.5 Hz, 1H), 8.25 (dd, J=2.6, 1.5 Hz, 1H), 8.08 (d, J=2.6 Hz, 1H), 7.66 (dd, J=4.7, 1.5 Hz, 2H).

To a solution of (3) (37 mg, 0.17 mmol) in anhydrous DMF (2 mL) was added KO^{t}Bu (1M in THF, 0.26 mL) at 0° C. The solution was stirred for 10 min before the addition of (4) (74 mg, 0.24 mmol) and allowed to warm up to rt over 1 h. It was then poured into a brine solution (50%, 10 mL) and extracted with EtOAc (3×10 mL). The combined organic extracts were dried over MgSO₄, filtered and concentrated in vacuo. Purification by silica gel column chromatography with hexane/EtOAc (1:0-0:1) yielded (AR) as an orange oil (47 mg, 55%).

LCMS (ES): Found 404.8 [M+H]⁺.

¹H NMR (300 MHz, DMSO-d₆), δ: 8.53 (d, J=1.5 Hz, 1H), 8.38-8.49 (m, 3H), 8.24 (d, J=2.6 Hz, 1H), 7.75 (d, J=3.8 Hz, 1H), 7.34 (dd, J=4.7, 1.5 Hz, 2H), 7.25 (d, J=3.8 Hz, 1H), 5.53 (s, 2H).

¹⁹F NMR (282 MHz, DMSO-d), −64.80 (s, 3F).

### Example AS

### N-(Pyrimidin-2-yl)-N-({5-[5-(trifluoromethyl)-1,2,4-oxadiazol-3-yl]thiophen-2-yl}methyl)pyrazin-2-amine

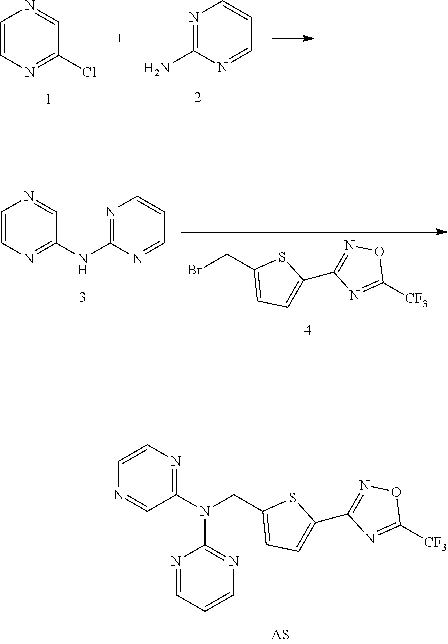

NaH (60%, 13 mg, 0.32 mmol) was added to a suspension of (3) (46 mg, 0.27 mmol) in DMF (2 mL) at 0° C. The reaction mixture was stirred for 25 min then (4) (100 mg, 0.32 mmol) was added as a solid. It was allowed to warm up to rt over 2 h, poured into a mixture of H₂O (5 mL) and brine (5 mL), then extracted with EtOAc (3×15 mL). The combined organics were washed with brine (3×5 mL), dried over MgSO₄, filtered and concentrated in vacuo. Purification by silica gel column chromatography with hexane/EtOAc (9:1-1:1) yielded (AS) as a yellow oil (79 mg, 73%), which solidified on standing.

LCMS (ES): Found 406.0 [M+H]⁺.

¹H NMR (300 MHz, DMSO-d₆), δ: 9.11 (d, J=1.5 Hz, 1H), 8.65 (d, J=4.9 Hz, 2H), 8.51 (dd, J=2.6, 1.7 Hz, 1H), 8.31 (d, J=2.6 Hz, 1H), 7.73 (d, J=3.8 Hz, 1H), 7.26 (d, J=3.8 Hz, 1H), 7.11 (t, J=4.8 Hz, 1H), 5.69 (s, 2H).

¹⁹F NMR (282 MHz, DMSO-d₆), δ: −64.79 (s, 3F).

### Example AT

### 4-Methyl-N-(pyrazin-2-yl)-N-({5-[5-(trifluoromethyl)-1,2,4-oxadiazol-3-yl]thiophen-2-yl}methyl)pyrimidin-2-amine

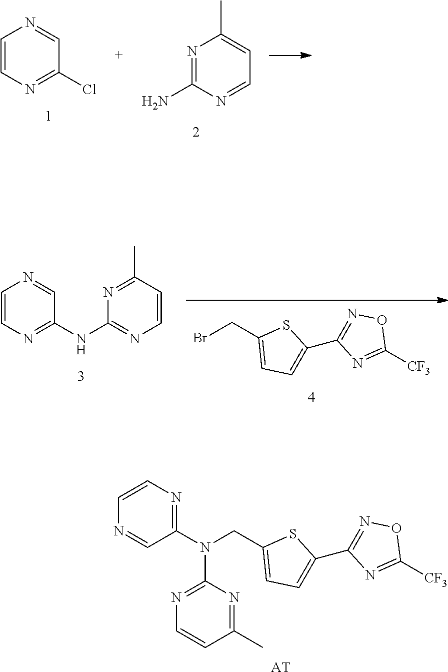

Under Ar(g), to a mixture of 2-chloropyrazine (1) (252 mg, 2.2 mmol), 2-amino-4-methylpyrimidine (2) (218 mg, 2.0 mmol), Cs₂CO₃ (1.3 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (112 mg, 30%).

LCMS (ES): Found 188.2 [M+H]⁺.

Under Ar(g), to a solution of (3) (56 mg, 0.30 mmol) in dry DMF (1.3 mL) was added NaH (60%, 14 mg, 0.36 mmol) in one portion at 0° C. After 5 min, a solution of (4) (112 mg, 0.36 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (AT) as a solid (78 mg, 62%).

LCMS (ES): Found 420.4 [M+H]⁺.

¹H NMR (400 MHz, DMSO-d₆), δ: 9.14 (d, J=1.5 Hz, 1H), 8.47-8.53 (m, 2H), 8.29 (d, J=2.6 Hz, 1H), 7.73 (d, J=3.8 Hz, 1H), 7.27 (d, J=3.8 Hz, 1H), 7.00 (d, J=5.0 Hz, 1H), 5.68 (s, 2H), 2.43 (s, 3H).

### Example AU

### 4-Methoxy-N-(pyrazin-2-yl)-N-({5-[5-(trifluoromethyl)-1,2,4-oxadiazol-3-yl]thiophen-2-yl}methyl)pyrimidin-2-amine

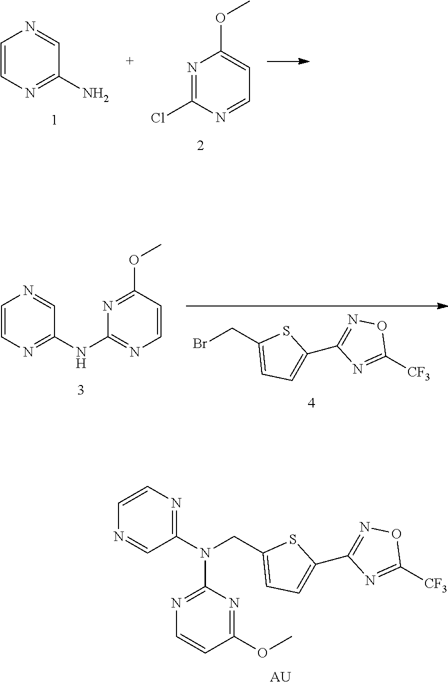

Under Ar(g), to a mixture of pyrazin-2-amine (1) (209 mg, 2.2 mmol), 2-chloro-4-methoxypyrimidine (2) (289 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (55 mg, 14%).

NaH (60%, 13 mg, 0.32 mmol) was added to a solution of (3) (55 mg, 0.27 mmol) in dry DMF (1.2 mL). After 5 min, a solution of (4) (101 mg, 0.32 mmol) in dry DMF (1 mL) was added. The reaction mixture was stirred overnight. Water (0.01 mL) was then added to quench the remaining NaH. The reaction mixture was purified by acidic prep LCMS to yield (AU) as a solid (58 mg, 50%).

LCMS (ES): Found 436.4 [M+H]⁺.

¹H NMR (400 MHz, DMSO-d₆), δ: 9.20 (d, J=1.5 Hz, 1H), 8.52 (dd, J=2.7, 1.5 Hz, 1H), 8.35 (d, J=5.7 Hz, 1H), 8.32 (d, J=2.6 Hz, 1H), 7.75 (d, J=3.8 Hz, 1H), 7.28 (d, J=3.8 Hz, 1H), 6.53 (d, J=5.7 Hz, 1H), 5.69 (s, 2H), 3.89 (s, 3H).

### Example AV

### N-(Pyrazin-2-yl)-4-(pyrrolidin-1-yl)-N-({5-[5-(trifluoromethyl)-1,2,4-oxadiazol-3-yl]thiophen-2-yl}methyl)pyrimidin-2-amine

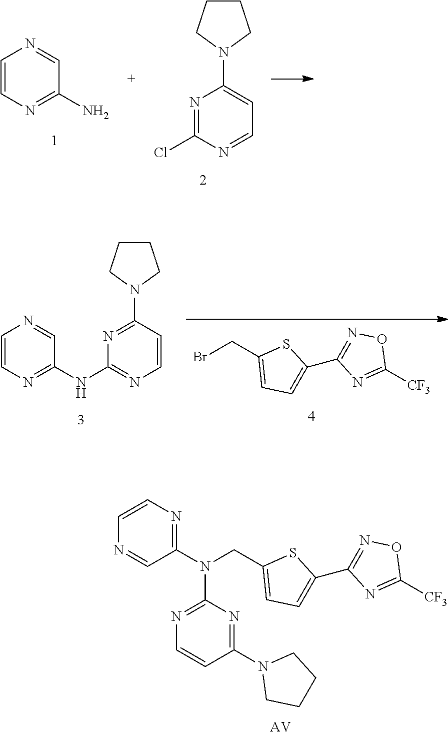

Under Ar(g), to a mixture of pyrazin-2-amine (1) (209 mg, 2.2 mmol), 2-chloro-4-(pyrrolidin-1-yl)pyrimidine (2) (367 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (18 mg, 4%).

Under Ar(g), to a solution of (3) (18 mg, 0.07 mmol) in dry DMF (0.7 mL) was added NaH (60%, 3.5 mg, 0.09 mmol) in one portion at 0° C. After 5 min, a solution of (4) (27 mg, 0.08 mmol) in dry DMF (0.6 mL) was added dropwise. The reaction mixture was allowed to warm up to rt overnight. Water (0.01 mL) was then added to quench the remaining NaH. The reaction mixture was purified by acidic prep LCMS to yield (AV) as a solid (14 mg, 40%).

LCMS (ES): Found 475.4 [M+H]⁺.

¹H NMR (400 MHz, DMSO-d₆), δ: 9.22 (d, J=1.3 Hz, 1H), 8.44 (dd, J=2.8, 1.4 Hz, 1H), 8.20 (d, J=2.6 Hz, 1H), 8.03 (d, J=6.0 Hz, 1H), 7.73 (d, J=3.8 Hz, 1H), 7.24 (d, J=4.0 Hz, 1H), 6.17 (d, J=6.0 Hz, 1H), 5.66 (s, 2H), 3.49 (m, 4H), 1.93 (m, 4H).

### Example AW

### 5-Methyl-N-(pyrazin-2-yl)-N-({5-[5-(trifluoromethyl)-1,2,4-oxadiazol-3-yl]thiophen-2-yl}methyl)pyrimidin-2-amine

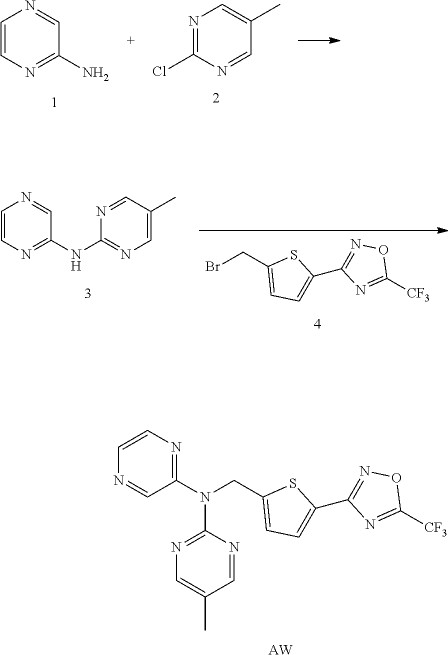

Under Ar(g), to a mixture of pyrazin-2-amine (1) (209 mg, 2.2 mmol), 2-chloro-5-methylpyrimidine (2) (257 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (7.7 mg, 2%).

Under Ar(g), to a solution of (3) (7.7 mg, 0.04 mmol) in dry DMF (0.7 mL) was added NaH (60%, 2.0 mg, 0.05 mmol) in one portion at 0° C. After 5 min, a solution of (4) (15 mg, 0.05 mmol) in dry DMF (0.6 mL) was added dropwise. The reaction mixture was allowed to warm up to rt overnight. Water (0.01 mL) was then added to quench the remaining NaH. The reaction mixture was purified by acidic prep LCMS to yield (AW) as a solid (6.7 mg, 39%).

LCMS (ES): Found 420.2 [M+H]⁺.

¹H NMR (400 MHz, DMSO-d₆), δ: 9.09 (d, J=1.5 Hz, 1H), 8.52 (s, 2H), 8.48 (dd, J=2.7, 1.5 Hz, 1H), 8.27 (d, J=2.7 Hz, 1H), 7.73 (d, J=3.8 Hz, 1H), 7.24 (d, J=3.8 Hz, 1H), 5.68 (s, 2H), 2.22 (s, 3H).

### Example AX

### 5-Fluoro-N-(pyrazin-2-yl)-N-({5-[5-(trifluoromethyl)-1,2,4-oxadiazol-3-yl]thiophen-2-yl}methyl)pyrimidin-2-amine

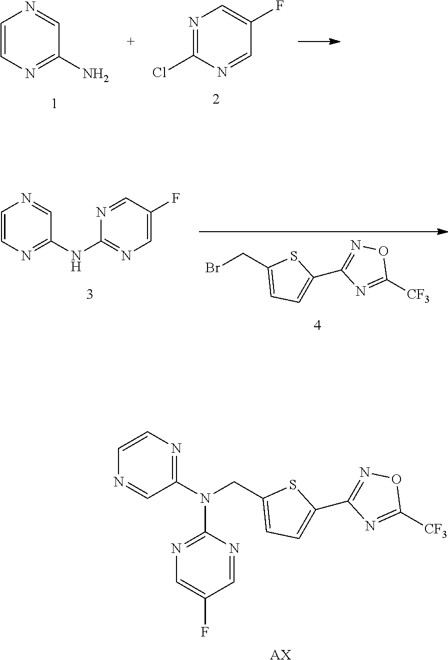

Under Ar(g), to a mixture of pyrazin-2-amine (1) (197 mg, 2.1 mmol), 2-chloro-5-fluoropyrimidine (2) (250 mg, 1.9 mmol), Cs₂CO₃ (1.23 g, 3.8 mmol) was added degassed dry 1,4-dioxane (13 mL). The reaction mixture was then flushed with Ar(g) for 1 min before Pd₂(dba)₃ (86 mg, 0.09 mmol) and Xantphos (120 mg, 0.21 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (16 mg, 4%).

Under Ar(g), to a solution of (3) (16.2 mg, 0.09 mmol) in dry DMF (0.7 mL) was added NaH (60%, 4.1 mg, 0.1 mmol) in one portion at 0° C. After 5 min, a solution of (4) (32 mg, 0.1 mmol) in dry DMF (0.6 mL) was added dropwise. The reaction mixture was allowed to warm up to rt overnight. Water (0.01 mL) was then added to quench the remaining NaH. The reaction mixture was purified by acidic prep LCMS to yield (AX) as a solid (7 mg, 20%).

LCMS (ES): Found 424.2 [M+H]⁺.

¹H NMR (400 MHz, DMSO-d₆), δ: 9.08 (d, J=1.5 Hz, 1H), 8.75 (m, 2H), 8.51 (dd, J=2.7, 1.5 Hz, 1H), 8.31 (d, J=2.6 Hz, 1H), 7.74 (d, J=3.8 Hz, 1H), 7.25 (d, J=3.8 Hz, 1H), 5.66 (s, 2H).

### Example AY

### 5-Methoxy-N-(pyrazin-2-yl)-N-({5-[5-(trifluoromethyl)-1,2,4-oxadiazol-3-yl]thiophen-2-yl}methyl)pyrimidin-2-amine

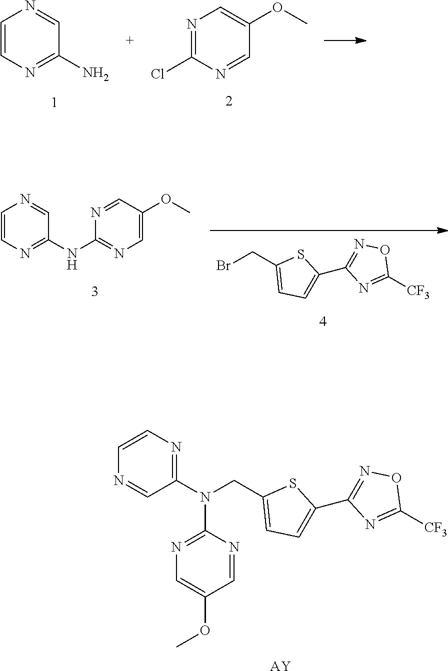

Under Ar(g), to a mixture of pyrazin-2-amine (1) (289 mg, 3.0 mmol), 2-chloro-5-methoxypyrimidine (2) (289 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (70 mg, 17%).

Under Ar(g), to a solution of (3) (70 mg, 0.35 mmol) in dry DMF (1.3 mL) was added NaH (60%, 17 mg, 0.42 mmol) in one portion at 0° C. After 5 min, a solution of (4) (129 mg, 0.41 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (AY) as a solid (77 mg, 52%).

LCMS (ES): Found 436.1 [M+H]⁺.

¹H NMR (400 MHz, DMSO-d₆), δ: 9.04 (s, 1H), 8.40-8.52 (m, 3H), 8.22 (s, 1H), 7.68-7.77 (m, 1H), 7.19-7.27 (m, 1H), 5.67 (s, 2H), 3.88 (s, 3H).

### Example AZ

### 4,6-Dimethyl-N-(pyrazin-2-yl)-N-({5-[5-(trifluoromethyl)-1,2,4-oxadiazol-3-yl]thiophen-2-yl}methyl)pyrimidin-2-amine

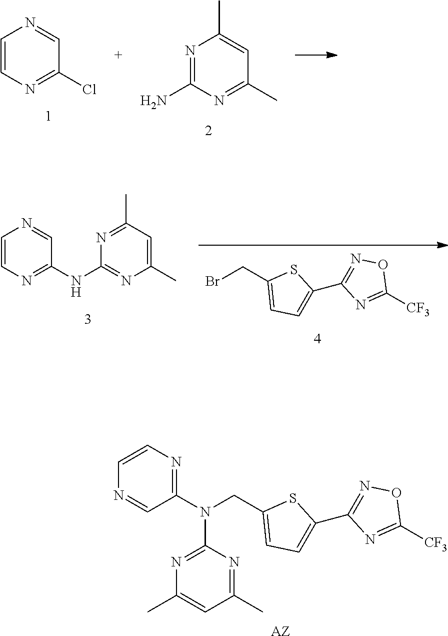

Under Ar(g), to a mixture of 2-chloropyrazine (1) (252 mg, 2.2 mmol), 2-amino-4,6-dimethylpyrimidine (2) (246 mg, 2.0 mmol), Cs₂CO₃ (1.3 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (159 mg, 39%).

Under Ar(g), to a solution of (3) (60 mg, 0.3 mmol) in dry DMF (1.25 mL) was added NaH (60%, 14 mg, 0.36 mmol) in one portion at 0° C. After 5 min, a solution of (4) (112 mg, 0.36 mmol) in dry DMF (1.0 mL) was added dropwise. The reaction mixture was allowed to warm up to rt overnight. Water (0.01 mL) was then added to quench the remaining NaH. The reaction mixture was purified by acidic prep LCMS to yield (AZ) as a solid (11 mg, 9%).

LCMS (ES): Found 434.4 [M+H]⁺.

¹H NMR (400 MHz, DMSO-d₆), δ: 9.17 (d, J=1.5 Hz, 1H), 8.49 (dd, J=2.7, 1.5 Hz, 1H), 8.27 (d, J=2.6 Hz, 1H), 7.73 (d, J=3.8 Hz, 1H), 7.27 (d, J=3.8 Hz, 1H), 6.89 (s, 1H), 5.68 (s, 2H), 2.38 (s, 6H).

### Example BA

### N-(Pyrazin-2-yl)-N-({5-[5-(trifluoromethyl)-1,2,4-oxadiazol-3-yl]thiophen-2-yl}methyl)pyrazin-2-amine

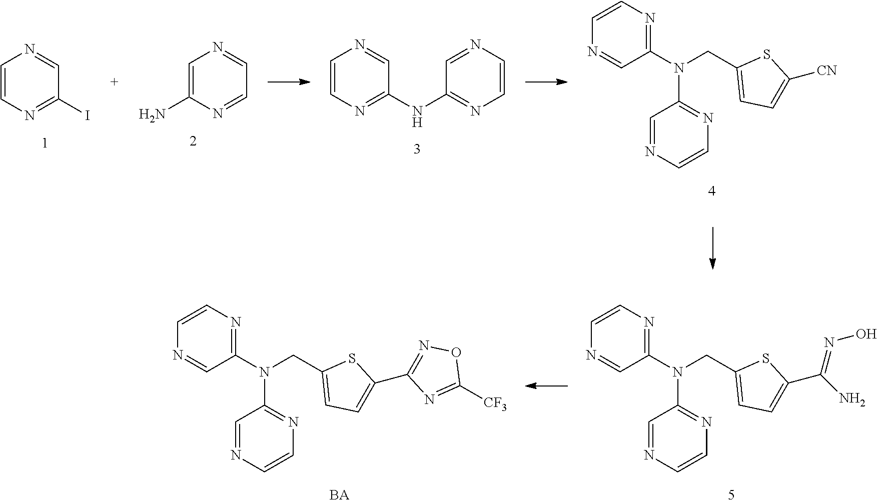 [SMILES: Ic1cnccn1 ; Nc1cnccn1 ; c1cnc(Nc2cnccn2)cn1 ; N#Cc1ccc(CN(c2cnccn2)c2cnccn2)s1 ; FC(F)(F)c1nc(-c2ccc(CN(c3cnccn3)c3cnccn3)s2)no1 ; N/C(=N\O)c1ccc(CN(c2cnccn2)c2cnccn2)s1]

NaH (60%, 51 mg, 1.27 mmol) was added to a solution of (3) (200 mg, 1.15 mmol) in DMF (2.5 mL) at 0° C. under Ar(g). The reaction mixture was stirred for 2 h then 5-(bromomethyl)thiophene-2-carbonitrile (257 mg, 1.27 mmol) was added dropwise as a solution in DMF (1 mL). The reaction mixture was allowed to warm up to rt overnight, poured into a mixture of H₂O (5 mL) and brine (10 mL), then extracted with EtOAc (5×20 mL). The combined organics were washed with brine (10 mL), dried over MgSO₄, filtered and concentrated in vacuo. Purification by silica gel column chromatography with hexane/EtOAc (1:1-0:1) yielded (4) as a light brown solid (254 mg, 75%).

¹H NMR (300 MHz, DMSO-d₆), δ: 8.78 (d, J=1.3 Hz, 2H), 8.35-8.43 (m, 2H), 8.29 (d, J=2.6 Hz, 2H), 7.78 (d, J=3.8 Hz, 1H), 7.29 (d, J=4.0 Hz, 1H), 5.61 (s, 2H).

LCMS (ES): Found 295.1 [M+H]⁺.

To a solution of (4) (250 mg, 0.85 mmol) in EtOH (5 mL) was added NH₂OH (50% in H₂O. 0.057 mL, 0.93 mmol) at rt. The reaction mixture was heated up to 80° C. for 1.5 h, then concentrated in vacuo to yield (5) as a tan solid (288 mg, ˜quant.), which was carried as such into the next step.

To a solution of (5) (100 mg, 0.31 mmol) in dry THF (2 mL) was added Et₃N (0.13 mL, 0.93 mmol) followed by trifluoroacetic anhydride (0.085 mL, 0.87 mmol) at 0° C. under Ar(g). The reaction mixture was allowed to warm up to rt overnight, then quenched with NaOH solution (5%, 1 mL). The reaction mixture was diluted with H₂O (5 mL) and brine (10 mL), then extracted with EtOAc (3×10 mL). The combined organics were washed with brine (10 mL), dried over MgSO₄, filtered and concentrated in vacuo. Purification by silica gel column chromatography with hexane/EtOAc (1:1-0:1) yielded (BA) as an off-white solid (67 mg, 55%).

LCMS (ES): Found 406.0 [M+H]⁺.

¹H NMR (300 MHz, DMSO-d₆), δ: 8.80 (d, J=1.3 Hz, 2H), 8.36-8.43 (m, 2H), 8.28 (d, J=2.4 Hz, 2H), 7.74 (d, J=3.8 Hz, 1H), 7.32 (d, J=3.6 Hz, 1H), 5.64 (s, 2H). ¹⁹F NMR (282 MHz, DMSO-d₆), δ: −64.79 (s, 3F).

### Example BB

### N-(Pyrazin-2-yl)-N-(1-{5-[5-(trifluoromethyl)-1,2,4-oxadiazol-3-yl]thiophen-2-yl}ethyl)pyrazin-2-amine

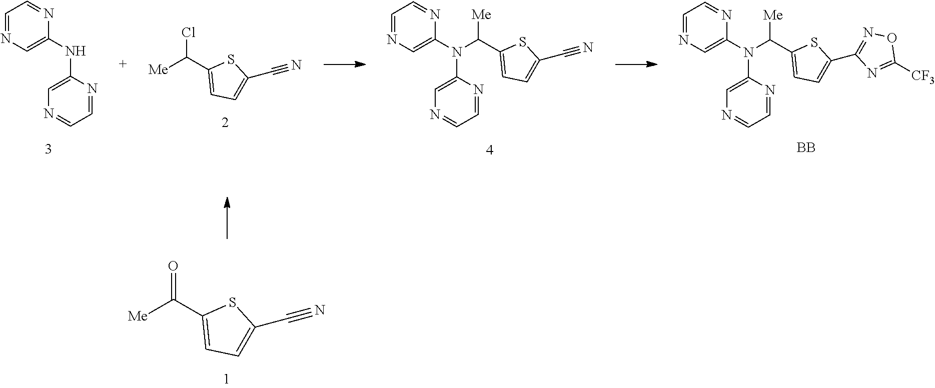 [SMILES: c1cnc(Nc2cnccn2)cn1 ; CC(Cl)c1ccc(C#N)s1 ; CC(c1ccc(C#N)s1)N(c1cnccn1)c1cnccn1 ; CC(c1ccc(-c2noc(C(F)(F)F)n2)s1)N(c1cnccn1)c1cnccn1 ; "BB" ; CC(=O)c1ccc(C#N)s1]

To a solution of 2-acetyl-5-cyanothiophene (1) (1.0 g, 6.7 mmol) in THF/MeOH (1:1, 10 mL) was added NaBH₄ (253 mg, 6.7 mmol) at 0° C. The reaction mixture was allowed to warm up to rt over 1 h, then concentrated in vacuo. The residue was re-dissolved in EtOAc (20 mL), quenched with 1M HCl solution until effervescence ceased, then poured into H₂O (10 mL) and extracted with EtOAc (2×20 mL). The combined organic extracts were dried over MgSO₄, filtered and concentrated in vacuo. A portion of this intermediate (555 mg, 3.62 mmol) was re-dissolved in MeCN (20 mL) and cooled down to 0° C. before addition of thionyl chloride (2.64 mL, 36.2 mmol). The reaction mixture was allowed to warm up to rt for 5.5 h, then concentrated in vacuo. Purification by silica gel column chromatography with hexane/EtOAc (1:0-9:1) yielded (2) as an orange oil (465 mg, 70%).

¹H NMR (300 MHz, Chloroform-d), δ: 7.49 (d, J=3.8 Hz, 1H), 7.07 (d, J=3.8 Hz, 1H), 5.30 (q, J=6.8 Hz, 1H), 1.95 (d, J=6.8 Hz, 3H).

¹³C NMR (75 MHz, Chloroform-d), δ: 155.5, 137.3, 125.3, 113.9, 109.6, 51.9, 26.9.

To a solution of (3) (81 mg, 0.47 mmol) in anhydrous DMF (1.5 mL) was added NaH (60%, 22 mg, 0.56 mmol) at 0° C. over 15 min. A solution of (2) (120 mg, 0.7 mmol) in DMF (1 mL) was then added and the reaction mixture was allowed to warm up to rt for 1.5 h. It was heated up to 40° C. overnight. Once cooled down to 0° C., additional portions of NaH (60%, 9 mg, 0.2 mmol) and a solution of (2) (37 mg, 0.2 mmol) in DMF (1 mL) were added and the reaction mixture was heated up to 40° C. for 3.5 h. Once cooled down to rt, it was poured into a solution of brine (50%, 10 mL) and extracted with EtOAc (3×10 mL). The combined organic extracts were dried over MgSO₄, filtered and concentrated in vacuo. Purification by silica gel column chromatography with hexane/EtOAc (1:0-0:1 then 1:0-1:1 then 1:0-2:1) yielded (4) as an oil (40 mg, 28%).

It was re-dissolved in EtOH (1 mL) and NH₂OH (50% in H₂O, 0.01 mL, 0.2 mmol) was added. The reaction mixture was heated up to 70° C. for 2 h. It was re-charged with NH₂OH (50% in H₂, 0.01 mL, 0.2 mmol) and stirred at 70° C. overnight. Once cooled down to rt, it was concentrated in vacuo. The residue was re-dissolved in THF (1 mL) and Et₃N (0.04 mL, 0.29 mmol) and trifluoroacetic anhydride (0.027 mL, 0.19 mmol) were added. The reaction mixture was stirred for 7 h, re-charged with trifluoroacetic anhydride (0.013 mL, 0.1 mmol) after 2 h and 5 h. The reaction mixture was then quenched with 1M NaOH solution to reach pH=10, concentrated in vacuo, re-dissolved in EtOAc (5 mL), poured into a brine solution (50%, 10 mL) and extracted with EtOAc (3×10 mL). The combined organic extracts were dried over MgSO₄, filtered and concentrated in vacuo. Purification by silica gel column chromatography with hexane/EtOAc (1:0-4:1) yielded (BB) as an off-white solid (21 mg, 11%).

LCMS (ES): Found 419.8 [M+H]⁺.

¹H NMR (300 MHz, DMSO-d₆), δ: 8.41 (dd, J=2.6, 1.5 Hz, 2H), 8.38 (d, J=1.5 Hz, 2H), 8.30 (d, J=2.6 Hz, 2H), 7.76 (d, J=3.8 Hz, 1H), 7.24 (dd, J=3.8, 1.1 Hz, 1H), 6.42 (q, J=7.0 Hz, 1H), 1.76 (d, J=7.0 Hz, 3H).

¹⁹F NMR (282 MHz, DMSO-d₆), δ: −64.79 (s, 3F).

### Example BE

### 3-Methyl-N-(pyrazin-2-yl)-N-({5-[5-(trifluoromethyl)-1,2,4-oxadiazol-3-yl]thiophen-2-yl}methyl)pyrazin-2-amine

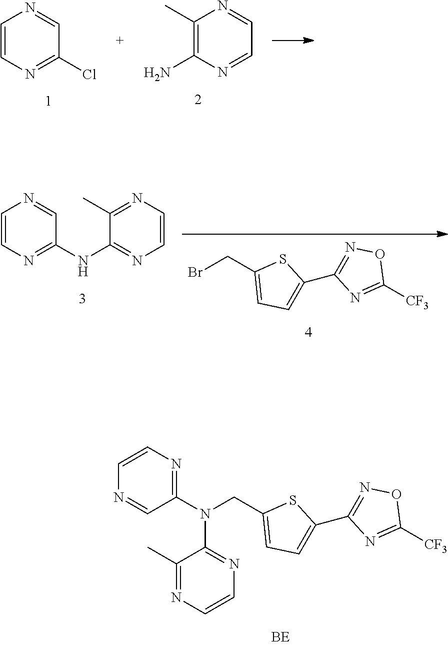

A suspension of 2-chloropyrazine (1) (250 mg, 2.3 mmol), 2-amino-3-methylpyrazine (2) (0.23 mL, 2.5 mmol), Cs₂CO₃ (1.5 g, 4.6 mmol), Pd₂(dba)₃ (105 mg, 0.12 mmol) and Xantphos (148 mg, 0.26 mmol) in dioxane (15 mL) was degassed with Ar(g) for 20 min. The reaction mixture was then heated up to 90° C. overnight. Once cooled down to rt, it was partitioned between EtOAc (35 mL), H₂O (10 mL) and brine (5 mL). The organic phase was separated and the aqueous phase extracted with EtOAc (2×10 mL). The combined organics extracts were dried (MgSO₄) and concentrated in vacuo. Purification by SCX-2 with CH₂Cl₂/MeOH (1:0-1:4) then 0.3M NH₃ gave a residue. Pre-washed MP-TMT (ca. 400 mg, 1.3 mmol/g) was added to the residue dissolved in CH₂Cl₂/MeOH (1:1, 30 mL), which was then agitated overnight. The resin was removed by filtration and the solution concentrated in vacuo. Purification by silica gel column chromatography with hexane/EtOAc (1:0-3:7) yielded (3) as a light yellow solid (310 mg, 72%).

LCMS (ES): Found 188.1 [M+H]⁺.

¹H NMR (300 MHz, DMSO-d₆), δ: 9.24 (s, 1H), 9.10 (d, J=1.3 Hz, 1H), 8.30 (dd, J=2.6, 1.5 Hz, 1H), 8.18 (d, J=2.6 Hz, 1H), 8.08-8.16 (m, 2H), 2.55 (s, 3H).

Under Ar(g), to a solution of (3) (50 mg, 0.27 mmol) in DMF (1.5 mL) was added NaH (60%, 13 mg, 0.32 mmol) in one portion at 0° C. After 5 min, a solution of (4) (100 mg, 0.32 mmol) in DMF (0.5 mL) was added dropwise over 1 min. The reaction mixture was allowed to warm up to rt overnight. Water (8 mL), brine (5 mL) and EtOAc (25 mL) were added. The organic phase was separated, extracting the aqueous phase with EtOAc (2×10 mL). The combined organic extracts were dried over MgSO₄ and concentrated in vacuo. Purification by silica gel column chromatography with hexane/EtOAc (1:0-3:7) yielded (BE) as a yellow oil (37 mg, 32%).

LCMS (ES): Found 420.0 [M+H]⁺.

¹H NMR (300 MHz, DMSO-d₆), δ: 8.45-8.52 (m, 2H), 8.25 (dd, J=2.6, 1.5 Hz, 1H), 8.19-8.23 (m, 1H), 8.11 (d, J=2.6 Hz, 1H), 7.74 (d, J=3.8 Hz, 1H), 7.26 (d, J=3.8 Hz, 1H), 5.49 (s, 2H), 2.20 (s, 3H).

¹⁹F NMR (282 MHz, DMSO-d₆), δ: −64.79 (s, 3F).

### Example BF

### 3-Methoxy-N-(pyrazin-2-yl)-N-({5-[5-(trifluoromethyl)-1,2,4-oxadiazol-3-yl]thiophen-2-yl}methyl)pyrazin-2-amine

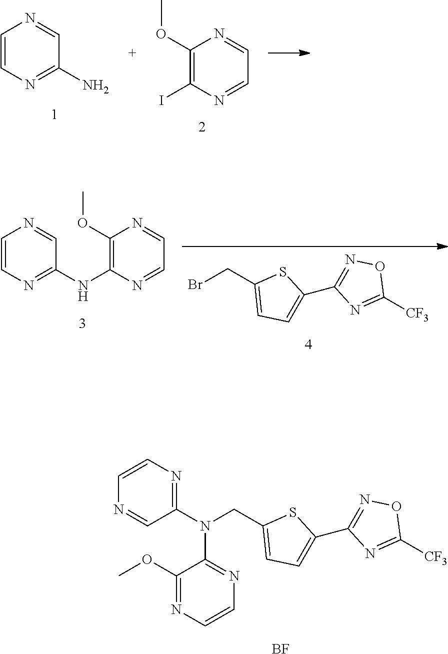

A suspension of pyrazin-2-amine (1) (168 mg, 1.77 mmol), 2-iodo-3-methoxypyrazine (2) (500 mg, 2.12 mmol), Cs₂CO₃ (1.15 g, 3.53 mmol) and Xantphos (148 mg, 0.26 mmol) in dioxane (15 mL) was purged with Ar(g) for 20 min. Pd₂(dba)₃ (81 mg, 0.09 mmol) was added and the reaction mixture was heated up to 90° C. overnight. Once cooled down to rt, it was poured into a mixture of H₂O (15 mL) and brine (15 mL), then extracted with EtOAc (3×20 mL). The combined organics were washed with brine (3×10 mL), dried over MgSO₄, filtered and concentrated in vacuo. The residue was re-dissolved in CH₂Cl₂/MeOH (4:1, 100 mL) and Pd-scavenged with MP-TMT resin (1.36 g, 1.3 mmol/g) at rt for 2 h. The reaction mixture was filtered and concentrated in vacuo. Purification by silica gel column chromatography with hexane/EtOAc (1:0-1:1) yielded (3) as a purple solid (302 mg, 84%).

NaH (60%, 18 mg, 0.46 mmol) was added to a suspension of (3) (50 mg, 0.25 mmol) in DMF (1.0 mL) at 0° C. under Ar(g). The reaction mixture was stirred for 35 min then (4) (92 mg, 0.64 mmol) was added in one portion. It was allowed to warm up to rt for 1 h, poured into a mixture of H₂O (10 mL) and brine (10 mL), then extracted with EtOAc (4×10 mL). The combined organics were dried over MgSO₄, filtered and concentrated in vacuo. Purification by silica gel column chromatography with CH₂Cl₂/EtOAc (1:0-4:1) yielded (BF) as a pale yellow solid (76 mg, 71%).

LCMS (ES): Found 436.0 [M+H]⁺.

¹H NMR (300 MHz, DMSO-d₆), δ: 8.27 (dd, J=2.5, 1.4 Hz, 1H), 8.24 (d, J=1.1 Hz, 1H), 8.07-8.15 (m, 3H), 7.71 (d, J=3.8 Hz, 1H), 7.22 (d, J=3.8 Hz, 1H), 5.53 (s, 2H), 3.82 (s, 3H).

¹⁹F NMR (282 MHz, DMSO-d₆), δ: −64.79 (s, 3F).

### Example BG

### 3-(Methoxymethyl)-N-(pyrazin-2-yl)-N-({5-[5-(trifluoromethyl)-1,2,4-oxadiazol-3-yl]thiophen-2-yl}methyl)pyrazin-2-amine

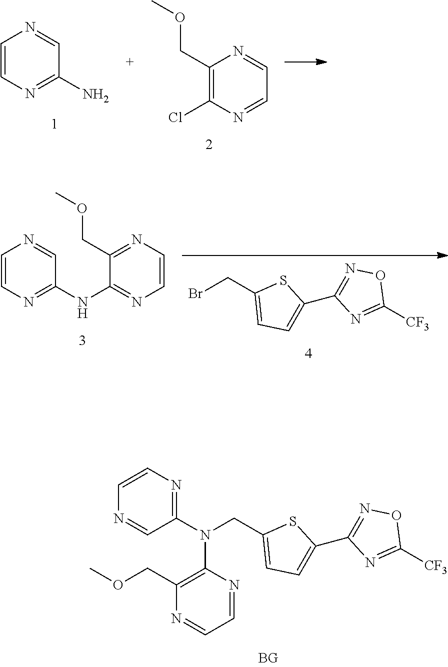

Under Ar(g), to a mixture of pyrazin-2-amine (1) (165 mg, 1.7 mmol), 2-chloro-3-(methoxymethyl)pyrazine (2) (250 mg, 1.6 mmol), Cs₂CO₃ (1.03 g, 3.1 mmol) was added degassed dry 1,4-dioxane (13 mL). The reaction mixture was then flushed with Ar(g) for 1 min before Pd₂(dba)₃ (72 mg, 0.08 mmol) and Xantphos (100 mg, 0.17 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (177 mg, 52%).

NaH (60%, 24 mg, 0.6 mmol) was added to a solution of (3) (65 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (BG) as a solid (45 mg, 39%).

LCMS (ES): Found 450.4 [M+H]⁺.

¹H NMR (400 MHz, DMSO-d₆), δ: 8.60 (d, J=2.5 Hz, 1H), 8.59 (d, J=2.5 Hz, 1H), 8.24 (dd, J=2.6, 1.5 Hz, 1H), 8.21 (d, J=1.5 Hz, 1H), 8.11 (d, J=2.7 Hz, 1H), 7.74 (d, J=3.7 Hz, 1H), 7.26 (d, J=3.8 Hz, 1H), 5.49 (s, 2H), 4.29 (s, 2H), 3.10 (s, 3H).

### Example BH

### 5-Methyl-N-(pyrazin-2-yl)-N-({5-[5-(trifluoromethyl)-1,2,4-oxadiazol-3-yl]thiophen-2-yl}methyl)pyrazin-2-amine

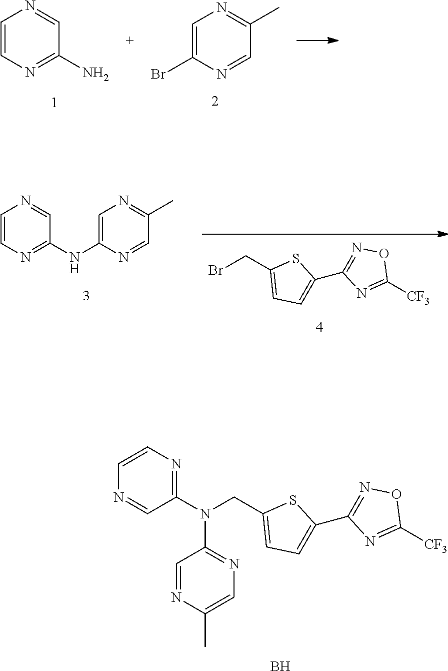

A suspension of pyrazin-2-amine (1) (343 mg, 3.6 mmol), 2-bromo-5-methylpyrazine (2) (567 mg, 3.3 mmol), Cs₂CO₃ (2.1 g, 6.6 mmol), Pd₂(dba)₃ (150 mg, 0.16 mmol) and Xantphos (209 mg, 0.36 mmol) in dioxane (15 mL) was degassed with Ar(g) for 20 min. The reaction mixture was then heated up to 90° C. overnight. Once cooled down to rt, it was partitioned between H₂O (10 mL), brine (5 mL) and EtOAc (2×10 mL). The combined organics were dried over MgSO₄, filtered and concentrated in vacuo. The residue was dissolved in CH₂Cl₂/MeOH (1:1, 20 mL) and Pd-scavenged with MP-TMT resin (˜150 mg) overnight. The suspension was then filtered and solvent removed in vacuo. Purification by silica gel column chromatography with hexane/EtOAc (1:0-0:1) followed by SCX-2 with CH₂Cl₂/MeOH (1:0-0:1+0.3M NH₃) yielded (3) as a yellow solid (242 mg, 40%).

LCMS (ES): Found 188.1 [M+H]⁺.

¹H NMR (300 MHz, DMSO-d₆), δ: 10.22 (s, 1H), 8.96 (d, J=1.5 Hz, 1H), 8.92 (d, J=1.5 Hz, 1H), 8.27 (dd, J=2.6, 1.5 Hz, 1H), 8.20 (d, J=0.8 Hz, 1H), 8.12 (d, J=2.6 Hz, 1H), 2.41 (s, 3H).

NaH (60%, 12 mg, 0.29 mmol) was added to a solution of (3) (50 mg, 0.27 mmol) in DMF (3 mL) at 0° C. under Ar(g). The reaction mixture was stirred for 20 min then (4) (92 mg, 0.29 mmol) was added as a solution in DMF (1 mL). The reaction mixture was allowed to warm up to rt overnight. The reaction mixture was partitioned between H₂O (10 mL), brine (10 mL) and EtOAc (4×10 mL). The combined organics were washed with brine (5 mL), then dried over MgSO₄, filtered and concentrated in vacuo. Purification by silica gel column chromatography with hexane/EtOAc (1:0-0:1) yielded (BH) as a yellow oil (51 mg, 45%).

LCMS (ES): Found 420.0 [M+H]⁺.

¹H NMR (300 MHz, DMSO-d₆), δ: 8.70 (d, J=1.5 Hz, 1H), 8.68 (d, J=1.5 Hz, 1H), 8.36 (dd, J=2.6, 1.5 Hz, 1H), 8.31 (d, J=0.8 Hz, 1H), 8.21 (d, J=2.6 Hz, 1H), 7.74 (d, J=3.8 Hz, 1H), 7.29 (d, J=3.8 Hz, 1H), 5.61 (s, 2H), 2.45 (s, 3H).

¹⁹F NMR (282 MHz, DMSO-d₆), δ: −64.80 (s, 3F).

### Example BI

### 5-Methoxy-N-(pyrazin-2-yl)-N-({5-[5-(trifluoromethyl)-1,2,4-oxadiazol-3-yl]thiophen-2-yl}methyl)pyrazin-2-amine

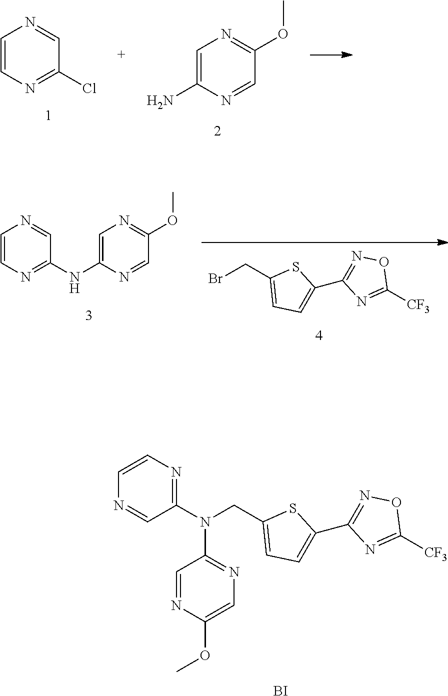

Dioxane (15 mL) was degassed with Ar(g) for 0.5 h before 2-amino-5-methoxypyrazine (2) (503 mg, 4.0 mmol), 2-chloropyrazine (1) (0.45 mL, 4.8 mmol), Pd₂(dba)₃ (184 mg, 0.20 mmol), XantPhos (233 mg, 0.40 mmol) and Cs₂CO₃ (2.6 g, 8.0 mmol) were added sequentially with continued degassing. The reaction mixture was degassed with Ar(g) for a further 15 min then heated up to 90° C. overnight. Once cooled down to rt, the reaction mixture was poured into H₂O (20 mL) and extracted with EtOAc (5×30 mL). The combined organic extracts were dried over MgSO₄, filtered and concentrated in vacuo. Purification by silica gel column chromatography with hexane/EtOAc (1:0-0:1) then CH₂Cl₂/EtOAc (1:0-1:1), then re-dissolved in CH₂Cl₂/MeOH (4:1, 50 mL) and swirled with MP-TMT resin (1.2 g, 1.1 mmol/g) at rt overnight. The solution was filtered, the resin washed with CH₂Cl₂/MeOH (4:1, 100 mL) and the filtrate concentrated in vacuo to yield (3) as an off-white solid (600 mg, 73%).

LCMS (ES): Found 204.1 [M+H]⁺.

¹H NMR (300 MHz, DMSO-d₆), δ: 10.05 (s, 1H), 8.81 (d, J=1.3 Hz, 1H), 8.65 (d, J=1.5 Hz, 1H), 8.19 (dd, J=2.6, 1.5 Hz, 1H), 8.06 (d, J=1.3 Hz, 1H), 8.03 (d, J=2.6 Hz, 1H), 3.88 (s, 3H).

To a solution of (3) (52 mg, 0.25 mmol) in anhydrous DMF (2 mL) was added NaH (60%, 12 mg, 0.30 mmol) at 0° C. The reaction mixture was stirred for 15 min before the addition of (4) (95 mg, 0.31 mmol), then it was allowed to warm up to rt overnight. Once cooled down to 0° C. the reaction was re-charged twice with NaH (60%, 3 mg, 0.076 mmol; then 5 mg, 0.13 mmol) and (4) (24 mg, 0.076 mmol; then 80 mg, 0.25 mmol) and stirred for 4 h. After a further 1.5 h, the reaction was quenched by the addition of H₂O (1 mL), poured into a brine solution (50%, 15 mL) and extracted with EtOAc (3×15 mL). The combined organic extracts were washed with brine (20 mL), dried over MgSO₄, filtered and concentrated in vacuo. Purification by silica gel column chromatography with hexane/EtOAc (1:0-7:3) yielded (BI) as an orange solid (26 mg, 23%).

LCMS (ES): Found 436.0 [M+H]⁺.

¹H NMR (300 MHz, DMSO-d₆), δ: 8.40 (d, J=1.3 Hz, 1H), 8.38 (d, J=1.5 Hz, 1H), 8.29 (dd, J=2.6, 1.5 Hz, 1H), 8.21 (d, J=1.5 Hz, 1H), 8.09 (d, J=2.6 Hz, 1H), 7.74 (d, J=3.6 Hz, 1H), 7.24 (d, J=3.8 Hz, 1H), 5.50 (s, 2H), 3.92 (s, 3H).

¹⁹F NMR (282 MHz, DMSO-d₆), δ: −64.79 (s, 3F).

### Example BJ

### 5-(Furan-2-yl)-N-(pyrazin-2-yl)-N-({5-[5-(trifluoromethyl)-1,2,4-oxadiazol-3-yl]thiophen-2-yl}methyl)pyrazin-2-amine

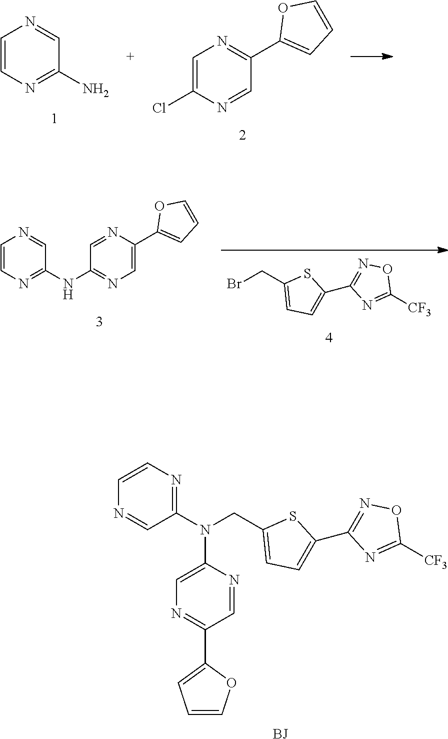 [SMILES: Nc1cnccn1 ; Clc1cnc(-c2ccco2)cn1 ; c1coc(-c2cnc(Nc3cnccn3)cn2)c1 ; FC(F)(F)c1nc(-c2ccc(CBr)s2)no1 ; FC(F)(F)c1nc(-c2ccc(CN(c3cnccn3)c3cnc(-c4ccco4)cn3)s2)no1 ; "BJ"]

Under Ar(g), to a mixture of pyrazin-2-amine (1) (145 mg, 1.5 mmol), 2-chloro-5-furan-2-yl-pyrazine (2) (250 mg, 1.4 mmol), Cs₂CO₃ (0.9 g, 2.8 mmol) was added degassed dry 1,4-dioxane (13 mL). The reaction mixture was then flushed with Ar(g) for 1 min before Pd₂(dba)₃ (63 mg, 0.07 mmol) and Xantphos (88 mg, 0.15 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (69 mg, 21%).

NaH (60%, 14 mg, 0.35 mmol) was added to a solution of (3) (69 mg, 0.29 mmol) in dry DMF (1.2 mL). After 5 min, a solution of (4) (108 mg, 0.35 mmol) in dry DMF (1 mL) was added. The reaction mixture was stirred overnight. Water (0.01 mL) was then added to quench the remaining NaH. The reaction mixture was purified by acidic prep LCMS to yield (BJ) as a solid (59 mg, 44%).

LCMS (ES): Found 472.4 [M+H]⁺.

¹H NMR (400 MHz, DMSO-d₆), δ: 8.83 (d, J=1.4 Hz, 1H), 8.80 (d, J=1.5 Hz, 1H), 8.75 (d, J=1.4 Hz, 1H), 8.42 (dd, J=2.7, 1.5 Hz, 1H), 8.28 (d, J=2.7 Hz, 1H), 7.86-7.90 (m, 1H), 7.76 (d, J=3.8 Hz, 1H), 7.34 (d, J=3.8 Hz, 1H), 7.11 (d, J=3.4 Hz, 1H), 6.69 (dd, J=3.4, 1.8 Hz, 1H), 5.67 (s, 2H).

### Example BK

### N-(Pyrazin-2-yl)-5-(trifluoromethyl)-N-({5-[5-(trifluoromethyl)-1,2,4-oxadiazol-3-yl]thiophen-2-yl}methyl)pyrazin-2-amine

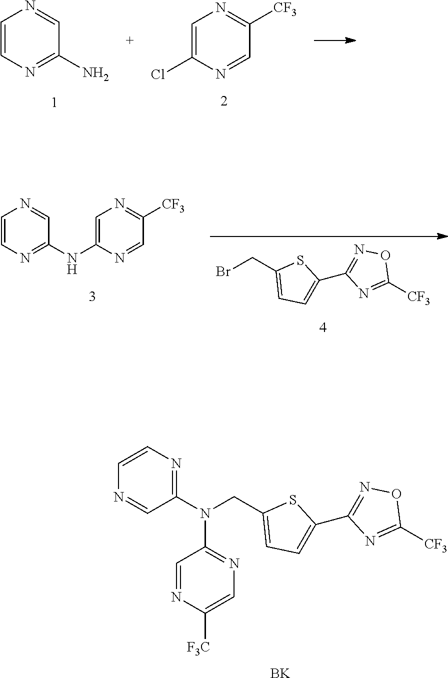 [SMILES: Nc1cnccn1 ; FC(F)(F)c1cnc(Cl)cn1 ; FC(F)(F)c1cnc(Nc2cnccn2)cn1 ; FC(F)(F)c1nc(-c2ccc(CBr)s2)no1 ; FC(F)(F)c1cnc(N(Cc2ccc(-c3noc(C(F)(F)F)n3)s2)c2cnccn2)cn1 ; "BK"]

Dioxane (15 mL) was degassed with Ar(g) for 25 min before 5-chloro-2-trifluoromethylpyrazine (2) (0.34 mL, 2.74 mmol), pyrazin-2-amine (1) (312 mg, 3.28 mmol), Pd₂(dba)₃ (125 mg, 0.14 mmol), XantPhos (159 mg, 0.27 mmol) and Cs₂CO₃ (1.78 g, 5.47 mmol) were added sequentially with continued degassing. The reaction mixture was degassed with Ar(g) for a further 15 min then heated up to 90° C. overnight. Once cooled down to rt, it was poured into H₂O (20 mL) and extracted with CH₂Cl₂ (3×30 mL). The combined organic extracts were dried over MgSO₄, filtered and concentrated in vacuo. The residue was purified by silica gel column chromatography with CH₂Cl₂/MeOH (1:0-24:1), CH₂Cl₂/EtOAc (1:0-4:1) and hexane/EtOAc (1:0-4:1). It was then re-dissolved in CH₂Cl₂/MeOH (4:1, 50 mL) and swirled with MP-TMT resin (1.02 g, 1.33 mmol) at rt overnight.

The solution was filtered, the resin washed with CH₂Cl₂/MeOH (4:1, 100 mL) and the filtrate concentrated in vacuo to yield (3) as an off-white solid (498 mg, 63%).

LCMS (ES): Found 242.1 [M+H]⁺.

¹H NMR (300 MHz, DMSO-d₆), δ: 10.11 (s, 1H), 8.30 (d, J=1.5 Hz, 1H), 8.16 (d, J=1.3 Hz, 1H), 7.87-7.94 (m, 1H), 7.54 (dd, J=2.6, 1.5 Hz, 1H), 7.44 (d, J=2.6 Hz, 1H).

¹⁹F NMR (282 MHz, DMSO-d₆), δ: −65.25 (s, 3F).

To a solution of (3) (52 mg, 0.22 mmol) in anhydrous DMF (2 mL) was added NaH (60%, 10 mg, 0.26 mmol) at 0° C. The solution was stirred for 15 min before the addition of (4) (81 mg, 0.26 mmol) and it was allowed to warm up to rt overnight. Once cooled down to 0° C., it was then quenched by the addition of H₂O (1.5 mL), poured into a brine solution (50%, 15 mL) and extracted with EtOAc (3×20 mL). The combined organic extracts were dried over MgSO₄, filtered and concentrated in vacuo. Purification by silica gel column chromatography with CH₂Cl₂/EtOAc (1:0-4:1) then hexane/EtOAc (1:0-3:2) yielded (BK) as an orange solid (77 mg, 75%).

LCMS (ES): Found 474.0 [M+H]⁺.

¹H NMR (300 MHz, DMSO-d₆), δ: 8.99 (d, J=1.3 Hz, 1H), 8.82-8.86 (m, 2H), 8.51 (dd, J=2.5, 1.3 Hz, 1H), 8.44 (d, J=2.4 Hz, 1H), 7.76 (d, J=3.8 Hz, 1H), 7.34 (d, J=3.8 Hz, 1H), 5.70 (s, 2H).

¹⁹F NMR (282 MHz, DMSO-d₆), δ: −64.79 (s, 3F), −65.34 (s, 3F).

### Example BL

### 5-Methanesulfonyl-N-(pyrazin-2-yl)-N-({5-[5-(trifluoromethyl)-1,2,4-oxadiazol-3-yl]thiophen-2-yl}methyl)pyrazin-2-amine

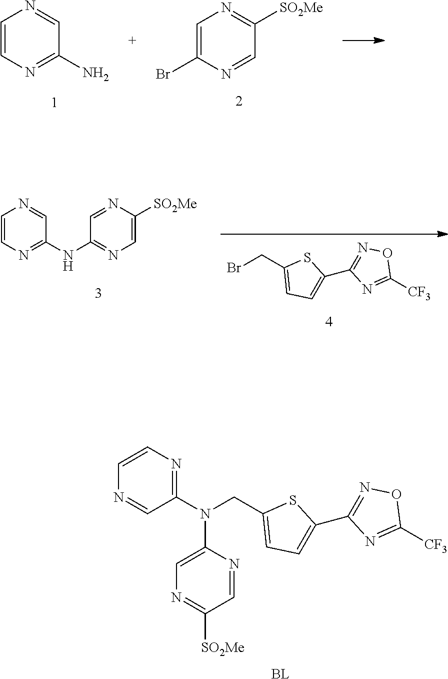 [SMILES: Nc1cnccn1 ; CS(=O)(=O)c1cnc(Br)cn1 ; CS(=O)(=O)c1cnc(Nc2cnccn2)cn1 ; FC(F)(F)c1nc(-c2ccc(CBr)s2)no1 ; CS(=O)(=O)c1cnc(N(Cc2ccc(-c3noc(C(F)(F)F)n3)s2)c2cnccn2)cn1 ; "BL"]

Under Ar(g), to a mixture of pyrazin-2-amine (1) (110 mg, 1.2 mmol), 2-bromo-5-methylsulfonylpyrazine (2) (250 mg, 1.1 mmol), Cs₂CO₃ (0.69 g, 2.1 mmol) was added degassed dry 1,4-dioxane (13 mL). The reaction mixture was then flushed with Ar(g) for 1 min before Pd₂(dba)₃ (48 mg, 0.05 mmol) and Xantphos (67 mg, 0.12 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (185 mg, 70%).

LCMS (ES): Found 252.3 [M+H]⁺.

NaH (60%, 24 mg, 0.6 mmol) was added to a solution of (3) (75 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (BL) as a solid (28 mg, 19%).

LCMS (ES): Found 484.3 [M+H]⁺.

¹H NMR (400 MHz, DMSO-d₆), δ: 9.01 (d, J=1.4 Hz, 1H), 8.86 (d, J=1.4 Hz, 1H), 8.82 (d, J=1.3 Hz, 1H), 8.53 (dd, J=2.7, 1.4 Hz, 1H), 8.48 (d, J=2.5 Hz, 1H), 7.77 (d, J=3.8 Hz, 1H), 7.36 (d, J=3.8 Hz, 1H), 5.72 (s, 2H), 3.27 (s, 3H).

### Example BM

### 5-(Morpholin-4-yl)-N-(pyrazin-2-yl)-N-({5-[5-(trifluoromethyl)-1,2,4-oxadiazol-3-yl]thiophen-2-yl}methyl)pyrazin-2-amine

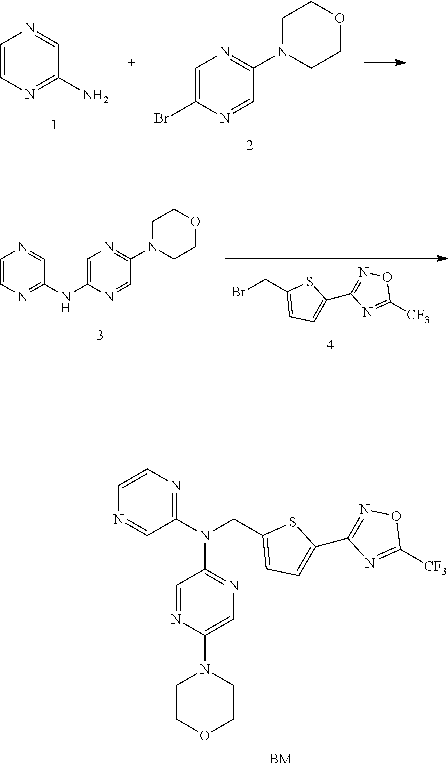

Dioxane (15 mL) was degassed with Ar(g) for 0.5 h before 2-bromo-5-morpholinopyrazine (2) (501 mg, 2.05 mmol), pyrazin-2-amine (1) (976 mg, 10.3 mmol), Pd₂(dba)₃ (94 mg, 0.103 mmol), XantPhos (119 mg, 0.205 mmol) and Cs₂CO₃ (3.34 g, 10.3 mmol) were added sequentially with continued degassing. The reaction mixture was degassed with Ar(g) for a further 1 h then heated up to 90° C. overnight. Once cooled down to, the reaction mixture was poured into a brine solution (50%, 25 mL) and extracted with EtOAc (4×35 mL). The combined organic extracts were dried over MgSO₄, filtered and concentrated in vacuo. The residue was purified by silica gel column chromatography with hexane/EtOAc (1:0-0:1), then re-dissolved in CH₂Cl₂/MeOH (4:1, 50 mL) and swirled with MP-TMT resin (1.0 g, 1.3 mmol/g) at rt overnight. The solution was filtered, the resin washed with CH₂Cl₂/MeOH (4:1, 100 mL) and the filtrate concentrated in vacuo. Purification by silica gel column chromatography with CH₂Cl₂/EtOAc (1:0-0:1) then reverse-phase column chromatography with H₂O/MeCN (19:1-3:2) yielded (3) as a yellow solid (163 mg, 31%).

LCMS (ES): Found 259.1 [M+H]⁺.

¹H NMR (300 MHz, DMSO-d₆), δ: 9.88 (s, 1H), 8.77 (d, J=1.3 Hz, 1H), 8.63 (d, J=1.3 Hz, 1H), 8.16 (dd, J=2.6, 1.5 Hz, 1H), 8.06 (d, J=1.3 Hz, 1H), 7.98 (d, J=2.6 Hz, 1H), 3.68-3.78 (m 4H), 3.35-3.45 (m 4H).

To a solution of (3) (52 mg, 0.202 mmol) in anhydrous DMF (2 mL) was added NaH (60%, 10 mg, 0.243 mmol) at 0° C. The reaction mixture solution was stirred for 15 min before the addition of (4) (76 mg, 0.243 mmol). It was then allowed to warm up to rt overnight. Once cooled down to 0° C., it was quenched by the addition of H₂O (1 mL), poured into a brine solution (50%, 10 mL) and extracted with EtOAc (3×10 mL). The combined organic extracts were dried over MgSO₄, filtered and concentrated in vacuo. Purification three times by silica gel column chromatography with CH₂Cl₂/EtOAc (1:0-3:2) then hexane/EtOAc (1:0-3:7) then CH₂Cl₂/MeOH (1:0-19:1) yielded (BM) as a yellow solid (29 mg, 29%).

LCMS (ES): Found 491.0 [M+H]⁺.

¹H NMR (300 MHz, DMSO-d₆), δ: 8.29 (d, J=1.3 Hz, 1H), 8.24-8.27 (m, 1H), 8.22 (d, J=1.5 Hz, 1H), 8.20 (d, J=1.5 Hz, 1H), 8.03 (d, J=2.6 Hz, 1H), 7.74 (d, J=3.8 Hz, 1H), 7.22 (d, J=3.8 Hz, 1H), 5.44 (s, 2H), 3.66-3.77 (m, 4H), 3.46-3.57 (m, 4H).

¹⁹F NMR (282 MHz, DMSO-d₆), δ: −64.78 (s, 3F).

### Example BN

### N-{5H-Pyrrolo[2,3-b]pyrazin-2-yl}-N-({5-[5-(trifluoromethyl)-1,2,4-oxadiazol-3-yl]thiophen-2-yl}methyl)pyrazin-2-amine

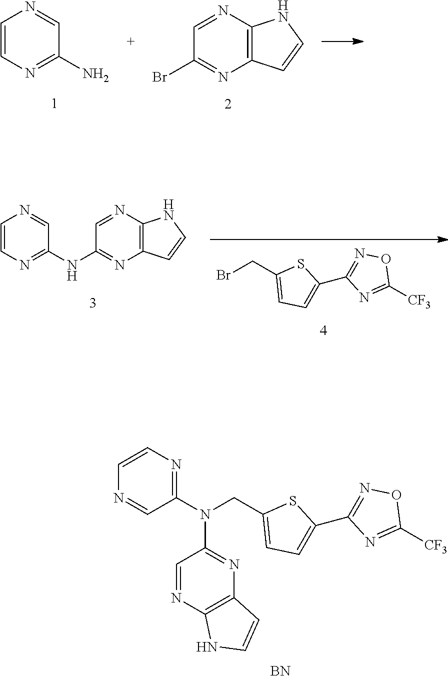

Under Ar(g), to a mixture of pyrazin-2-amine (1) (209 mg, 2.2 mmol), 2-bromo-5H-pyrrolo[2,3-b]pyrazine (2) (396 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (22 mg, 5%).

NaH (60%, 5.0 mg, 0.12 mmol) was added to a solution of (3) (22 mg, 0.1 mmol) in dry DMF 0.7 mL). After 5 min, a solution of (4) (39 mg, 0.12 mmol) in dry DMF (0.6 mL) was added. The reaction mixture was stirred overnight. Water (0.01 mL) was then added to quench the remaining NaH. The reaction mixture was purified by acidic prep LCMS to yield (BN) as a solid (9.8 mg, 21%).

LCMS (ES): Found 445.2 [M+H]⁺.

¹H NMR (400 MHz, DMSO-d₆), δ: 10.20 (s, 1H), 9.20 (d, J=1.5 Hz, 1H), 8.65 (s, 1H), 8.25 (dd, J=2.6, 1.5 Hz, 1H), 8.10 (d, J=2.6 Hz, 1H), 7.97 (d, J=3.6 Hz, 1H), 7.79 (d, J=3.8 Hz, 1H), 7.28 (d, J=3.8 Hz, 1H), 6.59 (d, J=3.6 Hz, 1H), 5.76 (s, 2H).

### Example BO

### N-{5-Methyl-5H-pyrrolo[2,3-b]pyrazin-2-yl}-N-({5-[5-(trifluoromethyl)-1,2,4-oxadiazol-3-yl]thiophen-2-yl}methyl)pyrazin-2-amine

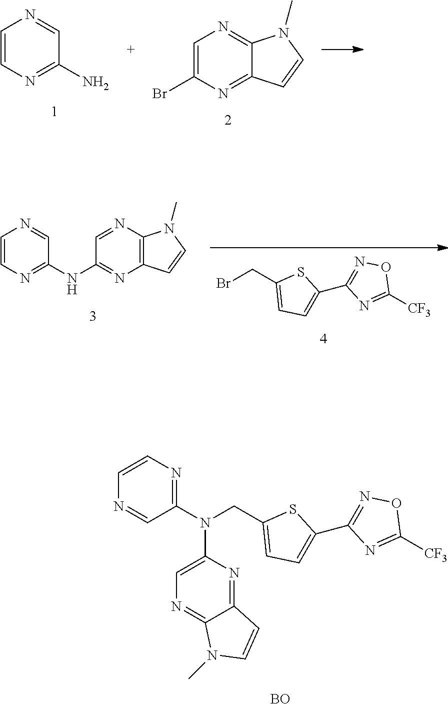

Under Ar(g), to a mixture of pyrazin-2-amine (1) (123 mg, 1.3 mmol), N-methyl-5-bromo-4,7-diazaindole (2) (250 mg, 1.2 mmol), Cs₂CO₃ (0.77 g, 2.4 mmol) was added degassed dry 1,4-dioxane (13 mL). The reaction mixture was then flushed with Ar(g) for 1 min before Pd₂(dba)₃ (54 mg, 0.06 mmol) and Xantphos (75 mg, 0.13 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (147 mg, 55%).

LCMS (ES): Found 227.2 [M+H]⁺.

Under Ar(g), to a solution of (3) (68 mg, 0.3 mmol) in dry DMF (1.3 mL) was added NaH (60%, 14 mg, 0.36 mmol) in one portion at 0° C. After 5 min, a solution of (4) (110 mg, 0.35 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (BO) as a solid (59 mg, 43%).

LCMS (ES): Found 459.4 [M+H]⁺.

¹H NMR (400 MHz, DMSO-d₆), δ: 8.43 (s, 1H), 8.38 (d, J=1.5 Hz, 1H), 8.30 (dd, J=2.8, 1.5 Hz, 1H), 8.09 (d, J=2.6 Hz, 1H), 7.92 (d, J=3.5 Hz, 1H), 7.72 (d, J=3.8 Hz, 1H), 7.24 (d, J=3.7 Hz, 1H), 6.60 (d, J=3.6 Hz, 1H), 5.60 (s, 2H), 3.86 (s, 3H).

### Example BP

### 6-Methyl-N-(pyrazin-2-yl)-N-({5-[5-(trifluoromethyl)-1,2,4-oxadiazol-3-yl]thiophen-2-yl}methyl)pyrazin-2-amine

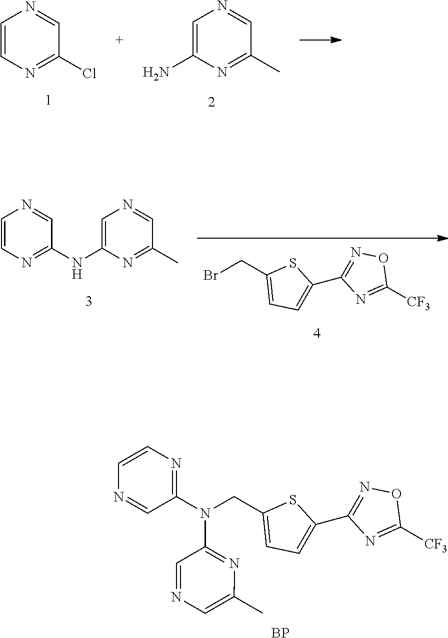

Dioxane (15 mL) was degassed with Ar(g) for 20 min before 2-amino-6-methylpyrazine (2) (407 mg, 3.73 mmol), 2-chloropyrazine (1) (0.4 mL, 4.48 mmol), Pd₂(dba)₃ (103 mg, 0.112 mmol), XantPhos (130 mg, 0.22 mmol) and Cs₂CO₃ (2.43 g, 7.46 mmol) were added sequentially with continued degassing. The reaction mixture was degassed with Ar(g) for a further 25 min then heated up to 90° C. for 20 h. After cooling to rt, the reaction mixture was poured into a brine solution (50%, 20 mL) and extracted with EtOAc (4×30 mL). The combined organic extracts were dried over MgSO₄, filtered and concentrated in vacuo. The residue was re-dissolved in CH₂Cl₂/MeOH (4:1, 50 mL) and swirled with MP-TMT resin (2 g, 1.3 mmol/g) at rt for 6 h. The solution was filtered, the resin washed with CH₂Cl₂/MeOH (4:1, 150 mL) and the filtrate concentrated in vacuo. Purification by silica gel column chromatography with hexane/EtOAc (1:0-1:1) yielded (3) as an off-white solid (256 mg, 37%).

LCMS (ES): Found 188.1 [M+H]⁺.

¹H NMR (300 MHz, DMSO-d₆), δ: 10.32 (s, 1H), 8.99 (d, J=1.5 Hz, 1H), 8.88 (s, 1H), 8.28 (dd, J=2.6, 1.5 Hz, 1H), 8.14 (d, J=2.6 Hz, 1H), 8.07 (s, 1H), 2.43 (s, 3H).

To a solution of (3) (56 mg, 0.296 mmol) in anhydrous DMF (2 mL) was added NaH (60%, 14 mg, 0.36 mmol) at 0° C. The reaction mixture was stirred at 0° C. for 15 min before the addition of (4) (111 mg, 0.36 mmol). It was stirred for 3.5 h then quenched by the addition of H₂O (2 mL), poured into a brine solution (50%, 10 mL) and extracted with EtOAc (3×10 mL). The combined organic extracts were dried over MgSO₄, filtered and concentrated in vacuo. Purification by silica gel column chromatography with hexane/EtOAc (1:0-3:2) yielded (BP) as a white solid (35 mg, 28%).

LCMS (ES): Found 420.0 [M+H]⁺.

¹H NMR (300 MHz, DMSO-d₆), δ: 8.79 (d, J=1.5 Hz, 1H), 8.58 (s, 1H), 8.40 (dd, J=2.6, 1.5 Hz, 1H), 8.25 (d, J=2.6 Hz, 1H), 8.18 (s, 1H), 7.74 (d, J=3.6 Hz, 1H), 7.32 (d, J=4.0 Hz, 1H), 5.62 (s, 2H), 2.45 (s, 3H).

¹⁹F NMR (282 MHz, DMSO-d₆), δ: −64.79 (s, 3F).

### Example BQ

### 6-Methoxy-N-(pyrazin-2-yl)-N-({5-[5-(trifluoromethyl)-1,2,4-oxadiazol-3-yl]thiophen-2-yl}methyl)pyrazin-2-amine

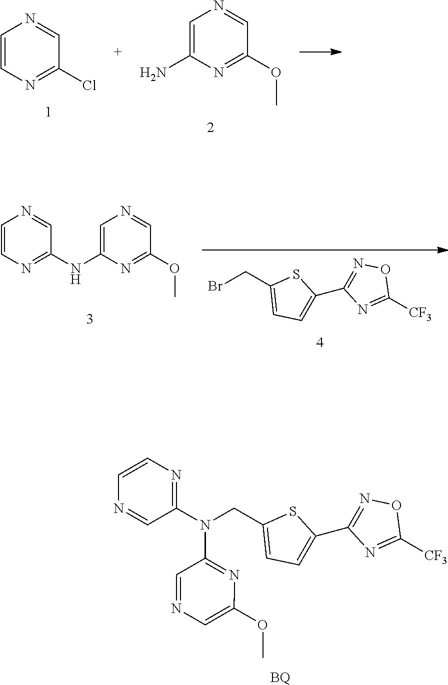

Dioxane (10 mL) was degassed with Ar(g) for 20 min before 2-amino-6-methoxypyrazine (2) (310 mg, 2.48 mmol), 2-chloropyrazine (1) (0.026 mL, 2.97 mmol), Pd₂(dba)₃ (68 mg, 0.074 mmol), XantPhos (86 mg, 0.15 mmol) and Cs₂CO₃ (1.6 g, 4.96 mmol) were added sequentially with continued degassing. The reaction mixture was degassed with Ar(g) for a further 10 min then heated up to 90° C. for 21 h. After cooling to rt, the reaction mixture was poured into a brine solution (50%, 20 mL) and extracted with EtOAc (4×30 mL). The combined organic extracts were dried over MgSO₄, filtered and concentrated in vacuo. The residue was purified by silica gel column chromatography with hexane/EtOAc (1:0-1:4), then re-dissolved in CH₂Cl₂/MeOH (4:1, 50 mL) and swirled with MP-TMT resin (600 mg, 1.1 mmol/g) at rt overnight. The solution was filtered, the resin washed with CH₂Cl₂/MeOH (4:1, 100 mL) and the filtrate concentrated in vacuo. A second purification by silica gel column chromatography with hexane/EtOAc (1:0-2:1) yielded (3) as an off-white solid (385 mg, 64%).

LCMS (ES): Found 204.1 [M+H]⁺.

¹H NMR (300 MHz, DMSO-d₆), δ: 10.27 (s, 1H), 9.03 (d, J=1.5 Hz, 1H), 8.59 (s, 1H), 8.30 (dd, J=2.6, 1.5 Hz, 1H), 8.16 (d, J=2.6 Hz, 1H), 7.82 (s, 1H), 3.94 (s, 3H).

To a solution of (3) (53 mg, 0.26 mmol) in anhydrous DMF (2 mL) was added NaH (60%, 13 mg, 0.31 mmol) at 0° C. The reaction mixture was stirred for 15 min before the addition of (4) (98 mg, 0.31 mmol). It was then stirred for a further 2 h. Additional portions of NaH (60%, 5 mg, 0.13 mmol) and (4) (41 mg, 0.13 mmol) were added and the reaction mixture was stirred at 0° C. for a further 1.5 h. It was then quenched by the addition of H₂O (1 mL), poured into a brine solution (50%, 10 mL) and extracted with EtOAc (3×10 mL). The combined organic extracts were dried over MgSO₄, filtered and concentrated in vacuo. Purification by silica gel column chromatography with hexane/EtOAc (1:0-7:3) yielded (BQ) as a pale yellow solid (60 mg, 53%).

LCMS (ES): Found 436.0 [M+H]⁺.

¹H NMR (300 MHz, DMSO-d₆), δ: 8.88 (d, J=1.3 Hz, 1H), 8.45 (dd, J=2.6, 1.5 Hz, 1H), 8.29 (s, 1H), 8.28 (d, J=2.6 Hz, 1H), 7.92 (s, 1H), 7.76 (d, J=3.8 Hz, 1H), 7.34 (d, J=3.8 Hz, 1H), 5.63 (s, 2H), 3.87 (s, 3H).

¹⁹F NMR (282 MHz, DMSO-d), −64.79 (s, 3F).

### Example BR

### N-(Pyrazin-2-yl)-6-(trifluoromethyl)-N-({5-[5-(trifluoromethyl)-1,2,4-oxadiazol-3-yl]thiophen-2-yl}methyl)pyrazin-2-amine

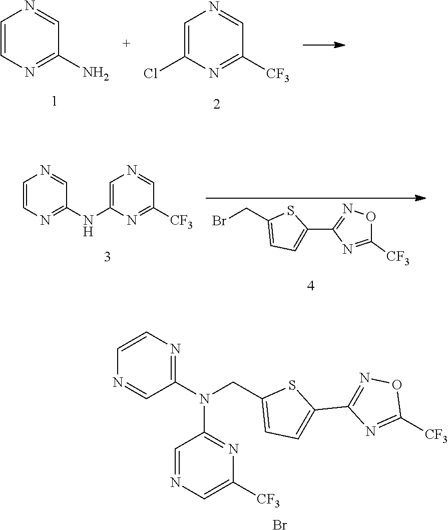 [SMILES: Nc1cnccn1 ; FC(F)(F)c1cncc(Cl)n1 ; FC(F)(F)c1cncc(Nc2cnccn2)n1 ; FC(F)(F)c1nc(-c2ccc(CBr)s2)no1 ; FC(F)(F)c1cncc(N(Cc2ccc(-c3noc(C(F)(F)F)n3)s2)c2cnccn2)n1 ; Br]

A suspension of pyrazin-2-amine (1) (99 mg, 1.05 mmol), 2-chloro-6-trifluoromethylpyrazine (2) (175 mg, 0.95 mmol), Cs₂CO₃ (620 mg, 1.9 mmol), Pd₂(dba)₃ (40 mg, 0.04 mmol) and Xantphos (50 mg, 0.08 mmol) in dioxane (10 mL) was degassed with Ar(g) for 20 min. The reaction mixture was then heated up to 90° C. overnight. Once cooled down to rt, it was partitioned between H₂O (10 mL), brine (5 mL) and EtOAc (35 mL). The combined organics were dried over MgSO₄, filtered and concentrated in vacuo. The residue was dissolved in CH₂Cl₂/MeOH (1:1, 20 mL) and Pd-scavenged with MP-TMT resin (˜150 mg) overnight. The suspension was then filtered and solvent removed in vacuo. Purification by silica gel column chromatography with hexane/EtOAc (1:0-1:1) yielded (3) as a pale red solid (104 mg, 45%).

LCMS (ES): Found 242.1 [M+H]⁺.

¹H NMR (300 MHz, DMSO-d₆), δ: 10.94 (s, 1H), 9.31 (s, 1H), 8.98-9.04 (m, 1H), 8.63 (s, 1H), 8.35-8.39 (m, 1H), 8.27 (d, J=2.6 Hz, 1H).

¹⁹F NMR (282 MHz, DMSO-d₆), δ: −66.83 (s, 3F).

NaH (60%, 9 mg, 0.23 mmol) was added to a solution of (3) (51 mg, 0.21 mmol) in DMF (3 mL) at 0° C. under Ar(g). The reaction mixture was stirred for 20 min then (4) (92 mg, 0.29 mmol) was added as a solution in DMF (1 mL). The reaction mixture was allowed to warm up to rt overnight. It was then partitioned between H₂O (10 mL), brine (10 mL) and EtOAc (4×10 mL). The combined organics were washed with brine (5 mL), dried over MgSO₄, filtered and concentrated in vacuo. Purification by silica gel column chromatography with hexane/EtOAc (1:0-0:1) yielded (BR) as a yellow solid (79 mg, 79%).

LCMS (ES): Found 474.0 [M+H]⁺.

¹H NMR (300 MHz, DMSO-d₆), δ: 9.05 (s, 1H), 8.95-9.00 (m, 1H), 8.70 (s, 1H), 8.47 (dd, J=2.2, 1.2 Hz, 1H), 8.41 (d, J=2.4 Hz, 1H), 7.75 (d, J=3.8 Hz, 1H), 7.35 (d, J=3.8 Hz, 1H), 5.66 (s, 2H).

¹⁹F NMR (282 MHz, DMSO-d₆), δ: −64.80 (s, 3F), −66.77 (s, 3F).

### Example BS

### N,N-Dimethyl-6-[(pyrazin-2-yl)({5-[5-(trifluoromethyl)-1,2,4-oxadiazol-3-yl]thiophen-2-yl}methyl)amino]pyrazine-2-carboxamide

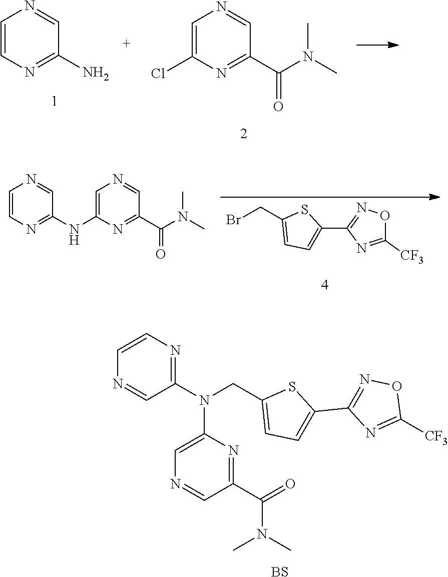

Under Ar(g), to a mixture of pyrazin-2-amine (1) (141 mg, 1.5 mmol), 6-chloro-N,N-dimethylpyrazine-2-carboxamide (2) (250 mg, 1.3 mmol), Cs₂CO₃ (0.88 g, 2.7 mmol) was added degassed dry 1,4-dioxane (13 mL). The reaction mixture was then flushed with Ar(g) for 1 min before Pd₂(dba)₃ (62 mg, 0.07 mmol) and Xantphos (86 mg, 0.15 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (232 mg, 71%).

LCMS (ES): Found 245.2 [M+H]⁺.

NaH (60%, 14 mg, 0.36 mmol) was added to a solution of (3) (73 mg, 0.3 mmol) in dry DMF (1.25 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (BS) as a solid (66 mg, 46%).

LCMS (ES): Found 477.4 [M+H]⁺.

¹H NMR (400 MHz, DMSO-d₆), δ: 8.86 (d, J=1.5 Hz, 1H), 8.85 (s, 1H), 8.44 (dd, J=2.7, 1.5 Hz, 1H), 8.39 (s, 1H), 8.32 (d, J=2.6 Hz, 1H), 7.75 (d, J=3.8 Hz, 1H), 7.32 (d, J=3.8 Hz, 1H), 5.65 (s, 2H), 2.99 (s, 3H), 2.95 (s, 3H).

### Example BT

### 5,6-Dimethyl-N-(pyrazin-2-yl)-N-({5-[5-(trifluoromethyl)-1,2,4-oxadiazol-3-yl]thiophen-2-yl}methyl)pyrazin-2-amine

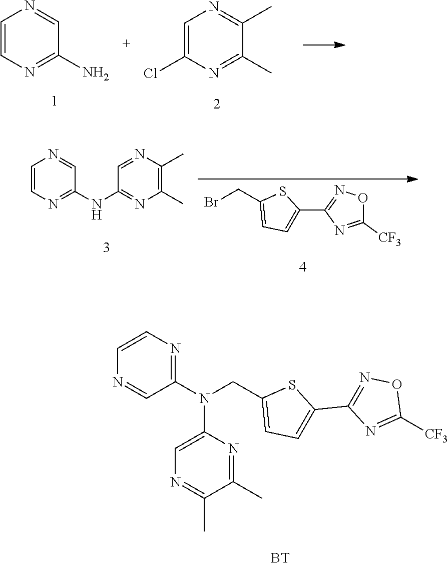

Under Ar(g), to a mixture of pyrazin-2-amine (1) (183 mg, 1.9 mmol), 5-chloro-2,3-dimethylpyrazine (2) (250 mg, 1.8 mmol), Cs₂CO₃ (1.14 g, 3.5 mmol) was added degassed dry 1,4-dioxane (13 mL). The reaction mixture was then flushed with Ar(g) for 1 min before Pd₂(dba)₃ (80 mg, 0.09 mmol) and Xantphos (111 mg, 0.19 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (56 mg, 16%).

NaH (60%, 13 mg, 0.33 mmol) was added to a solution of (3) (55 mg, 0.28 mmol) in dry DMF (1.2 mL). After 5 min, a solution of (4) (103 mg, 0.33 mmol) in dry DMF (1 mL) was added. The reaction mixture was stirred overnight. Water (0.01 mL) was then added to quench the remaining NaH. The reaction mixture was purified by acidic prep LCMS to yield (BT) as a solid (36 mg, 30%).

LCMS (ES): Found 434.4 [M+H]⁺.

¹H NMR (400 MHz, DMSO-d₆), δ: 8.67 (d, J=1.6 Hz, 1H), 8.50 (s, 1H), 8.35 (dd, J=2.7, 1.5 Hz, 1H), 8.18 (d, J=2.7 Hz, 1H), 7.74 (d, J=3.8 Hz, 1H), 7.31 (d, J=3.8 Hz, 1H), 5.58 (s, 2H), 2.45 (s, 6H).

### Example BU

### 3,6-Dimethyl-N-(pyrazin-2-yl)-N-({5-[5-(trifluoromethyl)-1,2,4-oxadiazol-3-yl]thiophen-2-yl}methyl)pyrazin-2-amine

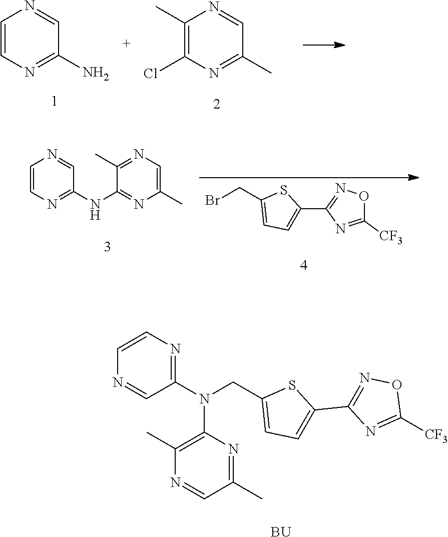

Under Ar(g), to a mixture of pyrazin-2-amine (1) (209 mg, 2.2 mmol), 3-chloro-2,5-dimethylpyrazine (2) (285 mg, 2.0 mmol), Cs₂CO₃ (1.3 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (185 mg, 46%).

LCMS (ES): Found 202.4 [M+H]⁺.

NaH (60%, 24 mg, 0.6 mmol) was added to a solution of (3) (60 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (BU) as a solid (19 mg, 14%).

LCMS (ES): Found 434.4 [M+H]⁺.

¹H NMR (400 MHz, DMSO-d₆), δ: 8.39 (s, 1H), 8.25 (dd, J=2.7, 1.5 Hz, 1H), 8.16 (d, J=1.5 Hz, 1H), 8.09 (d, J=2.6 Hz, 1H), 7.74 (d, J=3.8 Hz, 1H), 7.28 (d, J=3.8 Hz, 1H), 5.46 (s, 2H), 2.52 (s, 3H), 2.12 (s, 3H).

### Example BV

### 3,5-Dimethyl-N-(pyrazin-2-yl)-N-({5-[5-(trifluoromethyl)-1,2,4-oxadiazol-3-yl]thiophen-2-yl}methyl)pyrazin-2-amine

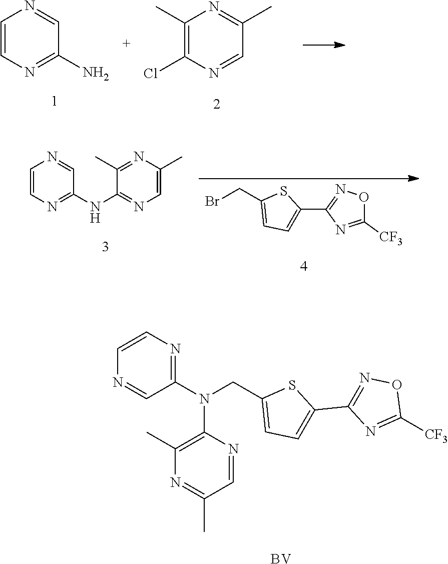

Under Ar(g), to a mixture of pyrazin-2-amine (1) (209 mg, 2.2 mmol), 2-chloro-3,5-dimethylpyrazine (2) (285 mg, 2.0 mmol), Cs₂CO₃ (1.3 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (179 mg, 45%).

LCMS (ES): Found 202.3 [M+H]⁺.

NaH (60%, 24 mg, 0.6 mmol) was added to a solution of (3) (60 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (BV) as a solid (63 mg, 49%).

LCMS (ES): Found 434.4 [M+H]⁺.

¹H NMR (400 MHz, DMSO-d₆), δ: 8.36 (s, 1H), 8.25 (dd, J=2.6, 1.5 Hz, 1H), 8.03-8.09 (m, 2H), 7.74 (d, J=3.7 Hz, 1H), 7.24 (d, J=3.8 Hz, 1H), 5.43 (s, 2H), 2.52 (s, 3H), 2.17 (s, 3H).

### Example BW

### 5,6-Dimethoxy-N-(pyrazin-2-yl)-N-({5-[5-(trifluoromethyl)-1,2,4-oxadiazol-3-yl]thiophen-2-yl}methyl)pyrazin-2-amine

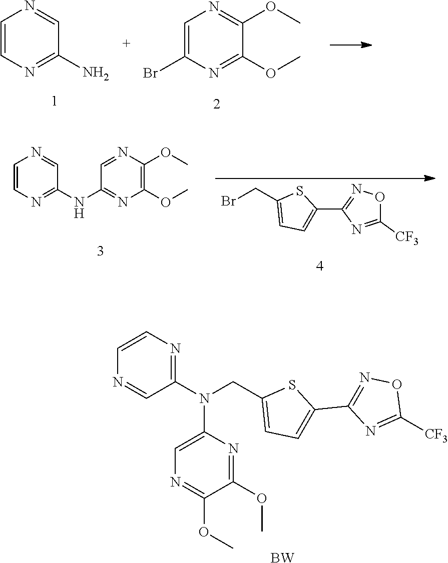

Under Ar(g), to a mixture of pyrazin-2-amine (1) (119 mg, 1.2 mmol), 5-bromo-2,3-dimethoxypyrazine (2) (250 mg, 1.1 mmol), Cs₂CO₃ (0.7 g, 2.2 mmol) was added degassed dry 1,4-dioxane (13 mL). The reaction mixture was then flushed with Ar(g) for 1 min before Pd₂(dba)₃ (52 mg, 0.6 mmol) and Xantphos (73 mg, 0.13 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (157 mg, 59%).

LCMS (ES): Found 234.3 [M+H]⁺.

NaH (60%, 24 mg, 0.6 mmol) was added to a solution of (3) (70 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (BW) as a solid (36 mg, 26%).

LCMS (ES): Found 466.4 [M+H]⁺.

¹H NMR (400 MHz, DMSO-d₆), δ: 8.42 (d, J=1.5 Hz, 1H), 8.30 (dd, J=2.7, 1.5 Hz, 1H), 8.07 (d, J=2.8 Hz, 1H), 7.80 (s, 1H), 7.75 (d, J=3.8 Hz, 1H), 7.26 (d, J=3.8 Hz, 1H), 5.46 (s, 2H), 3.92 (s, 3H), 3.87 (s, 3H).

### Example BX

### 6-Methyl-N-(6-methylpyrazin-2-yl)-N-({5-[5-(trifluoromethyl)-1,2,4-oxadiazol-3-yl]thiophen-2-yl}methyl)pyrazin-2-amine

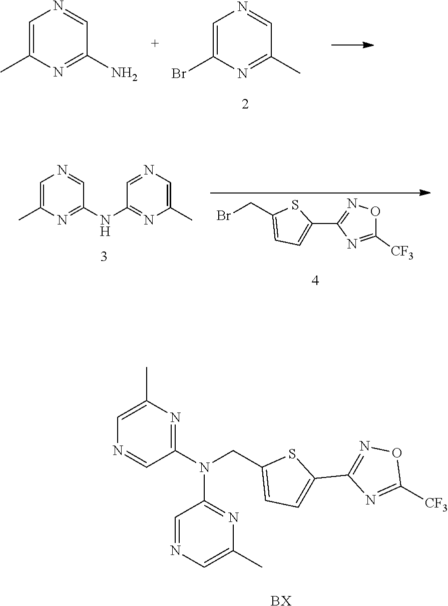

To a solution of (3) (46 mg, 0.23 mmol) in anhydrous DMF (2 mL) was added NaH (60%, 10 mg, 0.25 mmol) at 0° C. The reaction mixture was stirred for 20 min then (4) (87 mg, 0.28 mmol) was added as a solid. It was then allowed to warm up to rt overnight. An additional portion of NaH (1.8 mg, 0.046 mmol) was added at 0° C. The reaction mixture was stirred for 10 min then (4) (14 mg, 0.05 mmol) was added as a solid. It was then allowed to warm up to rt overnight. The reaction mixture was poured into a brine solution (25%, 10 mL) and extracted with EtOAc (3×10 mL). The combined organic extracts were dried over MgSO₄, filtered and concentrated in vacuo. Purification by silica gel column chromatography with CH₂Cl₂/EtOAc (1:0-1:1) yielded (BX) as an off-white solid (35 mg, 35%).

LCMS (ES): Found 433.7 [M+H]⁺.

¹H NMR (300 MHz, DMSO-d₆), δ: 8.57 (s, 2H), 8.15 (s, 2H), 7.74 (d, J=3.8 Hz, 1H), 7.33 (d, J=3.8 Hz, 1H), 5.60 (s, 2H), 2.45 (s, 6H).

¹⁹F NMR (282 MHz, DMSO-d₆), δ: −64.78 (s, 3F).

### Example BY

### 6-Methoxy-N-(6-methoxypyrazin-2-yl)-N-({5-[5-(trifluoromethyl)-1,2,4-oxadiazol-3-yl]thiophen-2-yl}methyl)pyrazin-2-amine

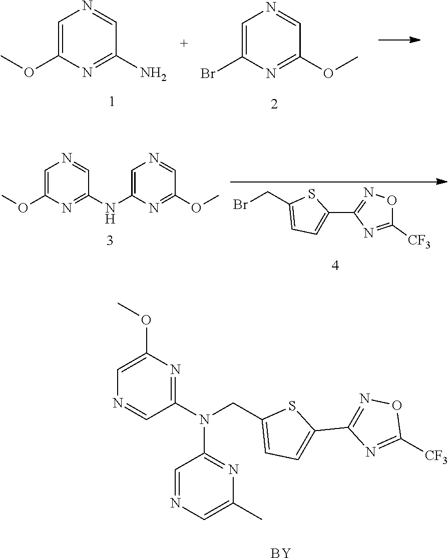

To a solution of (3) (42 mg, 0.18 mmol) in anhydrous DMF (2 mL) was added NaH (60%, 7.8 mg, 0.20 mmol) at 0° C. The reaction mixture was stirred for 0.5 h then (4) (67 mg, 0.21 mmol) was added as a solid. It was then allowed to warm up to rt overnight. The reaction mixture was poured into a brine solution (50%, 10 mL) and extracted with EtOAc (3×10 mL). The combined organic extracts were dried over MgSO₄, filtered and concentrated in vacuo. Purification by silica gel column chromatography with CH₂Cl₂/EtOAc (1:0-4:1) then hexane/EtOAc (1:0-3:1) yielded (BY) as an off-white solid (35 mg, 43%).

LCMS (ES): Found 465.7 [M+H]⁺.

¹H NMR (300 MHz, DMSO-d₆), δ: 8.35 (s, 2H), 7.92 (s, 2H), 7.77 (d, J=3.8 Hz, 1H), 7.37 (d, J=3.8 Hz, 1H), 5.63 (s, 2H), 3.91 (s, 6H).

¹⁹F NMR (282 MHz, DMSO-d₆), δ: −64.79 (s, 3F).

### Example BZ

### N-(Pyrazin-2-yl)-N-({5-[5-(trifluoromethyl)-1,2,4-oxadiazol-3-yl]thiophen-2-yl}methyl)pyrimidin-4-amine

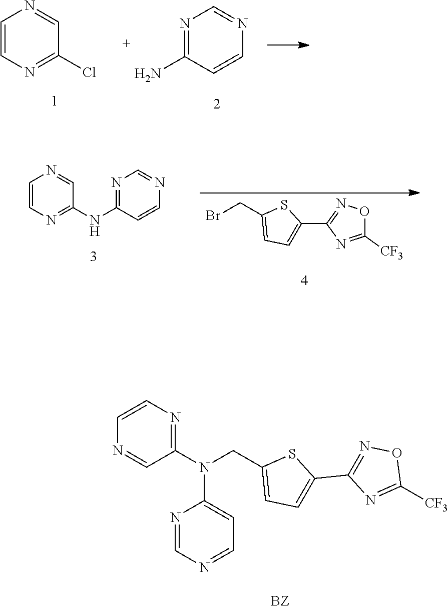 [SMILES: Clc1cnccn1 ; Nc1ccncn1 ; c1cc(Nc2cnccn2)ncn1 ; FC(F)(F)c1nc(-c2ccc(CBr)s2)no1 ; FC(F)(F)c1nc(-c2ccc(CN(c3ccncn3)c3cnccn3)s2)no1 ; "BZ"]

To a solution of (3) (55 mg, 0.315 mmol) in anhydrous DMF (2 mL) was added NaH (60%, 15 mg, 0.378 mmol) at 0° C. The reaction mixture was stirred for 25 min before the addition of (4) (118 mg, 0.378 mmol). It was then allowed to warm up to rt overnight. Once cooled down to 0° C., it was quenched by the addition of H₂O (2 mL). The reaction mixture was poured into brine (10 mL) and extracted with EtOAc (3×10 mL). The combined organic extracts were dried over MgSO₄, filtered and concentrated in vacuo. Purification by silica gel column chromatography with hexane/EtOAc (1:0-6:1) then CH₂Cl₂/EtOAc (1:0-1:1) yielded (BZ) as an off-white solid (60 mg, 47%).

LCMS (ES): Found 405.9 [M+H]⁺.

¹H NMR (300 MHz, DMSO-d₆), δ: 8.94 (d, J=1.3 Hz, 1H), 8.81 (br s, 1H), 8.56 (dd, J=2.6, 1.5 Hz, 1H), 8.48 (d, J=6.0 Hz, 1H), 8.43 (d, J=2.6 Hz, 1H), 7.75 (d, J=3.8 Hz, 1H), 7.31 (d, J=3.8 Hz, 1H), 7.28 (dd, J=6.0, 1.3 Hz, 1H), 5.62 (s, 2H).

¹⁹F NMR (282 MHz, DMSO-d), −64.79 (s, 3F).

### Example CA

### 2-Methyl-N-(pyrazin-2-yl)-N-({5-[5-(trifluoromethyl)-1,2,4-oxadiazol-3-yl]thiophen-2-yl}methyl)pyrimidin-4-amine

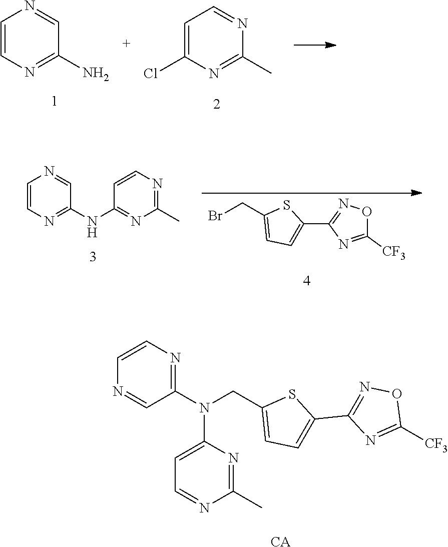

Under Ar(g), to a mixture of pyrazin-2-amine (1) (209 mg, 2.2 mmol), 4-chloro-2-methylpyrimidine (2) (257 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (184 mg, 49%).

LCMS (ES): Found 188.1 [M+H]⁺.

NaH (60%, 14 mg, 0.36 mmol) was added to a solution of (3) (56 mg, 0.3 mmol) in dry DMF (1.25 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (CA) as a solid (28 mg, 23%).

LCMS (ES): Found 420.2 [M+H]⁺.

¹H NMR (400 MHz, DMSO-d₆), δ: 8.94 (d, J=1.5 Hz, 1H), 8.55 (dd, J=2.7, 1.5 Hz, 1H), 8.41 (d, J=2.6 Hz, 1H), 8.38 (d, J=6.0 Hz, 1H), 7.75 (d, J=3.9 Hz, 1H), 7.31 (d, J=3.8 Hz, 1H), 7.09 (d, J=6.0 Hz, 1H), 5.60 (s, 2H), 2.52 (s, 3H).

### Example CB

### 2-Methoxy-N-(pyrazin-2-yl)-N-({5-[5-(trifluoromethyl)-1,2,4-oxadiazol-3-yl]thiophen-2-yl}methyl)pyrimidin-4-amine

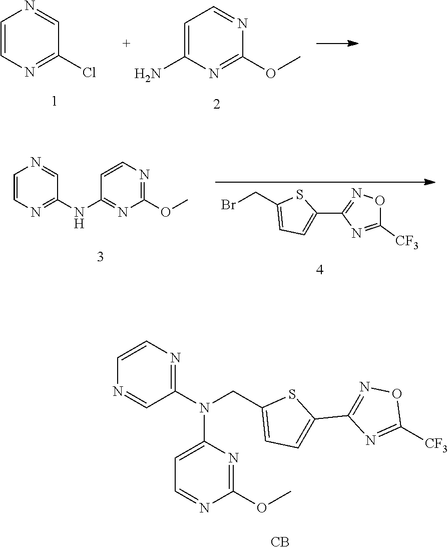

Under Ar(g), to a mixture of 2-chloropyrazine (1) (252 mg, 2.2 mmol), 4-Amino-2-methoxypyrimidine (2) (250 mg, 2.0 mmol), Cs₂CO₃ (1.3 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (45 mg, 11%).

NaH (60%, 11 mg, 0.28 mmol) was added to a solution of (3) (45 mg, 0.22 mmol) in dry DMF (1.3 mL). After 5 min, a solution of (4) (84 mg, 0.27 mmol) in dry DMF (1 mL) was added. The reaction mixture was stirred overnight. Water (0.01 mL) was then added to quench the remaining NaH. The reaction mixture was purified by acidic prep LCMS to yield (CB) as a solid (39 mg, 40%).

LCMS (ES): Found 436.4 [M+H]⁺.

¹H NMR (400 MHz, DMSO-d₆), δ: 8.96 (d, J=1.5 Hz, 1H), 8.58 (dd, J=2.7, 1.5 Hz, 1H), 8.45 (d, J=2.7 Hz, 1H), 8.28 (d, J=5.9 Hz, 1H), 7.76 (d, J=3.7 Hz, 1H), 7.31 (d, J=3.8 Hz, 1H), 6.86 (d, J=5.9 Hz, 1H), 5.59 (s, 2H), 3.85 (s, 3H).

### Example CC

### N2,N2-Dimethyl-N4-(pyrazin-2-yl)-N4-({5-[5-(trifluoromethyl)-1,2,4-oxadiazol-3-yl]thiophen-2-yl}methyl)pyrimidine-2,4-diamine

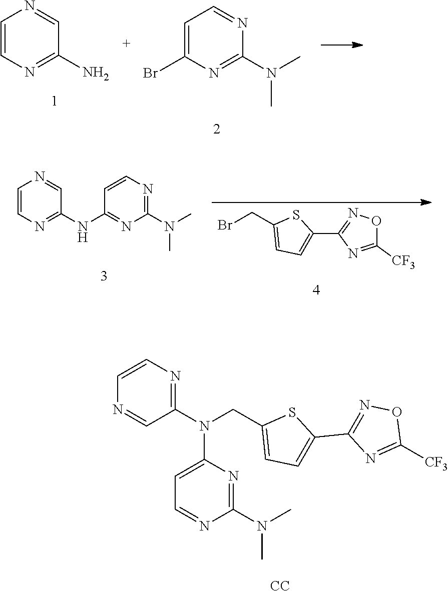

Under Ar(g), to a mixture of pyrazin-2-amine (1) (129 mg, 1.4 mmol), 4-bromo-N,N-dimethylpyrimidin-2-amine (2) (250 mg, 1.2 mmol), Cs₂CO₃ (0.81 g, 2.5 mmol) was added degassed dry 1,4-dioxane (13 mL). The reaction mixture was then flushed with Ar(g) for 1 min before Pd₂(dba)₃ (57 mg, 0.06 mmol) and Xantphos (79 mg, 0.14 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (169 mg, 63%).

LCMS (ES): Found 217.1 [M+H]⁺.

NaH (60%, 24 mg, 0.6 mmol) was added to a solution of (3) (65 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (CC) as a solid (27 mg, 20%).

LCMS (ES): Found 449.4 [M+H]⁺.

¹H NMR (400 MHz, DMSO-d₆), δ: 8.91 (d, J=1.5 Hz, 1H), 8.52 (dd, J=2.6, 1.5 Hz, 1H), 8.35 (d, J=2.6 Hz, 1H), 8.10 (d, J=5.8 Hz, 1H), 7.75 (d, J=3.8 Hz, 1H), 7.28 (d, J=3.8 Hz, 1H), 6.39 (d, J=5.8 Hz, 1H), 5.56 (s, 2H), 3.08 (s, 6H).

### Example CD

### N-(Pyrazin-2-yl)-2-(trifluoromethyl)-N-({5-[5-(trifluoromethyl)-1,2,4-oxadiazol-3-yl]thiophen-2-yl}methyl)pyrimidin-4-amine

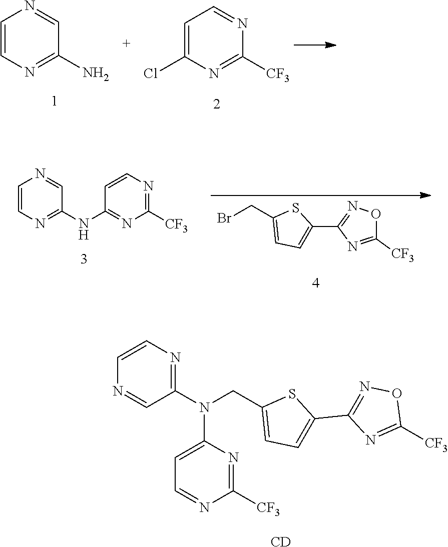

Under Ar(g), to a mixture of pyrazin-2-amine (1) (209 mg, 2.2 mmol), 4-chloro-2-(trifluoromethyl)pyrimidine (2) (365 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (181 mg, 37%).

LCMS (ES): Found 242.1 [M+H]⁺.

Under Ar(g), to a solution of (3) (72 mg, 0.30 mmol) in dry DMF (1.25 mL) was added NaH (60%, 14 mg, 0.36 mmol) in one portion at 0° C. After 5 min, a solution of (4) (112 mg, 0.36 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (CD) as a solid (129 mg, 91%).

LCMS (ES): Found 474.3 [M+H]⁺.

¹H NMR (400 MHz, DMSO-d₆), δ: 9.02 (d, J=1.5 Hz, 1H), 8.61-8.66 (m, 2H), 8.54 (d, J=2.7 Hz, 1H), 7.77 (d, J=3.8 Hz, 1H), 7.46 (d, J=6.1 Hz, 1H), 7.33 (d, J=3.8 Hz, 1H), 5.63 (s, 2H).

### Example CE

### 2-Methanesulfonyl-N-(pyrazin-2-yl)-N-({5-[5-(trifluoromethyl)-1,2,4-oxadiazol-3-yl]thiophen-2-yl}methyl)pyrimidin-4-amine

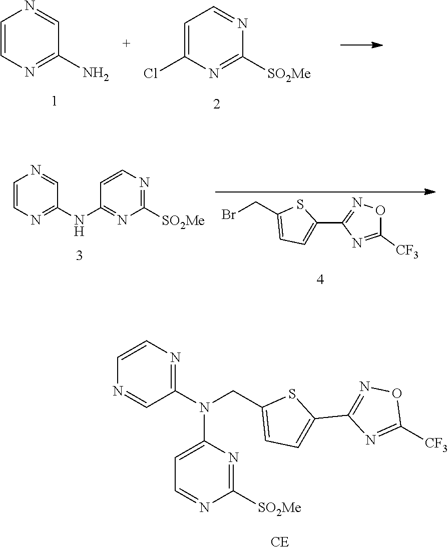

Under Ar(g), to a mixture of pyrazin-2-amine (1) (209 mg, 2.2 mmol), 4-chloro-2-(methylsulfonyl)pyrimidine (2) (385 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (97 mg, 19%).

LCMS (ES): Found 252.1 [M+H]⁺.

Under Ar(g), to a solution of (3) (75 mg, 0.3 mmol) in dry DMF (1.3 mL) was added NaH (60%, 14 mg, 0.35 mmol) in one portion at 0° C. After 5 min, a solution of (4) (112 mg, 0.36 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (CE) as a solid (99 mg, 68%).

LCMS (ES): Found 484.1 [M+H]⁺.

¹H NMR (400 MHz, DMSO-d₆), δ: 9.05 (d, J=1.4 Hz, 1H), 8.61-8.67 (m, 2H), 8.55 (d, J=2.7 Hz, 1H), 7.78 (d, J=3.8 Hz, 1H), 7.46 (d, J=6.1 Hz, 1H), 7.37 (d, J=3.8 Hz, 1H), 5.67 (s, 2H), 3.34 (s, 3H).

### Example CF

### N-{Pyrazolo[1,5-a]pyrimidin-7-yl}-N-({5-[5-(trifluoromethyl)-1,2,4-oxadiazol-3-yl]thiophen-2-yl}methyl)pyrazin-2-amine

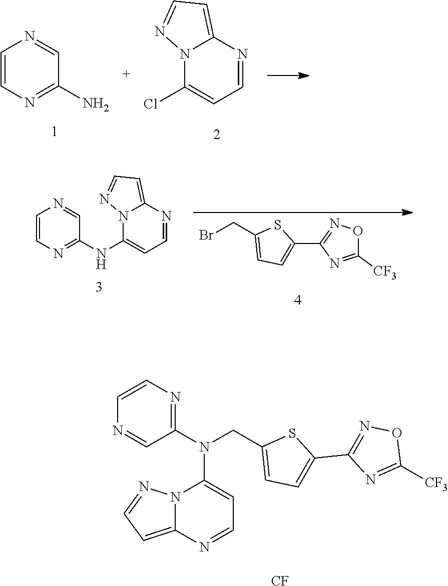

Under Ar(g), to a mixture of pyrazin-2-amine (1) (209 mg, 2.2 mmol), 7-chloropyrazolo[1,5-a]pyrimidine (2) (307 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (34 mg, 8%).

NaH (60%, 7.7 mg, 0.19 mmol) was added to a solution of (3) (34 mg, 0.16 mmol) in dry DMF (0.7 mL). After 5 min, a solution of (4) (60 mg, 0.19 mmol) in dry DMF (0.6 mL) was added. The reaction mixture was stirred overnight. Water (0.01 mL) was then added to quench the remaining NaH. The reaction mixture was purified by acidic prep LCMS to yield (CF) as a solid (14 mg, 20%).

LCMS (ES): Found 445.3 [M+H]⁺.

¹H NMR (400 MHz, DMSO-d₆), δ: 8.54 (d, J=4.7 Hz, 1H), 8.40 (dd, J=2.6, 1.5 Hz, 1H), 8.26 (d, J=2.6 Hz, 1H), 8.16 (d, J=2.3 Hz, 1H), 8.11 (d, J=1.5 Hz, 1H), 7.72 (d, J=3.8 Hz, 1H), 7.22 (d, J=3.8 Hz, 1H), 7.20 (d, J=4.7 Hz, 1H), 6.78 (d, J=2.3 Hz, 1H), 5.76 (s, 2H).

### Example CG

### 6-Methyl-N-(pyrazin-2-yl)-N-({5-[5-(trifluoromethyl)-1,2,4-oxadiazol-3-yl]thiophen-2-yl}methyl)pyrimidin-4-amine

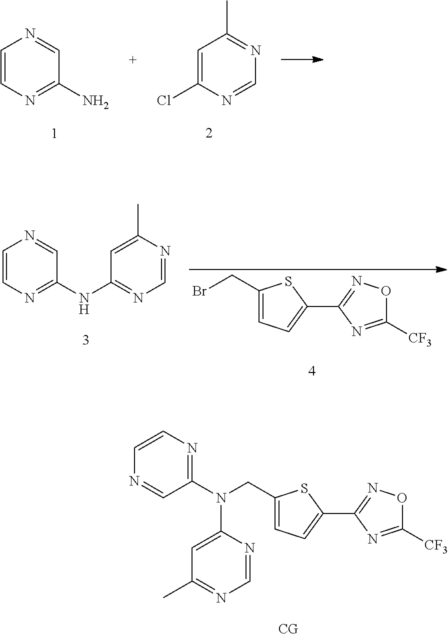

Under Ar(g), to a mixture of pyrazin-2-amine (1) (209 mg, 2.2 mmol), 4-chloro-6-methylpyrimidine (2) (257 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (279 mg, 74%).

LCMS (ES): Found 188.1 [M+H]⁺.

Under Ar(g), to a solution of (3) (51 mg, 0.27 mmol) in dry DMF (1.3 mL) was added NaH (60%, 22 mg, 0.55 mmol) in one portion at 0° C. After 5 min, a solution of (4) (102 mg, 0.33 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (CG) as a solid (39 mg, 34%).

LCMS (ES): Found 420.4 [M+H]⁺.

¹H NMR (400 MHz, DMSO-d₆), δ: 8.91 (d, J=1.5 Hz, 1H), 8.69 (d, J=1.0 Hz, 1H), 8.55 (dd, J=2.6, 1.5 Hz, 1H), 8.41 (d, J=2.7 Hz, 1H), 7.75 (d, J=3.8 Hz, 1H), 7.31 (d, J=3.8 Hz, 1H), 7.18 (s, 1H), 5.60 (s, 2H), 2.36 (s, 3H).

### Example CH

### 6-Methoxy-N-(pyrazin-2-yl)-N-({5-[5-(trifluoromethyl)-1,2,4-oxadiazol-3-yl]thiophen-2-yl}methyl)pyrimidin-4-amine

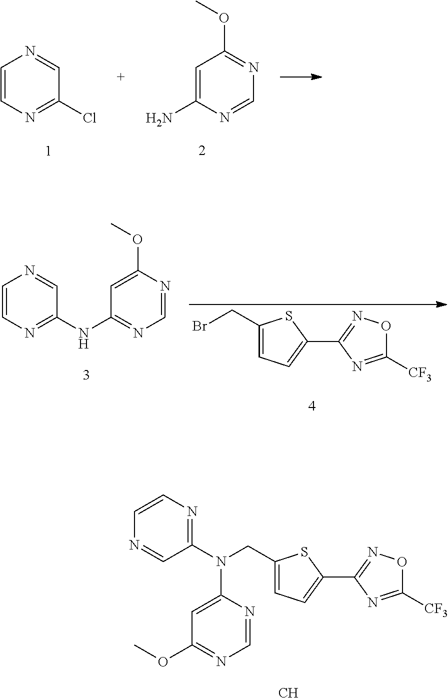

Under Ar(g), to a mixture of 2-chloropyrazine (1) (252 mg, 2.2 mmol), 4-amino-6-methoxypyrimidine (2) (250 mg, 2.0 mmol), Cs₂CO₃ (1.3 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (177 mg, 44%).

LCMS (ES): Found 204.2 [M+H]⁺.

NaH (60%, 24 mg, 0.6 mmol) was added to a solution of (3) (61 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (CH) as a solid (7.1 mg, 5%).

LCMS (ES): Found 436.3 [M+H]⁺.

¹H NMR (400 MHz, DMSO-d₆), δ: 8.90 (d, J=1.5 Hz, 1H), 8.53 (dd, J=2.7, 1.5 Hz, 1H), 8.47-8.51 (m, 1H), 8.37 (d, J=2.7 Hz, 1H), 7.76 (d, J=3.8 Hz, 1H), 7.32 (d, J=3.8 Hz, 1H), 6.65 (d, J=0.9 Hz, 1H), 5.58 (s, 2H), 3.88 (s, 3H).

### Example CI

### N4,N4-Dimethyl-N6-(pyrazin-2-yl)-N6-({5-[5-(trifluoromethyl)-1,2,4-oxadiazol-3-yl]thiophen-2-yl}methyl)pyrimidine-4,6-diamine

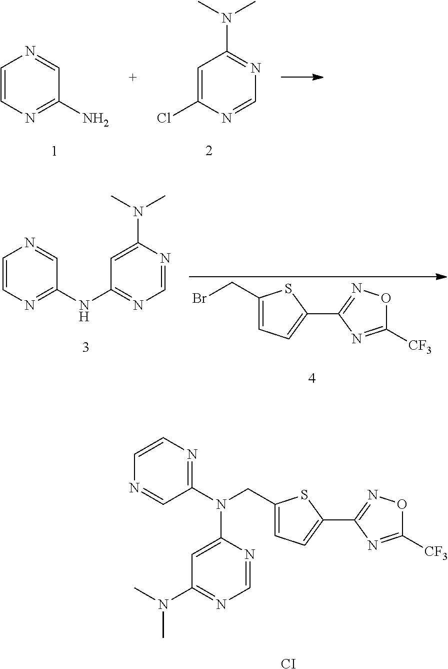

Under Ar(g), to a mixture of pyrazin-2-amine (1) (166 mg, 1.8 mmol), 6-chloro-N,N-dimethylpyrimidin-4-amine (2) (250 mg, 1.6 mmol), Cs₂CO₃ (1.03 g, 3.2 mmol) was added degassed dry 1,4-dioxane (13 mL). The reaction mixture was then flushed with Ar(g) for 1 min before Pd₂(dba)₃ (73 mg, 0.08 mmol) and Xantphos (101 mg, 0.17 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (203 mg, 59%).

LCMS (ES): Found 217.2 [M+H]⁺.

NaH (60%, 24 mg, 0.6 mmol) was added to a solution of (3) (65 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (CI) as a solid (21 mg, 16%).

LCMS (ES): Found 449.4 [M+H]⁺.

¹H NMR (400 MHz, DMSO-d₆), δ: 8.84 (d, J=1.5 Hz, 1H), 8.46 (dd, J=2.7, 1.5 Hz, 1H), 8.26 (d, J=2.7 Hz, 1H), 8.24 (s, 1H), 7.75 (d, J=3.7 Hz, 1H), 7.30 (d, J=3.8 Hz, 1H), 6.33 (s, 1H), 5.58 (s, 2H), 3.03 (s, 6H).

### Example CJ

### N-(Pyrazin-2-yl)-6-(trifluoromethyl)-N-({5-[5-(trifluoromethyl)-1,2,4-oxadiazol-3-yl]thiophen-2-yl}methyl)pyrimidin-4-amine

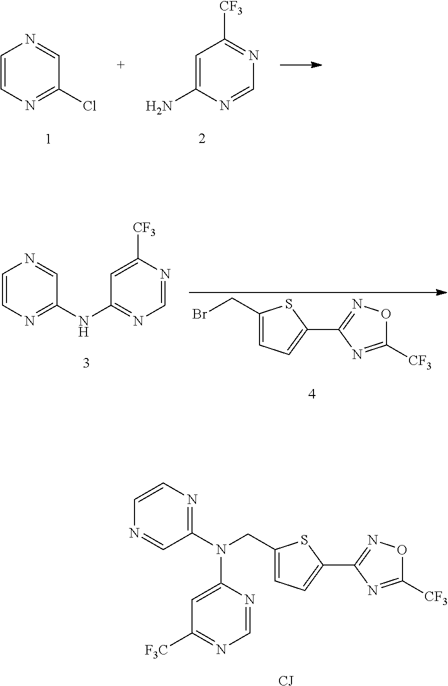

Under Ar(g), to a mixture of 2-chloropyrazine (1) (252 mg, 2.2 mmol), 4-amino-6-(trifluoromethyl)pyrimidine (2) (326 mg, 2.0 mmol), Cs₂CO₃ (1.3 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (314 mg, 65%).

LCMS (ES): Found 242.2 [M+H]⁺.

NaH (60%, 14 mg, 0.36 mmol) was added to a solution of (3) (72 mg, 0.3 mmol) in dry DMF (1.25 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (CJ) as a solid (74 mg, 52%).

LCMS (ES): Found 474.3 [M+H]⁺.

¹H NMR (400 MHz, DMSO-d₆), δ: 9.01 (d, J=1.5 Hz, 1H), 8.98 (s, 1H), 8.61 (dd, J=2.6, 1.4 Hz, 1H), 8.53 (d, J=2.5 Hz, 1H), 7.77 (d, J=3.8 Hz, 1H), 7.67 (d, J=1.1 Hz, 1H), 7.33 (d, J=3.8 Hz, 1H), 5.70 (s, 2H).

### Example CK

### 6-Methanesulfonyl-N-(pyrazin-2-yl)-N-({5-[5-(trifluoromethyl)-1,2,4-oxadiazol-3-yl]thiophen-2-yl}methyl)pyrimidin-4-amine

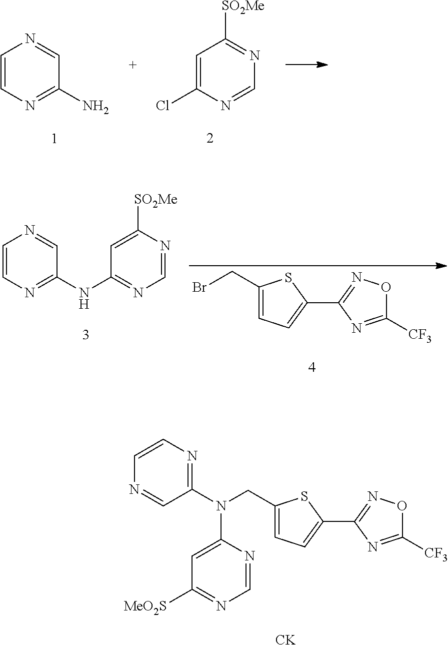

Under Ar(g), to a mixture of pyrazin-2-amine (1) (136 mg, 1.4 mmol), 4-chloro-6-(methylsulfonyl)pyrimidine (2) (250 mg, 1.3 mmol), Cs₂CO₃ (0.85 g, 2.6 mmol) was added degassed dry 1,4-dioxane (13 mL). The reaction mixture was then flushed with Ar(g) for 1 min before Pd₂(dba)₃ (59 mg, 0.06 mmol) and Xantphos (83 mg, 0.14 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (43 mg, 13%).

Under Ar(g), to a solution of (3) (43 mg, 0.17 mmol) in dry DMF (1.3 mL) was added NaH (60%, 8 mg, 0.21 mmol) in one portion at 0° C. After 5 min, a solution of (4) (64 mg, 0.21 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (CK) as a solid (21 mg, 26%).

LCMS (ES): Found 484.3 [M+H]⁺.

¹H NMR (400 MHz, DMSO-d₆), δ: 8.99-9.06 (m, 2H), 8.63 (dd, J=2.6, 1.5 Hz, 1H), 8.57 (d, J=2.6 Hz, 1H), 7.78 (d, J=3.8 Hz, 1H), 7.63 (d, J=1.2 Hz, 1H), 7.32 (d, J=3.8 Hz, 1H), 5.72 (s, 2H), 3.30 (s, 3H).

### Example CL

### 6-(Morpholin-4-yl)-N-(pyrazin-2-yl)-N-({5-[5-(trifluoromethyl)-1,2,4-oxadiazol-3-yl]thiophen-2-yl}methyl)pyrimidin-4-amine

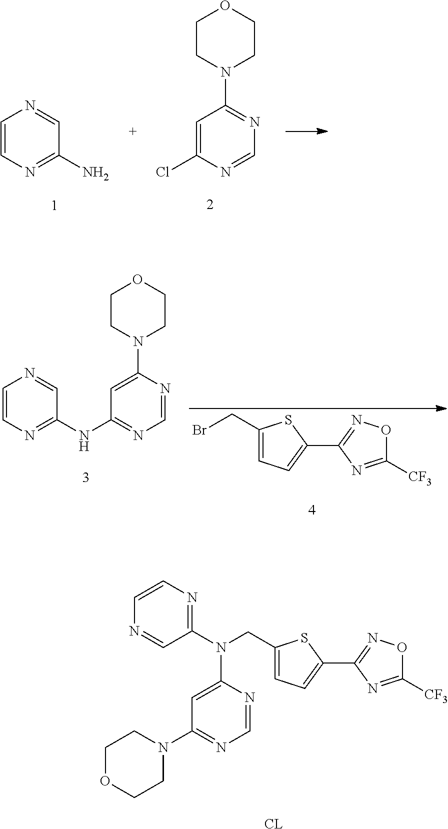

Under Ar(g), to a mixture of pyrazin-2-amine (1) (209 mg, 2.2 mmol), 4-(6-chloro-4-pyrimidinyl)morpholine (2) (399 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (318 mg, 62%).

LCMS (ES): Found 259.2 [M+H]⁺.

NaH (60%, 14 mg, 0.36 mmol) was added to a solution of (3) (77 mg, 0.3 mmol) in dry DMF (1.25 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (CL) as a solid (46 mg, 31%).

LCMS (ES): Found 491.5 [M+H]⁺.

¹H NMR (400 MHz, DMSO-d₆), δ: 8.82 (d, J=1.6 Hz, 1H), 8.47 (dd, J=2.7, 1.4 Hz, 1H), 8.25-8.31 (m, 2H), 7.75 (d, J=3.8 Hz, 1H), 7.30 (d, J=3.8 Hz, 1H), 6.53 (s, 1H), 5.57 (s, 2H), 3.61-3.69 (m, 4H), 3.50-3.59 (m, 4H).

### Example CM

### N-{7-Methyl-7H-pyrrolo[2,3-d]pyrimidin-4-yl}-N-({5-[5-(trifluoromethyl)-1,2,4-oxadiazol-3-yl]thiophen-2-yl}methyl)pyrazin-2-amine

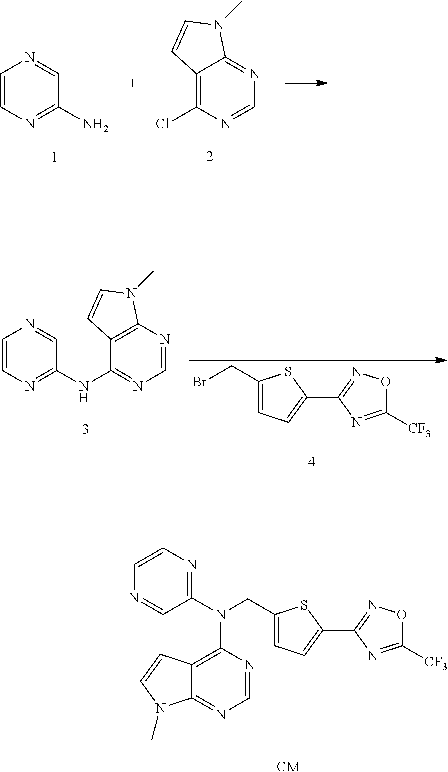

Under Ar(g), to a mixture of pyrazin-2-amine (1) (209 mg, 2.2 mmol), 4-chloro-7-methyl-7H-pyrrolo[2,3-d]pyrimidine (2) (335 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (72 mg, 16%).

Under Ar(g), to a solution of (3) (72 mg, 0.32 mmol) in dry DMF (1.3 mL) was added NaH (60%, 15 mg, 0.38 mmol) in one portion at 0° C. After 5 min, a solution of (4) (119 mg, 0.38 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (CM) as a solid (86 mg, 59%).

LCMS (ES): Found 459.5 [M+H]⁺.

¹H NMR (400 MHz, DMSO-d₆), δ: 8.60 (s, 1H), 8.57 (d, J=1.4 Hz, 1H), 8.53 (dd, J=2.6, 1.5 Hz, 1H), 8.40 (d, J=2.7 Hz, 1H), 7.71 (d, J=3.8 Hz, 1H), 7.34 (d, J=3.6 Hz, 1H), 7.20 (d, J=3.8 Hz, 1H), 5.74 (s, 2H), 5.45 (d, J=3.7 Hz, 1H), 3.78 (s, 3H).

### Example CN

### 6-Methoxy-N-(pyrazin-2-yl)-N-({5-[5-(trifluoromethyl)-1,2,4-oxadiazol-3-yl]thiophen-2-yl}methyl)quinazolin-4-amine

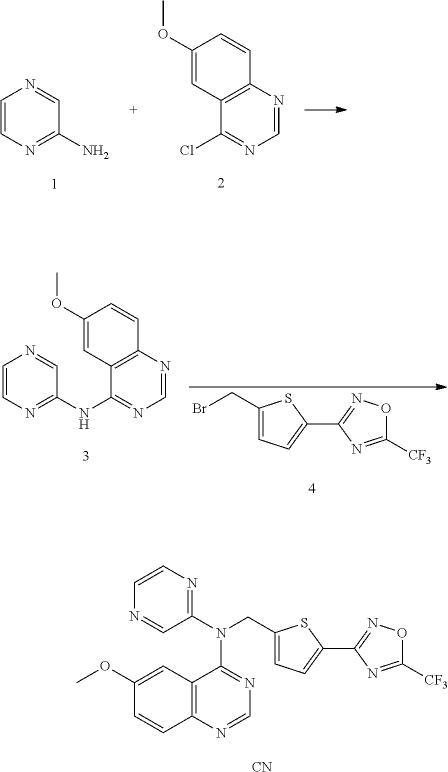

Under Ar(g), to a mixture of pyrazin-2-amine (1) (209 mg, 2.2 mmol), 4-chloro-6-methoxyquinazoline (2) (389 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (194 mg, 38%). LCMS (ES): Found 254.2 [M+H]⁺.

NaH (60%, 24.0 mg, 0.6 mmol) was added to a solution of (3) (76 mg, 0.3 mmol) in dry DMF (1.3 mL). After 5 min, a solution of (4) (112 mg, 0.36 mmol) in dry DMF (1.0 mL) was added. The reaction mixture was stirred overnight. Water (0.01 mL) was then added to quench the remaining NaH. The reaction mixture was purified by acidic prep LCMS to yield (CN) as a solid (27 mg, 19%).

LCMS (ES): Found 486.5 [M+H]⁺.

¹H NMR (400 MHz, DMSO-d₆), δ: 9.03 (s, 1H), 8.49 (d, J=1.5 Hz, 1H), 8.38 (dd, J=2.7, 1.5 Hz, 1H), 8.30 (d, J=2.6 Hz, 1H), 7.94 (d, J=9.2 Hz, 1H), 7.71 (d, J=3.7 Hz, 1H), 7.57 (dd, J=9.1, 2.8 Hz, 1H), 7.27 (d, J=3.8 Hz, 1H), 6.51 (d, J=2.8 Hz, 1H), 5.76 (s, 2H), 3.50 (s, 3H).

### Example CO

### 5-Methyl-N-(pyrazin-2-yl)-N-({5-[5-(trifluoromethyl)-1,2,4-oxadiazol-3-yl]thiophen-2-yl}methyl)pyrimidin-4-amine

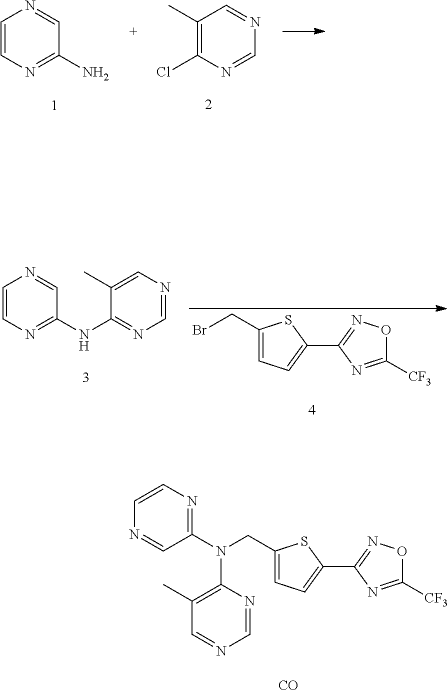

Under Ar(g), to a mixture of pyrazin-2-amine (1) (203 mg, 2.1 mmol), 4-chloro-5-methylpyrimidine (2) (250 mg, 1.9 mmol), Cs₂CO₃ (1.27 g, 3.9 mmol) was added degassed dry 1,4-dioxane (13 mL). The reaction mixture was then flushed with Ar(g) for 1 min before Pd₂(dba)₃ (89 mg, 0.1 mmol) and Xantphos (124 mg, 0.21 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (139 mg, 38%).

LCMS (ES): Found 188.1 [M+H]⁺.

NaH (60%, 24 mg, 0.6 mmol) was added to a solution of (3) (56 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (CO) as a solid (11 mg, 21%).

LCMS (ES): Found 420.3 [M+H]⁺.

¹H NMR (400 MHz, DMSO-d₆), δ: 8.97 (s, 1H), 8.57 (s, 1H), 8.44 (d, J=1.6 Hz, 1H), 8.34 (dd, J=2.7, 1.4 Hz, 1H), 8.23 (d, J=2.7 Hz, 1H), 7.72 (d, J=3.8 Hz, 1H), 7.26 (d, J=3.8 Hz, 1H), 5.60 (s, 2H), 1.79 (s, 3H).

### Example CP

### 5-Methoxy-N-(pyrazin-2-yl)-N-({5-[5-(trifluoromethyl)-1,2,4-oxadiazol-3-yl]thiophen-2-yl}methyl)pyrimidin-4-amine

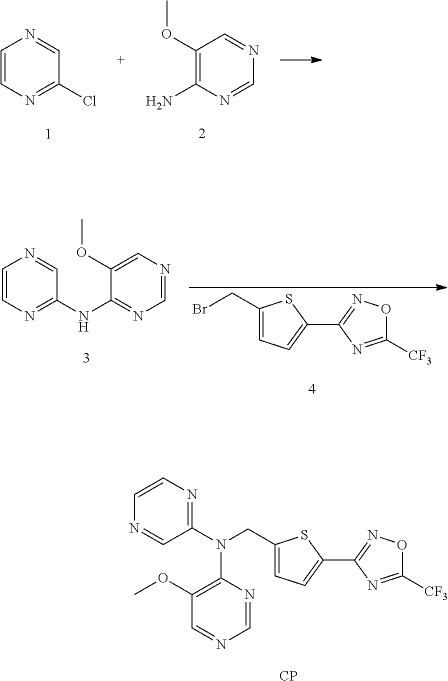

Under Ar(g), to a mixture of 2-chloropyrazine (1) (252 mg, 2.2 mmol), 5-methoxypyrimidin-4-amine (2) (250 mg, 2.0 mmol), Cs₂CO₃ (1.3 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (180 mg, 44%).

LCMS (ES): Found 204.2 [M+H]⁺.

NaH (60%, 11 mg, 0.36 mmol) was added to a solution of (3) (61 mg, 0.3 mmol) in dry DMF (1.25 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (CP) as a solid (55 mg, 42%).

LCMS (ES): Found 436.4 [M+H]⁺.

¹H NMR (400 MHz, DMSO-d₆), δ: 8.67 (s, 1H), 8.49 (s, 1H), 8.35 (dd, J=2.7, 1.5 Hz, 1H), 8.29 (d, J=1.5 Hz, 1H), 8.22 (d, J=2.6 Hz, 1H), 7.71 (d, J=3.7 Hz, 1H), 7.20 (d, J=3.8 Hz, 1H), 5.61 (s, 2H), 3.75 (s, 3H).

### Example CQ

### 2-Methyl-N-(pyrazin-2-yl)-6-(pyrrolidin-1-yl)-N-({5-[5-(trifluoromethyl)-1,2,4-oxadiazol-3-yl]thiophen-2-yl}methyl)pyrimidin-4-amine

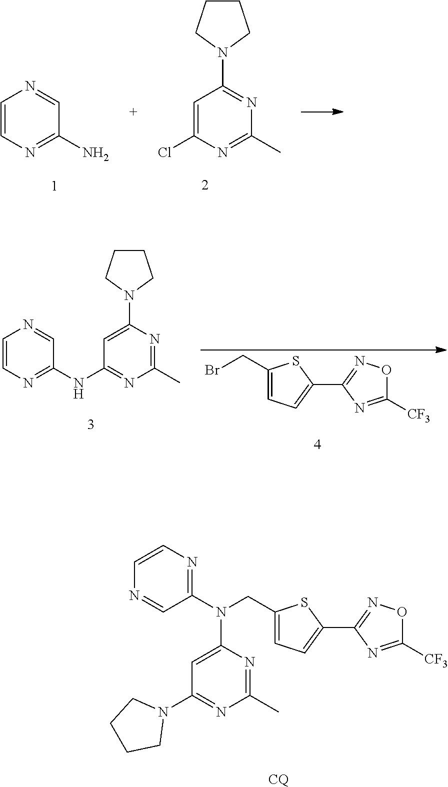

Under Ar(g), to a mixture of pyrazin-2-amine (1) (209 mg, 2.2 mmol), 4-chloro-2-methyl-6-(1-pyrrolidinyl)pyrimidine (2) (395 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (362 mg, 71%).

LCMS (ES): Found 257.4 [M+H]⁺.

Under Ar(g), to a solution of (3) (82 mg, 0.32 mmol) in dry DMF (1.3 mL) was added NaH (60%, 25 mg, 0.64 mmol) in one portion at 0° C. After 5 min, a solution of (4) (119 mg, 0.38 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (CQ) as a solid (40 mg, 26%).

LCMS (ES): Found 489.4 [M+H]⁺.

¹H NMR (400 MHz, DMSO-d₆), δ: 8.86 (d, J=1.5 Hz, 1H), 8.45 (dd, J=2.7, 1.5 Hz, 1H), 8.23 (d, J=2.7 Hz, 1H), 7.75 (d, J=3.8 Hz, 1H), 7.29 (d, J=3.8 Hz, 1H), 6.01 (s, 1H), 5.55 (s, 2H), 3.40 (m 4H), 2.29 (s, 3H), 1.90 (m 4H).

### Example CR

### N-(Pyrazin-2-yl)-N-({5-[5-(trifluoromethyl)-1,2,4-oxadiazol-3-yl]thiophen-2-yl}methyl)pyrimidin-5-amine

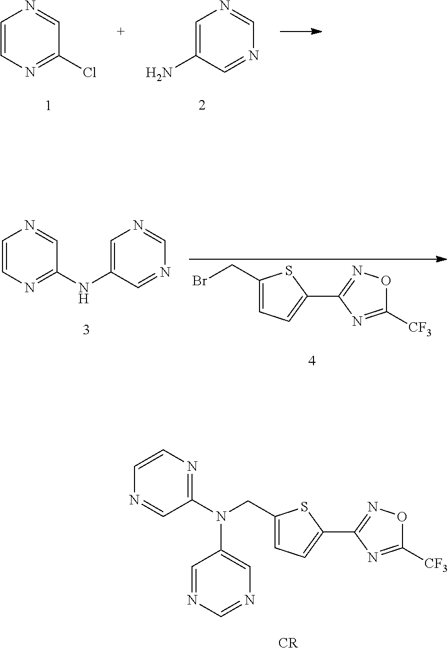

Dioxane (15 mL) was degassed with Ar(g) for 1 h before 5-aminopyrimidine (2) (504 mg, 5.0 mmol), 2-chloropyrazine (1) (0.54 mL, 6.0 mmol), Pd₂(dba)₃ (138 mg, 0.15 mmol), XantPhos (175 mg, 0.30 mmol) and Cs₂CO₃ (3.29 g, 10.1 mmol) were added sequentially with continued degassing. The reaction mixture was degassed with Ar(g) for a further 10 min then heated up to 90° C. overnight. Once cooled down to rt, it was poured into H₂O (25 mL) and extracted with EtOAc (3×35 mL). The combined organic extracts were dried over MgSO₄, filtered, concentrated in vacuo and purified by silica gel column chromatography with CH₂Cl₂/MeOH (1:0-12:1). The residue was re-dissolved in CH₂Cl₂/MeOH (4:1, 50 mL) and swirled with MP-TMT resin (1.2 g, 1.3 mmol/g) at rt overnight. The solution was filtered, the resin washed with CH₂Cl₂/MeOH (4:1, 100 mL) and the filtrate concentrated in vacuo. Purification by silica gel column chromatography with EtOAc/MeOH (1:0-16:1) then reverse-phase column chromatography with H₂O/MeCN (1:0-9:1) yielded (3) as an off-white solid (66 mg, 8%).

LCMS (ES): Found 174.1 [M+H]⁺.

¹H NMR (300 MHz, DMSO-d₆), δ: 9.13 (s, 2H), 8.77 (s, 1H), 8.30 (d, J=1.3 Hz, 1H), 8.20 (dd, J=2.8, 1.3 Hz, 1H), 8.04 (d, J=2.8 Hz, 1H).

To a solution of (3) (43 mg, 0.25 mmol) in anhydrous DMF (2 mL) was added NaH (60%, 12 mg, 0.30 mmol) at 0° C. The reaction mixture was stirred for 20 min before the addition of (4) (93 mg, 0.30 mmol). It was then allowed to warm up to rt over 3 h. Once cooled down to 0° C., it was quenched by the addition of H₂O (1 mL). The reaction mixture was poured into a brine solution (50%, 10 mL) and extracted with EtOAc (3×10 mL). The combined organic extracts were dried over MgSO₄, filtered and concentrated in vacuo. Purification by silica gel column chromatography with hexane/EtOAc (1:0-1:4), CH₂Cl₂/EtOAc (1:0-1:1) and CH₂Cl₂/MeOH (1:0-16:1) yielded (CR) as a white solid (15 mg, 15%).

LCMS (ES): Found 405.9 [M+H]⁺.

¹H NMR (300 MHz, DMSO-d₆), δ: 9.06 (s, 1H), 8.88 (s, 2H), 8.29 (dd, J=2.6, 1.5 Hz, 1H), 8.24 (d, J=1.5 Hz, 1H), 8.12 (d, J=2.6 Hz, 1H), 7.76 (d, J=3.8 Hz, 1H), 7.23 (d, J=3.8 Hz, 1H), 5.50 (s, 2H).

¹⁹F NMR (282 MHz, DMSO-d₆), δ: −64.78 (s, 3F).

### Example CS

### 2-Methyl-N-(pyrazin-2-yl)-N-({5-[5-(trifluoromethyl)-1,2,4-oxadiazol-3-yl]thiophen-2-yl}methyl)pyrimidin-5-amine

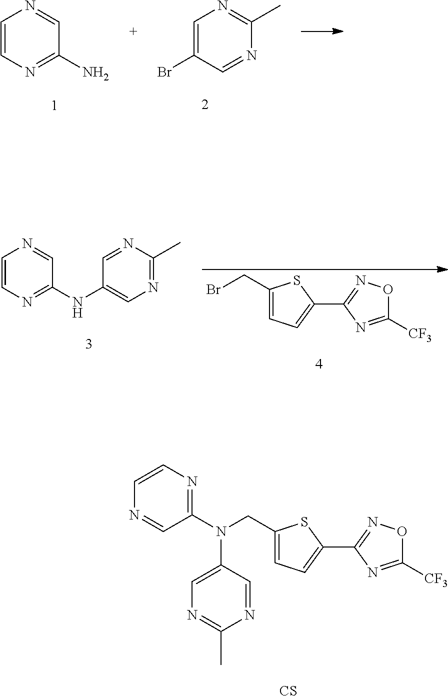

Under Ar(g), to a mixture of pyrazin-2-amine (1) (151 mg, 1.6 mmol), 5-bromo-2-methylpyrimidine (2) (250 mg, 1.4 mmol), Cs₂CO₃ (0.94 g, 2.9 mmol) was added degassed dry 1,4-dioxane (13 mL). The reaction mixture was then flushed with Ar(g) for 1 min before Pd₂(dba)₃ (66 mg, 0.07 mmol) and Xantphos (92 mg, 0.16 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (54 mg, 20%).

NaH (60%, 14 mg, 0.35 mmol) was added to a solution of (3) (54 mg, 0.3 mmol) in dry DMF (1.2 mL). After 5 min, a solution of (4) (109 mg, 0.35 mmol) in dry DMF (1 mL) was added. The reaction mixture was stirred overnight. Water (0.01 mL) was then added to quench the remaining NaH. The reaction mixture was purified by acidic prep LCMS to yield (CS) as a solid (39 mg, 32%).

LCMS (ES): Found 420.4 [M+H]⁺.

¹H NMR (400 MHz, DMSO-d₆), δ: 8.73 (s, 2H), 8.27 (dd, J=2.7, 1.5 Hz, 1H), 8.13 (d, J=1.6 Hz, 1H), 8.08 (d, J=2.8 Hz, 1H), 7.76 (d, J=3.8 Hz, 1H), 7.20 (d, J=3.8 Hz, 1H), 5.44 (s, 2H), 2.64 (s, 3H).

### Example CT

### 2-Methoxy-N-(pyrazin-2-yl)-N-({5-[5-(trifluoromethyl)-1,2,4-oxadiazol-3-yl]thiophen-2-yl}methyl)pyrimidin-5-amine

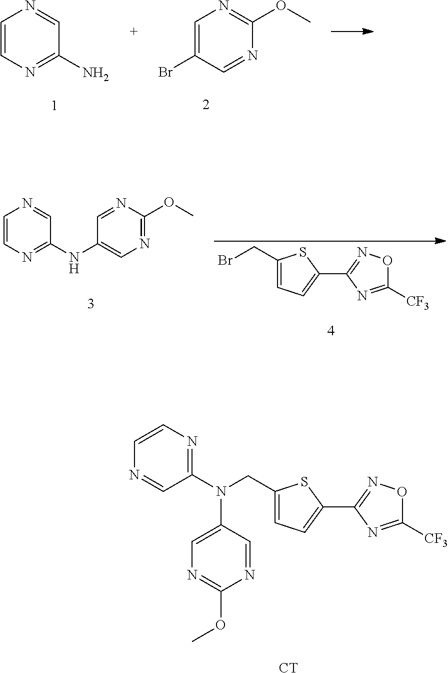

Under Ar(g), to a mixture of pyrazin-2-amine (1) (209 mg, 2.2 mmol), 5-bromo-2-methoxypyrimidine (2) (378 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (18 mg, 4%).

Under Ar(g), to a solution of (3) (18 mg, 0.09 mmol) in dry DMF (0.7 mL) was added NaH (60%, 4.3 mg, 0.11 mmol) in one portion at 0° C. After 5 min, a solution of (4) (34 mg, 0.11 mmol) in dry DMF (0.6 mL) was added dropwise. The reaction mixture was allowed to warm up to rt overnight. Water (0.01 mL) was then added to quench the remaining NaH. The reaction mixture was purified by acidic prep LCMS to yield (CT) as a solid (4 mg, 10%).

LCMS (ES): Found 436.2 [M+H]⁺.

### Example CU 2-Cyclopropyl-N-(pyrazin-2-yl)-N-({5-[5-(trifluoromethyl)-1,2,4-oxadiazol-3-yl]thiophen-2-yl}methyl)pyrimidin-5-amine

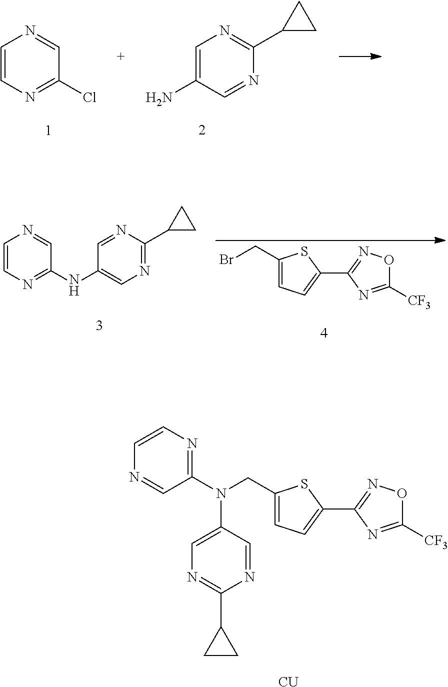

Under Ar(g), to a mixture of 2-chloropyrazine (1) (233 mg, 2.0 mmol), 2-cyclopropylpyrimidin-5-amine (2) (250 mg, 1.8 mmol), Cs₂CO₃ (1.20 g, 3.7 mmol) was added degassed dry 1,4-dioxane (13 mL). The reaction mixture was then flushed with Ar(g) for 1 min before Pd₂(dba)₃ (85 mg, 0.09 mmol) and Xantphos (118 mg, 0.20 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (174 mg, 44%).

LCMS (ES): Found 214.3 [M+H]⁺.

NaH (60%, 24 mg, 0.6 mmol) was added to a solution of (3) (64 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (CU) as a solid (60 mg, 45%).

LCMS (ES): Found 446.4 [M+H]⁺.

¹H NMR (400 MHz, DMSO-d₆), δ: 8.65 (s, 2H), 8.26 (dd, J=2.7, 1.5 Hz, 1H), 8.10 (d, J=1.5 Hz, 1H), 8.06 (d, J=2.7 Hz, 1H), 7.76 (d, J=3.8 Hz, 1H), 7.20 (d, J=3.8 Hz, 1H), 5.41 (s, 2H), 2.17-2.29 (m, 1H), 1.03-1.11 (m, 2H), 0.95-1.03 (m, 2H).

### Example CV

### N-(Pyrazin-2-yl)-2-(trifluoromethyl)-N-({5-[5-(trifluoromethyl)-1,2,4-oxadiazol-3-yl]thiophen-2-yl}methyl)pyrimidin-5-amine

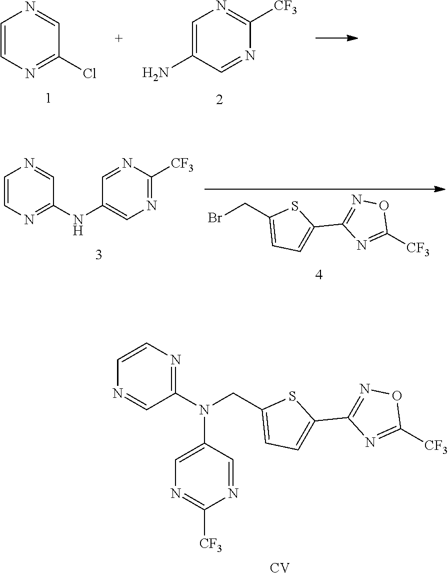 [SMILES: Clc1cnccn1 ; Nc1cnc(C(F)(F)F)nc1 ; FC(F)(F)c1ncc(Nc2cnccn2)cn1 ; FC(F)(F)c1nc(-c2ccc(CBr)s2)no1 ; FC(F)(F)c1ncc(N(Cc2ccc(-c3noc(C(F)(F)F)n3)s2)c2cnccn2)cn1 ; "CV"]

Under Ar(g), to a mixture of 2-chloropyrazine (1) (193 mg, 1.7 mmol), 2-(trifluoromethyl)pyrimidin-5-amine (2) (250 mg, 1.5 mmol), Cs₂CO₃ (0.99 g, 3.0 mmol) was added degassed dry 1,4-dioxane (13 mL). The reaction mixture was then flushed with Ar(g) for 1 min before Pd₂(dba)₃ (70 mg, 0.08 mmol) and Xantphos (98 mg, 0.17 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (17 mg, 5%).

Under Ar(g), to a solution of (3) (17 mg, 0.07 mmol) in dry DMF (0.7 mL) was added NaH (60%, 3.5 mg, 0.09 mmol) in one portion at 0° C. After 5 min, a solution of (4) (27 mg, 0.09 mmol) in dry DMF (0.6 mL) was added dropwise. The reaction mixture was allowed to warm up to rt overnight. Water (0.01 mL) was then added to quench the remaining NaH. The reaction mixture was purified by acidic prep LCMS to yield (CV) as a solid (10 mg, 29%).

LCMS (ES): Found 474.2 [M+H]⁺.

¹H NMR (400 MHz, DMSO-d₆), δ: 9.14 (s, 2H), 8.60 (d, J=1.5 Hz, 1H), 8.37 (dd, J=2.6, 1.4 Hz, 1H), 8.26 (d, J=2.6 Hz, 1H), 7.78 (d, J=3.8 Hz, 1H), 7.31 (d, J=3.8 Hz, 1H), 5.62 (s, 2H).

### Example CW

### 2-Methanesulfonyl-N-(pyrazin-2-yl)-N-({5-[5-(trifluoromethyl)-1,2,4-oxadiazol-3-yl]thiophen-2-yl}methyl)pyrimidin-5-amine

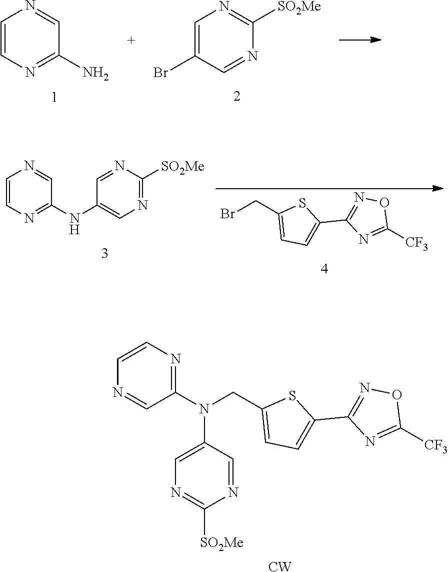 [SMILES: Nc1cnccn1 ; CS(=O)(=O)c1ncc(Br)cn1 ; CS(=O)(=O)c1ncc(Nc2cnccn2)cn1 ; FC(F)(F)c1nc(-c2ccc(CBr)s2)no1 ; CS(=O)(=O)c1ncc(N(Cc2ccc(-c3noc(C(F)(F)F)n3)s2)c2cnccn2)cn1 ; "CW"]

Under Ar(g), to a mixture of pyrazin-2-amine (1) (209 mg, 2.2 mmol), 5-bromo-2-(methylsulfonyl)pyrimidine (2) (474 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (13.5 mg, 3%).

Under Ar(g), to a solution of (3) (13.5 mg, 0.05 mmol) in dry DMF (0.7 mL) was added NaH (60%, 2.6 mg, 0.07 mmol) in one portion at 0° C. After 5 min, a solution of (4) (20 mg, 0.06 mmol) in dry DMF (0.6 mL) was added dropwise. The reaction mixture was allowed to warm up to rt overnight. Water (0.01 mL) was then added to quench the remaining NaH. The reaction mixture was purified by acidic prep LCMS to yield (CW) as a solid (3.1 mg, 12%).

LCMS (ES): Found 484.2 [M+H]⁺.

### Example CX

### 4-Methyl-N-(pyrazin-2-yl)-N-({5-[5-(trifluoromethyl)-1,2,4-oxadiazol-3-yl]thiophen-2-yl}methyl)pyrimidin-5-amine

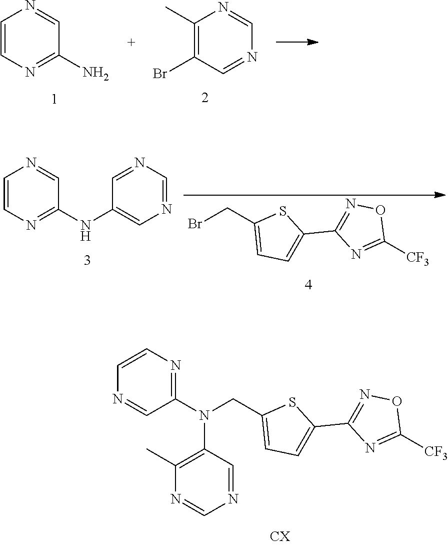

Under Ar(g), to a mixture of pyrazin-2-amine (1) (151 mg, 1.6 mmol), 5-bromo-4-methylpyrimidine (2) (250 mg, 1.4 mmol), Cs₂CO₃ (0.94 g, 2.9 mmol) was added degassed dry 1,4-dioxane (13 mL). The reaction mixture was then flushed with Ar(g) for 1 min before Pd₂(dba)₃ (66 mg, 0.07 mmol) and Xantphos (92 mg, 0.16 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (35 mg, 13%).

NaH (60%, 9 mg, 0.23 mmol) was added to a solution of (3) (35 mg, 0.19 mmol) in dry DMF (0.7 mL). After 5 min, a solution of (4) (70 mg, 0.22 mmol) in dry DMF (0.6 mL) was added. The reaction mixture was stirred overnight. Water (0.01 mL) was then added to quench the remaining NaH. The reaction mixture was purified by acidic prep LCMS to yield (CX) as a solid (19 mg, 25%).

LCMS (ES): Found 420.4 [M+H]⁺.

¹H NMR (400 MHz, DMSO-d₆), δ: 9.06 (s, 1H), 8.58 (s, 1H), 8.30 (dd, J=2.7, 1.5 Hz, 1H), 8.06 (d, J=2.6 Hz, 1H), 7.83 (d, J=1.5 Hz, 1H), 7.77 (d, J=3.8 Hz, 1H), 7.17 (d, J=3.8 Hz, 1H), 5.34 (s, 2H), 2.18 (s, 3H).

### Example CY

### 4-Methoxy-N-(pyrazin-2-yl)-N-({5-[5-(trifluoromethyl)-1,2,4-oxadiazol-3-yl]thiophen-2-yl}methyl)pyrimidin-5-amine

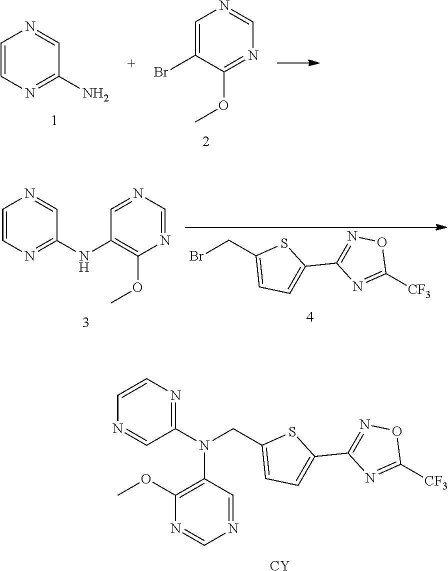

Under Ar(g), to a mixture of pyrazin-2-amine (1) (138 mg, 1.5 mmol), 5-bromo-4-methoxypyrimidine (2) (250 mg, 1.3 mmol), Cs₂CO₃ (0.86 g, 2.6 mmol) was added degassed dry 1,4-dioxane (13 mL). The reaction mixture was then flushed with Ar(g) for 1 min before Pd₂(dba)₃ (61 mg, 0.07 mmol) and Xantphos (84 mg, 0.15 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (102 mg, 38%).

Under Ar(g), to a solution of (3) (61 mg, 0.3 mmol) in dry DMF (1.3 mL) was added NaH (60%, 14 mg, 0.36 mmol) in one portion at 0° C. After 5 min, a solution of (4) (112 mg, 0.36 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (CY) as a solid (69 mg, 53%).

LCMS (ES): Found 436.4 [M+H]⁺.

¹H NMR (400 MHz, DMSO-d₆), δ: 8.80 (s, 1H), 8.54 (s, 1H), 8.25 (dd, J=2.7, 1.6 Hz, 1H), 8.03 (d, J=2.7 Hz, 1H), 7.89 (d, J=1.5 Hz, 1H), 7.75 (d, J=3.7 Hz, 1H), 7.17 (d, J=3.8 Hz, 1H), 5.30 (s, 2H), 3.87 (s, 3H).

### Example CZ

### 4-Cyclopropyl-N-(pyrazin-2-yl)-N-({5-[5-(trifluoromethyl)-1,2,4-oxadiazol-3-yl]thiophen-2-yl}methyl)pyrimidin-5-amine

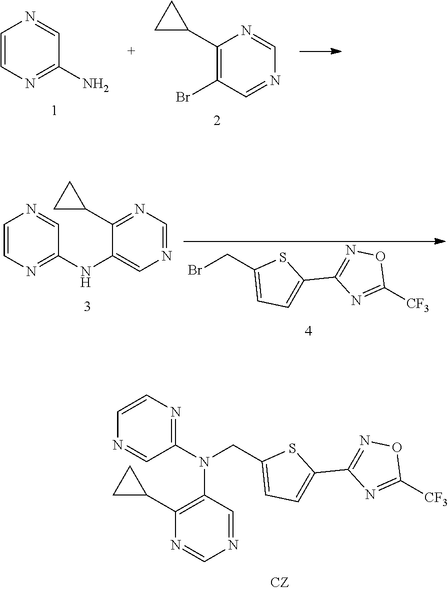

Under Ar(g), to a mixture of pyrazin-2-amine (1) (131 mg, 1.4 mmol), 5-bromo-4-cyclopropylpyrimidine (2) (250 mg, 1.3 mmol), Cs₂CO₃ (0.82 g, 2.5 mmol) was added degassed dry 1,4-dioxane (13 mL). The reaction mixture was then flushed with Ar(g) for 1 min before Pd₂(dba)₃ (58 mg, 0.06 mmol) and Xantphos (80 mg, 0.14 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (104 mg, 39%).

LCMS (ES): Found 214.3 [M+H]⁺.

Under Ar(g), to a solution of (3) (64 mg, 0.3 mmol) in dry DMF (1.3 mL) was added NaH (60%, 14 mg, 0.36 mmol) in one portion at 0° C. After 5 min, a solution of (4) (112 mg, 0.36 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (CZ) as a solid (77 mg, 58%).

LCMS (ES): Found 446.4 [M+H]⁺.

¹H NMR (400 MHz, DMSO-d₆), δ: 8.98 (s, 1H), 8.55 (s, 1H), 8.32 (dd, J=2.8, 1.5 Hz, 1H), 8.06 (d, J=2.8 Hz, 1H), 7.79 (d, J=1.5 Hz, 1H), 7.76 (d, J=3.8 Hz, 1H), 7.18 (d, J=3.8 Hz, 1H), 5.38 (s, 2H), 1.71-1.80 (m, 1H), 0.98-1.06 (m, 2H), 0.83-0.91 (m, 2H).

### Example DA

### N-(Pyrazin-2-yl)-4-(trifluoromethyl)-N-({5-[5-(trifluoromethyl)-1,2,4-oxadiazol-3-yl]thiophen-2-yl}methyl)pyrimidin-5-amine

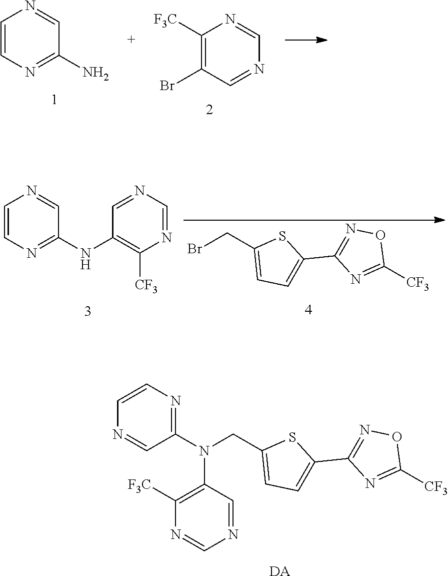

Under Ar(g), to a mixture of pyrazin-2-amine (1) (131 mg, 1.2 mmol), 5-bromo-4-(trifluoromethyl)pyrimidine (2) (250 mg, 1.1 mmol), Cs₂CO₃ (0.72 g, 2.2 mmol) was added degassed dry 1,4-dioxane (13 mL). The reaction mixture was then flushed with Ar(g) for 1 min before Pd₂(dba)₃ (50 mg, 0.05 mmol) and Xantphos (70 mg, 0.12 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (76 mg, 29%).

Under Ar(g), to a solution of (3) (76 mg, 0.31 mmol) in dry DMF (1.3 mL) was added NaH (60%, 15 mg, 0.38 mmol) in one portion at 0° C. After 5 min, a solution of (4) (118 mg, 0.38 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (DA) as a solid (97 mg, 65%).

LCMS (ES): Found 474.4 [M+H]⁺.

¹H NMR (400 MHz, DMSO-d₆), δ: 9.49 (s, 1H), 8.86 (s, 1H), 8.29 (dd, J=2.8, 1.5 Hz, 1H), 8.11 (d, J=2.8 Hz, 1H), 8.04-8.09 (m, 1H), 7.79 (d, J=3.7 Hz, 1H), 7.24 (d, J=3.8 Hz, 1H), 5.34 (s, 2H).

### Example DB

### 2,4-Dimethyl-N-(pyrazin-2-yl)-N-({5-[5-(trifluoromethyl)-1,2,4-oxadiazol-3-yl]thiophen-2-yl}methyl)pyrimidin-5-amine

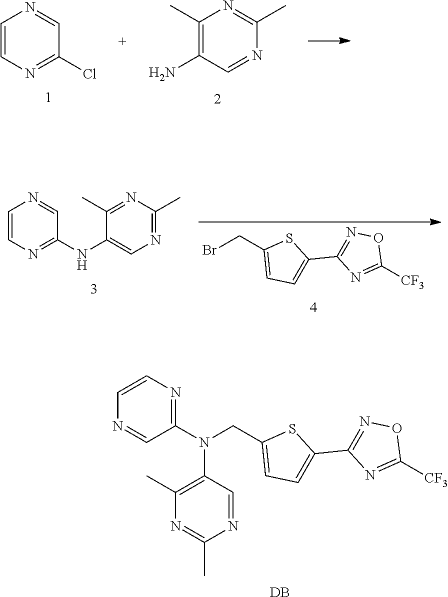

Under Ar(g), to a mixture of 2-chloropyrazine (1) (252 mg, 2.2 mmol), 2,4-dimethyl-5-pyrimidinamine (2) (246 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (92 mg, 23%).

LCMS (ES): Found 202.4 [M+H]⁺.

Under Ar(g), to a solution of (3) (60 mg, 0.3 mmol) in dry DMF (1.3 mL) was added NaH (60%, 14 mg, 0.36 mmol) in one portion at 0° C. After 5 min, a solution of (4) (112 mg, 0.36 mmol) in dry DMF (1.2 mL) was added dropwise. The reaction mixture was allowed to warm up to rt overnight. Water (0.01 mL) was then added to quench the remaining NaH. The reaction mixture was purified by acidic prep LCMS to yield (DB) as a solid (56 mg, 43%).

LCMS (ES): Found 434.4 [M+H]⁺.

¹H NMR (400 MHz, DMSO-d₆), δ: 8.41 (s, 1H), 8.29 (dd, J=2.7, 1.5 Hz, 1H), 8.04 (d, J=2.7 Hz, 1H), 7.74-7.80 (m, 2H), 7.16 (d, J=3.8 Hz, 1H), 5.29 (s, 2H), 2.63 (s, 3H), 2.12 (s, 3H).

### Example DC

### 4,6-Dimethyl-N-(pyrazin-2-yl)-N-({5-[5-(trifluoromethyl)-1,2,4-oxadiazol-3-yl]thiophen-2-yl}methyl)pyrimidin-5-amine

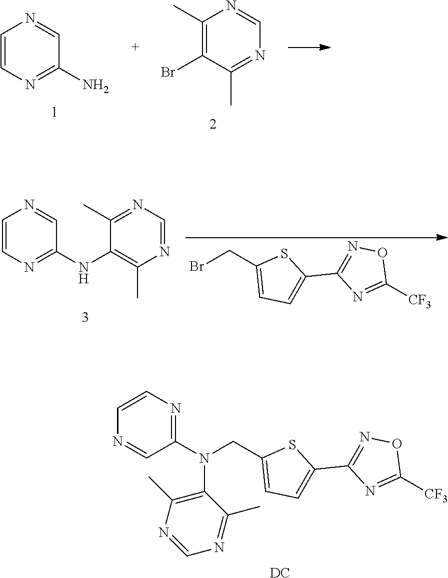

Under Ar(g), to a mixture of pyrazin-2-amine (1) (209 mg, 2.2 mmol), 5-bromo-4,6-dimethylpyrimidine (2) (374 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (43 mg, 11%).

NaH (60%, 10 mg, 0.26 mmol) was added to a solution of (3) (43 mg, 0.2 mmol) in dry DMF (0.7 mL). After 5 min, a solution of (4) (112 mg, 0.26 mmol) in dry DMF (0.6 mL) was added. The reaction mixture was stirred overnight. Water (0.01 mL) was then added to quench the remaining NaH. The reaction mixture was purified by acidic prep LCMS to yield (DC) as a solid (17 mg, 19%).

LCMS (ES): Found 434.4 [M+H]⁺.

¹H NMR (400 MHz, DMSO-d₆), δ: 8.93 (s, 1H), 8.36 (dd, J=2.6, 1.6 Hz, 1H), 8.06 (d, J=2.8 Hz, 1H), 7.78 (d, J=3.8 Hz, 1H), 7.67 (s, 1H), 7.17 (d, J=3.8 Hz, 1H), 5.23 (s, 2H), 2.07 (s, 6H).

### Example DD

### N-(Pyrazin-2-yl)-N-({5-[5-(trifluoromethyl)-1,2,4-oxadiazol-3-yl]thiophen-2-yl}methyl)pyridazin-3-amine

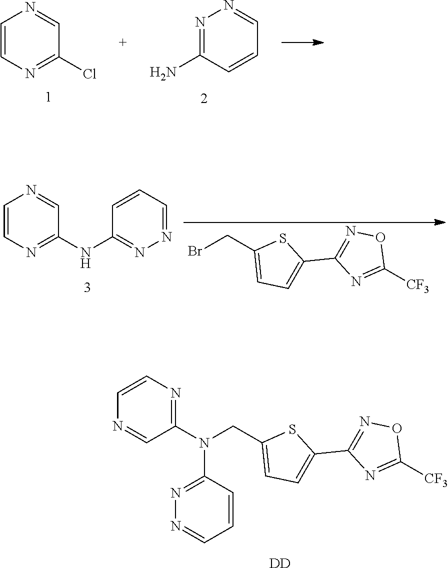

Under Ar(g), to a mixture of 2-chloropyrazine (1) (252 mg, 2.2 mmol), 3-aminopyridazine (2) (190 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (14 mg, 4%).

Under Ar(g), to a solution of (3) (14 mg, 0.08 mmol) in dry DMF (0.7 mL) was added NaH (60%, 3.9 mg, 0.1 mmol) in one portion at 0° C. After 5 min, a solution of (4) (30 mg, 0.1 mmol) in dry DMF (0.6 mL) was added dropwise. The reaction mixture was allowed to warm up to rt overnight. Water (0.01 mL) was then added to quench the remaining NaH. The reaction mixture was purified by acidic prep LCMS to yield (DD) as a solid (2.1 mg, 6%).

LCMS (ES): Found 406.2 [M+H]⁺.

### Example DE

### N-(Pyrazin-2-yl)-N-({5-[5-(trifluoromethyl)-1,2,4-oxadiazol-3-yl]thiophen-2-yl}methyl)-1,2,4-triazin-3-amine

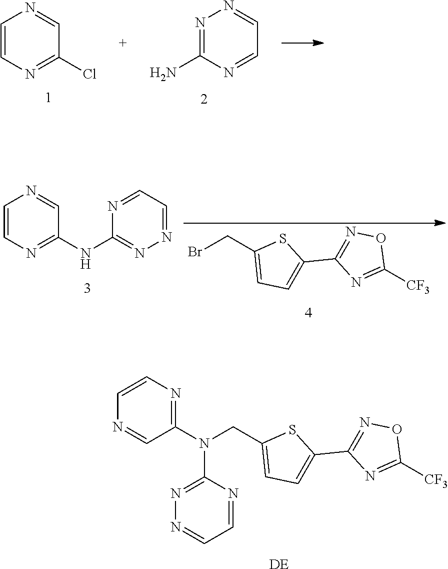

To degassed dioxane (10 mL) were added sequentially 3-amino-1,2,4-triazine (2) (205 mg, 2.1 mmol), 2-chloropyrazine (1) (0.23 mL, 2.6 mmol), Pd₂(dba)₃ (59 mg, 0.06 mmol), XantPhos (74 mg, 0.13 mmol) and Cs₂CO₃ (1.4 g, 4.3 mmol) with continued degassing. The reaction mixture was heated up to 90° C. overnight. Once cooled down to rt, it was poured into a brine solution (50%, 20 mL) and extracted with EtOAc (3×20 mL). The combined organic extracts were dried over MgSO₄, filtered and concentrated in vacuo. Purification by silica gel column chromatography with EtOAc/MeOH (1:0-0:1) afforded a residue. It was re-dissolved in CH₂Cl₂/MeOH (4:1, 50 mL) and swirled with MP-TMT resin (500 mg, 1.3 mmol/g) at rt overnight. The solution was filtered, the resin washed with CH₂Cl₂/MeOH (4:1, 100 mL) and the filtrate concentrated in vacuo to yield (3) as an orange solid (69 mg, 18%).

LCMS (ES): Found 175.0 [M+H]⁺.

¹H NMR (300 MHz, DMSO-d₆), δ: 10.86 (s, 1H), 9.38 (d, J=1.5 Hz, 1H), 8.99 (d, J=2.3 Hz, 1H), 8.61 (d, J=2.3 Hz, 1H), 8.40 (dd, J=2.5, 1.6 Hz, 1H), 8.30 (d, J=2.6 Hz, 1H).

To a solution of (3) (35 mg, 0.20 mmol) in anhydrous DMF (2 mL) was added KO^{t}Bu (1M in THF, 0.24 mL) at 0° C. The solution was stirred for 20 min before the addition of (4) (70 mg, 0.22 mmol) and allowed to warm up to rt over 1.5 h. It was then poured into a brine solution (50%, 10 mL) and extracted with EtOAc (3×10 mL). The combined organic extracts were dried over MgSO₄, filtered and concentrated in vacuo. Purification by silica gel column chromatography with hexane/EtOAc (1:0-0:1) yielded (DE) as an orange solid (47 mg, 57%).

LCMS (ES): Found 406.8 [M+H]⁺.

¹H NMR (300 MHz, DMSO-d₆), δ: 9.06 (d, J=2.3 Hz, 2H), 8.63 (d, J=2.3 Hz, 1H), 8.58 (dd, J=2.7, 1.5 Hz, 1H), 8.41 (d, J=2.6 Hz, 1H), 7.75 (d, J=3.8 Hz, 1H), 7.29 (d, J=3.8 Hz, 1H), 5.74 (s, 2H).

¹⁹F NMR (282 MHz, DMSO-d₆), −64.78 (s, 3F).

### Example DF

### 6-Methyl-N-(pyrazin-2-yl)-N-({5-[5-(trifluoromethyl)-1,2,4-oxadiazol-3-yl]thiophen-2-yl}methyl)pyridazin-3-amine

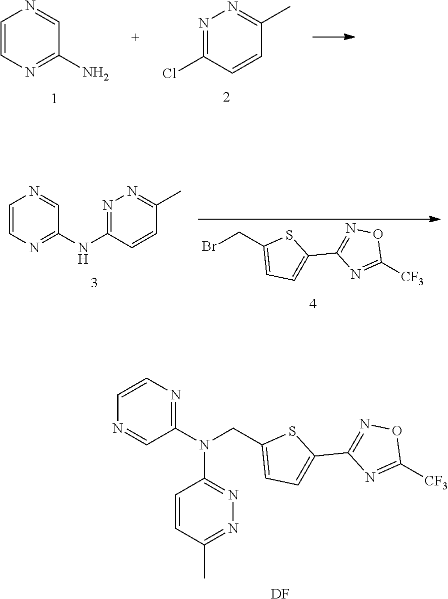

Under Ar(g), to a mixture of pyrazin-2-amine (1) (209 mg, 2.2 mmol), 3-chloro-6-methylpyridazine (2) (257 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (45 mg, 12%).

NaH (60%, 11 mg, 0.28 mmol) was added to a solution of (3) (45 mg, 0.24 mmol) in dry DMF (0.7 mL). After 5 min, a solution of (4) (90 mg, 0.29 mmol) in dry DMF (0.6 mL) was added. The reaction mixture was stirred overnight. Water (0.01 mL) was then added to quench the remaining NaH. The reaction mixture was purified by acidic prep LCMS to yield (DF) as a solid (15 mg, 15%).

LCMS (ES): Found 420.4 [M+H]⁺.

¹H NMR (400 MHz, DMSO-d₆), δ: 8.30 (d, J=1.5 Hz, 1H), 8.25 (dd, J=2.7, 1.4 Hz, 1H), 8.11 (d, J=2.7 Hz, 1H), 8.08 (d, J=9.7 Hz, 1H), 7.80 (d, J=3.8 Hz, 1H), 7.43 (d, J=3.8 Hz, 1H), 7.25 (d, J=9.8 Hz, 1H), 5.66 (s, 2H), 2.28 (s, 3H).

### Example DG

### 6-Methoxy-N-(pyrazin-2-yl)-N-({5-[5-(trifluoromethyl)-1,2,4-oxadiazol-3-yl]thiophen-2-yl}methyl)pyridazin-3-amine

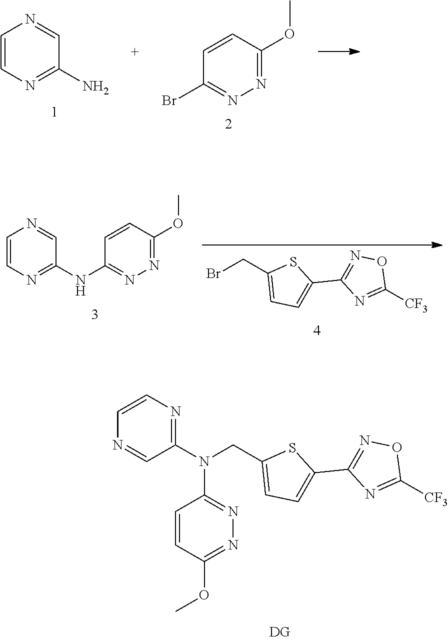

Under Ar(g), to a mixture of pyrazin-2-amine (1) (209 mg, 2.2 mmol), 3-bromo-6-methoxypyridazine (2) (378 mg, 1.4 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (5.4 mg, 1%).

Under Ar(g), to a solution of (3) (5.4 mg, 0.027 mmol) in dry DMF (1.3 mL) was added NaH (60%, 1.3 mg, 0.03 mmol) in one portion at 0° C. After 5 min, a solution of (4) (10 mg, 0.03 mmol) in dry DMF (1.2 mL) was added dropwise. The reaction mixture was allowed to warm up to rt overnight. The reaction mixture was purified by acidic prep LCMS to yield (DG) as a solid (6.5 mg, 56%).

LCMS (ES): Found 436.2 [M+H]⁺.

¹H NMR (400 MHz, DMSO-d₆), δ: 8.58 (d, J=1.5 Hz, 1H), 8.30 (dd, J=2.6, 1.5 Hz, 1H), 8.18 (d, J=2.6 Hz, 1H), 7.77 (d, J=9.4 Hz, 1H), 7.74 (d, J=3.8 Hz, 1H), 7.29 (d, J=3.8 Hz, 1H), 7.24 (d, J=9.4 Hz, 1H), 5.63 (s, 2H), 4.02 (s, 3H).

¹⁹F NMR (282 MHz, DMSO-d₆), δ: −64.79 (s, 3F).

### Example DH

### N-(Pyrazin-2-yl)-6-(trifluoromethyl)-N-({5-[5-(trifluoromethyl)-1,2,4-oxadiazol-3-yl]thiophen-2-yl}methyl)pyridazin-3-amine

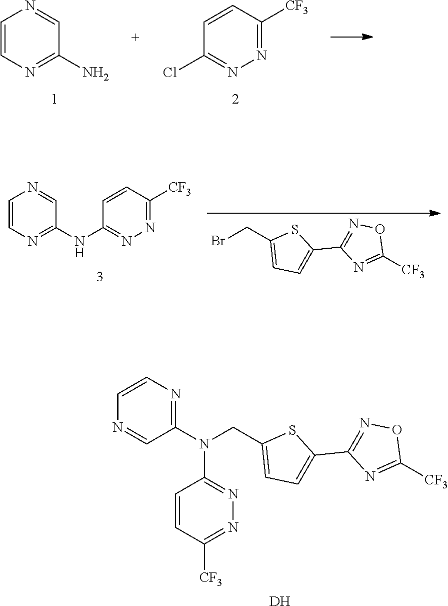 [SMILES: Nc1cnccn1 ; FC(F)(F)c1ccc(Cl)nn1 ; FC(F)(F)c1ccc(Nc2cnccn2)nn1 ; FC(F)(F)c1nc(-c2ccc(CBr)s2)no1 ; FC(F)(F)c1ccc(N(Cc2ccc(-c3noc(C(F)(F)F)n3)s2)c2cnccn2)nn1 ; "DH"]

Under Ar(g), to a mixture of pyrazin-2-amine (1) (143 mg, 1.5 mmol), 3-chloro-6-trifluoromethylpyridazine (2) (250 mg, 1.4 mmol), Cs₂CO₃ (0.89 g, 2.7 mmol) was added degassed dry 1,4-dioxane (13 mL). The reaction mixture was then flushed with Ar(g) for 1 min before Pd₂(dba)₃ (63 mg, 0.07 mmol) and Xantphos (87 mg, 0.15 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (211 mg, 64%).

LCMS (ES): Found 242.1 [M+H]⁺.

NaH (60%, 14.4 mg, 0.36 mmol) was added to a solution of (3) (72 mg, 0.3 mmol) in dry DMF (1.3 mL). After 5 min, a solution of (4) (112 mg, 0.36 mmol) in dry DMF (1 mL) was added. The reaction mixture was stirred overnight. Water (0.01 mL) was then added to quench the remaining NaH. The reaction mixture was purified by acidic prep LCMS to yield (DH) as a solid (28 mg, 20%).

LCMS (ES): Found 474.3 [M+H]⁺.

¹H NMR (400 MHz, DMSO-d₆), δ: 8.94 (d, J=1.5 Hz, 1H), 8.50 (dd, J=2.6, 1.6 Hz, 1H), 8.43 (d, J=2.5 Hz, 1H), 8.07 (d, J=9.4 Hz, 1H), 7.92 (d, J=9.5 Hz, 1H), 7.76 (d, J=3.8 Hz, 1H), 7.33 (d, J=3.8 Hz, 1H), 5.79 (s, 2H).

### Example DI

### N-(Pyrazin-2-yl)-6-(pyrrolidin-1-yl)-N-({5-[5-(trifluoromethyl)-1,2,4-oxadiazol-3-yl]thiophen-2-yl}methyl)pyridazin-3-amine

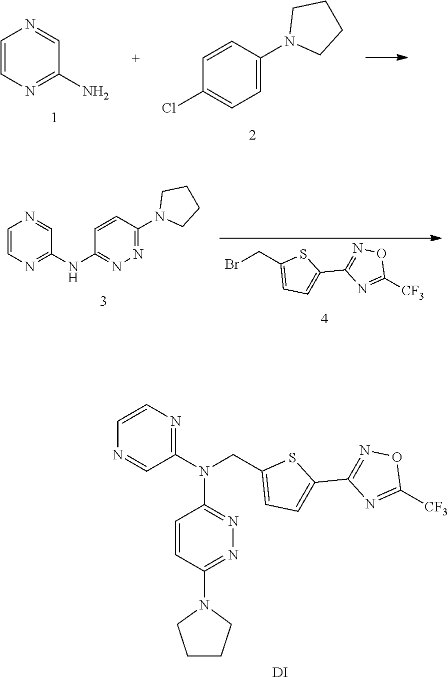

Under Ar(g), to a mixture of pyrazin-2-amine (1) (209 mg, 2.2 mmol), 3-chloro-6-(pyrrolidin-1-yl)pyridazine (2) (367.3 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (71 mg, 15%).

LCMS (ES): Found 243.0 [M+H]⁺.

Under Ar(g), to a solution of (3) (71 mg, 0.29 mmol) in dry DMF (1.3 mL) was added NaH (60%, 14 mg, 0.35 mmol) in one portion at 0° C. After 5 min, a solution of (4) (109 mg, 0.35 mmol) in dry DMF (1.2 mL) was added dropwise. The reaction mixture was allowed to warm up to rt overnight. The reaction mixture was purified by acidic prep LCMS to yield (DI) as a solid (4 mg, 3%).

LCMS (ES): Found 475.4 [M+H]⁺.

¹H NMR (400 MHz, DMSO-d₆), δ: 8.23-8.28 (m, 2H), 8.05 (d, J=2.7 Hz, 1H), 7.74 (d, J=3.6 Hz, 1H), 7.51 (d, J=9.6 Hz, 1H), 7.24 (d, J=3.8 Hz, 1H), 6.98 (d, J=9.6 Hz, 1H), 5.52 (s, 2H), 3.43-3.51 (m, 4H), 1.93-2.01 (m, 4H).

### Example DJ

### 6-(Morpholin-4-yl)-N-(pyrazin-2-yl)-N-({5-[5-(trifluoromethyl)-1,2,4-oxadiazol-3-yl]thiophen-2-yl}methyl)pyridazin-3-amine

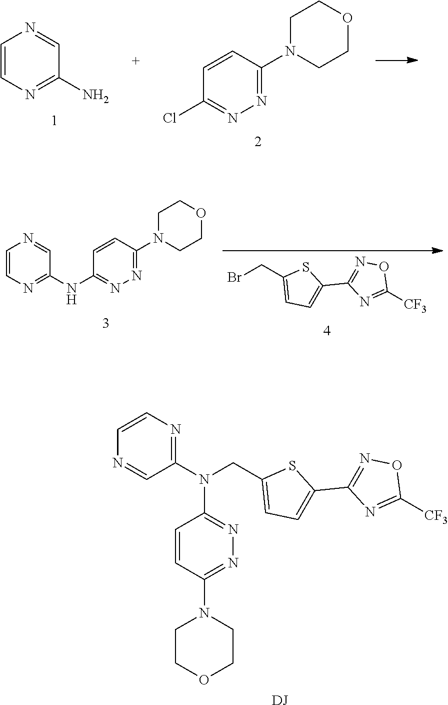 [SMILES: Nc1cnccn1 ; Clc1ccc(N2CCOCC2)nn1 ; c1cnc(Nc2ccc(N3CCOCC3)nn2)cn1 ; FC(F)(F)c1nc(-c2ccc(CBr)s2)no1 ; FC(F)(F)c1nc(-c2ccc(CN(c3cnccn3)c3ccc(N4CCOCC4)nn3)s2)no1 ; "DJ"]

Under Ar(g), to a mixture of pyrazin-2-amine (1) (209 mg, 2.2 mmol), 3-chloro-6-morpholinopyridazine (2) (399 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (10.2 mg, 2%).

Under Ar(g), to a solution of (3) (10.2 mg, 0.04 mmol) in dry DMF (0.7 mL) was added NaH (60%, 1.9 mg, 0.05 mmol) in one portion at 0° C. After 5 min, a solution of (4) (14.8 mg, 0.05 mmol) in dry DMF (0.6 mL) was added dropwise. The reaction mixture was allowed to warm up to rt overnight. The reaction mixture was purified by acidic prep LCMS to yield (DJ) as a solid (4.6 mg, 24%).

LCMS (ES): Found 491.3 [M+H]⁺.

¹H NMR (400 MHz, DMSO-d₆), δ: 8.41 (d, J=1.5 Hz, 1H), 8.27 (dd, J=1.6 Hz, 1H), 8.10 (d, J=2.7 Hz, 1H), 7.74 (d, J=3.7 Hz, 1H), 7.61 (d, J=9.7 Hz, 1H), 7.38 (d, J=9.8 Hz, 1H), 7.26 (d, J=3.8 Hz, 1H), 5.57 (s, 2H), 3.74 (m, 4H), 3.53 (m, 4H).

### Example DK

### 6-(4-Methylpiperazin-1-yl)-N-(pyrazin-2-yl)-N-({5-[5-(trifluoromethyl)-1,2,4-oxadiazol-3-yl]thiophen-2-yl}methyl)pyridazin-3-amine

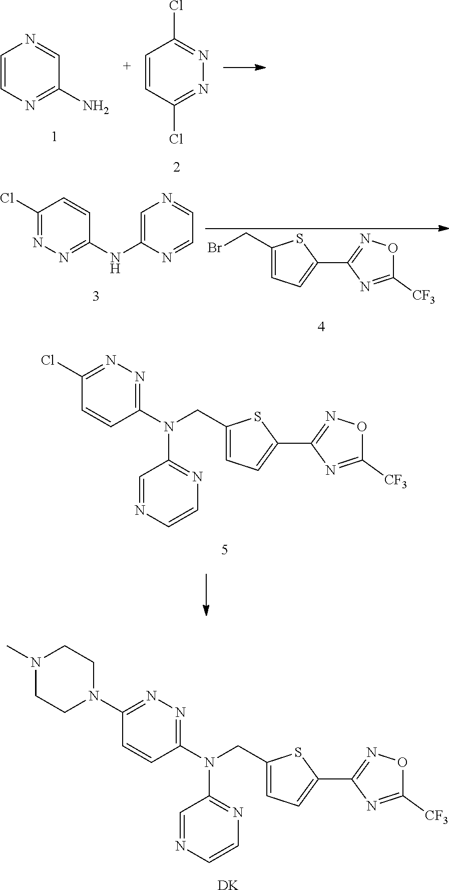

A suspension of pyrazin-2-amine (1) (1.0 g, 10.5 mmol), 3,6-dichloropyridazine (2) (1.72 g, 11.6 mmol), Cs₂CO₃ (6.9 g, 21 mmol) and Xantphos (610 mg, 1.05 mmol) in 1,4-dioxane (30 mL) was purged with Ar(g) for 0.5 h. Pd₂(dba)₃ (245 mg, 0.53 mmol) was added and the reaction mixture was heated up to 90° C. overnight. It was then cooled down to rt and partitioned between EtOAc (100 mL) and H₂O (100 mL). The solids were isolated by filtration and re-slurried in hot EtOAc. The reaction mixture was filtered to yield (3) as a green solid (1.35 g, 62%).

NaH (60%, 185 mg, 4.62 mmol) was added to a suspension of (3) (800 mg, 3.85 mmol) in DMF (16 mL) under Ar(g). The reaction mixture was stirred for 20 min, cooled down to 0° C. and (4) (1.45 g, 4.62 mmol) was added in one portion. It was then allowed to warm up to rt overnight, poured into a mixture of H₂O (30 mL) and brine (30 mL), then extracted with EtOAc (4×25 mL). The combined organics were dried over MgSO₄, filtered and concentrated in vacuo. Purification by silica gel column chromatography with hexane/EtOAc (9:1-3:7) yielded (5) as a yellow solid (540 mg, 32%).

A solution of (5) (80 mg, 0.18 mmol) in 1,4-dioxane (2.4 mL) was treated with N-methylpiperazine (0.08 mL, 0.73 mmol) and heated in a microwave reactor (150° C., 7×1 h). The reaction mixture was poured into H₂O (10 mL) and extracted with CH₂Cl₂ (3×10 mL). The combined organics were extracted with HCl solution (5%, 3×10 mL) and the combined acidic aqueous was subsequently basified with NaOH solution (5%, 40 mL) before extraction with EtOAc (3×15 mL). The combined organics were washed sequentially with NaOH solution (5%, 2×10 mL) and brine (10 mL), dried over MgSO₄, filtered and concentrated in vacuo. Purification by reverse-phase column chromatography with H₂O/MeCN (4:1-1:3) yielded (DK) as a brown film (8.6 mg, 9%).

LCMS (ES): Found 503.9 [M+H]⁺.

¹H NMR (300 MHz, DMSO-d₆), δ: 8.38 (d, J=1.3 Hz, 1H), 8.26 (dd, J=2.6, 1.5 Hz, 1H), 8.09 (d, J=2.6 Hz, 1H), 7.73 (d, J=3.8 Hz, 1H), 7.56 (d, J=9.8 Hz, 1H), 7.37 (d, J=9.8 Hz, 1H), 7.26 (d, J=3.8 Hz, 1H), 5.56 (s, 2H), 3.51-3.61 (m, 4H), 2.40-2.47 (m, 4H), 2.24 (s, 3H).

¹⁹F NMR (282 MHz, DMSO-d₆), δ: −64.79 (s, 3F).

### Example DL

### 6-Methanesulfonyl-N-(pyrazin-2-yl)-N-({5-[5-(trifluoromethyl)-1,2,4-oxadiazol-3-yl]thiophen-2-yl}methyl)pyridazin-3-amine

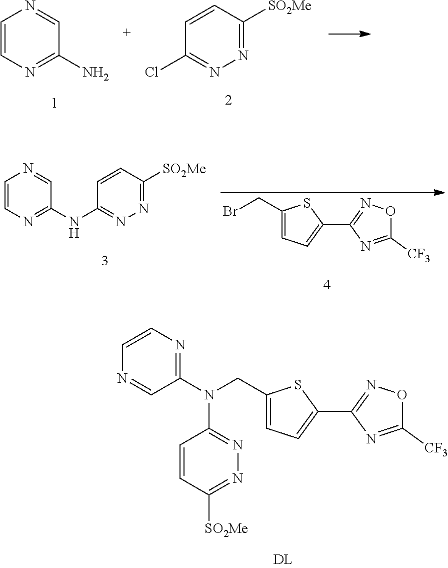 [SMILES: Nc1cnccn1 ; CS(=O)(=O)c1ccc(Cl)nn1 ; CS(=O)(=O)c1ccc(Nc2cnccn2)nn1 ; FC(F)(F)c1nc(-c2ccc(CBr)s2)no1 ; CS(=O)(=O)c1ccc(N(Cc2ccc(-c3noc(C(F)(F)F)n3)s2)c2cnccn2)nn1]

Under Ar(g), to a mixture of pyrazin-2-amine (1) (209 mg, 2.2 mmol), 3-chloro-6-(methylsulfonyl)pyridazine (2) (385.2 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (86 mg, 17%).

LCMS (ES): Found 252.3 [M+H]⁺.

Under Ar(g), to a solution of (3) (75 mg, 0.3 mmol) in dry DMF (1.3 mL) was added NaH (60%, 14.4 mg, 0.36 mmol) in one portion at 0° C. After 5 min, a solution of (4) (112 mg, 0.36 mmol) in dry DMF (1.2 mL) was added dropwise. The reaction mixture was allowed to warm up to rt overnight. The reaction mixture was purified by acidic prep LCMS to yield (DL) as a solid (46 mg, 32%).

LCMS (ES): Found 484.3 [M+H]⁺.

¹H NMR (400 MHz, DMSO-d₆), δ: 8.96 (d, J=1.4 Hz, 1H), 8.52 (dd, J=2.5, 1.5 Hz, 1H), 8.46 (d, J=2.6 Hz, 1H), 8.09 (d, J=9.4 Hz, 1H), 7.92 (d, J=9.5 Hz, 1H), 7.76 (d, J=3.8 Hz, 1H), 7.34 (d, J=3.8 Hz, 1H), 5.79 (s, 2H), 3.44 (s, 3H).

### Example DM

### N-(Pyrazin-2-yl)-6-(1H-pyrazol-1-yl)-N-({5-[5-(trifluoromethyl)-1,2,4-oxadiazol-3-yl]thiophen-2-yl}methyl)pyridazin-3-amine

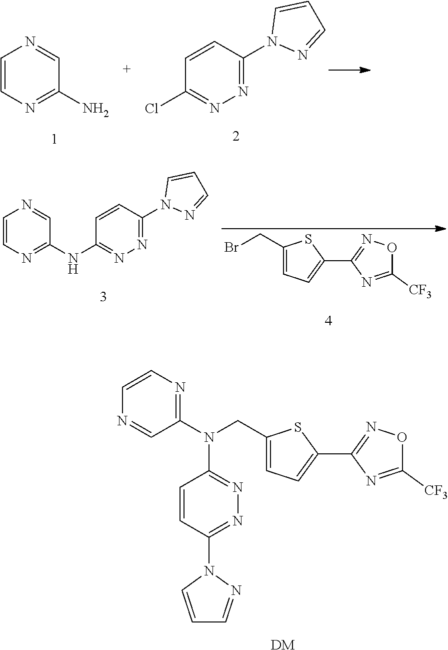

Under Ar(g), to a mixture of pyrazin-2-amine (1) (209 mg, 2.2 mmol), 3-chloro-6-(1H-pyrazol-1-yl)pyridazine (2) (361.2 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (29 mg, 6%).

NaH (60%, 5.8 mg, 0.14 mmol) was added to a solution of (3) (29 mg, 0.12 mmol) in dry DMF (0.7 mL). After 5 min, a solution of (4) (45 mg, 0.14 mmol) in dry DMF (0.6 mL) was added. The reaction mixture was stirred overnight. Water (0.01 mL) was then added to quench the remaining NaH. The reaction mixture was purified by acidic prep LCMS to yield (DM) as a solid (9.9 mg, 17%).

LCMS (ES): Found 472.3 [M+H]⁺.

¹H NMR (400 MHz, DMSO-d₆), δ: 8.80 (dd, J=3.8, 2.0 Hz, 2H), 8.41 (dd, J=2.7, 1.5 Hz, 1H), 8.30 (d, J=2.7 Hz, 1H), 8.16 (d, J=9.5 Hz, 1H), 8.00 (d, J=9.6 Hz, 1H), 7.92 (d, J=1.7 Hz, 1H), 7.75 (d, J=3.8 Hz, 1H), 7.33 (d, J=3.8 Hz, 1H), 6.67 (t, J=2.2 Hz, 1H), 5.74 (s, 2H).

### Example DN

### 6-Phenyl-N-(pyrazin-2-yl)-N-({5-[5-(trifluoromethyl)-1,2,4-oxadiazol-3-yl]thiophen-2-yl}methyl)pyridazin-3-amine

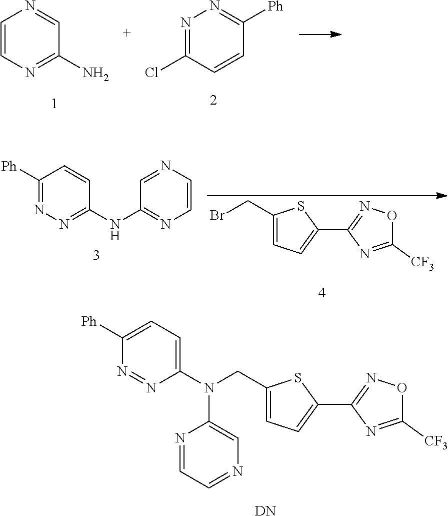

A suspension of 2-aminopyrazine (1) (55 mg, 0.58 mmol), 6-chloro-3-phenylpyridazine (2) (100 mg, 0.53 mmol), Cs₂CO₃ (341 mg, 1.05 mmol) and Xantphos (30 mg, 0.05 mmol) in 1,4-dioxane (3 mL) was purged with Ar(g) for 20 min. Pd₂(dba)₃ (24 mg, 0.03 mmol) was added and the reaction mixture was heated up to 90° C. overnight. Once cooled down to rt, it was poured into a mixture of H₂O (20 mL) and brine (10 mL), then extracted with EtOAc (2×20 mL) and CH₂Cl₂ (3×20 mL). The combined organics were dried over MgSO₄, filtered and concentrated in vacuo. Purification by trituration with EtOAc (5 mL) yielded (3) as a solid (40 mg, 31%).

KO^{t}Bu (1M in THF, 0.17 mL) was added to a solution of (3) (34 mg, 0.14 mmol) in DMF (1.4 mL) at 0° C. under Ar(g). The reaction mixture was stirred for 10 min then (4) (46 mg, 0.15 mmol) was added in one portion. It was allowed to warm up to rt over 3 h, poured into a mixture of H₂O (10 mL) and brine (10 mL), then extracted with EtOAc (2×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-1:1) yielded (DN) as a brown solid (29 mg, 24%).

LCMS (ES): Found 481.8 [M+H]⁺.

¹H NMR (300 MHz, DMSO-d₆), δ: 8.81 (d, J=1.5 Hz, 1H), 8.42 (dd, J=2.5, 1.4 Hz, 1H), 8.30 (d, J=2.6 Hz, 1H), 8.10-8.21 (m, 3H), 7.87 (d, J=9.4 Hz, 1H), 7.75 (d, J=3.8 Hz, 1H), 7.46-7.59 (m, 3H), 7.34 (d, J=4.0 Hz, 1H), 5.78 (s, 2H).

¹⁹F NMR (282 MHz, DMSO-d₆), δ: −64.80 (s, 3F).

### Example DO

### 6-(1-Methyl-1H-pyrazol-4-yl)-N-(pyrazin-2-yl)-N-({5-[5-(trifluoromethyl)-1,2,4-oxadiazol-3-yl]thiophen-2-yl}methyl)pyridazin-3-amine

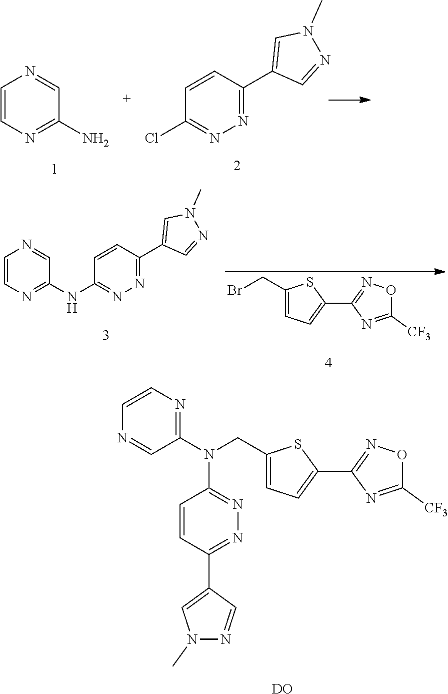 [SMILES: Nc1cnccn1 ; Cn1cc(-c2ccc(Cl)nn2)cn1 ; Cn1cc(-c2ccc(Nc3cnccn3)nn2)cn1 ; FC(F)(F)c1nc(-c2ccc(CBr)s2)no1 ; Cn1cc(-c2ccc(N(Cc3ccc(-c4noc(C(F)(F)F)n4)s3)c3cnccn3)nn2)cn1 ; "DO"]

Under Ar(g), to a mixture of pyrazin-2-amine (1) (134 mg, 1.4 mmol), 3-chloro-6-(1-methyl-1H-pyrazol-4-yl)pyridazine (2) (250 mg, 1.3 mmol), Cs₂CO₃ (0.84 g, 2.6 mmol) was added degassed dry 1,4-dioxane (13 mL). The reaction mixture was then flushed with Ar(g) for 1 min before Pd₂(dba)₃ (59 mg, 0.06 mmol) and Xantphos (82 mg, 0.14 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (7.4 mg, 2%).

Under Ar(g), to a solution of (3) (7.4 mg, 0.03 mmol) in dry DMF (0.7 mL) was added NaH (60%, 1.4 mg, 0.03 mmol) in one portion at 0° C. After 5 min, a solution of (4) (10.9 mg, 0.03 mmol) in dry DMF (0.6 mL) was added dropwise. The reaction mixture was allowed to warm up to rt overnight. The reaction mixture was purified by acidic prep LCMS to yield (DO) as a solid (3.4 mg, 24%).

LCMS (ES): Found 486.3 [M+H]⁺.

¹H NMR (400 MHz, DMSO-d₆), δ: 8.72 (d, J=1.4 Hz, 1H), 8.42 (s, 1H), 8.37 (dd, J=2.5, 1.5 Hz, 1H), 8.24 (d, J=2.6 Hz, 1H), 8.10 (s, 1H), 7.90 (d, J=9.3 Hz, 1H), 7.80 (d, J=9.3 Hz, 1H), 7.74 (d, J=3.8 Hz, 1H), 7.32 (d, J=3.8 Hz, 1H), 5.71 (s, 2H), 3.92 (s, 3H).

### Example DP

### 6-[(Pyrazin-2-yl)({5-[5-(trifluoromethyl)-1,2,4-oxadiazol-3-yl]thiophen-2-yl}methyl)amino]pyridazine-3-carboxylic acid

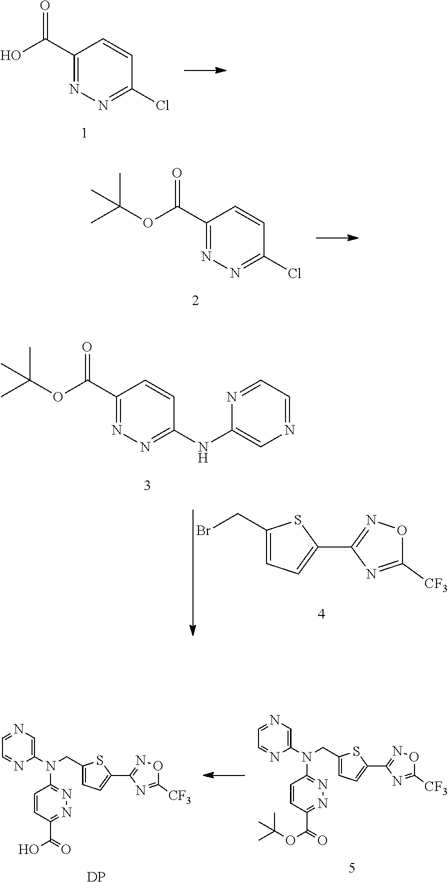

A suspension of (1) (2.0 g, 12.7 mmol) and 4-(dimethylamino)pyridine (776 mg, 6.3 mmol) in THF (50 mL) was treated with di-tert-butyl dicarbonate (3.6 g, 16.5 mmol). It was heated up to 50° C. for 1 h, then stirred at rt overnight. The reaction mixture was concentrated in vacuo, re-dissolved in EtOAc (20 mL), poured into HCl solution (1M, 20 mL) and extracted with EtOAc (2×20 mL). The combined organics were washed with NaHCO₃ solution (50 mL) and brine (50 mL), dried over MgSO₄, filtered and concentrated in vacuo. Purification by silica gel column chromatography with hexane/EtOAc (1:0-3:1) yielded (2) as a white solid (2.2 g, 81%).

LCMS (ES): Found 237.0 [M+Na]⁺.

¹H NMR (300 MHz, Chloroform-d), δ: 8.08 (d, J=8.7 Hz, 1H), 7.63 (d, J=8.9 Hz, 1H), 1.67 (s, 9H).

A suspension of (2) (1.38 g, 6.41 mmol), 2-aminopyrazine (508 mg, 5.34 mmol), Cs₂CO₃ (3.49 g, 1.14 mmol) and Xantphos (185 mg, 0.32 mmol) in dioxane (40 mL) was purged with Ar(g) for 10 min. Pd₂(dba)₃ (147 mg, 0.16 mmol) was added and mixture was heated up to 90° C. overnight. Once cooled down to rt, it was poured into H₂O (40 mL) and EtOAc (6×20 mL). The combined organics were washed dried over MgSO₄, filtered and concentrated in vacuo. Purification by silica gel column chromatography with hexane/CH₂Cl₂ (1:0-0:1) then CH₂Cl₂/MeOH (1:0-49:1) yielded (3) as an off-white solid (752 mg, 52%).

LCMS (ES): Found 274.0 [M+H]⁺.

¹H NMR (300 MHz, DMSO-d₆), δ: 10.98 (s, 1H), 9.01 (d, J=1.5 Hz, 1H), 8.29-8.39 (m, 1H), 8.16-8.27 (m 2H), 8.07 (d, J=9.3 Hz, 1H), 1.59 (s, 9H).

KO^{t}Bu (1M in THF, 2.1 mL) was added to a solution of (3) (557 mg, 2.0 mmol) in DMF (20 mL) at 0° C. under Ar(g). The reaction mixture was stirred for 55 min, cooled down to −10° C. then (4) (703 mg, 2.2 mmol) was added. It was stirred for a further 1.5 h, warmed up to rt and concentrated in vacuo to half volume. The reaction mixture was poured into brine (50%, 20 mL), then extracted with EtOAc (3×25 mL). The combined organics were dried over MgSO₄, filtered and concentrated in vacuo to afford a residue. Purification by silica gel column chromatography with hexane/EtOAc (1:0-1:3) yielded (5) as an orange solid (499 mg, 48%).

LCMS (ES): Found 505.9 [M+H]⁺.

¹H NMR (300 MHz, DMSO-d₆), δ: 8.88 (d, J=1.5 Hz, 1H), 8.49 (dd, J=2.6, 1.5 Hz, 1H), 8.40 (d, J=2.6 Hz, 1H), 7.99 (d, J=9.4 Hz, 1H), 7.78 (d, J=9.4 Hz, 1H), 7.74 (d, J=3.8 Hz, 1H), 7.31 (d, J=3.8 Hz, 1H), 5.77 (s, 2H), 1.58 (s, 9H).

¹⁹F NMR (282 MHz, DMSO-d₆), δ: −64.80 (s, 3F).

To a solution of (5) (488 mg, 0.97 mmol) in CH₂Cl₂ (10 mL) was added TFA (3.7 mL, 48.3 mmol) and the reaction mixture was stirred at rt overnight. It was diluted with toluene (3 mL) and concentrated in vacuo. The residue was triturated and washed with Et₂O (15 mL) to yield (DP) as a solid (318 mg, 73%).

LCMS (ES): Found 449.9 [M+H]⁺.

¹H NMR (300 MHz, DMSO-d₆), δ: 8.90 (d, J=1.3 Hz, 1H), 8.49 (dd, J=2.6, 1.5 Hz, 1H), 8.40 (d, J=2.4 Hz, 1H), 8.04 (d, J=9.4 Hz, 1H), 7.79 (d, J=9.2 Hz, 1H), 7.74 (d, J=3.8 Hz, 1H), 7.32 (d, J=3.8 Hz, 1H), 5.78 (s, 2H).

¹⁹F NMR (282 MHz, DMSO-d₆), δ: −64.79 (s, 3F).

### Example DQ

### 6-[(Pyrazin-2-yl)({5-[5-(trifluoromethyl)-1,2,4-oxadiazol-3-yl]thiophen-2-yl}methyl)amino]pyridazine-3-carboxamide

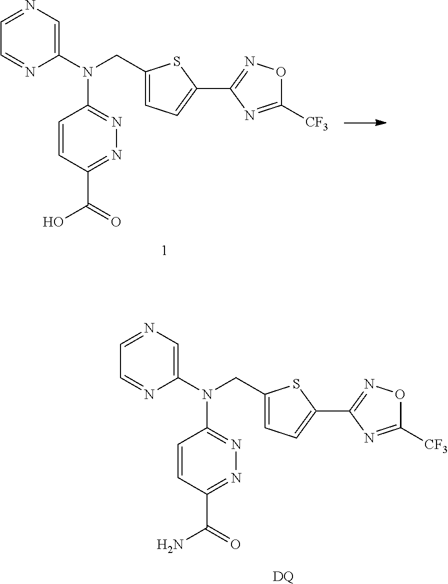

To a solution of (1) (49.4 mg, 0.11 mmol) in DMF (1.1 mL) was added diisopropylethylamine (0.08 mL, 0.44 mmol) followed by HATU (63 mg, 0.17 mmol) and ammonium chloride (11.8 mg, 0.22 mmol). The reaction mixture was stirred at rt overnight. It was then diluted with EtOAc (10 mL) and washed with HCl solution (1M, 2×10 mL), then brine (10 mL). The organics were dried over MgSO₄, filtered and concentrated in vacuo. Purification by silica gel column chromatography with hexane/EtOAc (1:0-0:1) yielded (DQ) as an off-white solid (41 mg, 84%).

LCMS (ES): Found 448.9 [M+H]⁺.

¹H NMR (300 MHz, DMSO-d₆), δ: 8.89 (d, J=1.3 Hz, 1H), 8.42-8.52 (m, 2H), 8.37 (d, J=2.4 Hz, 1H), 8.05 (d, J=9.4 Hz, 1H), 7.83 (d, J=9.4 Hz, 1H), 7.78 (br s, 1H), 7.73-7.76 (m, 1H), 7.31 (d, J=3.8 Hz, 1H), 5.77 (s, 2H).

¹⁹F NMR (282 MHz, DMSO-d₆), δ: −64.80 (s, 3F).

### Example DR

### 6-(3-Methoxyazetidine-1-carbonyl)-N-(pyrazin-2-yl)-N-({5-[5-(trifluoromethyl)-1,2,4-oxadiazol-3-yl]thiophen-2-yl}methyl)pyridazin-3-amine

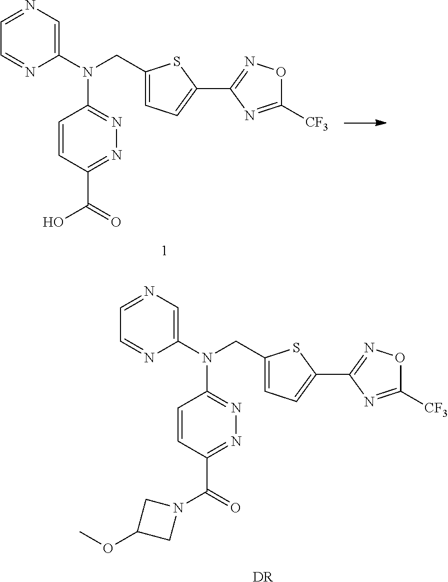 [SMILES: O=C(O)c1ccc(N(Cc2ccc(-c3noc(C(F)(F)F)n3)s2)c2cnccn2)nn1 ; COC1CN(C(=O)c2ccc(N(Cc3ccc(-c4noc(C(F)(F)F)n4)s3)c3cnccn3)nn2)C1 ; "DR"]

To a solution of (1) (51 mg, 0.11 mmol) in DMF (1 mL) was added diisopropylethylamine (0.08 mL, 0.45 mmol) followed by HATU (65 mg, 0.17 mmol) and 3-methoxyazetidine hydrochloride (28 mg, 0.22 mmol). The reaction mixture was stirred at rt overnight. It was then diluted with EtOAc (10 mL) and washed with HCl solution (1M, 2×10 mL), then brine (10 mL). The organics were dried over MgSO₄, filtered and concentrated in vacuo. Purification by silica gel column chromatography with EtOAc/MeOH (1:0-4:1) yielded (DR) as an off-white solid (45 mg, 77%).

LCMS (ES): Found 518.9 [M+H]⁺.

¹H NMR (300 MHz, DMSO-d₆), δ: 8.87 (d, J=1.1 Hz, 1H), 8.43-8.55 (m, 1H), 8.38 (d, J=2.4 Hz, 1H), 7.99 (d, J=9.2 Hz, 1H), 7.82 (d, J=9.4 Hz, 1H), 7.75 (d, J=3.8 Hz, 1H), 7.31 (d, J=3.8 Hz, 1H), 5.77 (s, 2H), 4.73-4.86 (m, 1H), 4.40 (m, 1H), 4.21-4.35 (m, 2H), 3.83-3.97 (m, 1H), 3.25 (s, 3H).

¹⁹F NMR (282 MHz, DMSO-d₆), δ: −64.79 (s, 3F).

### Example DS

### 6-(Morpholine-4-carbonyl)-N-(pyrazin-2-yl)-N-({5-[5-(trifluoromethyl)-1,2,4-oxadiazol-3-yl]thiophen-2-yl}methyl)pyridazin-3-amine

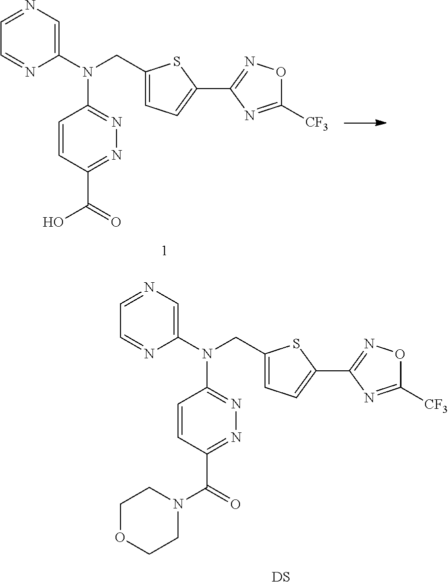 [SMILES: O=C(O)c1ccc(N(Cc2ccc(-c3noc(C(F)(F)F)n3)s2)c2cnccn2)nn1 ; O=C(c1ccc(N(Cc2ccc(-c3noc(C(F)(F)F)n3)s2)c2cnccn2)nn1)N1CCOCC1 ; "DS"]

To a solution of (1) (31 mg, 0.07 mmol) in DMF (1 mL) was added diisopropylethylamine (0.02 mL, 0.14 mmol) followed by HATU (39 mg, 0.10 mmol) and morpholine (0.01 mL, 0.01 mmol). The reaction mixture was stirred at rt overnight. It was then diluted with EtOAc (10 mL) and washed with HCl solution (1M, 2×10 mL), then brine (10 mL). The organics were dried over MgSO₄, filtered and concentrated in vacuo. Purification by silica gel column chromatography with EtOAc/MeOH (1:0-4:1) yielded (DS) as an off-white solid (30 mg, 85%).

LCMS (ES): Found 518.9 [M+H]⁺.

¹H NMR (300 MHz, DMSO-d₆), δ: 8.87 (d, J=1.1 Hz, 1H), 8.42-8.47 (m, 1H), 8.35 (d, J=2.6 Hz, 1H), 7.84 (d, J=9.1 Hz, 1H), 7.79 (d, J=9.4 Hz, 1H), 7.75 (d, J=3.8 Hz, 1H), 7.32 (d, J=3.6 Hz, 1H), 5.76 (s, 2H), 3.69 (m, 4H), 3.61 (m, 4H).

¹⁹F NMR (282 MHz, DMSO-d₆), δ: −64.79 (s, 3F).

### Example DT

### N-{Imidazo[1,2-b]pyridazin-6-yl}-N-({5-[5-(trifluoromethyl)-1,2,4-oxadiazol-3-yl]thiophen-2-yl}methyl)pyrazin-2-amine

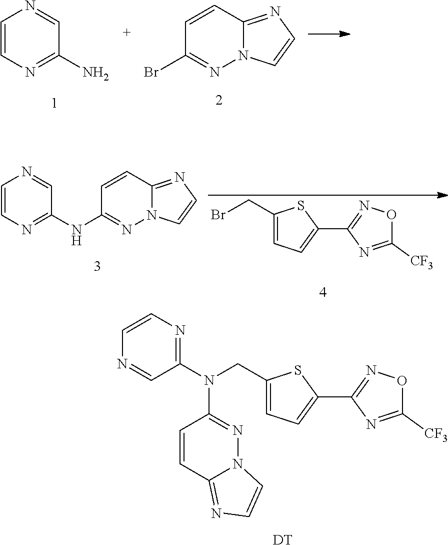

To degassed dioxane (10 mL) were added sequentially 2-aminopyrazine (1) (54 mg, 0.57 mmol), 6-bromoimidazo[1,2-b]pyridazine (2) (136 mg, 0.69 mmol), Pd₂(dba)₃ (26 mg, 0.03 mmol), XantPhos (33 mg, 0.06 mmol) and Cs₂CO₃ (371 mg, 1.1 mmol) with continued degassing. The reaction mixture was heated up to 90° C. for 2 h. Once cooled down to rt, it was poured into a brine solution (50%, 20 mL) and extracted with EtOAc (3×20 mL). The combined organic extracts were dried over MgSO₄, filtered and concentrated in vacuo. Purification by silica gel column chromatography with EtOAc/MeOH (1:0-0:1) afforded a residue. It was re-dissolved in CH₂Cl₂/MeOH (4:1, 20 mL) and swirled with MP-TMT resin (250 mg, 1.3 mmol/g) at rt overnight. The solution was filtered, the resin washed with CH₂Cl₂/MeOH (4:1, 50 mL) and the filtrate concentrated in vacuo to yield (3) as an off-white solid (94 mg, 77%).

LCMS (ES): Found 213.0 [M+H]⁺.

¹H NMR (300 MHz, DMSO-d₆), δ: 10.40 (s, 1H), 9.39 (d, J=1.3 Hz, 1H), 8.32-8.40 (m, 1H), 8.26 (d, J=2.4 Hz, 1H), 8.20 (s, 1H), 8.04 (d, J=9.6 Hz, 1H), 7.66 (d, J=0.6 Hz, 1H), 7.35 (d, J=9.6 Hz, 1H).

To a solution of (3) (30 mg, 0.14 mmol) in anhydrous DMF (2 mL) was added KO^{t}Bu (1M in THF, 0.17 mL) at 0° C. The solution was stirred for 25 min before the addition of (4) (49 mg, 0.16 mmol) and allowed to warm up to rt over 1.5 h. It was then quenched with H₂O (1 mL), poured into a brine solution (50%, 10 mL) and extracted with EtOAc (3×10 mL). The combined organic extracts were dried over MgSO₄, filtered and concentrated in vacuo. Purification by silica gel column chromatography with EtOAc/MeOH (1:0-32:1), then EtOAc/MeOH (1:0-99:1) and CH₂Cl₂/MeOH (1:0-39:1) yielded (DT) as a yellow solid (36 mg, 58%).

LCMS (ES): Found 444.8 [M+H]⁺.

¹H NMR (300 MHz, DMSO-d₆), δ: 8.69-8.76 (m, 1H), 8.36-8.44 (m, 1H), 8.27 (d, J=2.4 Hz, 1H), 8.16 (s, 1H), 8.05 (d, J=9.6 Hz, 1H), 7.74 (d, J=3.8 Hz, 1H), 7.69 (s, 1H), 7.25-7.36 (m, 2H), 5.62 (s, 2H).

¹⁹F NMR (282 MHz, DMSO-d₆), δ: −64.79 (s, 3F).

### Example DU

### N-{[1,2,4]Triazolo[4,3-b]pyridazin-6-yl}-N-({5-[5-(trifluoromethyl)-1,2,4-oxadiazol-3-yl]thiophen-2-yl}methyl)pyrazin-2-amine

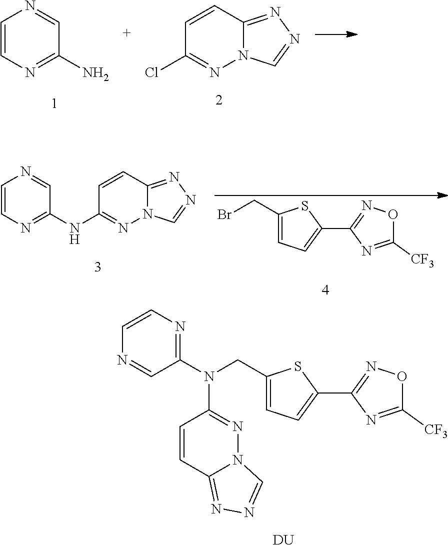

Under Ar(g), to a mixture of pyrazin-2-amine (1) (209 mg, 2.2 mmol), 6-chloro-1,2,4-triazolo[4,3-b]pyridazine (2) (309 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (2.8 mg, 1%).

LCMS (ES): Found 214.3 [M+H]⁺.

To a solution of (3) (2.7 mg, 0.17 mmol) in anhydrous DMF (0.5 mL) was added KO^{t}Bu (1M in THF, 0.015 mL) at 0° C. The reaction mixture was stirred for 20 min before the addition of (4) (6 mg, 0.02 mmol). It was then stirred for a further 1.5 h, quenched with H₂O (0.2 mL) and concentrated in vacuo. The residue was re-dissolved in CH₂Cl₂ (5 mL) and filtered through a short MgSO₄ pad. Purification by silica gel column chromatography with CH₂C₂/EtOAc/MeOH (1:0:0-0:1:0-0:19:1) yielded (DU) as an oil (2.2 mg, 39%).

LCMS (ES): Found 445.8 [M+H]⁺.

¹H NMR (400 MHz, Methanol-d₄), δ: 9.33 (s, 1H), 8.74 (d, J=1.5 Hz, 1H), 8.48 (dd, J=2.6, 1.5 Hz, 1H), 8.37 (d, J=2.6 Hz, 1H), 8.07 (dd, J=10.1, 0.7 Hz, 1H), 7.71 (d, J=3.8 Hz, 1H), 7.40 (d, J=10.0 Hz, 1H), 7.27 (d, J=3.8 Hz, 1H), 5.70 (s, 2H).

¹⁹F NMR (282 MHz, Methanol-d₄), δ: −67.43 (s, 3F).

### Example DV

### 5-Methoxy-N-(pyrazin-2-yl)-N-({5-[5-(trifluoromethyl)-1,2,4-oxadiazol-3-yl]thiophen-2-yl}methyl)pyridazin-3-amine

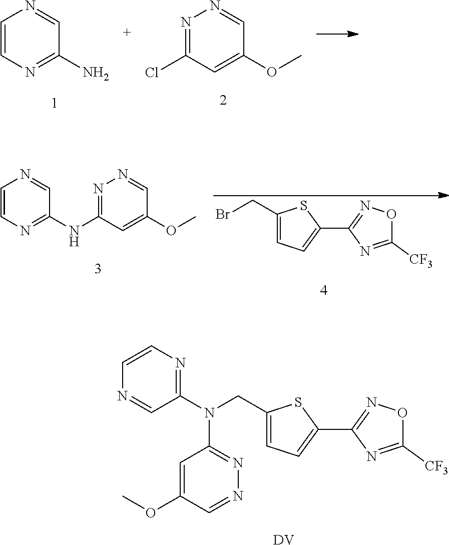

Under Ar(g), to a mixture of pyrazin-2-amine (1) (209 mg, 2.2 mmol), 3-chloro-5-methoxypyridazine (2) (289 mg, 2.0 mmol), Cs₂CO₃ (1.30 g, 4.0 mmol) was added degassed dry 1,4-dioxane (13 mL). The reaction mixture was then flushed with Ar(g) for 1 min before Pd₂(dba)₃ (92 mg, 0.1 mmol) and Xantphos (127 mg, 0.22 mmol) were added. The reaction mixture was heated up to 90° C. for 40 h. It was then cooled down to rt. EtOAc (15 mL), H₂O (10 mL) and brine (5 mL) were added to the reaction mixture. The organic phase was separated and the aqueous phase was extracted with EtOAc (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (3) as a solid (24 mg, 6%).

NaH (60%, 5.8 mg, 0.15 mmol) was added to a solution of (3) (24 mg, 0.12 mmol) in dry DMF (0.7 mL). After 5 min, a solution of (4) (45 mg, 0.14 mmol) in dry DMF (0.6 mL) was added. The reaction mixture was stirred overnight. Water (0.01 mL) was then added to quench the remaining NaH. The reaction mixture was purified by acidic prep LCMS to yield (DV) as a solid (5.7 mg, 11%).

LCMS (ES): Found 436.3 [M+H]⁺.

¹H NMR (400 MHz, DMSO-d₆), δ: 8.32 (d, J=1.5 Hz, 1H), 8.28 (dd, J=2.6, 1.4 Hz, 1H), 8.10 (d, J=2.7 Hz, 1H), 7.85 (d, J=3.0 Hz, 1H), 7.80 (d, J=3.8 Hz, 1H), 7.64 (d, J=3.0 Hz, 1H), 7.42 (d, J=3.8 Hz, 1H), 5.69 (s, 2H), 3.80 (s, 3H).

### Example DW

### 6-[(Pyrazin-2-yl)({5-[5-(trifluoromethyl)-1,2,4-oxadiazol-3-yl]thiophen-2-yl}methyl)amino]pyridazine-4-carboxylic acid

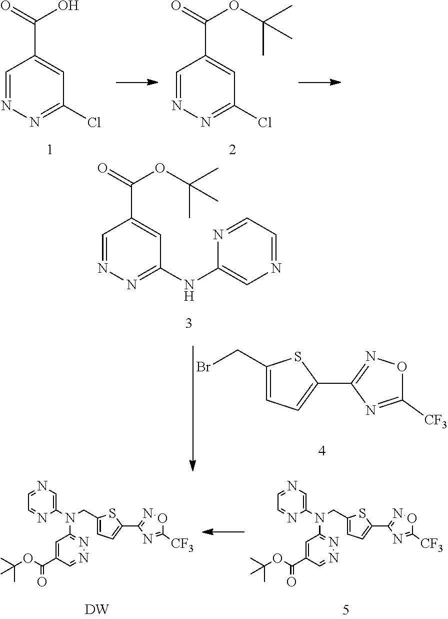

A suspension of (1) (1.0 g, 6.3 mmol) and 4-(dimethylamino)pyridine (385 mg, 3.2 mmol) in THF (30 mL) was treated with di-tert-butyl dicarbonate (1.72 g, 7.9 mmol) and heated gently with a heat gun until effervescence. Heating was withdrawn and the reaction mixture was cooled down to 0° C. Gas evolution ceased and the reaction mixture was concentrated in vacuo. The residue was dissolved in EtOAc and washed sequentially with HCl solution (5%, 3×10 mL), NaOH solution (5%, 3×10 mL) and brine (10 mL). The organic phase was dried over MgSO₄, filtered and concentrated in vacuo to yield (2) as a beige solid (1.25 g, 92%).

A suspension of (2) (400 mg, 1.86 mmol), 2-aminopyrazine (177 mg, 1.86 mmol), Cs₂CO₃ (1.21 g, 3.73 mmol) and Xantphos (108 mg, 0.19 mmol) in dioxane (6 mL) was purged with Ar(g) for 20 min. Pd₂(dba)₃ (85 mg, 0.09 mmol) was added and mixture was heated up to 90° C. overnight. Once cooled down to rt, it was partitioned between H₂O (25 mL) and brine (25 mL) and EtOAc (3×20 mL). The combined organics were washed with brine (10 mL), dried over MgSO₄, filtered and concentrated in vacuo. Purification by silica gel column chromatography with hexane/EtOAc (1:0-1:1) yielded (3) as a brown solid (315 mg, 62%).

KO^{t}Bu (1M in THF, 1.3 mL) was added to a solution of (3) (300 mg, 1.1 mmol) in DMF (12 mL) at 0° C. under Ar(g). The reaction mixture was stirred for 10 min then (4) (366 mg, 1.16 mmol) was added. It was stirred for a further 1.5 h, poured into a mixture of H₂O (25 mL) and brine (25 mL), then extracted with EtOAc (3×10 mL). The combined organics were washed with NaOH solution (5%, 3×10 mL), brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-2:3) yielded (5) as a red oil (206 mg, 37%).

To a solution of (5) (200 mg, 0.40 mmol) in CH₂Cl₂ (10 mL) was added TFA (0.91 mL, 11.9 mmol) and the reaction mixture was stirred at rt overnight. Additional TFA (0.91 mL, 11.9 mmol) was re-charged and stirring continued overnight. The reaction mixture was diluted with toluene (3 mL) and concentrated in vacuo. The residue was re-concentrated from toluene (5 mL) and purified by silica gel column chromatography with CH₂Cl₂/MeOH (1:0-4:1) to yield (DW) as a beige solid (111 mg, 63%).

LCMS (ES): Found 449.8 [M+H]⁺.

¹H NMR (300 MHz, DMSO-d₆), δ: 9.22 (d, J=1.7 Hz, 1H), 8.85 (d, J=1.5 Hz, 1H), 8.42 (dd, J=2.5, 1.4 Hz, 1H), 8.34 (d, J=2.6 Hz, 1H), 8.03 (d, J=1.7 Hz, 1H), 7.75 (d, J=3.6 Hz, 1H), 7.32 (d, J=3.8 Hz, 1H), 5.79 (s, 2H), 3.44 (br s, 1H).

¹⁹F NMR (282 MHz, DMSO-d₆), δ: −64.80 (s, 3F).

### Example DX

### 6-[(Pyrazin-2-yl)({5-[5-(trifluoromethyl)-1,2,4-oxadiazol-3-yl]thiophen-2-yl}methyl)amino]pyridazine-4-carboxamide

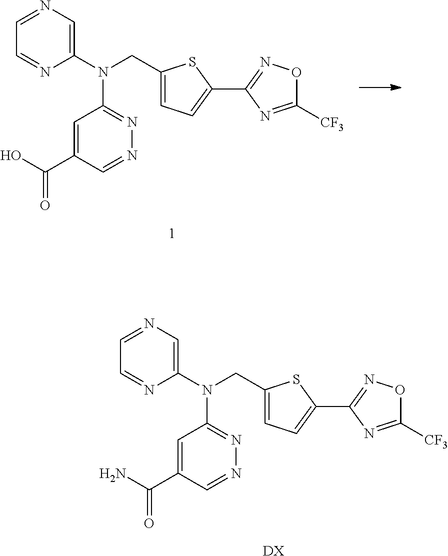

To a solution of (1) (50 mg, 0.11 mmol) in DMF (2 mL) was added diisopropylethylamine (0.08 mL, 0.45 mmol) followed by HATU (63 mg, 0.17 mmol). The reaction mixture was stirred for 0.5 h at rt, then ammonium chloride (12 mg, 0.22 mmol) was added in one portion. It was further stirred overnight. The reaction mixture was then diluted with EtOAc (30 mL) and washed with HCl solution (5%, 3×10 mL), then brine (10 mL). The organics were dried over MgSO₄, filtered and concentrated in vacuo. Purification by silica gel column chromatography with EtOAc/MeOH (1:0-19:1) yielded (DX) as a yellow solid (33 mg, 65%).

LCMS (ES): Found 448.9 [M+H]⁺.

¹H NMR (300 MHz, DMSO-d₆), δ: 9.24 (d, J=1.7 Hz, 1H), 8.82 (d, J=1.5 Hz, 1H), 8.43 (dd, J=2.5, 1.4 Hz, 1H), 8.37 (br s, 1H), 8.34 (d, J=2.6 Hz, 1H), 8.01 (d, J=1.9 Hz, 1H), 7.95 (br s, 1H), 7.74 (d, J=3.8 Hz, 1H), 7.30 (d, J=3.8 Hz, 1H), 5.75 (s, 2H).

¹⁹F NMR (282 MHz, DMSO-d₆), δ: −64.79 (s, 3F).

### Example DY

### N-Methyl-6-[(pyrazin-2-yl)({5-[5-(trifluoromethyl)-1,2,4-oxadiazol-3-yl]thiophen-2-yl}methyl)amino]pyridazine-4-carboxamide

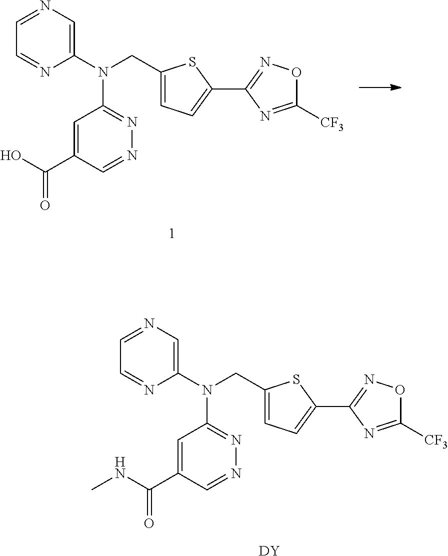 [SMILES: O=C(O)c1cnnc(N(Cc2ccc(-c3noc(C(F)(F)F)n3)s2)c2cnccn2)c1 ; CNC(=O)c1cnnc(N(Cc2ccc(-c3noc(C(F)(F)F)n3)s2)c2cnccn2)c1 ; "DY"]

To a solution of (1) (50 mg, 0.11 mmol) in DMF (1 mL) was added diisopropylethylamine (0.08 mL, 0.45 mmol) followed by HATU (63 mg, 0.17 mmol). The reaction mixture was stirred for 0.5 h at rt, then methylamine hydrochloride (15 mg, 0.22 mmol) was added in one portion. It was further stirred overnight. The reaction mixture was then diluted with EtOAc (30 mL) and washed with HCl solution (5%, 3×10 mL), then brine (10 mL). The organics were dried over MgSO₄, filtered and concentrated in vacuo. Purification by silica gel column chromatography with CH₂Cl₂/MeOH (1:0-19:1) yielded (DY) as a brown solid (31 mg, 60%).

LCMS (ES): Found 462.9 [M+H]⁺.

¹H NMR (300 MHz, DMSO-d₆), δ: 9.22 (d, J=1.7 Hz, 1H), 8.85-8.93 (m, 1H), 8.83 (d, J=1.3 Hz, 1H), 8.44 (dd, J=2.6, 1.5 Hz, 1H), 8.35 (d, J=2.4 Hz, 1H), 7.98 (d, J=1.7 Hz, 1H), 7.74 (d, J=3.8 Hz, 1H), 7.30 (d, J=4.0 Hz, 1H), 5.76 (s, 2H), 2.80 (d, J=4.7 Hz, 3H).

¹⁹F NMR (282 MHz, DMSO-d₆), δ: −64.79 (s, 3F).

### Example DZ

### 5-(3-Methoxyazetidine-1-carbonyl)-N-(pyrazin-2-yl)-N-({5-[5-(trifluoromethyl)-1,2,4-oxadiazol-3-yl]thiophen-2-yl}methyl)pyridazin-3-amine

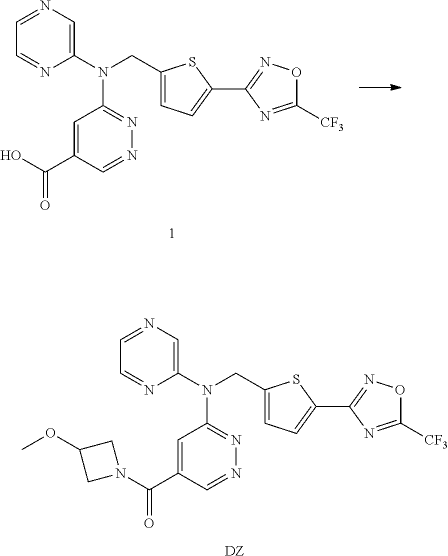 [SMILES: O=C(O)c1cnnc(N(Cc2ccc(-c3noc(C(F)(F)F)n3)s2)c2cnccn2)c1 ; COC1CN(C(=O)c2cnnc(N(Cc3ccc(-c4noc(C(F)(F)F)n4)s3)c3cnccn3)c2)C1 ; "DZ"]

To a solution of (1) (50 mg, 0.11 mmol) in DMF (1.5 mL) was added tetramethylfluoroformamidinium hexafluorophosphate (46 mg, 0.17 mmol) and diisopropylethylamine (0.12 mL, 0.67 mmol). The reaction mixture was stirred for 0.5 h at rt before 3-methoxyazetidine hydrochloride (28 mg, 0.22 mmol) was added in one portion. It was then heated up to 80° C. overnight. Once cooled down to rt, it was poured into a mixture of H₂O (10 mL) and brine (10 mL), then extracted with EtOAc (4×10 mL). The combined organics were washed with NaOH solution (5%, 2×10 mL) and brine (10 mL), dried over MgSO₄, filtered and concentrated in vacuo. Purification by silica gel column chromatography with CH₂Cl₂/EtOAc (1:0-0:1) yielded (DZ) as a yellow film (8 mg, 15%).

LCMS (ES): Found 518.8 [M+H]⁺.

¹H NMR (300 MHz, DMSO-d₆), δ: 9.04 (d, J=1.7 Hz, 1H), 8.83 (d, J=1.5 Hz, 1H), 8.40 (dd, J=2.5, 1.4 Hz, 1H), 8.32 (d, J=2.6 Hz, 1H), 7.85 (d, J=1.7 Hz, 1H), 7.74 (d, J=3.8 Hz, 1H), 7.31 (d, J=3.8 Hz, 1H), 5.77 (s, 2H), 4.40-4.49 (m, 1H), 4.16-4.30 (m, 3H), 3.81-3.91 (m, 1H), 3.21 (s, 3H).

¹⁹F NMR (282 MHz, DMSO-d₆), δ: −64.80 (s, 3F).

### Example EA

### N-(Pyrazin-2-yl)-5-(pyrrolidine-1-carbonyl)-N-({5-[5-(trifluoromethyl)-1,2,4-oxadiazol-3-yl]thiophen-2-yl}methyl)pyridazin-3-amine

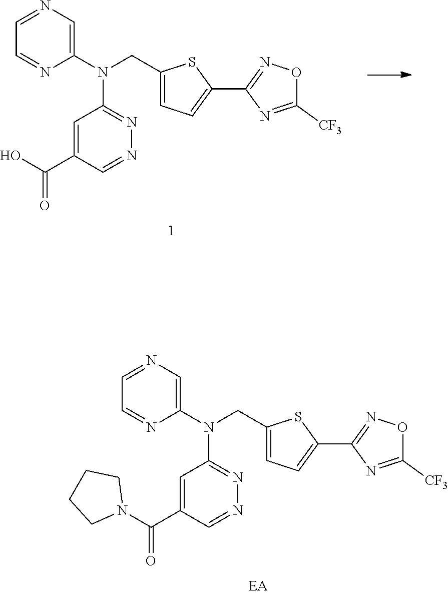 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CCCC1]

To a solution of (1) (50 mg, 0.11 mmol) in DMF (2 mL) was added diisopropylethylamine (0.03 mL, 0.17 mmol) followed by HATU (42 mg, 0.11 mmol). The reaction mixture was stirred for 0.5 h at rt, then pyrrolidine (0.011 mL, 0.13 mmol) was added in one portion. It was further stirred for 2.5 h before diisopropylethylamine (0.03 mL, 0.17 mmol), HATU (42 mg, 0.11 mmol) and pyrrolidine (0.011 mL, 0.13 mmol) were re-charged. The reaction mixture was then stirred overnight. It was diluted with EtOAc (30 mL) and washed with HCl solution (5%, 3×10 mL), then brine (10 mL). The organics were dried over MgSO₄, filtered and concentrated in vacuo. Purification by silica gel column chromatography with EtOAc/MeOH (1:0-19:1) yielded (EA) as a yellow solid (40 mg, 72%).

LCMS (ES): Found 502.9 [M+H]⁺.

¹H NMR (300 MHz, DMSO-d₆), δ: 8.98 (d, J=1.7 Hz, 1H), 8.81 (d, J=1.3 Hz, 1H), 8.40 (dd, J=2.4, 1.5 Hz, 1H), 8.31 (d, J=2.6 Hz, 1H), 7.83 (d, J=1.5 Hz, 1H), 7.74 (d, J=3.6 Hz, 1H), 7.31 (d, J=3.8 Hz, 1H), 5.68-5.79 (m, 2H), 3.46 (t, J=6.5 Hz, 2H), 3.38 (m, 2H), 1.77-1.91 (m, 4H).

¹⁹F NMR (282 MHz, DMSO-d₆), δ: −64.80 (s, 3F).

### Example EB

### 5-(Morpholine-4-carbonyl)-N-(pyrazin-2-yl)-N-({5-[5-(trifluoromethyl)-1,2,4-oxadiazol-3-yl]thiophen-2-yl}methyl)pyridazin-3-amine

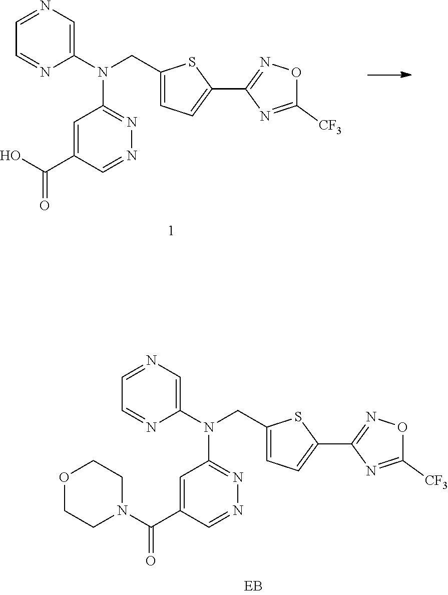 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CCOCC1]

To a solution of (1) (50 mg, 0.11 mmol) in DMF (1.5 mL) was added tetramethylfluoroformamidinium hexafluorophosphate (46 mg, 0.17 mmol) and diisopropylethylamine (0.08 mL, 0.45 mmol). The reaction mixture was stirred for 1 h at rt, then morpholine (0.015 mL, 0.17 mmol) was added in one portion. It was then heated up to 80° C. overnight. Once cooled down to rt, it was poured into a mixture of H₂O (10 mL) and brine (10 mL), then extracted with EtOAc (3×10 mL). The combined organics were washed with NaOH solution (5%, 2×10 mL) and brine (10 mL), dried over MgSO₄, filtered and concentrated in vacuo. Purification by silica gel column chromatography with CH₂Cl₂/EtOAc (1:0-0:1), then prep-TLC with EtOAc yielded (EB) as a beige solid (5 mg, 7.8%).

LCMS (ES): Found 518.8 [M+H]⁺.

¹H NMR (300 MHz, DMSO-d₆), δ: 8.95 (d, J=1.5 Hz, 1H), 8.84 (d, J=1.3 Hz, 1H), 8.40 (dd, J=2.6, 1.5 Hz, 1H), 8.32 (d, J=2.6 Hz, 1H), 7.79 (d, J=1.7 Hz, 1H), 7.75 (d, J=3.6 Hz, 1H), 7.32 (d, J=3.8 Hz, 1H), 5.75 (s, 2H), 3.51-3.70 (m, 6H), 3.27-3.37 (m, 2H).

¹⁹F NMR (282 MHz, DMSO-d₆), δ: −64.80 (s, 3F).

### Example EC

### 5-(4-Methylpiperazine-1-carbonyl)-N-(pyrazin-2-yl)-N-({5-[5-(trifluoromethyl)-1,2,4-oxadiazol-3-yl]thiophen-2-yl}methyl)pyridazin-3-amine

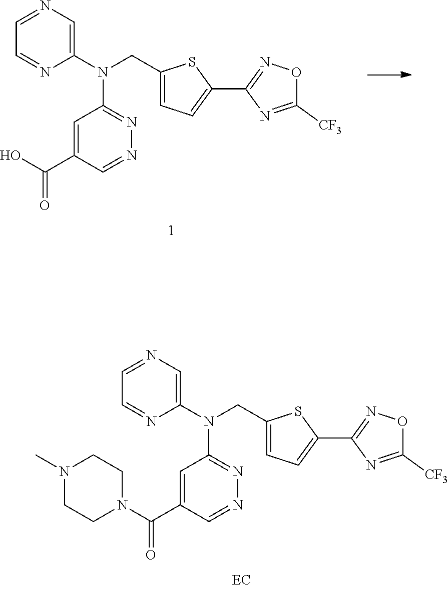 [SMILES: O=C(O)c1cnnc(N(Cc2ccc(-c3noc(C(F)(F)F)n3)s2)c2cnccn2)c1 ; CN1CCN(C(=O)c2cnnc(N(Cc3ccc(-c4noc(C(F)(F)F)n4)s3)c3cnccn3)c2)CC1]

To a solution of (1) (50 mg, 0.11 mmol) in DMF (1 mL) was added diisopropylethylamine (0.04 mL, 0.22 mmol) followed by HATU (63 mg, 0.17 mmol). The reaction mixture was stirred for 0.5 h at rt, then 4-methylpiperazine (0.025 mL, 0.22 mmol) was added in one portion. It was further stirred overnight. It was diluted with EtOAc (30 mL) and washed with brine (3×10 mL). The organics were dried over MgSO₄, filtered and concentrated in vacuo. Purification by silica gel column chromatography with CH₂Cl₂/MeOH (1:0-19:1) yielded (EC) as a brown solid (46 mg, 78%).

LCMS (ES): Found 531.9 [M+H]⁺.

¹H NMR (300 MHz, DMSO-d₆), δ: 8.92 (d, J=1.5 Hz, 1H), 8.84 (d, J=1.3 Hz, 1H), 8.40 (dd, J=2.5, 1.4 Hz, 1H), 8.31 (d, J=2.6 Hz, 1H), 7.72-7.79 (m, 2H), 7.32 (d, J=3.8 Hz, 1H), 5.75 (s, 2H), 3.54-3.65 (m, 2H), 3.24-3.32 (m, 2H), 2.32-2.41 (m, 2H), 2.23-2.32 (m, 2H), 2.19 (s, 3H).

¹⁹F NMR (282 MHz, DMSO-d₆), δ: −64.80 (s, 3F).

### Example ED

### 4-{6-[(Pyrazin-2-yl)({5-[5-(trifluoromethyl)-1,2,4-oxadiazol-3-yl]thiophen-2-yl}methyl)amino]pyridazine-4-carbonyl}-1λ⁶-thiomorpholine-1,1-dione

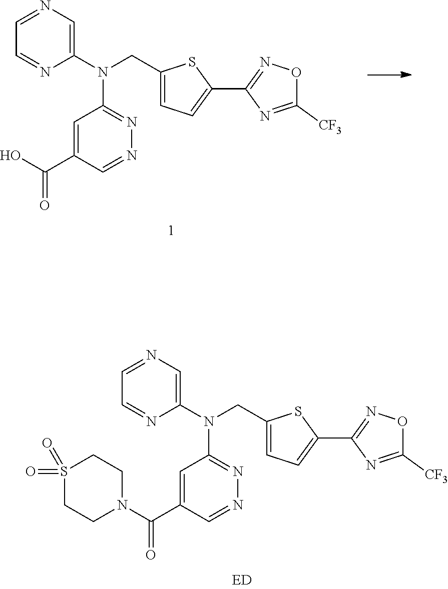 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CCS(=O)(=O)CC1]

To a solution of (1) (50 mg, 0.11 mmol) in DMF (1 mL) was added diisopropylethylamine (0.04 mL, 0.22 mmol) followed by HATU (63 mg, 0.17 mmol). The reaction mixture was stirred for 0.5 h at rt, then thiomorpholine-1,1-dioxide (30 mg, 0.22 mmol) was added in one portion. The reaction mixture was then further stirred overnight. It was diluted with EtOAc (30 mL) and washed with HCl solution (5%, 3×10 mL), then brine (3×10 mL). The organics were dried over MgSO₄, filtered and concentrated in vacuo. Purification by silica gel column chromatography with CH₂Cl₂/EtOAc (1:0-0:1) yielded (ED) as a yellow solid (46 mg, 74%).

LCMS (ES): Found 531.9 [M+H]⁺.

¹H NMR (300 MHz, DMSO-d₆), δ: 8.99 (d, J=1.5 Hz, 1H), 8.84-8.89 (m, 1H), 8.40-8.45 (m, 1H), 8.35 (d, J=2.4 Hz, 1H), 7.89 (d, J=1.5 Hz, 1H), 7.75 (d, J=3.8 Hz, 1H), 7.30 (d, J=3.8 Hz, 1H), 5.76 (s, 2H), 3.95-4.05 (m, 2H), 3.67-3.77 (m, 2H), 3.21-3.31 (m, 4H).

¹⁹F NMR (282 MHz, DMSO-d₆), δ: −64.79 (s, 3F).

### Example EE

### 6-[(Pyrazin-2-yl)({5-[5-(trifluoromethyl)-1,2,4-oxadiazol-3-yl]thiophen-2-yl}methyl)amino]pyridazine-4-carbonitrile

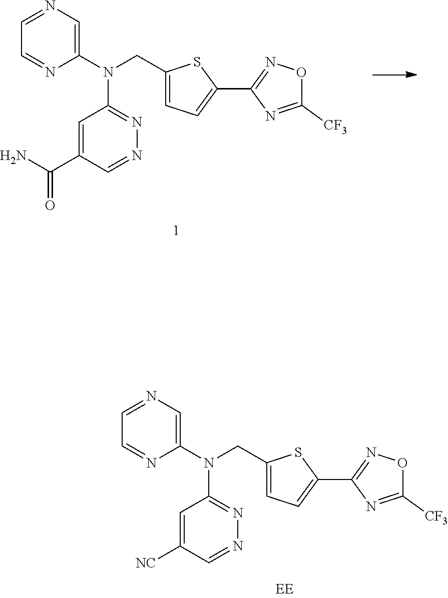 [SMILES: NC(=O)c1cnnc(N(Cc2ccc(-c3noc(C(F)(F)F)n3)s2)c2cnccn2)c1 ; N#Cc1cnnc(N(Cc2ccc(-c3noc(C(F)(F)F)n3)s2)c2cnccn2)c1]

To a solution of (1) (125 mg, 0.28 mmol) in 1,4-dioxane (6.5 mL) was added phosphoryl chloride (0.13 mL, 1.39 mmol). The reaction mixture was heated up to 100° C. overnight. Once cooled down to rt, ammonium chloride (12 mg, 0.22 mmol) was added and the reaction mixture was further stirred overnight. It was poured into a mixture of NaHCO₃ solution (5%, 20 mL), NaOH solution (5%, 20 mL) and brine (30 mL), then extracted with EtOAc (3×50 mL). The organics were washed with brine (20 mL), dried over MgSO₄, filtered and concentrated in vacuo. Purification by silica gel column chromatography with CH₂Cl₂/EtOAc (1:0-4:1) yielded (EE) as an orange solid (25 mg, 21%).

LCMS (ES): Found 430.9 [M+H]⁺.

¹H NMR (300 MHz, DMSO-d₆), δ: 9.26 (d, J=1.7 Hz, 1H), 8.89 (d, J=1.3 Hz, 1H), 8.46 (dd, J=2.6, 1.5 Hz, 1H), 8.37-8.41 (m, 2H), 7.75 (d, J=3.8 Hz, 1H), 7.32 (d, J=3.8 Hz, 1H), 5.75 (s, 2H).

¹⁹F NMR (282 MHz, DMSO-d₆), δ: −64.79 (s, 3F).

### Example EF

### N-(Pyrazin-2-yl)-5-(trifluoromethyl)-N-({5-[5-(trifluoromethyl)-1,2,4-oxadiazol-3-yl]thiophen-2-yl}methyl)pyridazin-3-amine

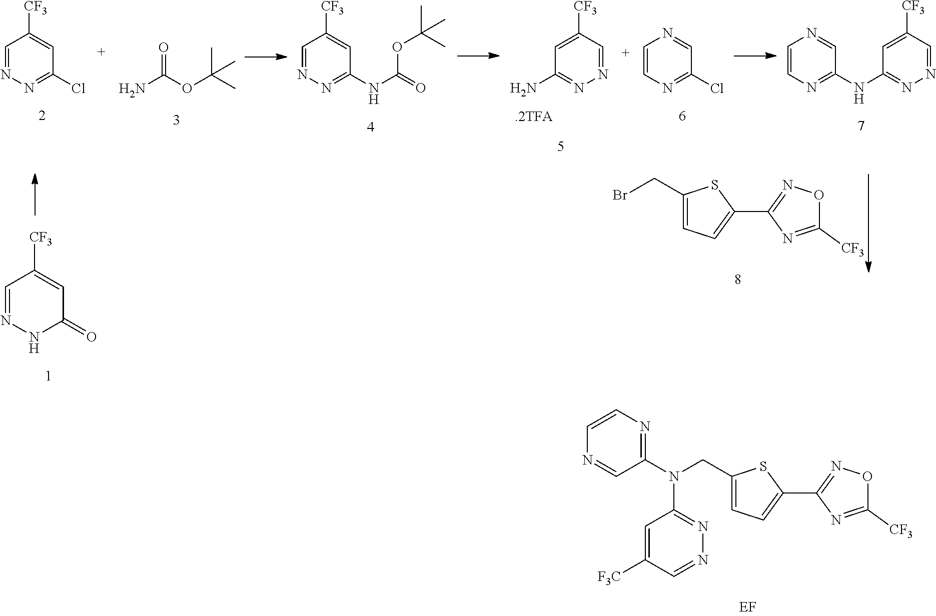

Phosphoryl chloride (2.75 mL, 29.3 mmol) was added to a solution of (1) (1.60 g, 9.8 mmol) in 1,4-dioxane (24 mL) and the reaction mixture was heated up to 80° C. for 4 h. It was then diluted with H₂O (24 mL) dropwise and extracted with CH₂Cl₂ (3×15 mL). The combined organics were washed sequentially with H₂O (15 mL) and NaHCO₃ solution (5%, 2×15 mL), dried over MgSO₄, filtered and concentrated in vacuo to yield (2) as an orange oil (1.78 g, 80%).

¹H NMR (300 MHz, DMSO-d₆), δ: 9.68-9.81 (m, 1H), 8.52-8.60 (m, 1H).

¹⁹F NMR (282 MHz, DMSO-d₆), δ: −62.90 (s, 3F).

A suspension of 3-chloro-5-(trifluoromethyl)pyridazine (2) (300 mg, 1.32 mmol), tert-butyl carbamate (3) (185 mg, 1.58 mmol), Cs₂CO₃ (857 mg, 2.63 mmol) and Xantphos (76 mg, 0.13 mmol) in 1,4-dioxane (9 mL) was purged with Ar(g) for 20 min. Pd₂(dba)₃ (60 mg, 0.02 mmol) was added and the reaction mixture was heated up to 90° C. for 3 h. It was then cooled down to rt and partitioned between H₂O (20 mL) and EtOAc (3×20 mL). The combined organics were washed with brine (10 mL), dried over MgSO₄, filtered and concentrated in vacuo. Purification by silica gel column chromatography with hexane/EtOAc (1:0-7:3) yielded (4) as a solid (274 mg, 25%).

TFA (9.2 mL, 120 mmol) was added to a solution of (4) (1.58 g, 6.0 mmol) in CH₂Cl₂ (47 mL) at rt under Ar(g). The reaction mixture was stirred overnight then concentrated in vacuo. The residue was re-concentrated from CH₂Cl₂ (2×20 mL) to yield (5) as a solid (2.33 g, 99%).

Under Ar(g), to a mixture of 2-chloropyrazine (6) (193 mg, 1.7 mmol), 5-(trifluoromethyl)pyridazin-3-amine (5) (250 mg, 1.5 mmol), Cs₂CO₃ (1.0 g, 3.1 mmol) was added degassed dry 1,4-dioxane (13 mL). The reaction mixture was then flushed with Ar(g) for 1 min before Pd₂(dba)₃ (70 mg, 0.08 mmol) and Xantphos (98 mg, 0.17 mmol) were added. The reaction mixture was heated up to 90° C. for 40 h. It was then cooled down to rt and concentrated in vacuo, CH₂Cl₂ (15 mL) and H₂O (15 mL) were added. The organic phase was separated and the water layer was extracted with CH₂Cl₂ (15 mL). The organic layers were combined and Pd-scavenger (MP-TMT, ˜400 mg, 1.3 mmol/g) was added. This was shaken for several hours followed by filtration. The filtrate was concentrated in vacuo, dissolved in DMSO (4 mL) and purified by basic prep LCMS to yield (7) as a solid (113 mg, 30%).

Under Ar(g), to a solution of (7) (72 mg, 0.3 mmol) in dry DMF (1.3 mL) was added NaH (60%, 14.4 mg, 0.36 mmol) in one portion at 0° C. After 5 min, a solution of (8) (113 mg, 0.36 mmol) in dry DMF (1.2 mL) was added dropwise. The reaction mixture was allowed to warm up to rt overnight. The reaction mixture was purified by acidic prep LCMS to yield (EF) as a solid (57 mg, 40%).

LCMS (ES): Found 474.3 [M+H]⁺.

¹H NMR (400 MHz, DMSO-d₆), δ: 9.33 (d, J=2.0 Hz, 1H), 8.91 (d, J=1.4 Hz, 1H), 8.43 (dd, J=2.7, 1.5 Hz, 1H), 8.38 (d, J=2.6 Hz, 1H), 8.18-8.23 (m, 1H), 7.75 (d, J=3.7 Hz, 1H), 7.33 (d, J=3.8 Hz, 1H), 5.80 (s, 2H).

### Example EG

### N-(Pyridin-2-yl)-5-(trifluoromethyl)-N-({5-[5-(trifluoromethyl)-1,2,4-oxadiazol-3-yl]thiophen-2-yl}methyl)pyridazin-3-amine

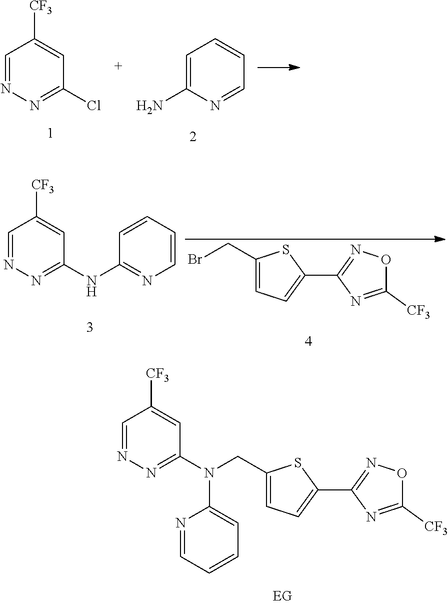

A suspension of 3-chloro-5-(trifluoromethyl)pyridazine (1) (100 mg, 0.44 mmol), 2-aminopyridine (2) (45 mg, 0.48 mmol), Cs₂CO₃ (285 mg, 0.88 mmol) and Xantphos (25 mg, 0.04 mmol) in 1,4-dioxane (3 mL) was purged with Ar(g) for 20 min. Pd₂(dba)₃ (20 mg, 0.02 mmol) was added and the reaction mixture was heated up to 90° C. for 7 h. Once cooled down to rt, it was poured into H₂O (20 mL) and extracted with EtOAc (3×20 mL). The combined organics were washed with brine (20 mL), dried over MgSO₄, filtered and concentrated in vacuo. Purification by silica gel column chromatography with hexane/EtOAc (1:0-1:1) yielded (3) as a solid (60 mg, 57%).

KO^{t}Bu (1M in THF, 0.17 mL) was added to a solution of (3) (34 mg, 0.14 mmol) in DMF (1.4 mL) at 0° C. under Ar(g). The reaction mixture was stirred for 15 min then (4) (47 mg, 0.16 mmol) was added in one portion. It was stirred for a further 3 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-7:3) then CH₂Cl₂/EtOAc (1:0-17:3) yielded (EG) as a beige solid (34 mg, 51%).

LCMS (ES): Found 472.8 [M+H]⁺.

¹H NMR (300 MHz, DMSO-d₆), δ: 9.23 (d, J=1.9 Hz, 1H), 8.35-8.43 (m, 1H), 7.82-7.94 (m, 2H), 7.70-7.77 (m, 1H), 7.54 (dd, J=8.3, 0.8 Hz, 1H), 7.27 (d, J=3.8 Hz, 1H), 7.16-7.24 (m, 1H), 5.77 (s, 2H).

¹⁹F NMR (282 MHz, DMSO-d₆), δ: −63.32 (s, 3F), −64.80 (s, 3F).

### Example EH

### N-[3-(Morpholin-4-yl)pyridin-2-yl]-5-(trifluoromethyl)-N-({5-[5-(trifluoromethyl)-1,2,4-oxadiazol-3-yl]thiophen-2-yl}methyl)pyridazin-3-amine

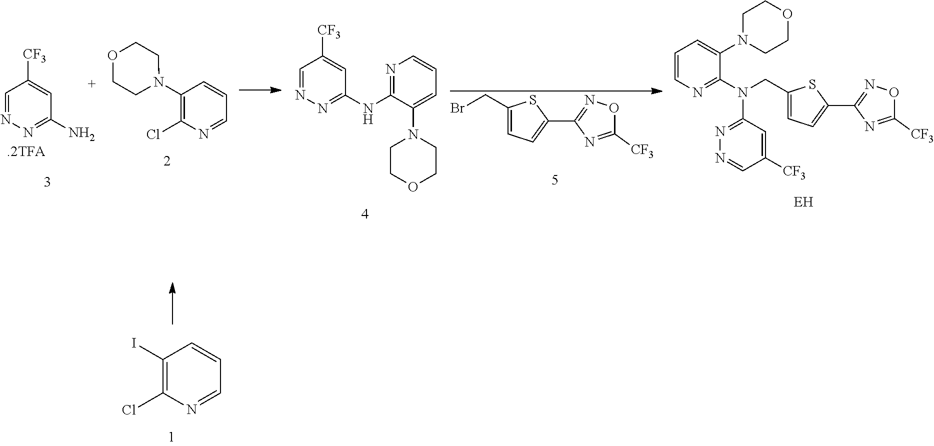

A suspension of 2-chloro-3-iodopyridine (1) (1.07 g, 4.46 mmol), morpholine (0.43 mL, 4.90 mmol), Cs₂CO₃ (2.90 gmg, 8.92 mmol) and Xantphos (260 mg, 0.4 mmol) in 1,4-dioxane (50 mL) was purged with Ar(g) for 0.5 h. Pd₂(dba)₃ (204 mg, 0.22 mmol) was added and the reaction mixture was heated up to 90° C. for 2 d. Once cooled down to rt, it was poured into a mixture of H₂O (50 mL) and brine (50 mL), then extracted with EtOAc (3×50 mL). The combined organics were washed with brine (50 mL), dried over MgSO₄, filtered and concentrated in vacuo. Purification by silica gel column chromatography with hexane/EtOAc (1:0-7:3) yielded (2) as a solid (628 mg, 71%).

A suspension of (3) (985 mg, 2.52 mmol), (2) (500 mg, 2.52 mmol), Cs₂CO₃ (3.28 g, 10.1 mmol) and Xantphos (146 mg, 0.25 mmol) in 1,4-dioxane (20 mL) was purged with Ar(g) for 25 min. Pd₂(dba)₃ (115 mg, 0.13 mmol) was added and the reaction mixture was heated up to 90° C. for 2 d. Once cooled down to rt, it was poured into a mixture of H₂O (25 mL) and brine (35 mL), then extracted with EtOAc (3×50 mL). The combined organics were washed with brine (50 mL), dried over MgSO₄, filtered and concentrated in vacuo. Purification by silica gel column chromatography with hexane/EtOAc (1:0-1:3) yielded (4) as a beige solid (495 mg, 61%).

KO^{t}Bu (1M in THF, 0.37 mL) was added to a solution of (4) (100 mg, 0.31 mmol) in DMF (3 mL) at 0° C. under Ar(g). The reaction mixture was stirred for 15 min then (5) (106 mg, 0.34 mmol) was added in one portion. It was stirred for a further 3 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-3:2) yielded (EH) as a beige solid (145 mg, 85%).

LCMS (ES): Found 557.9 [M+H]⁺.

¹H NMR (300 MHz, DMSO-d₆), δ: 9.12-9.19 (m, 1H), 8.27 (dd, J=4.5, 1.5 Hz, 1H), 7.72 (d, J=3.6 Hz, 1H), 7.55-7.62 (m, 1H), 7.32-7.40 (m, 1H), 7.23 (d, J=3.8 Hz, 1H), 7.06-7.11 (m, 1H), 5.77 (s, 2H), 3.10-3.22 (m, 4H), 2.65-2.77 (m, 4H).

¹⁹F NMR (282 MHz, DMSO-d₆), δ: −63.19 (s, 3F), −64.79 (s, 3F).

### Example EI

### N-(Pyridin-3-yl)-5-(trifluoromethyl)-N-({5-[5-(trifluoromethyl)-1,2,4-oxadiazol-3-yl]thiophen-2-yl}methyl)pyridazin-3-amine

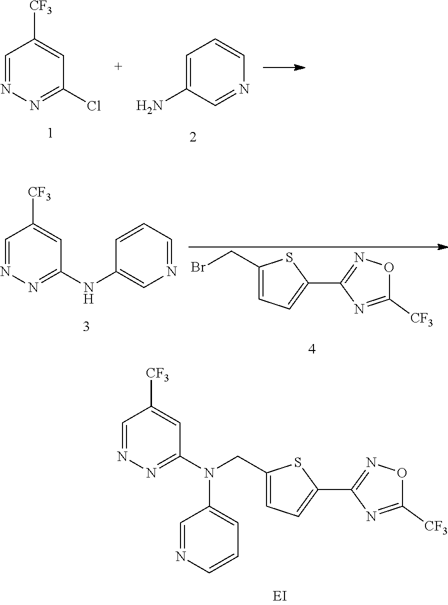

A suspension of 3-chloro-5-(trifluoromethyl)pyridazine (1) (100 mg, 0.44 mmol), 3-aminopyridine (2) (45 mg, 0.48 mmol), Cs₂CO₃ (285 mg, 0.88 mmol) and Xantphos (25 mg, 0.04 mmol) in 1,4-dioxane (3 mL) was purged with Ar(g) for 20 min. Pd₂(dba)₃ (20 mg, 0.02 mmol) was added and the reaction mixture was heated up to 90° C. for 7 h. Once cooled down to rt, it was poured into H₂O (20 mL) and extracted with EtOAc (3×20 mL). The combined organics were washed with brine (20 mL), dried over MgSO₄, filtered and concentrated in vacuo. Purification by silica gel column chromatography with hexane/EtOAc (1:0-1:9) yielded (3) as a beige solid (32 mg, 30%).

KO^{t}Bu (1M in THF, 0.14 mL) was added to a solution of (3) (29 mg, 0.12 mmol) in DMF (1.2 mL) at 0° C. under Ar(g). The reaction mixture was stirred for 0.5 h then (4) (40 mg, 0.13 mmol) was added in one portion. It was stirred for a further 3 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue.

Purification by silica gel column chromatography with hexane/EtOAc (1:0-1:1) yielded (EI) as a beige solid (24 mg, 43%).

LCMS (ES): Found 472.7 [M+H]⁺.

¹H NMR (300 MHz, DMSO-d₆), δ: 9.13 (d, J=1.7 Hz, 1H), 8.61 (d, J=2.3 Hz, 1H), 8.55 (dd, J=4.7, 1.5 Hz, 1H), 7.85 (ddd, J=8.2, 2.6, 1.4 Hz, 1H), 7.75 (d, J=3.6 Hz, 1H), 7.52 (ddd, J=8.2, 4.8, 0.6 Hz, 1H), 7.18 (d, J=3.8 Hz, 1H), 7.15 (dd, J=1.7, 0.9 Hz, 1H), 5.60 (s, 2H).

¹⁹F NMR (282 MHz, DMSO-d₆), δ: −63.52 (s, 3F), −64.79 (s, 3F).

### Example EJ

### N-(Pyridin-4-yl)-5-(trifluoromethyl)-N-({5-[5-(trifluoromethyl)-1,2,4-oxadiazol-3-yl]thiophen-2-yl}methyl)pyridazin-3-amine

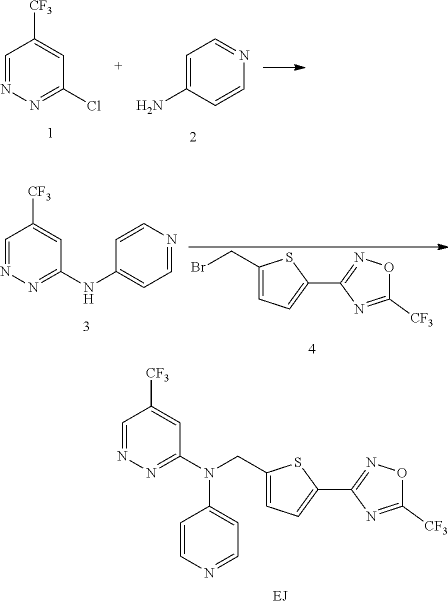

A suspension of 3-chloro-5-(trifluoromethyl)pyridazine (1) (100 mg, 0.44 mmol), 4-aminopyridine (2) (45 mg, 0.48 mmol), Cs₂CO₃ (285 mg, 0.88 mmol) and Xantphos (25 mg, 0.04 mmol) in 1,4-dioxane (3 mL) was purged with Ar(g) for 20 min. Pd₂(dba)₃ (20 mg, 0.02 mmol) was added and the reaction mixture was heated up to 90° C. overnight. Once cooled down to rt, it was poured into a mixture of H₂O (20 mL) and brine (10 mL), then extracted with EtOAc (4×20 mL). The combined organics were dried over MgSO₄, filtered and concentrated in vacuo. Purification by silica gel column chromatography with CH₂Cl₂/EtOAc (1:0-0:1) yielded (3) as a brown solid (35 mg, 33%).

KO^{t}Bu (1M in THF, 0.15 mL) was added to a solution of (3) (30 mg, 0.13 mmol) in DMF (1.2 mL) at 0° C. under Ar(g). The reaction mixture was stirred for 0.5 h then (4) (42 mg, 0.14 mmol) was added in one portion. It was stirred for a further 2.5 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 40 min. The reaction mixture was filtered and concentrated in vacuo to afford a residue. Purification by silica gel column chromatography with hexane/EtOAc (1:0-0:1) yielded (EJ) as a solid (23 mg, 38%).

LCMS (ES): Found 472.7 [M+H]⁺.

¹H NMR (300 MHz, DMSO-d₆), δ: 9.27 (m, 1H), 8.49-8.59 (m, 2H), 7.75 (d, J=3.8 Hz, 1H), 7.71 (m, 1H), 7.41-7.48 (m, 2H), 7.25 (d, J=3.8 Hz, 1H), 5.69 (s, 2H).

¹⁹F NMR (282 MHz, DMSO-d₆), δ: −63.32 (s, 3F), −64.80 (s, 3F).

### Example EK

### N-(Pyrimidin-2-yl)-5-(trifluoromethyl)-N-({5-[5-(trifluoromethyl)-1,2,4-oxadiazol-3-yl]thiophen-2-yl}methyl)pyridazin-3-amine

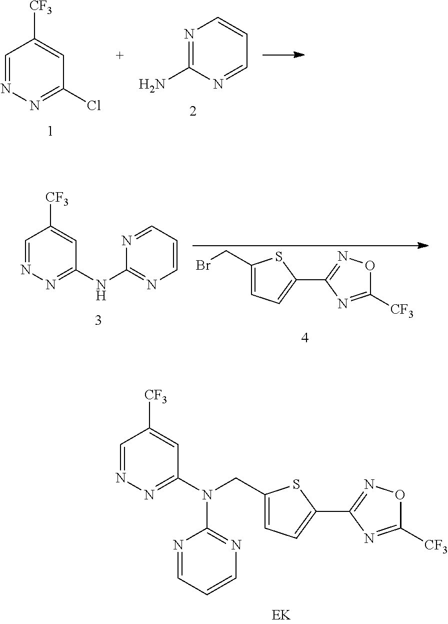

A suspension of 3-chloro-5-(trifluoromethyl)pyridazine (1) (100 mg, 0.44 mmol), 2-aminopyrimidine (2) (42 mg, 0.42 mmol), Cs₂CO₃ (285 mg, 0.88 mmol) and Xantphos (25 mg, 0.04 mmol) in 1,4-dioxane (3 mL) was purged with Ar(g) for 20 min. Pd₂(dba)₃ (20 mg, 0.02 mmol) was added and the reaction mixture was heated up to 90° C. overnight. Once cooled down to rt, it was poured into H₂O (20 mL) and extracted with EtOAc (4×15 mL). The combined organics were dried over MgSO₄, filtered and concentrated in vacuo. Purification by reverse-phase column chromatography with H₂O/MeCN (9:1-0:1) yielded (3) as a solid (40 mg, 38%).

KO^{t}Bu (1M in THF, 0.18 mL) was added to a solution of (3) (35 mg, 0.15 mmol) in DMF (1.4 mL) at 0° C. under Ar(g). The reaction mixture was stirred for 10 min then (4) (50 mg, 0.16 mmol) was added in one portion. It was stirred for a further 2.5 h, poured into a mixture of H₂O (10 mL) and brine (10 mL), then extracted with EtOAc (2×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-4:1) yielded (EK) as an off-white solid (49 mg, 71%).

LCMS (ES): Found 473.8 [M+H]⁺.

¹H NMR (300 MHz, DMSO-d₆), δ: 9.39 (d, J=1.9 Hz, 1H), 8.70 (d, J=4.9 Hz, 2H), 8.61 (dd, J=1.8, 0.8 Hz, 1H), 7.75 (d, J=3.8 Hz, 1H), 7.32 (d, J=3.8 Hz, 1H), 7.20 (t, J=4.8 Hz, 1H), 5.89 (s, 2H).

¹⁹F NMR (282 MHz, DMSO-d₆), δ: −63.07 (s, 3F), −64.79 (s, 3F).

### Example EL

### N-(Pyrimidin-4-yl)-5-(trifluoromethyl)-N-({5-[5-(trifluoromethyl)-1,2,4-oxadiazol-3-yl]thiophen-2-yl}methyl)pyridazin-3-amine

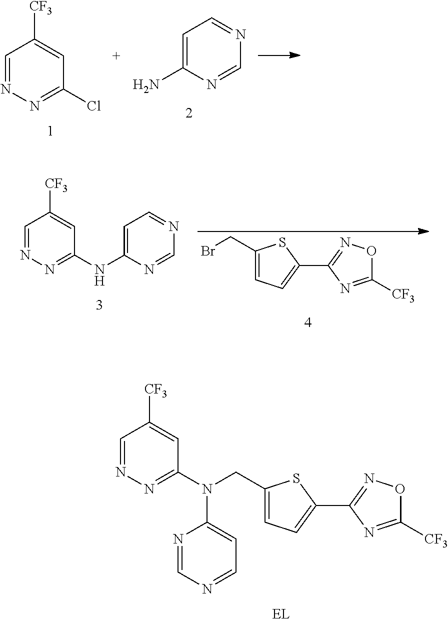

A suspension of 3-chloro-5-(trifluoromethyl)pyridazine (1) (100 mg, 0.44 mmol), 4-aminopyrimidine (2) (42 mg, 0.4 mmol), Cs₂CO₃ (285 mg, 0.88 mmol) and Xantphos (25 mg, 0.04 mmol) in 1,4-dioxane (3 mL) was purged with Ar(g) for 20 min. Pd₂(dba)₃ (20 mg, 0.02 mmol) was added and the reaction mixture was heated up to 90° C. overnight. Once cooled down to rt, it was poured into a mixture of H₂O (20 mL) and brine (10 mL), then extracted with EtOAc (4×20 mL). The combined organics were washed with brine (10 mL), dried over MgSO₄, filtered and concentrated in vacuo. Purification by silica gel column chromatography with hexane/EtOAc (1:0-0:1) yielded (3) as a brown solid (39 mg, 37%).

KO^{t}Bu (1M in THF, 0.15 mL) was added to a solution of (3) (30 mg, 0.12 mmol) in DMF (1.2 mL) at 0° C. under Ar(g). The reaction mixture was stirred for 15 min then (4) (43 mg, 0.14 mmol) was added in one portion. It was stirred for a further 1.5 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-1:3) yielded (EL) as a brown film (12 mg, 20%).

LCMS (ES): Found 473.8 [M+H]⁺.

¹H NMR (300 MHz, DMSO-d₆), δ: 9.48 (d, J=1.5 Hz, 1H), 8.83 (d, J=0.8 Hz, 1H), 8.59 (d, J=6.0 Hz, 1H), 8.46 (dd, J=1.8, 0.8 Hz, 1H), 7.77 (d, J=3.8 Hz, 1H), 7.46 (dd, J=6.0, 1.3 Hz, 1H), 7.37 (d, J=3.8 Hz, 1H), 5.78 (s, 2H).

¹⁹F NMR (282 MHz, DMSO-d₆), δ: −62.97 (s, 3F), −64.80 (s, 3F).

### Example EM

### N-(6-Methylpyrazin-2-yl)-5-(trifluoromethyl)-N-({5-[5-(trifluoromethyl)-1,2,4-oxadiazol-3-yl]thiophen-2-yl}methyl)pyridazin-3-amine

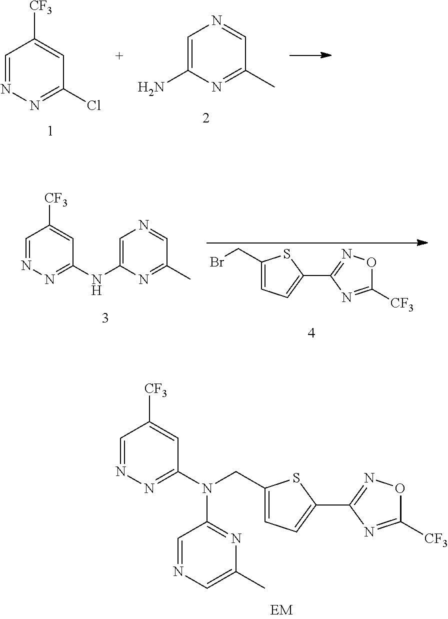

A suspension of 3-chloro-5-(trifluoromethyl)pyridazine (1) (100 mg, 0.44 mmol), 2-amino-6-methylpyrazine (2) (48 mg, 0.44 mmol), Cs₂CO₃ (285 mg, 0.88 mmol) and Xantphos (25 mg, 0.04 mmol) in 1,4-dioxane (3 mL) was purged with Ar(g) for 0.5 h. Pd₂(dba)₃ (20 mg, 0.02 mmol) was added and the reaction mixture was heated up to 90° C. for 4 h. Once cooled down to rt, it was poured into a mixture of H₂O (20 mL) and brine (10 mL), then extracted with EtOAc (5×20 mL). The combined organics were washed with brine (20 mL), dried over MgSO₄, filtered and concentrated in vacuo. Purification by silica gel column chromatography with hexane/EtOAc (1:0-2:3) yielded (3) as a solid (60 mg, 54%).

KO^{t}Bu (1M in THF, 0.19 mL) was added to a solution of (3) (40 mg, 0.16 mmol) in DMF (1.6 mL) at 0° C. under Ar(g). The reaction mixture was stirred for 15 min then (4) (54 mg, 0.17 mmol) was added in one portion. It was stirred for a further 2 h, poured into a mixture of H₂O (10 mL) and brine (10 mL), then extracted with EtOAc (2×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-1:1) yielded (EM) as a brown oil (34 mg, 44%).

LCMS (ES): Found 487.8 [M+H]⁺.

¹H NMR (300 MHz, DMSO-d₆), δ: 9.30 (d, J=1.5 Hz, 1H), 8.69 (m, 1H), 8.28 (m, 1H), 8.19 (dd, J=1.7, 0.9 Hz, 1H), 7.75 (d, J=3.8 Hz, 1H), 7.32-7.36 (m, 1H), 5.78 (s, 2H), 2.43 (s, 3H).

¹⁹F NMR (282 MHz, DMSO-d₆), δ: −63.22 (s, 3F), −64.80 (s, 3F).

### Example EN

### N-(6-Methoxypyrazin-2-yl)-5-(trifluoromethyl)-N-({5-[5-(trifluoromethyl)-1,2,4-oxadiazol-3-yl]thiophen-2-yl}methyl)pyridazin-3-amine

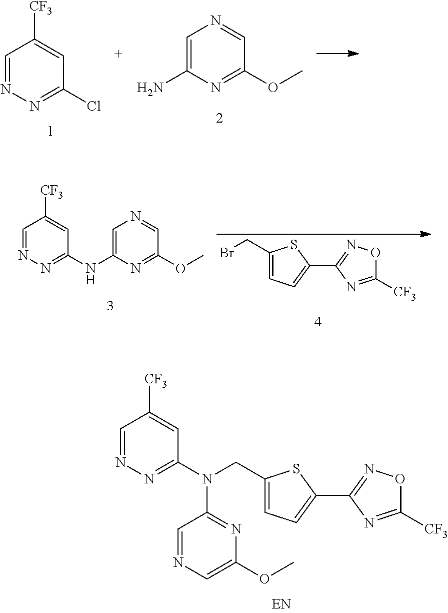

A suspension of 3-chloro-5-(trifluoromethyl)pyridazine (1) (100 mg, 0.44 mmol), 2-amino-6-methoxypyrazine (2) (55 mg, 0.44 mmol), Cs₂CO₃ (285 mg, 0.88 mmol) and Xantphos (25 mg, 0.04 mmol) in 1,4-dioxane (3 mL) was purged with Ar(g) for 0.5 h. Pd₂(dba)₃ (20 mg, 0.02 mmol) was added and the reaction mixture was heated up to 90° C. for 4 h. Once cooled down to rt, it was poured into a mixture of H₂O (20 mL) and brine (10 mL), then extracted with EtOAc (5×20 mL). The combined organics were washed with brine (20 mL), dried over MgSO₄, filtered and concentrated in vacuo. Purification by silica gel column chromatography with hexane/EtOAc (1:0-2:3) yielded (3) as a solid (58 mg, 49%).

KO^{t}Bu (1M in THF, 0.18 mL) was added to a solution of (3) (40 mg, 0.16 mmol) in DMF (1.6 mL) at 0° C. under Ar(g). The reaction mixture was stirred for 15 min then (4) (51 mg, 0.16 mmol) was added in one portion. It was stirred for a further 2 h, poured into a mixture of H₂O (10 mL) and brine (10 mL), then extracted with EtOAc (2×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-3:2) yielded (EN) as a brown oil (28 mg, 38%).

LCMS (ES): Found 503.7 [M+H]⁺.

¹H NMR (300 MHz, DMSO-d₆), δ: 9.33 (m, 1H), 8.41 (m, 1H), 8.38 (m, 1H), 8.02 (m, 1H), 7.77 (d, J=3.8 Hz, 1H), 7.39 (d, J=3.8 Hz, 1H), 5.81 (s, 2H), 3.80 (s, 3H).

¹⁹F NMR (282 MHz, DMSO-d₆), δ: −63.07 (s, 3F), −64.80 (s, 3F).

### Example EO

### N-(5-Methylpyrazin-2-yl)-5-(trifluoromethyl)-N-({5-[5-(trifluoromethyl)-1,2,4-oxadiazol-3-yl]thiophen-2-yl}methyl)pyridazin-3-amine

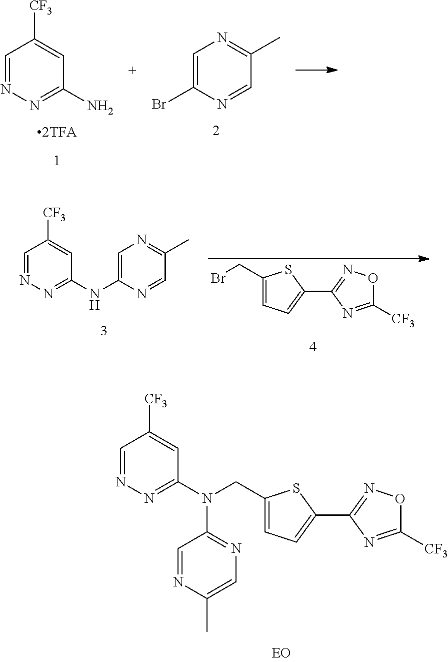

A suspension of (1) (171 mg, 0.44 mmol), 2-bromo-5-methylpyrazine (2) (76 mg, 0.44 mmol), Cs₂CO₃ (570 mg, 1.75 mmol) and Xantphos (25 mg, 0.04 mmol) in 1,4-dioxane (4 mL) was purged with Ar(g) for 1 h. Pd₂(dba)₃ (20 mg, 0.02 mmol) was added and the reaction mixture was heated up to 90° C. overnight. Once cooled down to rt, it was poured into a mixture of H₂O (20 mL) and brine (10 mL), then extracted with EtOAc (4×10 mL). The combined organics were washed with brine (20 mL), dried over MgSO₄, filtered and concentrated in vacuo. Purification by silica gel column chromatography with hexane/EtOAc (1:0-1:1) yielded (3) as a solid (75 mg, 67%).

KO^{t}Bu (1M in THF, 0.24 mL) was added to a solution of (3) (50 mg, 0.20 mmol) in DMF (2 mL) at 0° C. under Ar(g). The reaction mixture was stirred for 10 min then (4) (68 mg, 0.22 mmol) was added in one portion. It was stirred for a further 3 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with CH₂Cl₂/EtOAc (1:0-9:1) yielded (EO) as a solid (49 mg, 51%).

LCMS (ES): Found 487.8 [M+H]⁺.

¹H NMR (300 MHz, DMSO-d₆), δ: 9.26 (d, J=1.7 Hz, 1H), 8.79 (d, J=1.5 Hz, 1H), 8.34 (s, 1H), 8.00-8.05 (m, 1H), 7.74 (d, J=3.8 Hz, 1H), 7.30 (d, J=3.8 Hz, 1H), 5.76 (s, 2H), 2.48 (s, 3H).

¹⁹F NMR (282 MHz, DMSO-d₆), δ: −63.18 (s, 3F), −64.80 (s, 3F).

### Example EP

### N-(5-Methoxypyrazin-2-yl)-5-(trifluoromethyl)-N-({5-[5-(trifluoromethyl)-1,2,4-oxadiazol-3-yl]thiophen-2-yl}methyl)pyridazin-3-amine

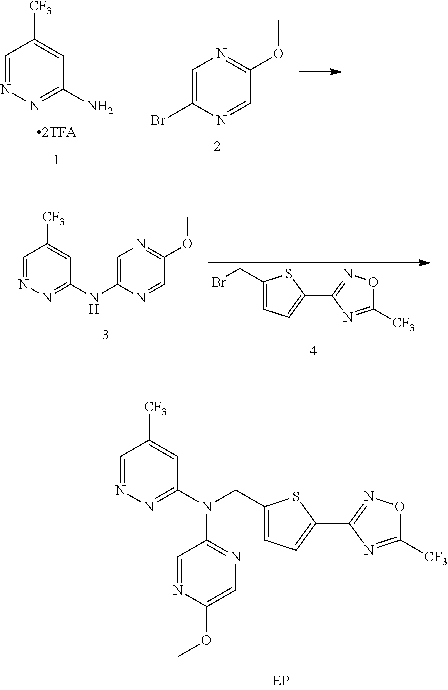

A suspension of (1) (171 mg, 0.44 mmol), 2-bromo-5-methoxypyrazine (2) (83 mg, 0.44 mmol), Cs₂CO₃ (570 mg, 1.75 mmol) and Xantphos (25 mg, 0.04 mmol) in 1,4-dioxane (4 mL) was purged with Ar(g) for 1 h. Pd₂(dba)₃ (20 mg, 0.02 mmol) was added and the reaction mixture was heated up to 90° C. overnight. Once cooled down to rt, it was poured into a mixture of H₂O (20 mL) and brine (10 mL), then extracted with EtOAc (4×10 mL). The combined organics were washed with brine (20 mL), dried over MgSO₄, filtered and concentrated in vacuo. Purification by silica gel column chromatography with hexane/EtOAc (1:0-1:1) yielded (3) as a solid (68 mg, 57%).

KO^{t}Bu (1M in THF, 0.24 mL) was added to a solution of (3) (53 mg, 0.20 mmol) in DMF (2 mL) at 0° C. under Ar(g). The reaction mixture was stirred for 10 min then (4) (68 mg, 0.22 mmol) was added in one portion. It was stirred for a further 3 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-4:1) yielded (EP) as a solid (55 mg, 56%).

LCMS (ES): Found 503.8 [M+H]⁺.

¹H NMR (300 MHz, DMSO-d₆), δ: 9.16 (d, J=1.5 Hz, 1H), 8.41-8.47 (m, 1H), 8.20-8.25 (m, 1H), 7.74 (d, J=3.8 Hz, 1H), 7.69-7.72 (m, 1H), 7.24 (d, J=3.8 Hz, 1H), 5.66 (s, 2H), 3.94 (s, 3H).

¹⁹F NMR (282 MHz, DMSO-d₆), δ: −63.20 (s, 3F), −64.79 (s, 3F).

### Example EQ

### N-(Pyrimidin-5-yl)-5-(trifluoromethyl)-N-({5-[5-(trifluoromethyl)-1,2,4-oxadiazol-3-yl]thiophen-2-yl}methyl)pyridazin-3-amine

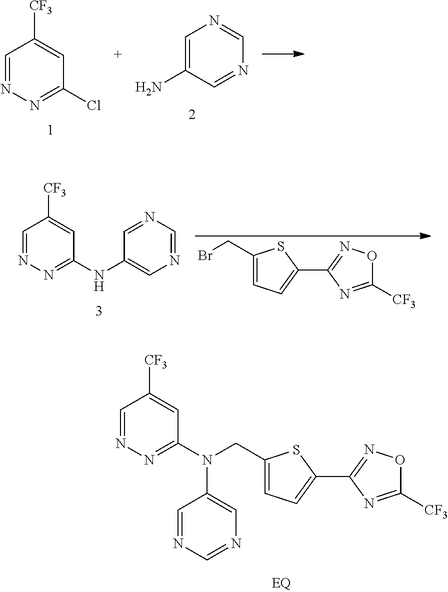

A suspension of 3-chloro-5-(trifluoromethyl)pyridazine (1) (100 mg, 0.44 mmol), 5-aminopyrimidine (2) (42 mg, 0.44 mmol), Cs₂CO₃ (285 mg, 0.88 mmol) and Xantphos (25 mg, 0.04 mmol) in 1,4-dioxane (3 mL) was purged with Ar(g) for 20 min. Pd₂(dba)₃ (20 mg, 0.02 mmol) was added and the reaction mixture was heated up to 90° C. overnight. Once cooled down to rt, it was poured into a mixture of H₂O (15 mL) and brine (5 mL), then extracted with EtOAc (3×20 mL). The combined organics were washed with brine (2×10 mL), dried over MgSO₄, filtered and concentrated in vacuo. Purification by silica gel column chromatography with hexane/EtOAc (1:0-0:1) yielded (3) as a solid (26 mg, 25%).

KO^{t}Bu (1M in THF, 0.11 mL) was added to a solution of (3) (22 mg, 0.09 mmol) in DMF (1.1 mL) at 0° C. under Ar(g). The reaction mixture was stirred for 10 min then (4) (31 mg, 0.10 mmol) was added in one portion. It was stirred for a further 2.5 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-1:1) yielded (EQ) as an orange solid (16 mg, 37%).

LCMS (ES): Found 473.8 [M+H]⁺.

¹H NMR (300 MHz, DMSO-d₆), δ: 9.18 (d, J=1.1 Hz, 1H), 9.09-9.15 (m, 1H), 8.90 (s, 2H), 7.76 (d, J=3.6 Hz, 1H), 7.54 (s, 1H), 7.23 (d, J=3.8 Hz, 1H), 5.64 (s, 2H).

¹⁹F NMR (282 MHz, DMSO-d₆), δ: −63.23 (s, 3F), −64.79 (s, 3F).

### Example ER

### N-(Pyridazin-3-yl)-5-(trifluoromethyl)-N-({5-[5-(trifluoromethyl)-1,2,4-oxadiazol-3-yl]thiophen-2-yl}methyl)pyridazin-3-amine

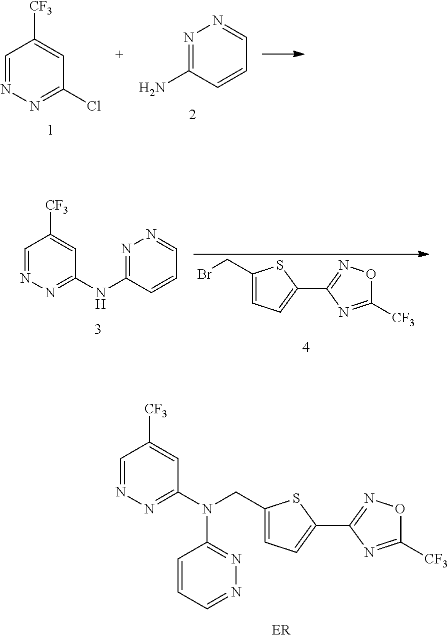

A suspension of 3-chloro-5-(trifluoromethyl)pyridazine (1) (100 mg, 0.44 mmol), 3-aminopyridazine (2) (46 mg, 0.48 mmol), Cs₂CO₃ (285 mg, 0.88 mmol) and Xantphos (25 mg, 0.04 mmol) in 1,4-dioxane (3 mL) was purged with Ar(g) for 20 min. Pd₂(dba)₃ (20 mg, 0.02 mmol) was added and the reaction mixture was heated up to 90° C. overnight. Once cooled down to rt, it was poured into a mixture of H₂O (20 mL) and brine (10 mL), then extracted with EtOAc (4×20 mL). The combined organics were washed with brine (2×10 mL), dried over MgSO₄, filtered and concentrated in vacuo. Purification by silica gel column chromatography with CH₂Cl₂/EtOAc (1:0-0:1) yielded (3) as a solid (27 mg, 26%).

KO^{t}Bu (1M in THF, 0.11 mL) was added to a solution of (3) (21 mg, 0.09 mmol) in DMF (0.9 mL) at 0° C. under Ar(g). The reaction mixture was stirred for 10 min then (4) (29 mg, 0.10 mmol) was added in one portion. It was stirred for a further 3 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with CH₂Cl₂/EtOAc (1:0-3:1) yielded (ER) as an orange solid (8 mg, 20%).

LCMS (ES): Found 473.8 [M+H]⁺.

¹H NMR (300 MHz, DMSO-d₆), δ: 9.32 (d, J=1.7 Hz, 1H), 8.98-9.05 (m, 1H), 8.15 (d, J=0.9 Hz, 1H), 7.90 (dd, J=9.0, 1.3 Hz, 1H), 7.69-7.77 (m, 2H), 7.31 (d, J=4.0 Hz, 1H), 5.84 (s, 2H).

¹⁹F NMR (282 MHz, DMSO-d₆), δ: −63.11 (s, 3F), −64.80 (s, 3F).

### Example ES

### 6-[({5-[5-(Trifluoromethyl)-1,2,4-oxadiazol-3-yl]thiophen-2-yl}methyl)[5-(trifluoromethyl)pyridazin-3-yl]amino]pyridazine-4-carboxylicacid

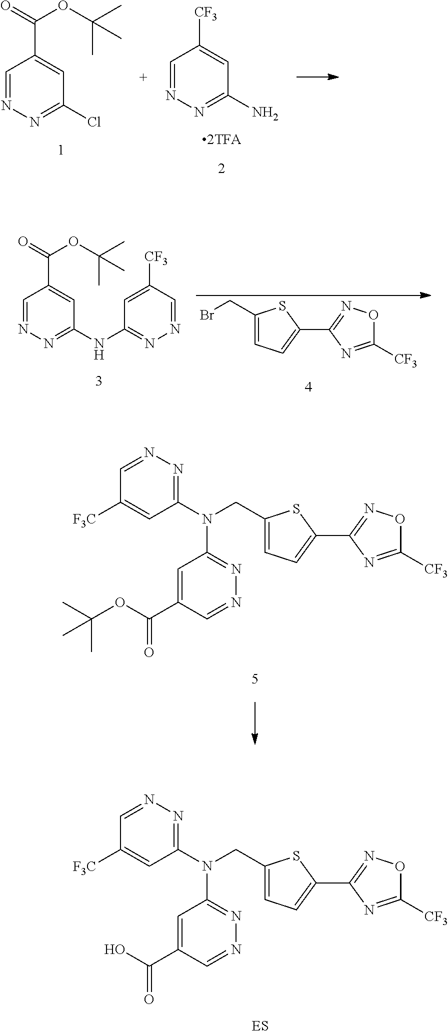

A suspension of (1) (274 mg, 1.28 mmol), (2) (500 mg, 1.28 mmol), Cs₂CO₃ (1.67 mg, 5.11 mmol) and Xantphos (74 mg, 0.13 mmol) in 1,4-dioxane (20 mL) was purged with Ar(g) for 0.5 h. Pd₂(dba)₃ (58 mg, 0.06 mmol) was added and the reaction mixture was heated up to 90° C. overnight. Once cooled down to rt, it was poured into a mixture of H₂O (25 mL) and brine (25 mL), then extracted with EtOAc (4×50 mL). The combined organics were washed with brine (50 mL), dried over MgSO₄, filtered and concentrated in vacuo. Purification by silica gel column chromatography with hexane/EtOAc (1:0-3:2) yielded (3) as a solid (245 mg, 56%).

KO^{t}Bu (1M in THF, 0.85 mL) was added to a solution of (3) (240 mg, 0.70 mmol) in DMF (10 mL) at 0° C. under Ar(g). The reaction mixture was stirred for 10 min then (4) (251 mg, 0.80 mmol) was added in one portion. It was stirred for a further 2 h, poured into a mixture of H₂O (10 mL) and brine (10 mL), then extracted with EtOAc (3×20 mL). The combined organics were washed with brine (10 mL), dried over MgSO₄ and treated with MP-TMT resin (170 mg, 1.3 mmol/g) for 0.5 h. The reaction mixture was filtered and concentrated in vacuo to afford a residue. Purification by silica gel column chromatography with hexane/EtOAc (1:0-2:3) yielded (5) as an orange solid (163 mg, 40%).

A solution of (5) (160 mg, 0.28 mmol) in TFA (8 mL) was stirred at rt overnight. The reaction mixture was concentrated in vacuo. It was diluted with CH₂Cl₂ (10 mL) and re-concentrated in vacuo. The residue was purified by silica gel column chromatography with CH₂Cl₂/MeOH (1:0-4:1) to give a residue which was re-concentrated from MeOH/Toluene (1:1, 10 mL), MeOH (10 mL) and CH₂Cl₂ (10 mL) to yield (ES) as a solid (86 mg, 60%).

LCMS (ES): Found 517.8 [M+H]⁺.

¹H NMR (300 MHz, DMSO-d₆), δ: 14.26 (br s, 1H), 9.36 (d, J=1.5 Hz, 1H), 9.29 (d, J=1.7 Hz, 1H), 8.25-8.29 (m, 1H), 8.21 (d, J=1.7 Hz, 1H), 7.75 (d, J=3.6 Hz, 1H), 7.32 (d, J=3.8 Hz, 1H), 5.89 (s, 2H).

¹⁹F NMR (282 MHz, DMSO-d₆), δ: −63.04 (s, 3F), −64.80 (s, 3F).

### Example ET

### N-[5-(3-Methoxyazetidine-1-carbonyl)pyridazin-3-yl]-5-(trifluoromethyl)-N-({5-[5-(trifluoromethyl)-1,2,4-oxadiazol-3-yl]thiophen-2-yl}methyl)pyridazin-3-amine

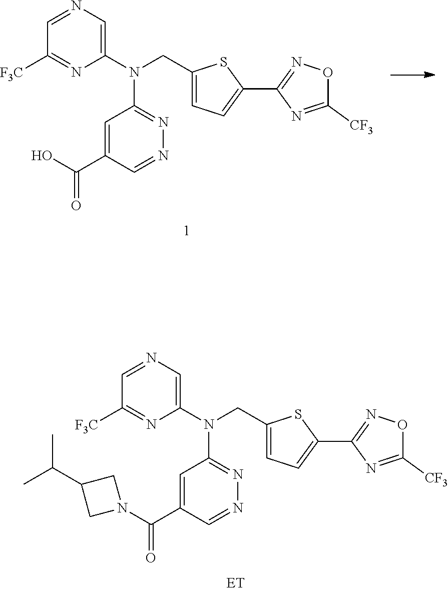 [SMILES: O=C(O)c1cnnc(N(Cc2ccc(-c3noc(C(F)(F)F)n3)s2)c2cncc(C(F)(F)F)n2)c1 ; CC(C)C1CN(C(=O)c2cnnc(N(Cc3ccc(-c4noc(C(F)(F)F)n4)s3)c3cncc(C(F)(F)F)n3)c2)C1]

To a suspension of (1) (50 mg, 0.10 mmol) and 3-methoxyazetidine hydrochloride (12 mg, 0.10 mmol) in MeCN (2 mL) was added diisopropylethylamine (0.02 mL, 0.10 mmol), followed by dicyclohexylcarbodiimide (22 mg, 0.11 mmol). The reaction mixture was stirred at rt overnight. It was then heated up to 60° C. overnight. Once cooled down to rt, it was filtered and concentrated in vacuo. Purification by silica gel column chromatography with CH₂Cl₂/EtOAc (1:0-0:1) yielded (ET) as an orange solid (13 mg, 23%).

LCMS (ES): Found 586.8 [M+H]⁺.

¹H NMR (300 MHz, DMSO-d₆), δ: 9.34-9.38 (m, 1H), 9.10 (d, J=1.7 Hz, 1H), 8.22-8.30 (m, 1H), 7.99 (d, J=1.5 Hz, 1H), 7.75 (d, J=3.8 Hz, 1H), 7.30 (d, J=3.8 Hz, 1H), 5.89 (s, 2H), 4.38-4.48 (m, 1H), 4.16-4.32 (m, 3H), 3.82-3.93 (m, 1H), 3.21 (s, 3H).

¹⁹F NMR (282 MHz, DMSO-d₆), δ: −63.04 (s, 3F), −64.81 (s, 3F).

### Example EU

### 5-(Trifluoromethyl)-N-({5-[5-(trifluoromethyl)-1,2,4-oxadiazol-3-yl]thiophen-2-yl}methyl)-N-[5-(trifluoromethyl)pyridazin-3-yl]pyridazin-3-amine

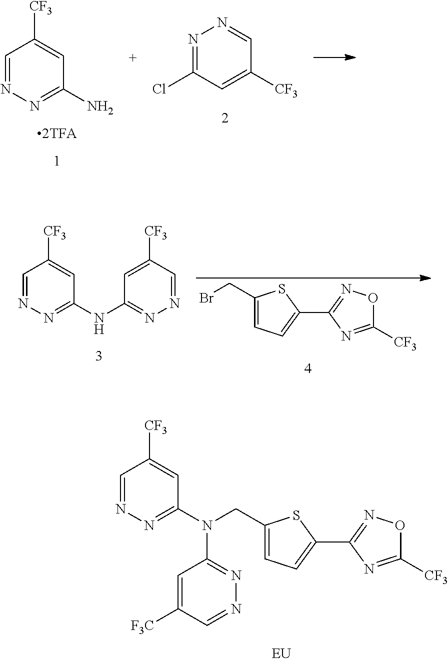

A suspension of (1) (171 mg, 0.44 mmol), 3-chloro-5-(trifluoromethyl)pyridazine (2) (100 mg, 0.44 mmol), Cs₂CO₃ (570 mg, 1.75 mmol) and Xantphos (25 mg, 0.04 mmol) in 1,4-dioxane (4 mL) was purged with Ar(g) for 0.5 h. Pd₂(dba)₃ (20 mg, 0.02 mmol) was added and the reaction mixture was heated up to 90° C. overnight. Once cooled down to rt, it was poured into a mixture of H₂O (50 mL) and brine (30 mL), then extracted with EtOAc (3×20 mL). The combined organics were washed with brine (20 mL), dried over MgSO₄, filtered and concentrated in vacuo. Purification by silica gel column chromatography with hexane/EtOAc (1:0-1:1) yielded (3) as a solid (35 mg, 26%).

KO^{t}Bu (1M in THF, 0.10 mL) was added to a solution of (3) (25 mg, 0.09 mmol) in DMF (1 mL) at 0° C. under Ar(g). The reaction mixture was stirred for 5 min then (4) (28 mg, 0.09 mmol) was added in one portion. It was stirred overnight, poured into a mixture of H₂O (10 mL) and brine (10 mL), then extracted with EtOAc (3×10 mL). The combined organics were washed with NaOH solution (5%, 3×5 mL) then brine (10 mL), dried over MgSO₄ and treated with MP-TMT resin (170 mg, 1.3 mmol/g) for 1.5 h. The reaction mixture was filtered and concentrated in vacuo to afford a residue. Purification by silica gel column chromatography with hexane/EtOAc (1:0-3:1) yielded (EU) as a solid (13 mg, 28%).

LCMS (ES): Found 541.7 [M+H]⁺.

¹H NMR (300 MHz, DMSO-d₆), δ: 9.41 (d, J=1.7 Hz, 2H), 8.37 (d, J=0.8 Hz, 2H), 7.75 (d, J=3.8 Hz, 1H), 7.33 (d, J=3.8 Hz, 1H), 5.90 (s, 2H).

¹⁹F NMR (282 MHz, DMSO-d₆), δ: −63.05 (s, 6F), −64.81 (s, 3F).

### Example EV

### 6-(Trifluoromethyl)-N-({5-[5-(trifluoromethyl)-1,2,4-oxadiazol-3-yl]thiophen-2-yl}methyl)-N-[5-(trifluoromethyl)pyridazin-3-yl]pyridazin-3-amine

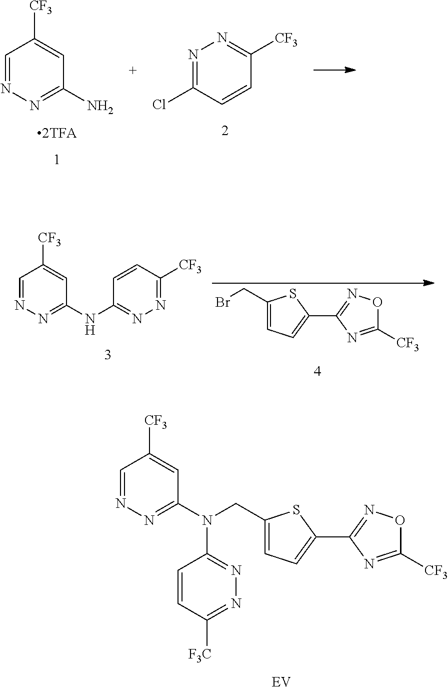 [SMILES: Nc1cc(C(F)(F)F)cnn1 ; FC(F)(F)c1ccc(Cl)nn1 ; FC(F)(F)c1cnnc(Nc2ccc(C(F)(F)F)nn2)c1 ; FC(F)(F)c1nc(-c2ccc(CBr)s2)no1 ; FC(F)(F)c1cnnc(N(Cc2ccc(-c3noc(C(F)(F)F)n3)s2)c2ccc(C(F)(F)F)nn2)c1]

A suspension of (1) (171 mg, 0.44 mmol), 3-chloro-6-(trifluoromethyl)pyridazine (2) (80 mg, 0.44 mmol), Cs₂CO₃ (570 mg, 1.75 mmol) and Xantphos (25 mg, 0.04 mmol) in 1,4-dioxane (4 mL) was purged with Ar(g) for 0.5 h. Pd₂(dba)₃ (20 mg, 0.02 mmol) was added and the reaction mixture was heated up to 90° C. overnight. Once cooled down to rt, it was poured into a mixture of H₂O (30 mL) and brine (30 mL), then extracted with EtOAc (3×20 mL). The combined organics were washed with brine (10 mL), dried over MgSO₄, filtered and concentrated in vacuo. Purification by silica gel column chromatography with hexane/EtOAc (1:0-1:1) yielded (3) as a solid (30 mg, 22%).

KO^{t}Bu (1M in THF, 0.10 mL) was added to a solution of (3) (25 mg, 0.09 mmol) in DMF (1 mL) at 0° C. under Ar(g). The reaction mixture was stirred for 5 min then (4) (28 mg, 0.09 mmol) was added in one portion. It was stirred overnight, poured into a mixture of H₂O (10 mL) and brine (10 mL), then extracted with EtOAc (3×10 mL). The combined organics were washed with NaOH solution (5%, 3×5 mL) then brine (10 mL), dried over MgSO₄ and treated with MP-TMT resin (170 mg, 1.3 mmol/g) for 1.5 h. The reaction mixture was filtered and concentrated in vacuo to afford a residue. Purification by silica gel column chromatography with hexane/EtOAc (1:0-7:3) yielded (EV) as a solid (12 mg, 26%).

LCMS (ES): Found 541.7 [M+H]⁺.

¹H NMR (300 MHz, DMSO-d₆), δ: 9.46 (d, J=1.5 Hz, 1H), 8.44 (dd, J=1.8, 0.8 Hz, 1H), 8.20 (d, J=9.4 Hz, 1H), 8.08 (d, J=9.2 Hz, 1H), 7.76 (d, J=3.8 Hz, 1H), 7.35 (d, J=3.8 Hz, 1H), 5.90 (s, 2H).

¹⁹F NMR (282 MHz, DMSO-d₆), δ: −62.96 (s, 3F), −64.80 (s, 3F), −65.10 (s, 3F).

### Example EW

### 6-(Pyrrolidin-1-yl)-N-({5-[5-(trifluoromethyl)-1,2,4-oxadiazol-3-yl]thiophen-2-yl}methyl)-N-[5-(trifluoromethyl)pyridazin-3-yl]pyridazin-3-amine

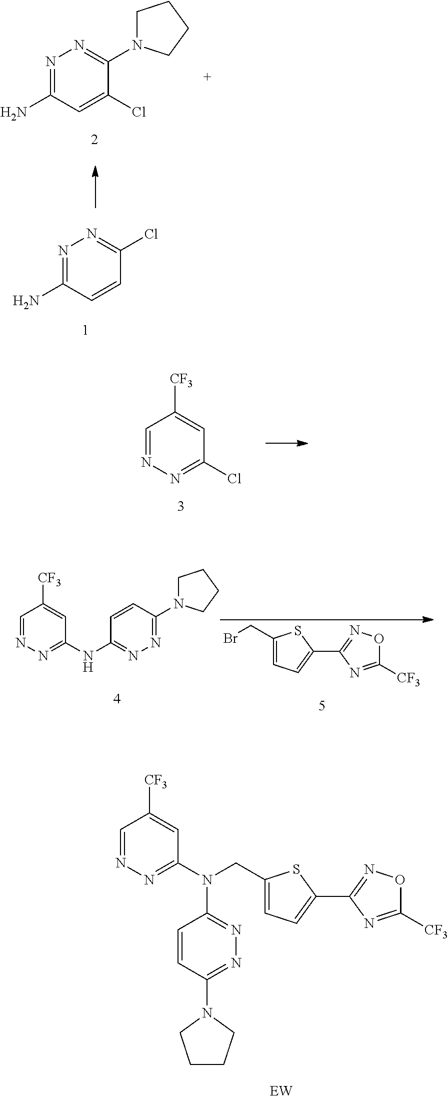

A solution of (1) (400 mg, 3.1 mmol) in NMP (4 mL) was treated with pyrrolidine (0.65 mL, 7.7 mmol) and heated in a microwave reactor (180° C., 4×1 h). The reaction mixture was concentrated in vacuo and purified by silica gel column chromatography with EtOAc/MeOH (1:0-4:1) to yield (2) as a tan solid (136 mg, 27%).

A suspension of (2) (100 mg, 0.61 mmol), 3-chloro-5-(trifluoromethyl)pyridazine (3) (192 mg, 0.61 mmol), Cs₂CO₃ (397 mg, 1.22 mmol) and Xantphos (35 mg, 0.06 mmol) in 1,4-dioxane (3 mL) was purged with Ar(g) for 0.5 h. Pd₂(dba)₃ (28 mg, 0.03 mmol) was added and the reaction mixture was heated up to 90° C. overnight. Once cooled down to rt, it was poured into a mixture of H₂O (10 mL) and brine (10 mL), then extracted with EtOAc (6×20 mL).

The combined organics were washed with brine (10 mL), dried over MgSO₄, filtered and concentrated in vacuo. Purification by silica gel column chromatography with hexane/EtOAc (1:0-3:7) yielded (4) as a yellow solid (34 mg, 18%).

KO^{t}Bu (1M in THF, 0.10 mL) was added to a solution of (4) (25 mg, 0.08 mmol) in DMF (1 mL) at 0° C. under Ar(g). The reaction mixture was stirred for 10 min then (5) (28 mg, 0.09 mmol) was added in one portion. It was stirred for 1.5 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 1 h. The reaction mixture was filtered and concentrated in vacuo to afford a residue. Purification by silica gel column chromatography with hexane/EtOAc (1:0-2:3) yielded (EW) as a yellow solid (11 mg, 25%).

LCMS (ES): Found 543.1 [M+H]⁺.

¹H NMR (300 MHz, DMSO-d₆), δ: 9.13 (d, J=1.7 Hz, 1H), 7.74 (d, J=3.8 Hz, 1H), 7.57 (d, J=9.6 Hz, 1H), 7.50-7.54 (m, 1H), 7.23 (d, J=3.8 Hz, 1H), 7.03 (m, 1H), 5.65 (s, 2H), 3.43-3.52 (m, 4H), 1.93-2.01 (m, 4H).

¹⁹F NMR (282 MHz, DMSO-d₆), δ: −63.34 (s, 3F), −64.79 (s, 3F).

### Example EX

### 6-(Morpholin-4-yl)-N-({5-[5-(trifluoromethyl)-1,2,4-oxadiazol-3-yl]thiophen-2-yl}methyl)-N-[5-(trifluoromethyl)pyridazin-3-yl]pyridazin-3-amine

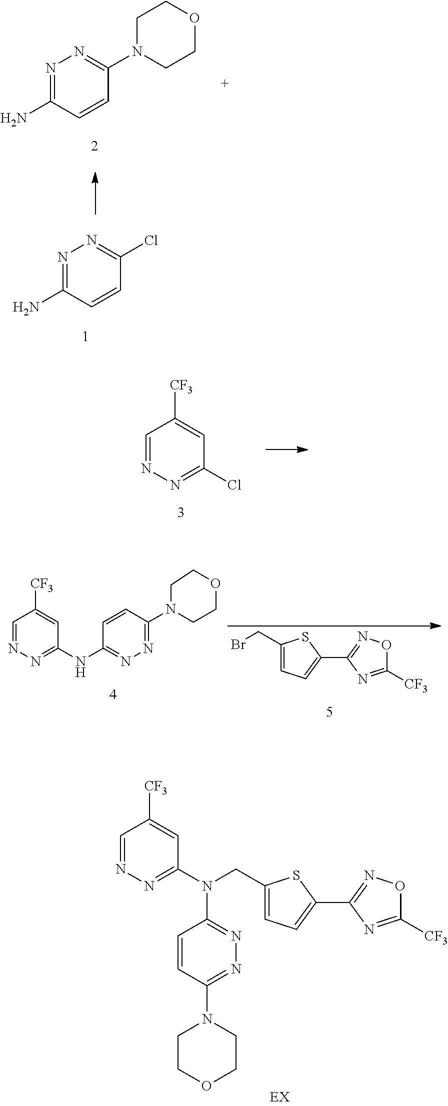

A solution of (1) (400 mg, 3.1 mmol) in NMP (4 mL) was treated with pyrrolidine (0.67 mL, 7.7 mmol) and heated in a microwave reactor (180° C., 4×1 h). The reaction mixture was concentrated in vacuo and purified by silica gel column chromatography with EtOAc/MeOH (1:0-4:1) to yield (2) as a yellow solid (363 mg, 65%).

A suspension of (2) (110 mg, 0.61 mmol), 3-chloro-5-(trifluoromethyl)pyridazine (3) (192 mg, 0.61 mmol), Cs₂CO₃ (397 mg, 1.22 mmol) and Xantphos (35 mg, 0.06 mmol) in 1,4-dioxane (3 mL) was purged with Ar(g) for 0.5 h. Pd₂(dba)₃ (28 mg, 0.03 mmol) was added and the reaction mixture was heated up to 90° C. overnight. Once cooled down to rt, it was poured into a mixture of H₂O (10 mL) and brine (10 mL), then extracted with EtOAc (6×20 mL). The combined organics were washed with brine (10 mL), dried over MgSO₄, filtered and concentrated in vacuo. Purification by silica gel column chromatography with hexane/EtOAc (1:0-1:4) yielded (4) as a yellow solid (85 mg, 43%).

KO^{t}Bu (1M in THF, 0.19 mL) was added to a solution of (4) (50 mg, 0.15 mmol) in DMF (2 mL) at 0° C. under Ar(g). The reaction mixture was stirred for 10 min then (5) (53 mg, 0.17 mmol) was added in one portion. It was stirred for 1.5 h, poured into a mixture of H₂O (10 mL) and brine (10 mL), then extracted with EtOAc (3×10 mL). The combined organics were washed with brine (10 mL), dried over MgSO₄ and treated with MP-TMT resin (170 mg, 1.3 mmol/g) for 1 h. The reaction mixture was filtered and concentrated in vacuo to afford a residue. Purification by silica gel column chromatography with hexane/EtOAc (1:0-2:3) yielded (EX) as a yellow solid (30 mg, 35%).

LCMS (ES): Found 559.0 [M+H]⁺.

¹H NMR (300 MHz, DMSO-d₆), δ: 9.16 (d, J=1.7 Hz, 1H), 7.74 (d, J=3.8 Hz, 1H), 7.70-7.73 (m, 1H), 7.68 (d, J=9.8 Hz, 1H), 7.43 (d, J=9.8 Hz, 1H), 7.25 (d, J=3.8 Hz, 1H), 5.69 (s, 2H), 3.69-3.78 (m, 4H), 3.50-3.60 (m, 4H).

¹⁹F NMR (282 MHz, DMSO-d₆), δ: −63.24 (s, 3F), −64.79 (s, 3F).

### Example EY

### N-({3-Fluoro-5-[5-(trifluoromethyl)-1,2,4-oxadiazol-3-yl]thiophen-2-yl}methyl)-N-(pyrazin-2-yl)pyrazin-2-amine

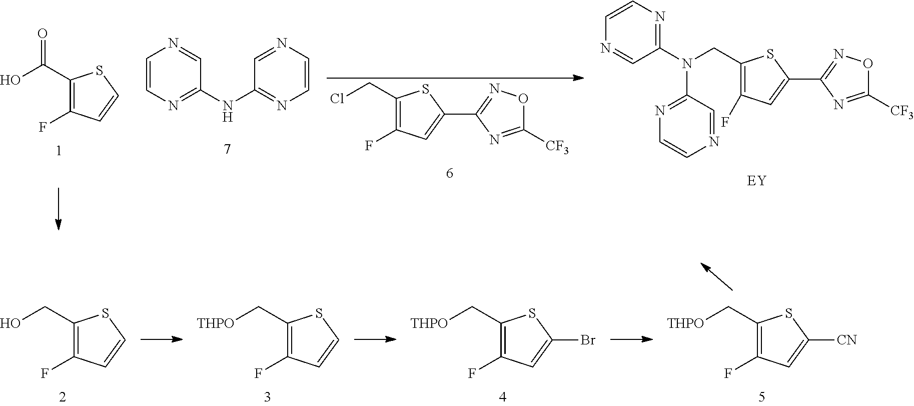

To a suspension of 3-fluoro-2-thiophenecarboxylic acid (1) (2.15 g, 14.7 mmol) in THF (15 mL) at 0° C. was slowly added lithium aluminium hydride solution (1M in THF, 20 mL). The reaction mixture was stirred at rt for 2 h, then warmed up to 40° C. and stirred for a further 0.5 h. Once cooled down to 0° C., it was quenched with a slow addition of H₂O (20 mL). The resulting slurry was filtered and washed with Et₂O (5×15 mL). It was further extracted with EtOAc (3×20 mL). The combined organics were dried over MgSO₄, filtered and concentrated in vacuo to yield (2) as an orange oil (1.82 g, 78%).

A solution of (2) (529 mg, 4.0 mmol) in CH₂Cl₂ (20 mL) was cooled down to 0° C. before the addition of 3,4-dihydro-2H-pyran (0.73 mL, 8.0 mmol) and p-toluenesulfonic acid monohydrate (23 mg, 0.12 mmol). The reaction mixture was stirred at rt for 1 h, cooled down to 0° C. and quenched with NaHCO₃ solution (5 mL). It was then extracted with CH₂Cl₂ (3×15 mL). The combined organics were dried over MgSO₄, filtered and concentrated in vacuo. Purification by silica gel column chromatography with hexane/EtOAc (1:0-2:1) then hexane/CH₂Cl₂ (1:0-2:1) yielded (3) as a colourless oil (724 mg, 84%).

To a solution of (3) (527 mg, 2.5 mmol) in DMF (2.5 mL) was added a solution of N-bromosuccinimide (486 mg, 2.7 mmol) in DMF (2.5 mL). The reaction mixture was stirred at rt for 2 h, cooled down to 0° C. and quenched with H₂O (10 mL). It was then extracted with hexane (3×10 mL). The combined organics were washed with brine (25 mL), dried over MgSO₄, filtered and concentrated in vacuo. Purification by silica gel column chromatography with hexane/CH₂Cl₂ (1:0-2:1) yielded (4) as an oil (439 mg, 60%).

To a reaction vessel charged with Zn(CN)₂ (95 mg, 0.81 mmol), ^{t}BuXPhos Pd G3 (49 mg, 0.06 mmol) and purged with Ar(g) was added a solution of (4) (361 mg, 1.2 mmol) in degassed THF (0.6 mL), followed by degassed H₂O (3 mL). The biphasic reaction mixture was stirred vigorously at 40° C. overnight. Once cooled down to rt, it was quenched with a NaHCO₃ solution (2 mL), extracted with EtOAc (3×5 mL), dried over MgSO₄, filtered and concentrated in vacuo. Purification by silica gel column chromatography with hexane/Et₂O (1:0-7:1) yielded (5) as an oil (239 mg, 81%).

LCMS (ES): Found 241.9 [M+H]⁺.

¹H NMR (300 MHz, Chloroform-d), δ: 7.30 (s, 1H), 4.85 (dd, J=13.4, 1.5 Hz, 1H), 4.74 (t, J=3.2 Hz, 1H), 4.71 (dd, J=13.4, 1.1 Hz, 1H), 3.80-3.90 (m, 1H), 3.53-3.62 (m, 1H), 1.47-1.91 (m, 6H).

¹⁹F NMR (282 MHz, DMSO-d₆), δ: −129.12 (d, J=1.0 Hz, 1F).

To a solution of (5) (56 mg, 0.23 mmol) in EtOH (1 mL) was added NH₂OH (50% in H₂, 0.03 mL, 0.47 mmol). The reaction mixture was heated up to 70° C. overnight. Once cooled down to rt, it was concentrated in vacuo then re-dissolved in anhydrous THF (2 mL). Triethylamine (0.32 mL, 2.3 mmol) and trifluoroacetic anhhydride (0.06 mL, 0.46 mmol) were added sequentially and the reaction mixture was stirred at rt for 2.5 h. Additional trifluoroacetic anhydride (0.06 mL, 0.46 mmol) was then re-charged and the reaction mixture was further stirred for 1.5 h. Additional trifluoroacetic anhydride (0.02 mL, 0.12 mmol) was then re-charged and the reaction mixture was further stirred for 1 h. It was then concentrated in vacuo, re-dissolved in EtOAc (10 mL), washed with H₂O (10 mL), HCl solution (1M, 10 mL) and brine (10 mL). The combined organics were dried over MgSO₄, filtered and concentrated in vacuo.

To a solution of the residue in MeOH (3 mL) was added p-toluenesulfonic acid monohydrate (40 mg, 0.21 mmol). The reaction mixture was stirred at rt for 3 h. It was then concentrated in vacuo, re-dissolved in CH₂Cl₂ (10 mL), poured into NaHCO₃ solution (15 mL) and extracted with CH₂Cl₂ (2×10 mL). The combined organics were dried over MgSO₄, filtered and concentrated in vacuo. To a solution of the residue in MeCN (1.2 mL) was added thionyl chloride (0.17 mL, 2.3 mmol). The reaction mixture was stirred at rt for 1 h. It was then concentrated in vacuo and purified by silica gel column chromatography with hexane/EtOAc (1:0-4:1) to yield (6) as an oil (323 mg, 49%).

¹H NMR (300 MHz, Chloroform-d), δ: 7.54 (s, 1H), 4.77 (s, 2H).

¹⁹F NMR (282 MHz, Chloroform-d), δ: −65.34 (s, 3F), −126.22 (m, 1F).

KO^{t}Bu (1M in THF, 0.13 mL) was added to a solution of (7) (20 mg, 0.12 mmol) in DMF (1 mL) at 0° C. under Ar(g). The reaction mixture was stirred for 0.5 h, then a solution of (6) (33 mg, 0.12 mmol) in DMF (1 mL) was added. It was stirred for 1.5 h, warmed up to rt, poured into a mixture of H₂O (10 mL) and brine (10 mL), then extracted with EtOAc (3×10 mL). The combined organics were washed with brine (10 mL), dried over MgSO₄ and concentrated in vacuo. Purification by silica gel column chromatography with hexane/EtOAc (1:0-3:2) yielded (EY) as a yellow solid (20 mg, 42%).

LCMS (ES): Found 423.9 [M+H]⁺.

¹H NMR (300 MHz, DMSO-d₆), δ: 8.76 (d, J=1.5 Hz, 2H), 8.43 (dd, J=2.6, 1.5 Hz, 2H), 8.30 (d, J=2.6 Hz, 2H), 7.79 (m, 1H), 5.56 (s, 2H).

¹⁹F NMR (282 MHz, DMSO-d₆), δ: −64.77 (s, 3F), −128.0 (m, 1F).

### Example EZ

### 6-[({3-Fluoro-5-[5-(trifluoromethyl)-1,2,4-oxadiazol-3-yl]thiophen-2-yl}methyl)(pyrazin-2-yl)amino]pyridazine-3-carboxylic acid

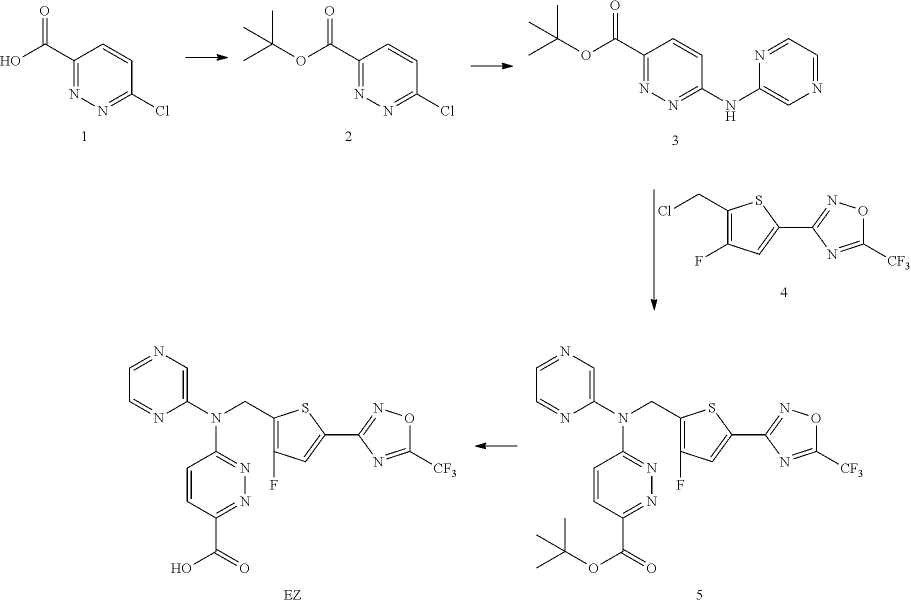 [SMILES: O=C(O)c1ccc(Cl)nn1 ; CC(C)(C)OC(=O)c1ccc(Cl)nn1 ; CC(C)(C)OC(=O)c1ccc(Nc2cnccn2)nn1 ; Fc1cc(-c2noc(C(F)(F)F)n2)sc1CCl ; O=C(O)c1ccc(N(Cc2sc(-c3noc(C(F)(F)F)n3)cc2F)c2cnccn2)nn1 ; CC(C)(C)OC(=O)c1ccc(N(Cc2sc(-c3noc(C(F)(F)F)n3)cc2F)c2cnccn2)nn1]

KO^{t}Bu (1M in THF, 0.2 mL) was added to a solution of (3) (57 mg, 0.21 mmol) in DMF (1 mL) at 0° C. under Ar(g). The reaction mixture was stirred for 20 min, cooled down to −10° C. then a solution of (4) (60 mg, 0.2 mmol) in DMF (1 mL) was added. It was then warmed up to 0° C. over 2 h, re-charged with KO^{t}Bu (1M in THF, 0.08 mL) and allowed to warm up to rt overnight. The reaction mixture was poured into brine (50%, 10 mL) and extracted with EtOAc (3×10 mL). The combined organics were dried over MgSO₄, filtered and concentrated in vacuo to afford a residue. Purification by silica gel column chromatography with hexane/EtOAc (1:0-0:1) yielded (5) as an orange solid (33 mg, 30%).

LCMS (ES): Found 523.8 [M+H]⁺.

¹H NMR (300 MHz, Chloroform-d), δ: 8.70 (d, J=1.5 Hz, 1H), 8.41 (t, J=2.0 Hz, 1H), 8.35 (d, J=2.6 Hz, 1H), 8.00 (d, J=9.2 Hz, 1H), 7.49 (d, J=9.2 Hz, 1H), 7.46 (br s, 1H), 5.73 (s, 2H), 1.67 (s, 9H).

¹⁹F NMR (282 MHz, Chloroform-d), δ: −65.36 (s, 3F), −128.32 (br s, 1F).

To a solution of (5) (33 mg, 0.06 mmol) in CH₂Cl₂ (2 mL) was added TFA (0.24 mL, 3.1 mmol) and the reaction mixture was stirred at rt overnight. It was then diluted with toluene (2 mL) and concentrated in vacuo. The residue was triturated and washed with Et₂O (10 mL) to yield (EZ) as a solid (15 mg, 50%).

LCMS (ES): Found 468.0 [M+H]⁺.

¹H NMR (300 MHz, Chloroform-d), δ: 8.75 (d, J=1.1 Hz, 1H), 8.43-8.52 (m, 2H), 8.15 (d, J=9.4 Hz, 1H), 7.56 (d, J=9.4 Hz, 1H), 7.50 (s, 1H), 5.73 (s, 2H).

¹⁹F NMR (282 MHz, Chloroform-d), δ: −65.31 (s, 3F), −127.76 (s, 1F).

### Example FA

### 6-[({3-fluoro-5-[5-(trifluoromethyl)-1,2,4-oxadiazol-3-yl]thiophen-2-yl}methyl)(pyrazin-2-yl)amino]pyridazine-4-carboxylic acid

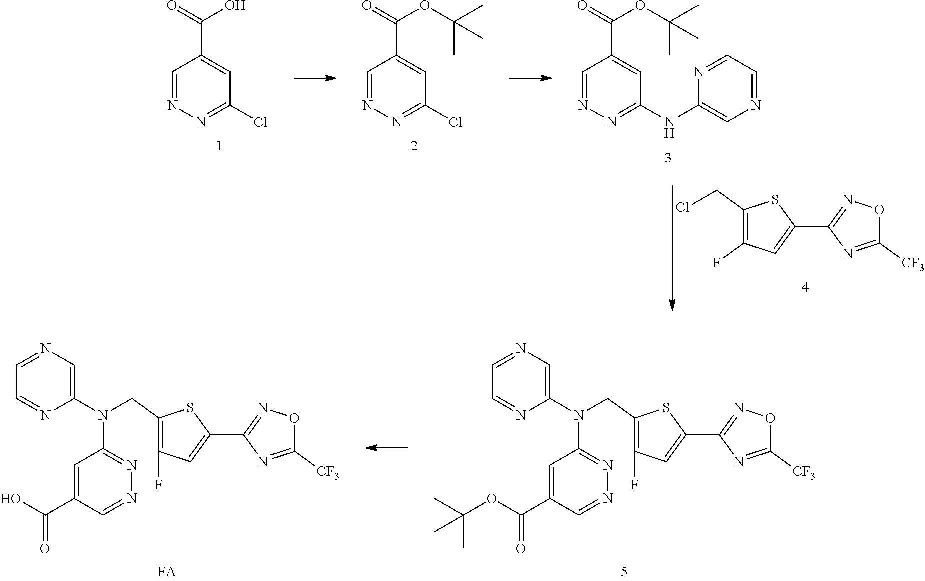

KO^{t}Bu (1M in THF, 0.2 mL) was added to a solution of (3) (58 mg, 0.21 mmol) in DMF (1 mL) at 0° C. under Ar(g). The reaction mixture was stirred for 0.5 h, cooled down to −10° C. then a solution of (4) (60 mg, 0.2 mmol) in DMF (1 mL) was added. It was then warmed up to 0° C. over 3.5 h, re-charged with KO^{t}Bu (1M in THF, 0.06 mL) and allowed to warm up to rt overnight. The reaction mixture was poured into brine (50%, 10 mL) and extracted with EtOAc (3×10 mL). The combined organics were dried over MgSO₄, filtered and concentrated in vacuo to afford a residue. Purification by silica gel column chromatography with hexane/EtOAc (1:0-3:1) yielded (5) as an orange oil (36 mg, 32%).

LCMS (ES): Found 523.9 [M+H]⁺.

¹H NMR (300 MHz, Chloroform-d), δ: 9.28 (d, J=1.5 Hz, 1H), 8.69 (d, J=1.1 Hz, 1H), 8.36-8.42 (m, 1H), 8.33 (d, J=2.4 Hz, 1H), 7.93 (d, J=1.5 Hz, 1H), 7.48 (br s, 1H), 5.71 (s, 2H), 1.60 (s, 9H).

To a solution of (5) (36 mg, 0.07 mmol) in CH₂Cl₂ (2 mL) was added TFA (0.27 mL, 3.5 mmol) and the reaction mixture was stirred at rt overnight. It was then diluted with toluene (2 mL) and concentrated in vacuo. Purification by silica gel column chromatography with hexane/EtOAc (1:0-0:1) yielded (FA) as a brown solid (14 mg, 43%).

LCMS (ES): Found 467.8 [M+H]⁺.

¹H NMR (300 MHz, Chloroform-d), δ: 9.20 (d, J=1.5 Hz, 1H), 8.77 (d, J=1.1 Hz, 1H), 8.45 (dd, J=2.6, 1.5 Hz, 1H), 8.35 (d, J=2.6 Hz, 1H), 7.89 (m, 1H), 7.79 (s, 1H), 5.66 (s, 2H).

¹⁹F NMR (282 MHz, Chloroform-d), δ: −64.78 (s, 3F), −128.11 (s, 1F).

### Example FB

### N-({5-[5-(Trifluoromethyl)-1,2,4-oxadiazol-3-yl]thiophen-2-yl}methyl)-N-[3-(trifluoromethyl)pyridin-2-yl]pyrazin-2-amine

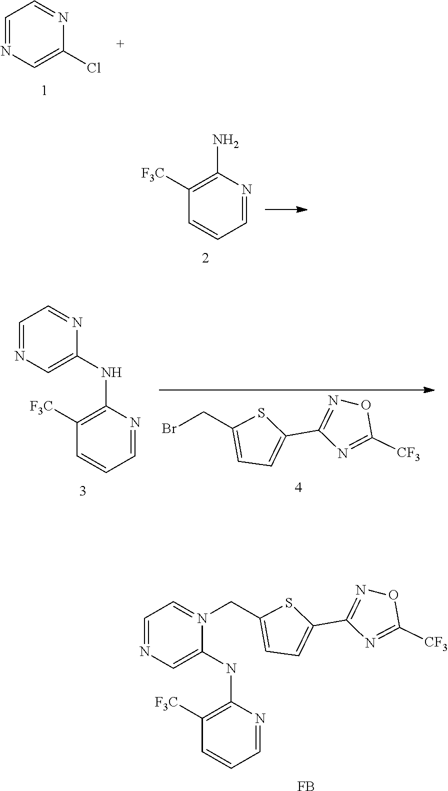

To a mixture of 2-chloropyrazine (1) (252 mg, 2.20 mmol, 1.1 eq), 3-(trifluoromethyl)pyridin-2-amine (2) (324 mg, 2.00 mmol, 1.0 eq) and Cs₂CO₃ (1.30 g, 4.00 mmol, 2.0 eq) was added predegassed 1,4-dioxane (13 mL) and the resulting suspension was degassed with Ar(g) for 1 min. Pd₂(dba)₃ (91.6 mg, 0.100 mmol, 5 mol %) and XantPhos (127 mg, 0.220 mmol, 11 mol %) were then added and the reaction was stirred at 90° C. for 40 h. After cooling to room temperature, H₂O (10 mL) and brine (5 mL) were added and the mixture was extracted with EtOAc (2×15 mL). To the combined organic extracts was added MP-TMT resin (1.3 mmol g⁻¹, ˜400 mg) and the mixture was swirled for several hours then filtered and the filtrate concentrated in vacuo. Purification by basic prep LCMS yielded intermediate 3 (138 mg, 29%).

LCMS (ES): Found 241.2 [M+H]⁺.

To a solution of intermediate 3 (72.0 mg, 0.300 mmol, 1.0 eq) in DMF (2.3 mL) was added NaH (24.0 mg, 0.360 mmol, 1.2 eq) and the reaction was stirred for 5 min before a solution of intermediate 4 (112 mg, 0.360 mmol, 1.2 eq) in DMF (2.3 mL) was added and the reaction stirred overnight. The reaction was quenched by the addition of H₂O (10 μL) and the mixture purified by acidic prep LCMS then silica gel chromatography using hexane/EtOAc (1:0-1:1) to yield compound FB as an off-white solid (30.3 mg, 21%).

LCMS (ES): Found 472.9 [M+H]⁺.

¹H NMR (300 MHz, DMSO-d₆), δ: 8.83-8.93 (m, 1H), 8.38 (dd, J=8.1, 1.5 Hz, 1H), 8.21 (dd, J=2.7, 1.5 Hz, 1H), 8.06 (d, J=2.6 Hz, 1H), 7.93 (d, J=1.5 Hz, 1H), 7.74 (d, J=3.6 Hz, 1H), 7.66-7.72 (m, 1H), 7.25 (d, J=3.8 Hz, 1H), 5.37 (s, 2H).

¹⁹F NMR (282 MHz, DMSO-d₆), δ: −60.32 (s, 3F), −64.79 (s, 3F).

### Example FC

### N-Methyl-6-[(pyrazin-2-yl)({5-[5-(trifluoromethyl)-1,2,4-oxadiazol-3-yl]thiophen-2-yl}methyl)amino]pyridazine-3-carboxamide

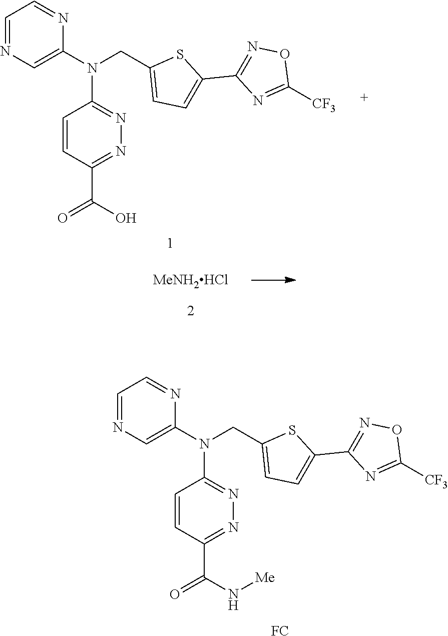

To a solution of compound 1 (29.6 mg, 0.0659 mmol, 1.0 eq) in DMF (1 mL) was added N,N-diisopropylethylamine (45.8 μL, 0.263 mmol, 4.0 eq), HATU (37.6 mg, 0.0988 mmol, 1.5 eq) and methylamine hydrochloride (2) (8.9 mg, 0.132 mmol, 2.0 eq). The reaction was stirred at room temperature for 18 h then diluted with EtOAc (10 mL), washed with 1 M HCl (2×10 mL) and brine (2×10 mL), dried over MgSO₄, filtered and concentrated in vacuo. After purification by silica gel chromatography using hexane/EtOAc (1:0-0:1), the product was redissolved in EtOAc (20 mL) and washed with NaHCO₃ (2×15 mL), H₂O (2×15 mL) and brine (2×15 mL), dried over MgSO₄, filtered and concentrated in vacuo to yield compound FC as an off-white solid (21.1 mg, 69%).

LCMS (ES): Found 462.9 [M+H]⁺.

¹H NMR (300 MHz, DMSO-d₆), δ: 9.10 (q, J=4.6 Hz, 1H), 8.88 (d, J=1.3 Hz, 1H), 8.47 (dd, J=2.6, 1.5 Hz, 1H), 8.37 (d, J=2.4 Hz, 1H), 8.04 (d, J=9.2 Hz, 1H), 7.84 (d, J=9.2 Hz, 1H), 7.74 (d, J=3.8 Hz, 1H), 7.31 (d, J=3.8 Hz, 1H), 5.77 (s, 2H), 2.84 (d, J=4.9 Hz, 3H).

¹⁹F NMR (282 MHz, DMSO-d₆), δ: −64.80 (s, 3F).

### Example FD

### 4-{6-[(Pyrazin-2-yl)({5-[5-(trifluoromethyl)-1,2,4-oxadiazol-3-yl]thiophen-2-yl}methyl)amino]pyridazine-3-carbonyl}-1lambda4-thiomorpholin-1-one

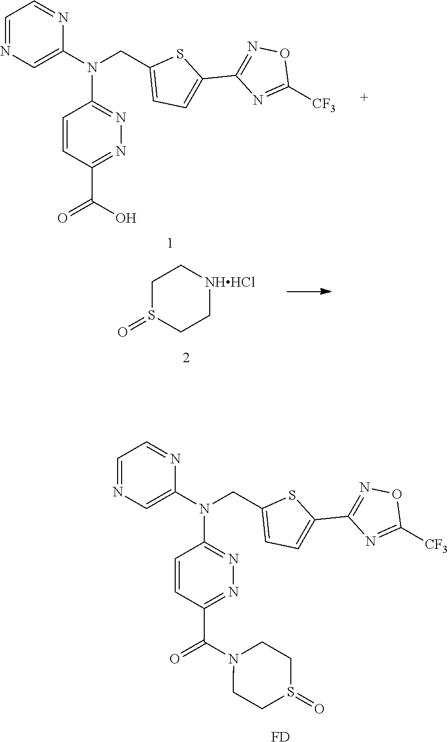

To a solution of compound 1 (21.0 mg, 0.0467 mmol, 1.0 eq) in DMF (1 mL) was added N,N-diisopropylethylamine (32.6 μL, 0.187 mmol, 4.0 eq), HATU (26.7 mg, 0.0701 mmol, 1.5 eq) and thiomorpholine-1-oxide hydrochloride (2) (14.6 mg, 0.0935 mmol, 2.0 eq). The reaction was stirred at room temperature for 17.5 h then diluted with EtOAc (10 mL), washed with 1 M HCl (2×10 mL) and brine (10 mL), dried over MgSO₄, filtered and concentrated in vacuo. Purification by silica gel chromatography using hexane/EtOAc/MeOH (1:0:0-0:1:0-0:9:1) yielded compound FD as a white solid (17.6 mg, 68%).

LCMS (ES): Found 550.9 [M+H]⁺.

¹H NMR (300 MHz, DMSO-d₆), δ: 8.88 (d, J=1.3 Hz, 1H), 8.45 (dd, J=2.6, 1.5 Hz, 1H), 8.36 (d, J=2.6 Hz, 1H), 7.85 (d, J=9.4 Hz, 1H), 7.81 (d, J=9.4 Hz, 1H), 7.75 (d, J=3.8 Hz, 1H), 7.32 (d, J=3.8 Hz, 1H), 5.72-5.80 (m, 2H), 4.34-4.48 (m, 1H), 3.89-4.06 (m, 2H), 3.66-3.84 (m, 1H), 2.75-3.11 (m, 4H).

¹⁹F NMR (282 MHz, DMSO-d₆), δ: −64.79 (s, 3F).

### Example FE

### 6-[(1S,4S)-2-Oxa-5-azabicyclo[2.2.1]heptane-5-carbonyl]-N-(pyrazin-2-yl)-N-({5-[5-(trifluoromethyl)-1,2,4-oxadiazol-3-yl]thiophen-2-yl}methyl)pyridazin-3-amine

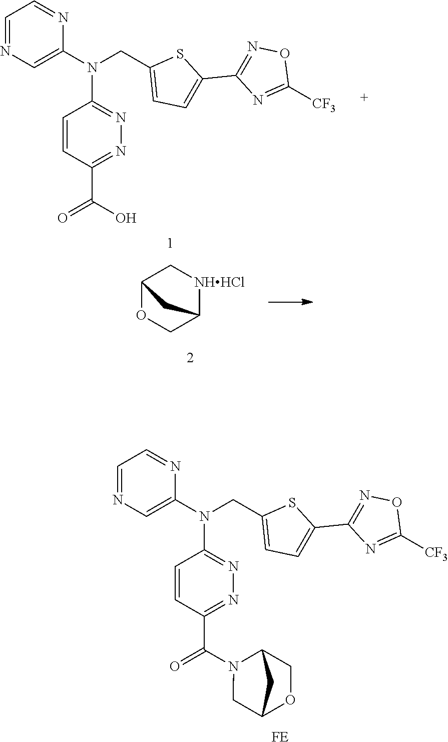

To a solution of compound 1 (21.0 mg, 0.0467 mmol, 1.0 eq) in DMF (1 mL) was added N,N-diisopropylethylamine (32.6 μL, 0.187 mmol, 4.0 eq), HATU (26.7 mg, 0.0701 mmol, 1.5 eq) and (1S,4S)-2-oxa-5-azabicyclo[2.2.1]heptane hydrochloride (2) (12.7 mg, 0.0935 mmol, 2.0 eq). The reaction was stirred at room temperature for 17.5 h then diluted with EtOAc (10 mL), washed with 1 M HCl (2×10 mL) and brine (10 mL), dried over MgSO₄, filtered and concentrated in vacuo. Purification by silica gel chromatography using hexane/EtOAc/MeOH (1:0:0-0:1:0-0:4:1) yielded compound FE as an off-white solid (22.2 mg, 68%).

LCMS (ES): Found 530.9 [M+H]⁺.

¹H NMR (300 MHz, DMSO-d₆), δ: Two rotamers in 3:2 ratio, 8.82-8.91 (m, 1H major rotamer+1H minor rotamer), 8.43-8.51 (m, 1H major+1H minor), 8.32-8.40 (m, 1H major+1H minor), 7.79-7.97 (m, 2H major+2H minor), 7.70-7.78 (m, 1H major+1H minor), 7.27-7.37 (m, 1H major+1H minor), 5.72-5.81 (m, 2H major+2H minor), 5.14-5.20 (m, 1H major), 4.94-5.01 (m, 1H minor), 4.66-4.70 (m, 1H major), 4.60-4.66 (m, 1H minor), 3.85-3.93 (m, 1H major+1H minor), 3.77-3.85 (m, 1H major+2H minor), 3.69-3.76 (m, 1H minor), 3.52-3.58 (m, 1H major), 3.38-3.43 (m, 1H major), 1.88-1.97 (m, 1H major+1H minor), 1.79-1.88 (m, 1H major+1H minor).

¹⁹F NMR (282 MHz, DMSO-d₆), δ: −64.79 (s, 3F).

### Example FF

### 6-(Azetidine-1-carbonyl)-N-(pyrazin-2-yl)-N-({5-[5-(trifluoromethyl)-1,2,4-oxadiazol-3-yl]thiophen-2-yl}methyl)pyridazin-3-amine

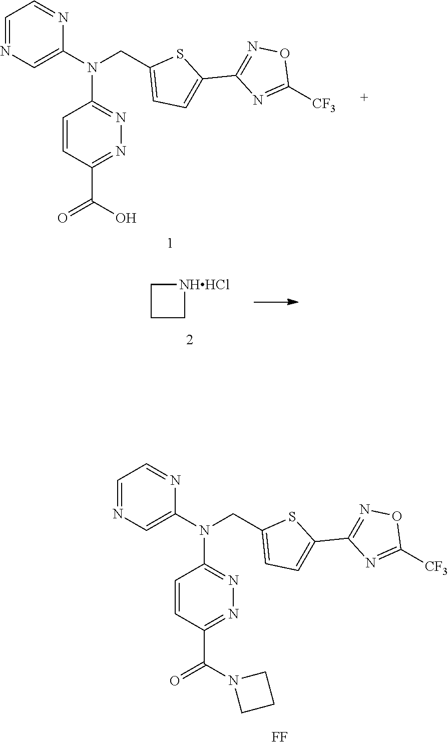

To a solution of compound 1 (22.5 mg, 0.0501 mmol, 1.0 eq) in DMF (1 mL) was added N,N-diisopropylethylamine (34.8 μL, 0.200 mmol, 4.0 eq), HATU (28.6 mg, 0.0751 mmol, 1.5 eq) and azetidine hydrochloride (2) (9.4 mg, 0.100 mmol, 2.0 eq). The reaction was stirred at room temperature for 23 h then diluted with EtOAc (10 mL), washed with 1 M HCl (2×10 mL) and brine (10 mL), dried over MgSO₄, filtered and concentrated in vacuo. Purification by silica gel chromatography using hexane/EtOAc (1:0-0:1) yielded compound FF as an off-white solid (17.6 mg, 72%).

LMCS (ES): Found 488.9 [M+H]⁺.

¹H NMR (300 MHz, DMSO-d₆), δ: 8.86 (d, J=1.3 Hz, 1H), 8.47 (dd, J=2.6, 1.4 Hz, 1H), 8.37 (d, J=2.6 Hz, 1H), 7.97 (d, J=9.4 Hz, 1H), 7.81 (d, J=9.4 Hz, 1H), 7.75 (d, J=3.8 Hz, 1H), 7.31 (d, J=3.8 Hz, 1H), 5.77 (s, 2H), 4.63 (t, J=7.6 Hz, 2H), 4.12 (t, J=7.7 Hz, 2H), 2.31 (q, J=7.7 Hz, 2H).

¹⁹F NMR (282 MHz, DMSO-d₆), δ: −64.79 (s, 3F).

### Example FG

### 6-(Piperidine-1-carbonyl)-N-(pyrazin-2-yl)-N-({5-[5-(trifluoromethyl)-1,2,4-oxadiazol-3-yl]thiophen-2-yl}methyl)pyridazin-3-amine

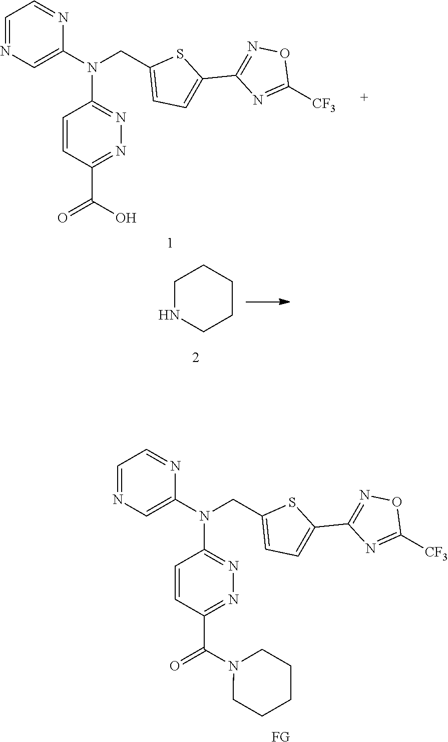

To a solution of compound 1 (21.0 mg, 0.0467 mmol, 1.0 eq) in DMF (1 mL) was added N,N-diisopropylethylamine (16.3 μL, 0.0935 mmol, 2.0 eq), HATU (26.7 mg, 0.0701 mmol, 1.5 eq) and piperidine (2) (9.2 μL, 0.0935 mmol, 2.0 eq). The reaction was stirred at room temperature for 16 h then diluted with EtOAc (10 mL), washed with 1 M HCl (2×10 mL) and brine (10 mL), dried over MgSO₄, filtered and concentrated in vacuo. Purification by silica gel chromatography using hexane/EtOAc (1:0-0:1) yielded compound FG as a yellow solid (20.8 mg, 86%).

MS (ES): Found 516.9 [M+H]⁺.

¹H NMR (300 MHz, DMSO-d₆), δ: 8.86 (d, J=1.5 Hz, 1H), 8.44 (dd, J=2.7, 1.5 Hz, 1H), 8.34 (d, J=2.4 Hz, 1H), 7.82 (d, J=9.3 Hz, 1H), 7.71-7.77 (m, 2H), 7.32 (d, J=3.8 Hz, 1H), 5.75 (s, 2H), 3.60-3.69 (m, 2H), 3.41-3.50 (m, 2H), 1.48-1.70 (m, 6H).

¹⁹F NMR (282 MHz, DMSO-d₆), δ: −64.80 (s, 3F).

### Example FH

### 6-{6-Oxa-1-azaspiro[3.3]heptane-1-carbonyl}-N-(pyrazin-2-yl)-N-({5-[5-(trifluoromethyl)-1,2,4-oxadiazol-3-yl]thiophen-2-yl}methyl)pyridazin-3-amine

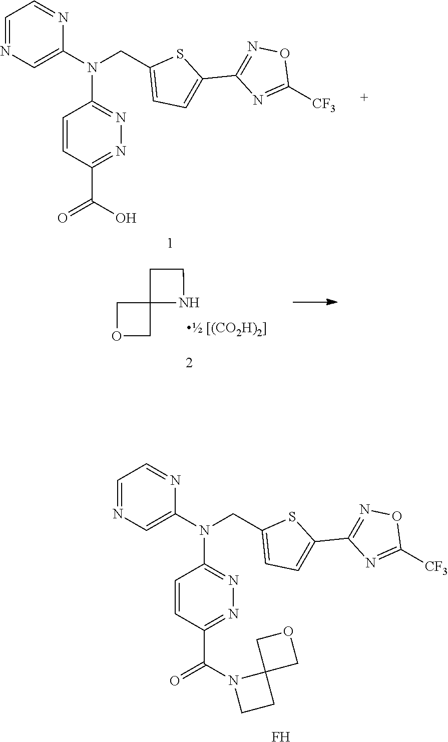

To a solution of compound 1 (20.4 mg, 0.0454 mmol, 1.0 eq) in DMF (1 mL) was added N,N-diisopropylethylamine (31.7 μL, 0.182 mmol, 4.0 eq), HATU (25.9 mg, 0.0681 mmol, 1.5 eq) and 6-oxa-1-azaspiro[3.3]heptane hemioxalate (2) (13.1 mg, 0.0908 mmol, 2.0 eq). The reaction was stirred at room temperature for 20 h then diluted with EtOAc (10 mL), washed with 1 M HCl (2×10 mL) and brine (10 mL), dried over MgSO₄, filtered and concentrated in vacuo. Purification twice by silica gel chromatography using hexane/EtOAc/MeOH (1:0:0-0:1:0-0:4:1) yielded compound FH as a white solid (15.9 mg, 66%).

LCMS (ES): Found 530.9 [M+H]⁺.

¹H NMR (300 MHz, DMSO-d₆), δ: 8.86 (d, J=1.5 Hz, 1H), 8.48 (dd, J=2.7, 1.5 Hz, 1H), 8.39 (d, J=2.6 Hz, 1H), 8.02 (d, J=9.2 Hz, 1H), 7.83 (d, J=9.4 Hz, 1H), 7.74 (d, J=3.8 Hz, 1H), 7.31 (d, J=3.8 Hz, 1H), 5.77 (s, 2H), 5.36 (d, J=6.8 Hz, 2H), 4.60 (d, J=7.0 Hz, 2H), 4.42-4.51 (m, 2H), 2.57-2.65 (m, 2H).

¹⁹F NMR (282 MHz, DMSO-d₆), δ: −64.79 (s, 3F).

### Example FI

### N-(Pyrazin-2-yl)-6-(pyrrolidine-1-carbonyl)-N-({5-[5-(trifluoromethyl)-1,2,4-oxadiazol-3-yl]thiophen-2-yl}methyl)pyridazin-3-amine

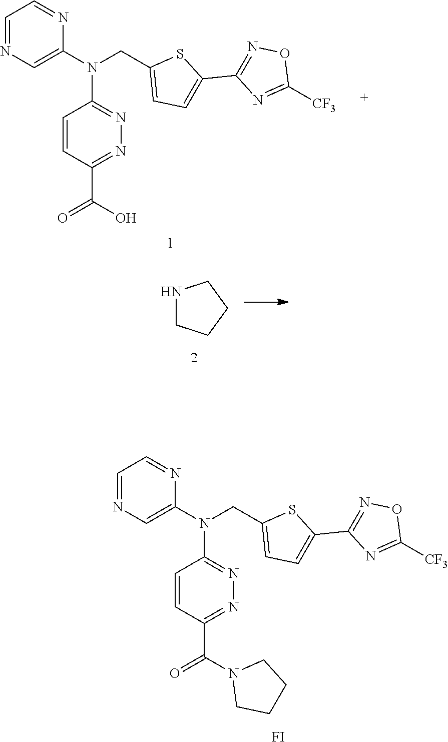

To a solution of compound 1 (19.1 mg, 0.0425 mmol, 1.0 eq) in DMF (1 mL) was added N,N-diisopropylethylamine (14.8 μL, 0.0850 mmol, 2.0 eq), HATU (24.3 mg, 0.0638 mmol, 1.5 eq) and pyrrolidine (2) (7.1 μL, 0.0850 mmol, 2.0 eq). The reaction was stirred at room temperature for 19 h then diluted with EtOAc (10 mL), washed with 1 M HCl (2×10 mL), H₂O (10 mL), NaHCO₃ (2×10 mL), H₂O again (10 mL) and brine (10 mL), dried over MgSO₄, filtered and concentrated in vacuo. Purification by silica gel chromatography using hexane/EtOAc (1:0-0:1) yielded compound FI as a yellow solid (14.4 mg, 68%).

MS (ES): Found 502.9 [M+H]⁺.

¹H NMR (300 MHz, DMSO-d₆), δ: 8.86 (d, J=1.3 Hz, 1H), 8.45 (dd, J=2.6, 1.5 Hz, 1H), 8.36 (d, J=2.4 Hz, 1H), 7.85 (d, J=9.3 Hz, 1H), 7.82 (d, J=9.3 Hz, 1H), 7.75 (d, J=3.8 Hz, 1H), 7.32 (d, J=3.8 Hz, 1H), 5.76 (s, 2H), 3.69-3.77 (m, 2H), 3.50-3.58 (m, 2H), 1.83-1.92 (m, 4H).

¹⁹F NMR (282 MHz, DMSO-d₆), δ: −64.79 (s, 3F).

### Example FJ

### 6-{2-Oxa-6-azaspiro[3.4]octane-6-carbonyl}-N-(pyrazin-2-yl)-N-({5-[5-(trifluoromethyl)-1,2,4-oxadiazol-3-yl]thiophen-2-yl}methyl)pyridazin-3-amine

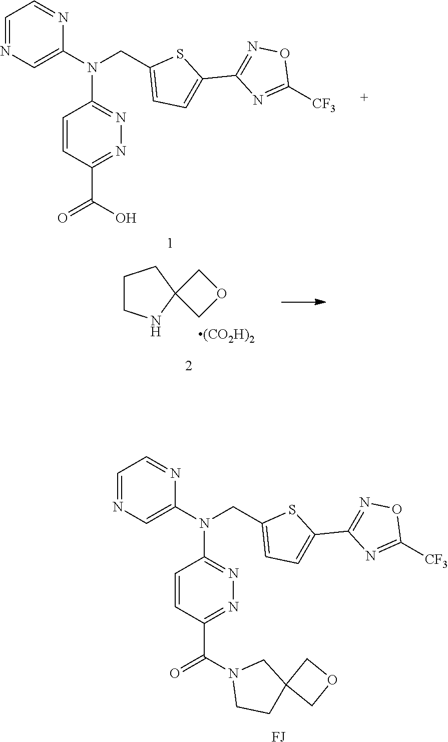

To a solution of compound 1 (19.3 mg, 0.0429 mmol, 1.0 eq) in DMF (1 mL) was added N,N-diisopropylethylamine (30.0 μL, 0.172 mmol, 4.0 eq), HATU (24.5 mg, 0.0644 mmol, 1.5 eq) and 2-oxa-6-azaspiro[3.4]octane oxalate (2) (13.6 mg, 0.0859 mmol, 2.0 eq). The reaction was stirred at room temperature for 19 h then diluted with EtOAc (10 mL), washed with 1 M HCl (2×10 mL), H₂O (10 mL), NaHCO₃ (2×10 mL), H₂O again (10 mL) and brine (10 mL), dried over MgSO₄, filtered and concentrated in vacuo. Purification by silica gel chromatography using hexane/EtOAcMeOH (1:0:0-0:1:0-0:94:6) yielded compound FJ as an off-white solid (18.8 mg, 80%).

LCMS (ES) 544.9 [M+H]⁺.

¹H NMR (300 MHz, DMSO-d₆), δ: Two rotamers in 1:1 ratio, 8.87 (dd, J=6.6, 1.3 Hz, 2×1H), 8.43-8.49 (m, 2×1H), 8.34-8.39 (m, 2×1H), 7.78-7.91 (m, 2×2H), 7.75 (t, J=3.9 Hz, 2×1H), 7.33 (dd, J=5.0, 3.9 Hz, 2×1H), 5.77 (app. d, J=5.8 Hz, 2×2H), 4.63 (d, J=6.0 Hz, 2H), 4.43-4.56 (m, 2H+4H), 4.04 (s, 1H), 3.74-3.84 (m, 4H), 3.56 (t, J=7.1 Hz, 2H), 2.19 (m, 2×2H).

¹⁹F NMR (282 MHz, DMSO-d₆), δ: Two rotamers in 1:1 ratio −64.79 (s, 3F), −64.79 (s, 3F).

### Example FK

### 1-{6-[(Pyrazin-2-yl)({5-[5-(trifluoromethyl)-1,2,4-oxadiazol-3-yl]thiophen-2-yl}methyl)amino]pyridazine-3-carbonyl}-3-(trifluoromethyl)azetidin-3-ol

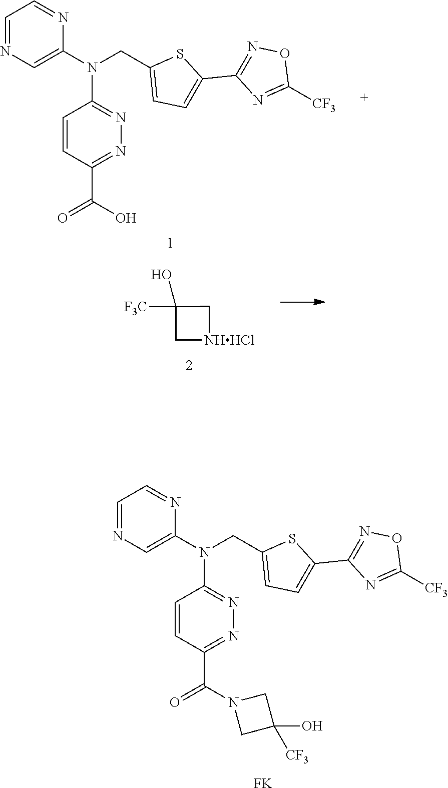

To a solution of compound 1 (28.8 mg, 0.0641 mmol, 1.0 eq) in DMF (1.5 mL) was added N,N-diisopropylethylamine (44.6 μL, 0.256 mmol, 4.0 eq), HATU (36.5 mg, 0.0961 mmol, 1.5 eq) and 3-(trifluoromethyl)azetidin-3-ol hydrochloride (2) (22.7 mg, 0.128 mmol, 2.0 eq). The reaction was stirred at room temperature for 2 h then diluted with EtOAc (10 mL), washed with 1 M HCl (2×10 mL), H₂O (10 mL), NaHCO₃ (2×10 mL), H₂O again (10 mL) and brine (10 mL), dried over MgSO₄, filtered and concentrated in vacuo. Purification by silica gel chromatography using hexane/EtOAc (1:0-1:3) yielded compound FK as an off-white solid (23.2 mg, 63%).

LCMS (ES−) 616.8 (100%, [M+formic acid-H]⁻, 570.8 (10%, [M−H]⁻.

¹H NMR (300 MHz, DMSO-d₆), δ: 8.88 (d, J=1.3 Hz, 1H), 8.49 (dd, J=2.6, 1.5 Hz, 1H), 8.39 (d, J=2.6 Hz, 1H), 8.02 (d, J=9.2 Hz, 1H), 7.83 (d, J=9.4 Hz, 1H), 7.75 (d, J=3.8 Hz, 1H), 7.49 (s, 1H), 7.32 (d, J=3.8 Hz, 1H), 5.77 (s, 2H), 4.86 (dd, J=11.3, 0.9 Hz, 1H), 4.63 (br d, J=11.5 Hz, 1H), 4.36 (dd, J=11.4, 1.0 Hz, 1H), 4.11 (br d, J=11.5 Hz, 1H).

¹⁹F NMR (282 MHz, DMSO-d₆), δ: −64.79 (s, 3F), −82.69 (s, 3F).

### Example FL

### N3-(Pyrazin-2-yl)-N3-({5-[5-(trifluoromethyl)-1,2,4-oxadiazol-3-yl]thiophen-2-yl}methyl)pyridazine-3,5-diamine

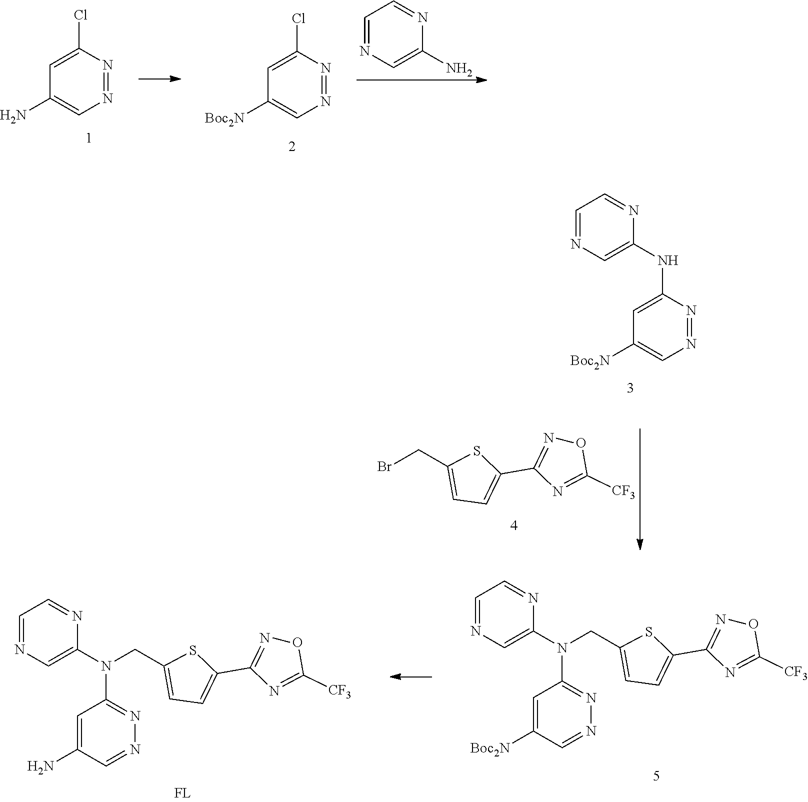

A suspension of 4-amino-6-chloropyridazine (1) (2.59 g, 22.3 mmol, 1.0 eq) in CH₂Cl₂ (65 mL) was cooled to 0° C. before the addition of triethylamine (10.9 mL, 77.9 mmol, 3.5 eq) and di-tert-butyl dicarbonate (12.2 g, 55.7 mmol, 2.5 eq). The suspension was warmed to room temperature and stirred for 17.5 h then DMAP (277 mg, 2.23 mmol, 0.1 eq) and additional di-tert-butyl dicarbonate (4.86 g, 22.3 mmol, 1.0 eq) were added. The reaction was stirred for 2.5 h then concentrated in vacuo. Purification twice by silica gel chromatography using hexane/EtOAc (1:0-4:1) yielded intermediate 2 as an orange solid (3.40 g, 46%).

LCMS (ES): Found 174.0 [M-CO₂tBu-tBu+3H]⁺.

¹H NMR (300 MHz, DMSO-d₆), δ: 9.33 (d, J=2.1 Hz, 1H), 8.14 (d, J=2.1 Hz, 1H), 1.41 (s, 18H).

A solution of intermediate 2 (3.40 g, 10.3 mmol, 1.0 eq) in 1,4-dioxane (100 mL) was degassed with Ar(g) for 1 h before 2-aminopyrazine (1.47 g, 15.46 mmol, 1.5 eq), Pd₂(dba)₃ (283 mg, 0.309 mmol, 3 mol %), XantPhos (358 mg, 0.618 mmol, 6 mol %) and Cs₂CO₃ (6.72 g, 20.6 mmol, 2.0 eq) were added sequentially with continued degassing. The mixture was degassed with Ar(g) for a further 10 min then stirred at 90° C. for 21 h. After cooling to room temperature, the reaction mixture was poured into H₂O (100 mL) and extracted with EtOAc (3×100 mL). The combined organic extracts were dried over MgSO₄, filtered, concentrated in vacuo and purified three times by silica gel chromatography using hexane/EtOAc (1:0-1:1) then CH₂Cl₂/MeOH (1:0-9:1) then CH₂Cl₂/EtOAc (1:0-0:1) to yield intermediate 3 as a pale yellow solid (594 mg, 15%).

LCMS (ES): Found 389.0 [M+H]⁺.

¹H NMR (300 MHz, DMSO-d₆), δ: 10.69 (s, 1H), 8.95 (d, J=1.5 Hz, 1H), 8.83 (d, J=2.3 Hz, 1H), 8.25 (dd, J=2.6, 1.5 Hz, 1H), 8.18 (d, J=2.6 Hz, 1H), 8.01 (d, J=2.3 Hz, 1H), 1.43 (s, 18H).

To a solution of intermediate 3 (514 mg, 1.32 mmol, 1.0 eq) in anhydrous DMF (15 mL) at 0° C. was added ˜1.65 M KO^{t}Bu in THF (0.962 mL, 1.59 mmol, 1.2 eq). The solution was stirred at 0° C. for 20 min then a solution of intermediate 4 (536 mg, 1.59 mmol, 1.2 eq) in DMF (10 mL) was added over 5 min. The reaction was stirred at 0° C. for 10 min then allowed to warm to room temperature and stirred for 1.5 h. After recooling to 0° C., the reaction was quenched by the addition of H₂O (5 mL) and approximately half the volume of DMF was removed in vacuo. The mixture was poured into 50% brine (25 mL) and extracted with EtOAc (3×25 mL). The combined organic extracts were dried over MgSO₄, filtered, concentrated in vacuo and purified by silica gel chromatography using hexane/EtOAc (1:0-2:1). The residue was redissolved in CH₂Cl₂ (15 mL) and swirled with MP-TMT resin (1.3 mmol g⁻¹, ˜1.0 g) for 5 h.

The mixture was filtered, the resin washed with CH₂Cl₂ (25 mL) and the filtrate concentrated in vacuo to yield intermediate 5 as an off-white solid (377 mg, 46%).

LCMS (ES): Found 620.9 [M+H]⁺.

¹H NMR (300 MHz, DMSO-d₆), δ: 8.94 (d, J=2.1 Hz, 1H), 8.68 (d, J=1.5 Hz, 1H), 8.40 (dd, J=2.7, 1.5 Hz, 1H), 8.31 (d, J=2.6 Hz, 1H), 7.86 (d, J=2.1 Hz, 1H), 7.74 (d, J=3.8 Hz, 1H), 7.31 (d, J=3.8 Hz, 1H), 5.73 (s, 2H), 1.37 (s, 18H).

¹⁹F NMR (282 MHz, DMSO-d₆), δ: −64.80 (s, 3F).

To a solution of intermediate 5 (355 mg, 0.572 mmol, 1.0 eq) in CH₂Cl₂ (20 mL) was added trifluoroacetic acid (0.881 mL, 11.4 mmol, 20 eq) and the reaction was stirred at room temperature for 1 h. Additional trifluoroacetic acid (1.32 mL, 17.2 mmol, 30 eq) was then added and the reaction was stirred for a further 23 h then diluted with toluene (15 mL) and concentrated in vacuo. The residue was dissolved in CH₂Cl₂ and passed through a pad of SCX eluting with NH₃ in CH₂Cl₂/5% MeOH (0.07-0.14-0.28 M). Product-containing fractions were concentrated in vacuo and purified by silica gel chromatography using EtOAc/MeOH (1:0-19:1) yielding compound FL as an off-white solid (190 mg, 79%).

LCMS (ES): Found 420.9 [M+H]⁺.

¹H NMR (300 MHz, DMSO-d₆), δ: 8.53 (d, J=1.5 Hz, 1H), 8.32-8.38 (m, 2H), 8.15 (d, J=2.6 Hz, 1H), 7.74 (d, J=3.8 Hz, 1H), 7.22 (d, J=3.8 Hz, 1H), 6.56 (d, J=2.3 Hz, 1H), 6.47 (br s, 2H), 5.55 (s, 2H).

¹⁹F NMR (282 MHz, DMSO-d₆), δ: −64.79 (s, 3F).

### Example FM

### N-{6-[(Pyrazin-2-yl)({5-[5-(trifluoromethyl)-1,2,4-oxadiazol-3-yl]thiophen-2-yl}methyl)amino]pyridazin-4-yl}acetamide

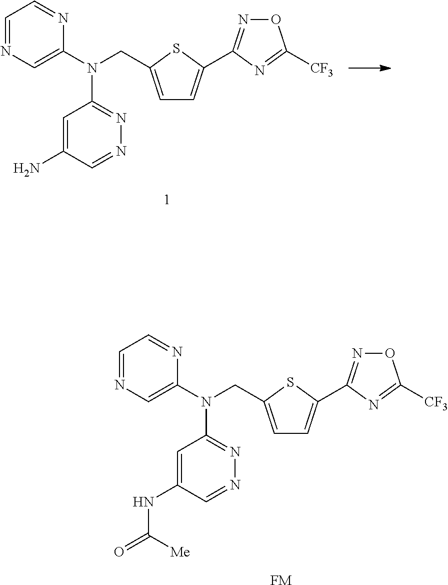

A solution of compound 1 (32.0 mg, 0.0762 mmol, 1.0 eq), acetic anhydride (25.8 μL, 0.381 mmol, 5.0 eq), triethylamine (53.1 μL, 0.381 mmol, 5.0 eq) and DMAP (4.7 mg, 0.0381, 0.5 eq) in 1,2-dichloroethane (1 mL) was stirred at 80° C. for 1 h under microwave irradiation. The reaction mixture was concentrated in vacuo and purified by silica gel chromatography using CH₂Cl₂/MeOH (1:0-32:1) to yield compound FM as an off-white solid (24.3 mg, 69%).

LCMS (ES): Found 462.9 [M+H]⁺.

¹H NMR (300 MHz, DMSO-d₆), δ: 10.60 (s, 1H), 8.97 (d, J=2.1 Hz, 1H), 8.72 (d, J=1.5 Hz, 1H), 8.42 (dd, J=2.6, 1.5 Hz, 1H), 8.28 (d, J=2.6 Hz, 1H), 7.94 (d, J=2.1 Hz, 1H), 7.74 (d, J=3.8 Hz, 1H), 7.26 (d, J=3.8 Hz, 1H), 5.67 (s, 2H), 2.10 (s, 3H).

¹⁹F NMR (282 MHz, DMSO-d₆), δ: −64.79 (s, 3F).

### Example FN

### 6-[(Morpholin-4-yl)methyl]-N-(pyrazin-2-yl)-N-({5-[5-(trifluoromethyl)-1,2,4-oxadiazol-3-yl]thiophen-2-yl}methyl)pyridazin-3-amine

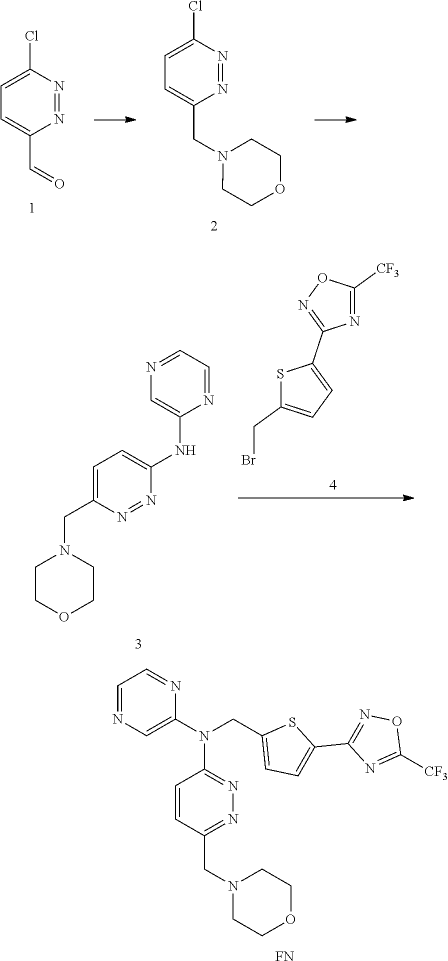

To a solution of 6-chloro-3-pyridazinecarboxaldehyde (1) (219 mg, 1.46 mmol, 1.0 eq) in 1,2-dichloroethane (4 mL) was added morpholine (255 μL, 2.91 mmol, 2.0 eq) and the mixture was stirred under Ar(g) for 10 min. To the suspension was added NaBH(OAc)₃ (636 mg, 2.91 mmol, 2.0 eq) and additional 1,2-dichloroethane (2 mL) and the reaction was stirred for 4 h. The reaction was quenched with 1 M NaOH (2 mL) and the resulting mixture extracted with CH₂Cl₂ (5 mL) then EtOAc (2×5 mL). The combined organic extracts were dried over MgSO₄, filtered and concentrated in vacuo. The residue (2) (1.2 eq), was then redissolved in 1,4-dioxane (15 mL) and the solution was degassed with Ar(g) for 40 mins. Aminopyrazine (116 mg, 1.22 mmol, 1.0 eq), Pd₂(dba)₃ (40.1 mg, 0.0438 mmol, 3 mol %), XantPhos (50.7 mg, 0.0876 mmol, 6 mol %) and Cs₂CO₃ (793 mg, 2.43 mmol, 2.0 eq) were added with continued degassing. The mixture was degassed with Ar(g) for a further 40 min then stirred at 90° C. for 21 h. After cooling to room temperature, the reaction mixture was poured into H₂O (20 mL) and extracted with EtOAc (3×20 mL) then CH₂Cl₂ (3×20 mL). The combined organic extracts were concentrated in vacuo then redissolved in CH₂Cl₂/MeOH (4:1, 50 mL) and swirled with MP-TMT resin (1.3 mmol g⁻¹, ˜1 g) at room temperature for 6 h. The resin was filtered off, washed with CH₂Cl₂/MeOH (4:1, 50 mL) and the filtrate concentrated in vacuo. Purification twice by silica gel chromatography using CH₂Cl₂/MeOH (1:0-5:1) then EtOAc/MeOH (1:0-93:7) yielded intermediate 3 as an off-white solid (55.4 mg, 14%).

LCMS (ES): Found 273.0 [M+H]⁺.

¹H NMR (300 MHz, DMSO-d₆), δ: 10.48 (s, 1H), 8.99 (d, J=1.5 Hz, 1H), 8.26 (dd, J=2.7, 1.5 Hz, 1H), 8.15 (d, J=2.6 Hz, 1H), 8.05 (d, J=9.2 Hz, 1H), 7.63 (d, J=9.2 Hz, 1H), 3.70 (s, 2H), 3.50-3.62 (m, 4H), 2.42 (s, 4H).

To a solution of intermediate 3 (56.3 mg, 0.206 mmol, 1.0 eq) in anhydrous DMF (4 mL) at 0° C. was added ˜1.65 M KO^{t}Bu in THF (150 μL, 0.248 mmol, 1.2 eq). The solution was stirred at 0° C. for 15 min then intermediate 4 (38.9 mg, 0.124 mmol, 0.6 eq) was added. The reaction was stirred at 0° C. for 5 min before the remaining intermediate 5 (38.9 mg, 0.124 mmol, 0.6 eq) was added. After stirring at 0 to 5° C. for 1.5 h, the reaction was quenched by the addition of H₂O (2 mL), poured into 50% saturated brine (15 mL) and extracted with EtOAc (3×15 mL). The combined organic extracts were dried over MgSO₄, filtered and concentrated in vacuo. Purification twice by silica gel chromatography using EtOAc/MeOH (1:0-19:1) yielded compound FN as an orange gum (19.7 mg, 19%).

LCMS (ES): Found 504.9 [M+H]⁺.

¹H NMR (300 MHz, DMSO-d₆), δ: 8.73 (d, J=1.5 Hz, 1H), 8.37 (dd, J=2.6, 1.5 Hz, 1H), 8.26 (d, J=2.6 Hz, 1H), 7.70-7.79 (m, 2H), 7.63 (d, J=9.2 Hz, 1H), 7.30 (d, J=3.8 Hz, 1H), 5.70 (s, 2H), 3.73 (s, 2H), 3.52-3.65 (m, 4H), 2.35-2.47 (m, 4H).

¹⁹F NMR (282 MHz, DMSO-d₆), δ: −64.79 (s, 3F).

### Example FO

### 4-({6-[(Pyrazin-2-yl)({5-[5-(trifluoromethyl)-1,2,4-oxadiazol-3-yl]thiophen-2-yl}methyl)amino]pyridazin-3-yl}methyl)-1lambda4-thiomorpholin-1-one

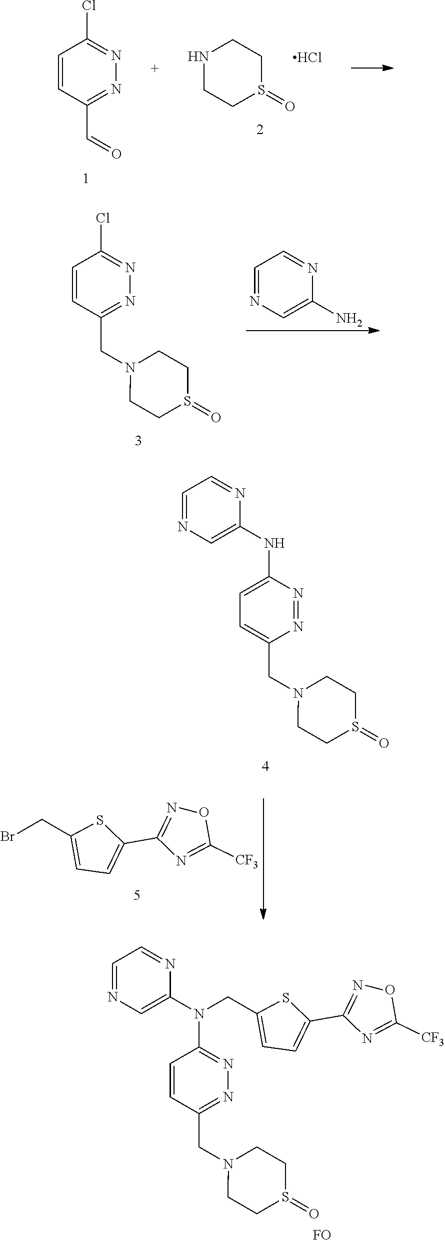

To a solution of 6-chloro-3-pyridazinecarboxaldehyde (1) (502 mg, 3.52 mmol, 1.0 eq) in 1,2-dichloroethane (4 mL) was added NaOAc (722 mg, 8.80 mmol, 2.5 eq) followed by CH₂Cl₂ (2 mL) and thiomorpholine-1-oxide hydrochloride (2) (1.37 g, 8.80 mmol, 2.5 eq). The mixture was stirred for 10 min before the addition of NaBH(OAc)₃ (1.12 g, 5.28 mmol, 1.5 eq) then stirred for 4 h at room temperature. The reaction was quenched with 1 M NaOH (5 mL) and the resulting mixture was poured into H₂O (10 mL) and extracted with CH₂Cl₂ (10 mL) then EtOAc (2×10 mL). The combined organic extracts were dried over MgSO₄, filtered and concentrated in vacuo. Purification by silica gel chromatography using CH₂Cl₂/MeOH (1:0-19:1) yielded intermediate 3 as an off-white solid (408 mg, 47%).

LCMS (ES): Found 245.9 [M+H]⁺.

¹H NMR (300 MHz, DMSO-d₆), δ: 7.91 (d, J=8.9 Hz, 1H), 7.81 (d, J=8.9 Hz, 1H), 3.89 (s, 2H), 2.82-3.04 (m, 4H), 2.59-2.79 (m, 4H).

A solution of intermediate 3 (402 mg, 1.64 mmol, 1.2 eq) in 1,4-dioxane (15 mL) was degassed with Ar(g) for 40 mins before the addition of aminopyrazine (129 mg, 1.36 mmol, 1.0 eq), Pd₂(dba)₃ (37.4 mg, 0.0409 mmol, 3 mol %), XantPhos (47.3 mg, 0.0818 mmol, 6 mol %) and Cs₂CO₃ (889 mg, 2.73 mmol, 2.0 eq) with continued degassing. The mixture was degassed with Ar(g) for a further 30 min then stirred at 90° C. for 21 h. After cooling to room temperature, the reaction mixture was concentrated in vacuo then slurried in H₂O (20 mL). The suspension was filtered and the solids washed with H₂O (50 mL). The aqueous washes were concentrated in vacuo then purified by reverse phase silica gel chromatography using H₂O/MeCN (19:1). The product was redissolved in CH₂Cl₂/MeOH (4:1, 50 mL) and swirled with MP-TMT resin (1.3 mmol g⁻¹, ˜500 mg) at room temperature overnight. The resin was filtered off, washed with CH₂Cl₂/MeOH (4:1, 50 mL) and the filtrate concentrated in vacuo to yield intermediate 4 as an off-white solid (81.8 mg, 20%).

LCMS (ES): Found 305.0 [M+H]⁺.

¹H NMR (300 MHz, DMSO-d₆), δ: 10.50 (s, 1H), 9.00 (d, J=1.5 Hz, 1H), 8.26 (dd, J=2.5, 1.4 Hz, 1H), 8.15 (d, J=2.8 Hz, 1H), 8.06 (d, J=9.2 Hz, 1H), 7.64 (d, J=9.2 Hz, 1H), 3.82 (br s, 2H), 2.94 (quin, J=12.1 Hz, 4H), 2.58-2.81 (m, 4H).

To a solution of intermediate 4 (78.9 mg, 0.259 mmol, 1.0 eq) in anhydrous DMF (10 mL) at 0° C. was added ˜1.7 M KO^{t}Bu in THF (183 μL, 0.311 mmol, 1.2 eq). The solution was stirred at 0° C. for 30 min then intermediate 5 (97.4 mg, 0.311 mmol, 1.2 eq) was added. After stirring at 0° C. for 1.5 h, the reaction mixture was poured into 50% saturated brine (15 mL) and extracted with EtOAc (3×15 mL). The combined organic extracts were dried over MgSO₄, filtered and concentrated in vacuo. Purification twice by silica gel chromatography using CH₂Cl₂/MeOH (1:0-19:1) yielded compound FO as an off-white solid (57.1 mg, 41%).

LCMS (ES): Found 536.9 [M+H]⁺.

¹H NMR (300 MHz, CDCl₃), δ: 8.75 (d, J=1.3 Hz, 1H), 8.38 (dd, J=2.6, 1.5 Hz, 1H), 8.27 (d, J=2.6 Hz, 1H), 7.71-7.80 (m, 2H), 7.64 (d, J=9.2 Hz, 1H), 7.30 (d, J=4.0 Hz, 1H), 5.71 (s, 2H), 3.84 (s, 2H), 2.82-3.06 (m, 4H), 2.62-2.81 (m, 4H).

¹⁹F NMR (282 MHz, DMSO-d₆), δ: −64.79 (s, 3F).

### Example FP

### 5-(Morpholin-4-yl)-N-(pyrazin-2-yl)-N-({5-[5-(trifluoromethyl)-1,2,4-oxadiazol-3-yl]thiophen-2-yl}methyl)pyridazin-3-amine

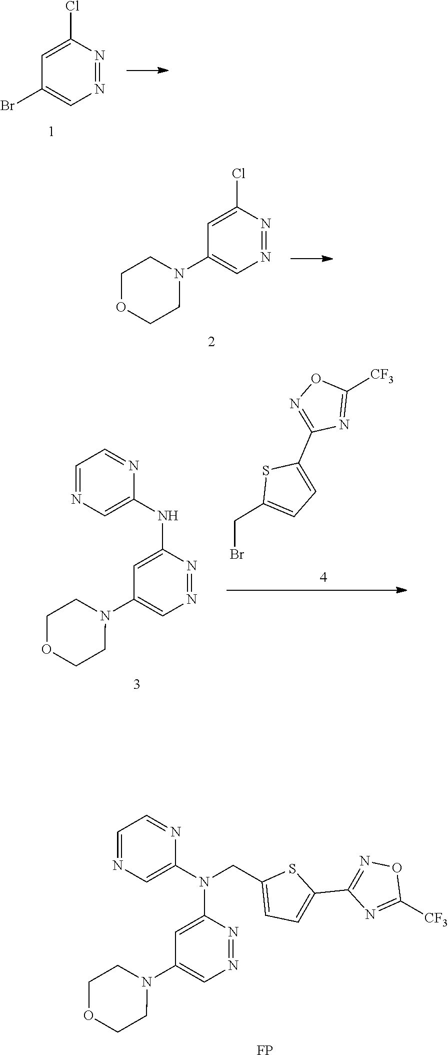

To a solution of 3-chloro-5-bromopyridazine (1) (86% w/w, 200 mg, 0.889 mmol, 1.0 eq) in DMF (3 mL) was added morpholine (116 μL, 1.33 mmol, 1.5 eq) and K₂CO₃ (246 mg, 1.78 mmol, 2.0 eq). The suspension was stirred at 40° C. for 5 h then cooled to room temperature and poured in to 50% saturated brine (10 mL). The resulting mixture was extracted with EtOAc (3×10 mL) and the combined organic extracts were dried over MgSO₄, filtered and concentrated in vacuo. Purification by silica gel chromatography using CH₂Cl₂/MeOH (1:0-97:3) yielded intermediate 2 as a yellow solid (142 mg, 80%).

LCMS (ES): Found 200.0 [M+H]⁺.

¹H NMR (300 MHz, DMSO-d₆), δ: 8.96 (d, J=2.8 Hz, 1H), 7.09 (d, J=2.8 Hz, 1H), 3.64-3.75 (m, 4H), 3.41-3.50 (m, 4H).

N,N-Dimethylacetamide (2 mL) was degassed with Ar(g) for 15 min before intermediate 2 (49.9 mg, 0.250 mmol, 1.0 eq), aminopyrazine (47.6 mg, 0.500 mmol, 2.0 eq), Pd₂(dba)₃ (11.4 mg, 0.0125 mmol, 5 mol %), XantPhos (14.5 mg, 0.0250 mmol, 10 mol %) and Cs₂CO₃ (163 mg, 0.500 mmol, 2.0 eq) were added with continued degassing. The resulting mixture was degassed with Ar(g) for a further 5 min then stirred at 140° C. for 4 h. After cooling to r.t., the reaction mixture was poured into 50% saturated brine (10 mL) and extracted with CH₂Cl₂ (2×10 mL) and EtOAc (3×10 mL). The combined organic extracts were dried over MgSO₄, filtered and concentrated in vacuo. Purification by silica gel chromatography using CH₂Cl₂/MeOH (1:0-93:7) yielded compound 3 as a pale orange solid (17.4 mg, 27%).

LCMS (ES): Found 259.0 [M+H]⁺.

¹H NMR (300 MHz, DMSO-d₆), δ: 10.05 (s, 1H), 9.00 (d, J=1.3 Hz, 1H), 8.69 (d, J=2.6 Hz, 1H), 8.23 (dd, J=2.7, 1.5 Hz, 1H), 8.09 (d, J=2.6 Hz, 1H), 7.34 (d, J=2.8 Hz, 1H), 3.70-3.79 (m, 4H), 3.32-3.40 (m, 4H).

To a solution of intermediate 3 (70.3 mg, 0.272 mmol, 1.0 eq) in anhydrous DMF (5 mL) at 0° C. was added ˜1.7 M KO^{t}Bu in THF (167 μL, 0.285 mmol, 1.05 eq). The solution was stirred at 0° C. for 15 min then solution of intermediate 4 (93.6 mg, 0.299 mmol, 1.1 eq) in DMF (2 mL) was added. After stirring at 0° C. for 2.5 h, additional portions of KO^{t}Bu (32 μL, 0.0544 mmol, 0.2 eq) and 4 (17.0 mg, 0.0544 mmol, 0.2 eq) were added and the reaction stirred at 0° C. for a further 2 h. After warming to r.t., the reaction was quenched with H₂O (2 mL) and the DMF was removed in vacuo. The mixture was poured into 50% saturated brine (20 mL) and extracted with EtOAc (3×20 mL). The combined organic extracts were dried over MgSO₄, filtered and concentrated in vacuo. Purification twice by silica gel chromatography using CH₂Cl₂/MeOH (1:0-19:1) then EtOAc/MeOH (1:0-97:3) yielded compound FP as a pale yellow solid (44.0 mg, 33%).

LCMS (ES): Found 491.0 [M+H]⁺.

¹H NMR (300 MHz, CDCl₃), δ: 8.80 (d, J=2.6 Hz, 1H), 8.54 (d, J=1.5 Hz, 1H), 8.36 (dd, J=2.7, 1.5 Hz, 1H), 8.15 (d, J=2.6 Hz, 1H), 7.73 (d, J=3.8 Hz, 1H), 7.24 (d, J=3.8 Hz, 1H), 6.96 (d, J=2.6 Hz, 1H), 5.61 (s, 2H), 3.62-3.79 (m, 4H), 3.35-3.46 (m, 4H).

¹⁹F NMR (282 MHz, DMSO-d₆), δ: −64.79 (s, 3F).

### Example FQ

### N-(Pyrazin-2-yl)-5-(pyrrolidin-1-yl)-N-({5-[5-(trifluoromethyl)-1,2,4-oxadiazol-3-yl]thiophen-2-yl}methyl)pyridazin-3-amine

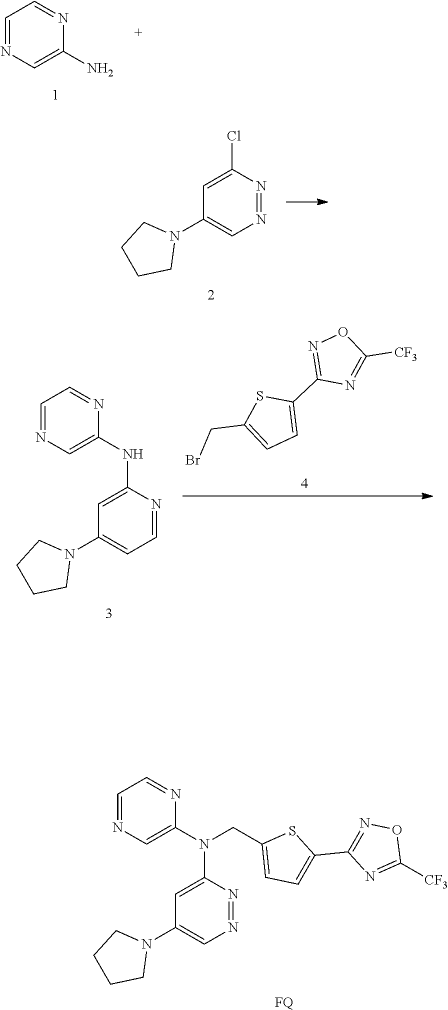

N,N-Dimethylacetamide (3 mL) was degassed with argon for 10 min before 3-chloro-5-(pyrrolidin-1-yl)pyridazine (2) (76.1 mg, 0.414 mmol, 1.0 eq), aminopyrazine (1) (78.7 mg, 0.828 mmol, 2.0 eq), Pd₂(dba)₃ (37.9 mg, 0.0414 mmol, 10 mol %), XantPhos (47.9 mg, 0.0828 mmol, 20 mol %) and Cs₂CO₃ (297 mg, 0.911 mmol, 2.2 eq) were added with continued degassing. The resulting mixture was degassed with argon for a further 10 min then stirred at 140° C. for 4 h. After cooling to r.t., the reaction mixture was concentrated in vacuo and the residue redissolved in CH₂Cl₂/MeOH (4:1, 50 mL) and swirled with MP-TMT resin (3.1 mmol g⁻¹, ˜1 g) at room temperature for 5 h. The resin was filtered off, washed with CH₂Cl₂/MeOH (4:1, 50 mL), and the filtrate concentrated in vacuo. Purification three times by silica gel chromatography using CH₂Cl₂/MeOH (1:0-19:1) yielded intermediate 3 as an off-white solid (35.6 mg, 35%).

LCMS (ES): Found 243.2 [M+H]⁺.

¹H NMR (300 MHz, DMSO-d₆), δ: 9.94 (br s, 1H), 8.98 (d, J=1.3 Hz, 1H), 8.34 (d, J=2.6 Hz, 1H), 8.22 (dd, J=2.6, 1.5 Hz, 1H), 8.07 (d, J=2.6 Hz, 1H), 7.02 (d, J=2.6 Hz, 1H), 3.33-3.36 (m, 4H), 1.94-2.02 (m, 4H).

To a solution of intermediate 3 (66.5 mg, 0.274 mmol, 1.0 eq) in anhydrous DMF (5 mL) at 0° C. was added ˜1.7 M KO^{t}Bu in THF (183 μL, 0.302 mmol, 1.1 eq). The solution was stirred at 0° C. for 10 min then intermediate 4 (94.6 mg, 0.302 mmol, 1.1 eq) was added. After stirring at 0° C. for 3 h, the reaction was quenched with H₂O (1 mL) and the DMF was removed in vacuo. The mixture was poured into 50% saturated brine (20 mL) and extracted with EtOAc (3×20 mL). The combined organic extracts were dried over MgSO₄, filtered and concentrated in vacuo. Purification twice by silica gel chromatography using EtOAc/MeOH (1:0-19:1) then CH₂Cl₂/MeOH (1:0-32:1) yielded compound FQ as a pale yellow solid (49.9 mg, 38%).

LCMS (ES): Found 475.2 [M+H]⁺.

¹H NMR (300 MHz, CDCl₃), δ: 8.53 (d, J=1.3 Hz, 1H), 8.46 (d, J=2.4 Hz, 1H), 8.35 (dd, J=2.7, 1.5 Hz, 1H), 8.13 (d, J=2.6 Hz, 1H), 7.72 (d, J=3.8 Hz, 1H), 7.24 (d, J=3.8 Hz, 1H), 6.56 (d, J=2.4 Hz, 1H), 5.60 (s, 2H), 3.33-3.44 (m, 4H), 1.87-2.02 (m, 4H).

¹⁹F NMR (282 MHz, DMSO-d₆), δ: −64.79 (s, 3F).

### Example FR

### (1S,2R,4S)-5-{6-[(Pyrazin-2-yl)({5-[5-(trifluoromethyl)-1,2,4-oxadiazol-3-yl]thiophen-2-yl}methyl)amino]pyridazine-3-carbonyl}-2-thia-5-azabicyclo[2.2.1]heptan-2-ium-2-olate

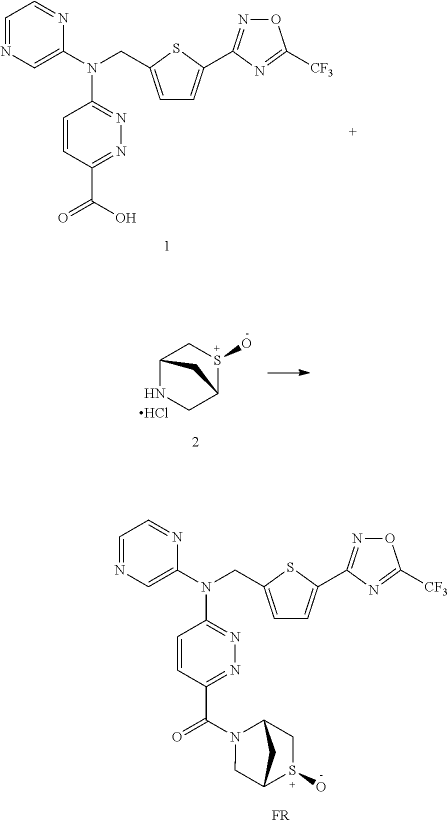

To a solution of compound 1 (52.3 mg, 0.116 mmol, 1.0 eq) in DMF (2 mL) was added N,N-diisopropylethylamine (81.1 μL, 0.466 mmol, 4.0 eq), HATU (66.5 mg, 0.175 mmol, 1.5 eq) and amine 2 (39.1 mg, 0.233 mmol, 2.0 eq), followed by additional DMF (1 mL). The reaction was stirred at room temperature for 1.5 h then concentrated in vacuo, redissolved in EtOAc (10 mL) and washed with 1 M HCl (3×10 mL), H₂O (15 mL), NaHCO₃ (3×10 mL) and brine (2×15 mL). The organic phase was concentrated in vacuo then purified by silica gel chromatography using EtOAc/MeOH (1:0-88:12) to yield compound FR as an off-white solid (51.0 mg, 78%).

LCMS (ES): Found 563.1 [M+H]⁺.

¹H NMR (300 MHz, DMSO-d₆), δ: Two rotamers in 3:2 ratio, 8.88 (d, J=1.3 Hz, 1H major rotamer), 8.85 (d, J=1.3 Hz, 1H minor rotamer), 8.45-8.49 (m, 1H major+1H minor), 8.38 (d, J=2.6 Hz, 1H major+1H minor), 7.78-7.94 (m, 2H major+2H minor), 7.72-7.77 (m, 1H major+1H minor), 7.28-7.35 (m, 1H major+1H minor), 5.78 (s, 2H major), 5.75 (s, 2H minor), 5.31 (br s, 1H major), 5.08 (br s, 1H minor), 4.12 (dd, J=13.0, 4.9 Hz, 1H minor), 3.93 (br d, J=4.3 Hz, 1H major+1H minor), 3.71-3.81 (m, 2H major), 3.61 (br d, J=13.2 Hz, 1H minor), 3.45 (d, J=12.8 Hz, 1H minor), 3.01 (d, J=14.1 Hz, 1H major), 2.29-2.48 (m, 3H major+3H minor).

¹⁹F NMR (282 MHz, DMSO-d₆), δ: −64.78 (s, 3F).

### Example FS

### 5-(Azetidine-1-carbonyl)-N-(pyrazin-2-yl)-N-({5-[5-(trifluoromethyl)-1,2,4-oxadiazol-3-yl]thiophen-2-yl}methyl)pyridazin-3-amine

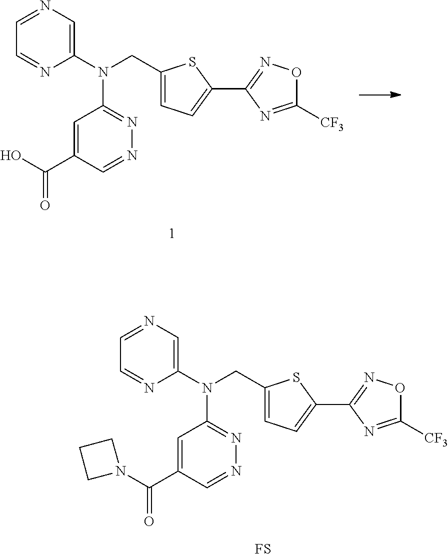 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CCC1 ; "FS"]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 30 min at rt before addition of azetidine hydrochloride (21 mg, 0.22 mmol). After 1 h 40 min the reaction mixture was diluted with EtOAc (30 mL), washed with HCl solution (5%, 3×10 mL) and brine (10 mL), before being dried over MgSO₄, filtered and concentrated in vacuo. Purification by flash column chromatography with EtOAc/MeOH (1:0 to 19:1) afforded FS as pale straw-coloured solids (39 mg, 72%).

LCMS (ES): Found 488.9 [M+H]⁺.

¹H NMR (300 MHz, DMSO-d₆) δ: 9.03 (d, J=1.7 Hz, 1H), 8.82 (d, J=1.5 Hz, 1H), 8.40 (dd, J=2.5, 1.4 Hz, 1H), 8.32 (d, J=2.4 Hz, 1H), 7.83 (d, J=1.9 Hz, 1H), 7.74 (d, J=3.8 Hz, 1H), 7.31 (d, J=3.8 Hz, 1H), 5.77 (s, 2H), 4.32 (t, J=7.6 Hz, 2H), 4.06 (t, J=7.7 Hz, 2H), 2.27 (quin, J=7.7 Hz, 2H).

¹⁹F NMR (282 MHz, DMSO-d₆) δ: −64.80 (s, 3F).

### Example FT

### 5-(3,3-Difluoroazetidine-1-carbonyl)-N-(pyrazin-2-yl)-N-({5-[5-(trifluoromethyl)-1,2,4-oxadiazol-3-yl]thiophen-2-yl}methyl)pyridazin-3-amine

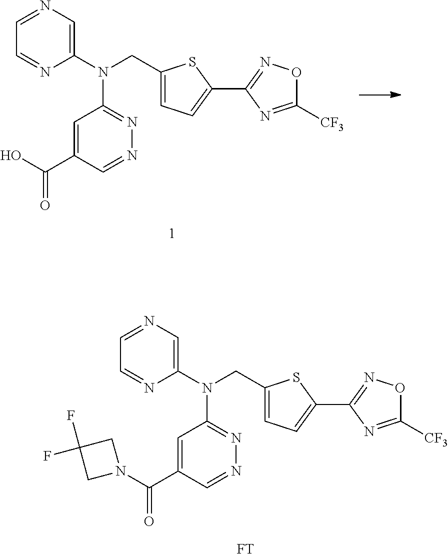 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CC(F)(F)C1 ; "FT"]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 30 min at rt before addition of 3,3-difluoroazetidine hydrochloride (29 mg, 0.22 mmol). After 3 h 30 min the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with DCM/EtOAc (1:0 to 1:2) afforded FT as pale orange solids (41 mg, 72%).

LCMS (ES): Found 524.8 [M+H]⁺.

¹H NMR (300 MHz, DMSO-d₆) δ: 9.07 (d, J=1.9 Hz, 1H), 8.83 (d, J=1.5 Hz, 1H), 8.42 (dd, J=2.6, 1.5 Hz, 1H), 8.33 (d, J=2.4 Hz, 1H), 7.92 (d, J=1.9 Hz, 1H), 7.75 (d, J=3.8 Hz, 1H), 7.31 (d, J=3.8 Hz, 1H), 5.77 (s, 2H), 4.85 (br t, J=12.6 Hz, 2H), 4.51 (br t, J=12.5 Hz, 2H).

¹⁹F NMR (282 MHz, DMSO-d₆) δ: −64.80 (s, 3F), −99.33 (quin, J=12.6 Hz, 2F).

### Example FU

### 4-{6-[(Pyrazin-2-yl)({5-[5-(trifluoromethyl)-1,2,4-oxadiazol-3-yl]thiophen-2-yl}methyl)amino]pyridazine-4-carbonyl}-1lambda4-thiomorpholin-1-one

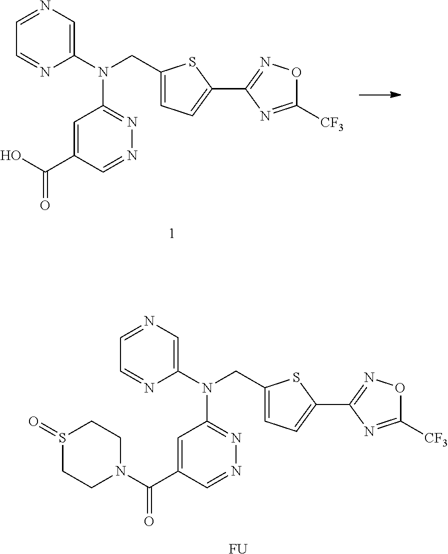 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CCS(=O)CC1 ; "FU"]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 30 min at rt before addition of thiomorpholine-1-oxide hydrochloride (35 mg, 0.22 mmol). After 3 h 30 min the reaction mixture was diluted with EtOAc (30 mL), washed with HCl solution (5%, 3×10 mL) and brine (10 mL), before being dried over MgSO₄, filtered and concentrated in vacuo. Purification by flash column chromatography with EtOAc/MeOH (1:0 to 4:1) and subsequent trituration with Et₂O afforded FU as pale beige solids (16 mg, 27%).

LCMS (ES): Found 550.9 [M+H]⁺.

¹H NMR (300 MHz, DMSO-d₆) δ: 8.97 (d, J=1.5 Hz, 1H), 8.86 (d, J=1.3 Hz, 1H), 8.42 (dd, J=2.5, 1.4 Hz, 1H), 8.33 (d, J=2.4 Hz, 1H), 7.84 (d, J=1.5 Hz, 1H), 7.75 (d, J=3.6 Hz, 1H), 7.31 (d, J=3.8 Hz, 1H), 5.76 (br s, 2H), 4.26-4.38 (m, 1H), 3.79-3.93 (m, 1H), 3.61-3.76 (m, 1H), 3.47-3.60 (m, 1H), 2.82-3.05 (m, 3H), 2.71-2.82 (m, 1H).

¹⁹F NMR (282 MHz, DMSO-d₆) δ: −64.80 (s, 3F).

### Example FV

### 5-[(1S,4S)-2-Oxa-5-azabicyclo[2.2.1]heptane-5-carbonyl]-N-(pyrazin-2-yl)-N-({5-[5-(trifluoromethyl)-1,2,4-oxadiazol-3-yl]thiophen-2-yl}methyl)pyridazin-3-amine

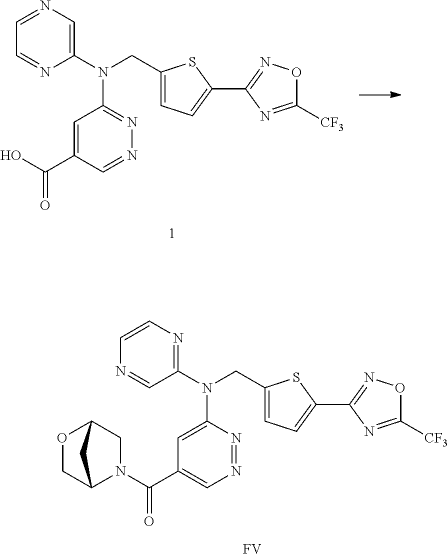 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1C[C@@H]2C[C@H]1CO2 ; "FV"]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 30 min at rt before addition of (1S,4S)-2-oxa-5-azabicyclo[2.2.1]heptane hydrochloride (30 mg, 0.22 mmol). After 2 h 20 min the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with EtOAc/MeOH (1:0 to 9:1) afforded FV as pale yellow solids (45 mg, 76%).

LCMS (ES): Found 530.9 [M+H]⁺.

¹H NMR (300 MHz, DMSO-d₆) δ: Two rotamers in 3:2 ratio, 9.01 (d, J=1.7 Hz, 1H minor), 8.99 (d, J=1.7 Hz, 1H major), 8.87 (d, J=1.5 Hz, 1H major), 8.81 (d, J=1.3 Hz, 1H minor), 8.36-8.43 (m, 1H major+1H minor), 8.31 (d, J=2.6 Hz, 1H major+1H minor), 7.87 (d, J=1.7 Hz, 1H minor), 7.82 (d, J=1.7 Hz, 1H major), 7.72-7.78 (m, 1H major+1H minor), 7.28-7.36 (m, 1H major+1H minor), 5.69-5.86 (m, 2H major+2H minor), 4.87 (br s, 1H minor), 4.67 (br s, 1H major), 4.61 (br s, 1H minor), 4.42 (br s, 1H major), 3.89 (d, J=7.7 Hz, 1H major), 3.81 (d, J=7.0 Hz, 1H minor), 3.75 (dd, J=7.9, 1.9 Hz, 1H minor), 3.64 (dd, J=7.6, 1.4 Hz, 1H major), 3.45-3.52 (m, 1H major+1H minor), 3.26-3.32 (m, 1H major+1H minor), 1.76-1.94 (m, 2H major+2H minor).

¹⁹F NMR (282 MHz, DMSO-d₆) δ: −64.80 (s, 3F).

### Example FW

### 5-[(1R,4R)-2-Oxa-5-azabicyclo[2.2.1]heptane-5-carbonyl]-N-(pyrazin-2-yl)-N-({5-[5-(trifluoromethyl)-1,2,4-oxadiazol-3-yl]thiophen-2-yl}methyl)pyridazin-3-amine

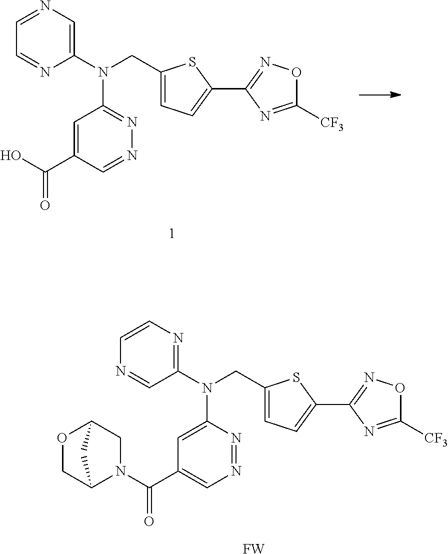 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1C[C@H]2C[C@@H]1CO2 ; "FW"]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 30 min at rt before addition of (1R,4R)-2-oxa-5-azabicyclo[2.2.1]heptane hydrochloride (30 mg, 0.22 mmol). After 2 h 20 min the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with EtOAc/MeOH (1:0 to 9:1) afforded FW as pale beige solids (44 mg, 75%).

LCMS (ES): Found 530.9 [M+H]⁺.

¹H NMR (300 MHz, DMSO-d₆) δ: Two rotamers in 3:2 ratio, 9.01 (d, J=1.7 Hz, 1H minor), 8.99 (d, J=1.7 Hz, 1H major), 8.87 (d, J=1.3 Hz, 1H major), 8.81 (d, J=1.5 Hz, 1H minor), 8.37-8.42 (m, 1H major+1H minor), 8.31 (d, J=2.6 Hz, 1H major+1H minor), 7.87 (d, J=1.7 Hz, 1H minor), 7.82 (d, J=1.7 Hz, 1H major), 7.72-7.78 (m, 1H major+1H minor), 7.28-7.35 (m, 1H major+1H minor), 5.68-5.86 (m, 2H major+2H minor), 4.87 (br s, 1H minor), 4.67 (br s, 1H major), 4.61 (br s, 1H minor), 4.42 (br s, 1H major), 3.89 (d, J=7.3 Hz, 1H major), 3.81 (d, J=6.6 Hz, 1H minor), 3.72-3.77 (m, 1H minor), 3.64 (dd, J=7.5, 1.3 Hz, 1H major), 3.44-3.52 (m, 1H major+1H minor), 3.26-3.32 (m, 1H major+1H minor), 1.75-1.96 (m, 2H major+2H minor).

¹⁹F NMR (282 MHz, DMSO-d₆) δ: −64.80 (s, 3F).

### Example FX

### 5-[3-(Dimethylamino)azetidine-1-carbonyl]-N-(pyrazin-2-yl)-N-({5-[5-(trifluoromethyl)-1,2,4-oxadiazol-3-yl]thiophen-2-yl}methyl)pyridazin-3-amine

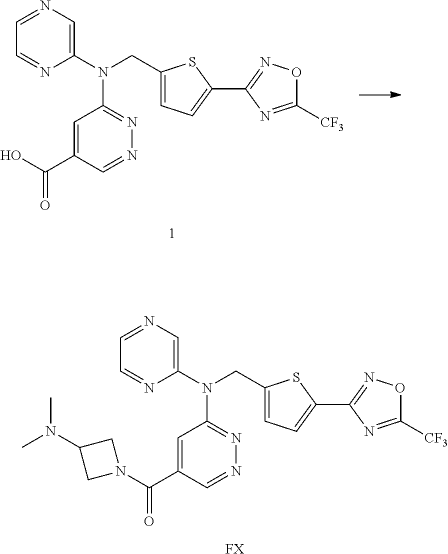

To a solution of acid 1 (50 mg, 0.11 mmol) in DMF (1 mL) was added N-methylmorpholine (61 μL, 0.56 mmol) followed by PyBOP (87 mg, 0.17 mmol). The reaction mixture was stirred for 40 min at rt before addition of 3-(dimethylamino)azetidine dihydrochloride (39 mg, 0.22 mmol). After 18 h the reaction mixture was diluted with NaOH solution (5%, 30 mL) and extracted with EtOAc (3×15 mL). The combined organics were washed with brine (10 mL) before being dried over MgSO₄, filtered and concentrated in vacuo. The residue was loaded onto an SCX-2 column, washed with DCM/MeOH (1:1) and product eluted with DCM/MeOH/7N NH₃(MeOH) (2:1:1). Subsequent purification by flash column chromatography with EtOAc/MeOH (1:0 to 4:1) afforded FX as light beige solids (19 mg, 32%).

LCMS (ES): Found 531.9 [M+H]⁺.

¹H NMR (300 MHz, DMSO-d₆) δ: 9.04 (d, J=1.7 Hz, 1H), 8.84 (d, J=1.3 Hz, 1H), 8.40 (dd, J=2.6, 1.5 Hz, 1H), 8.32 (d, J=2.6 Hz, 1H), 7.85 (d, J=1.7 Hz, 1H), 7.74 (d, J=3.8 Hz, 1H), 7.31 (d, J=3.8 Hz, 1H), 5.77 (s, 2H), 4.21-4.32 (m, 1H), 4.02-4.18 (m, 2H), 3.78-3.88 (m, 1H), 3.02-3.14 (m, 1H), 2.07 (s, 6H).

¹⁹F NMR (282 MHz, DMSO-d₆) δ: −64.80 (s, 3F).

### Example FY

### 5-(4,4-Difluoropiperidine-1-carbonyl)-N-(pyrazin-2-yl)-N-({5-[5-(trifluoromethyl)-1,2,4-oxadiazol-3-yl]thiophen-2-yl}methyl)pyridazin-3-amine

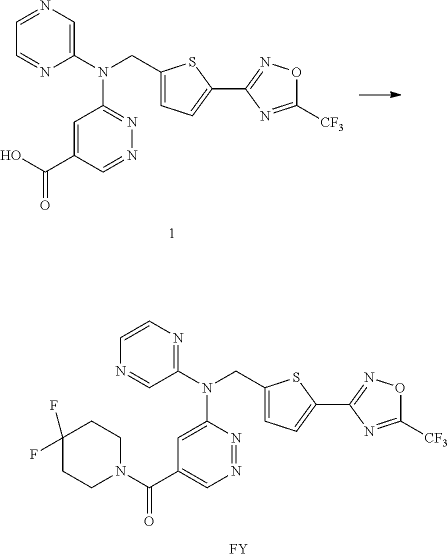 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CCC(F)(F)CC1 ; "FY"]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 30 min at rt before addition of 4,4-difluoropiperidine hydrochloride (35 mg, 0.22 mmol). After 19 h the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with hexanes/EtOAc (7:3 to 0:1) afforded FY as light beige solids (46 mg, 75%).

LCMS (ES): Found 552.8 [M+H]⁺.

¹H NMR (300 MHz, DMSO-d₆) δ: 8.97 (d, J=1.7 Hz, 1H), 8.85 (d, J=1.5 Hz, 1H), 8.40 (dd, J=2.6, 1.5 Hz, 1H), 8.33 (d, J=2.6 Hz, 1H), 7.85 (d, J=1.5 Hz, 1H), 7.75 (d, J=3.8 Hz, 1H), 7.31 (d, J=3.8 Hz, 1H), 5.76 (s, 2H), 3.65-3.77 (m, 2H), 3.37-3.47 (m, 2H), 1.96-2.16 (m, 4H).

¹⁹F NMR (282 MHz, DMSO-d₆) δ: −64.80 (s, 3F), −96.03-95.64 (m, 2F).

### Example FZ

### 5-{2-Oxa-6-azaspiro[3.3]heptane-6-carbonyl}-N-(pyrazin-2-yl)-N-({5-[5-(trifluoromethyl)-1,2,4-oxadiazol-3-yl]thiophen-2-yl}methyl)pyridazin-3-amine

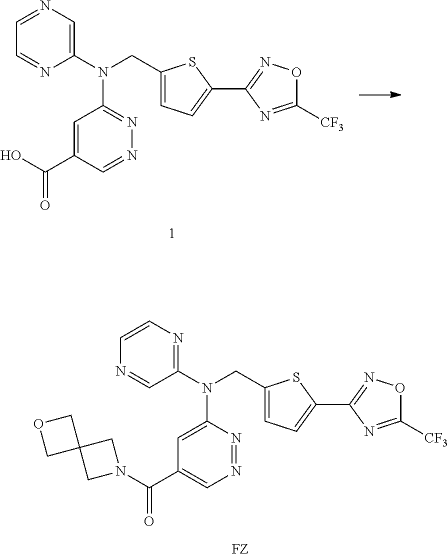 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CC2(COC2)C1 ; "FZ"]

To a solution of acid 1 (50 mg, 0.11 mmol) in DMF (1 mL) was added N-methylmorpholine (61 μL, 0.56 mmol) followed by PyBOP (87 mg, 0.17 mmol). The reaction mixture was stirred for 30 min at rt before addition of 2-oxa-6-azaspiro[3.3]heptane oxalate (42 mg, 0.22 mmol). After 20 h the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with EtOAc/MeOH (1:0 to 4:1) afforded FZ as pale yellow solids (27 mg, 46%).

LCMS (ES): Found 530.9 [M+H]⁺.

¹H NMR (300 MHz, DMSO-d₆) δ: 9.01 (d, J=1.7 Hz, 1H), 8.83 (d, J=1.3 Hz, 1H), 8.43 (dd, J=2.5, 1.4 Hz, 1H), 8.34 (d, J=2.6 Hz, 1H), 7.81 (d, J=1.7 Hz, 1H), 7.76 (d, J=3.8 Hz, 1H), 7.32 (d, J=3.8 Hz, 1H), 5.77 (s, 2H), 4.66 (dd, J=11.3, 7.0 Hz, 4H), 4.47 (s, 2H), 4.23 (s, 2H).

¹⁹F NMR (282 MHz, DMSO-d₆) δ: −64.80 (s, 3F).

### Example GA

### 5-{8-Oxa-3-azabicyclo[3.2.1]octane-3-carbonyl}-N-(pyrazin-2-yl)-N-({5-[5-(trifluoromethyl)-1,2,4-oxadiazol-3-yl]thiophen-2-yl}methyl)pyridazin-3-amine

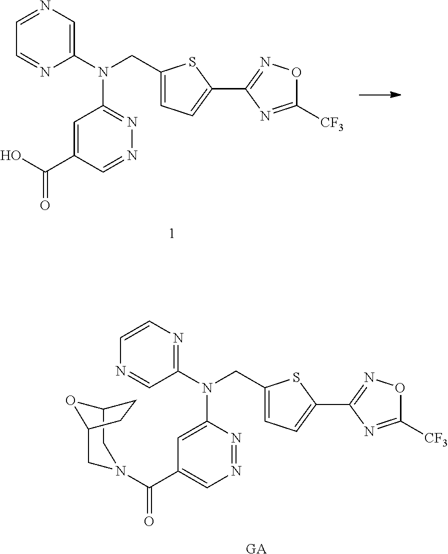 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CC2CCC(C1)O2 ; "GA"]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 30 min at rt before addition of 8-oxa-3-azabicyclo[3.2.1]octane hydrochloride (33 mg, 0.22 mmol). After 4 h 15 min the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with EtOAc/MeOH (1:0 to 19:1) afforded GA as light peach-coloured solids (35 mg, 58%).

LCMS (ES): Found 544.9 [M+H]⁺.

¹H NMR (300 MHz, DMSO-d₆) δ: 8.95 (d, J=1.7 Hz, 1H), 8.84 (d, J=1.5 Hz, 1H), 8.40 (dd, J=2.6, 1.5 Hz, 1H), 8.31 (d, J=2.6 Hz, 1H), 7.79 (d, J=1.7 Hz, 1H), 7.75 (d, J=3.8 Hz, 1H), 7.31 (d, J=3.8 Hz, 1H), 5.74 (s, 2H), 4.35-4.43 (m, 1H), 4.19-4.27 (m, 1H), 4.09 (br d, J=13.0 Hz, 1H), 3.36-3.42 (m, 1H), 3.16 (br d, J=12.4 Hz, 1H), 2.94-3.03 (m, 1H), 1.59-1.87 (m, 4H).

¹⁹F NMR (282 MHz, DMSO-d₆) δ: −64.80 (s, 3F).

### Example GB

### 5-{3-Oxa-8-azabicyclo[3.2.1]octane-8-carbonyl}-N-(pyrazin-2-yl)-N-({5-[5-(trifluoromethyl)-1,2,4-oxadiazol-3-yl]thiophen-2-yl}methyl)pyridazin-3-amine

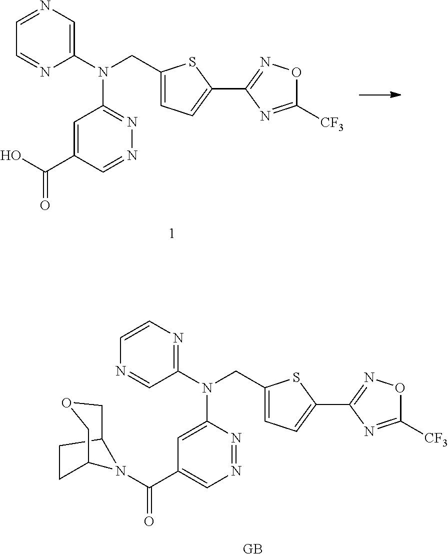 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1C2CCC1COC2 ; "GB"]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 30 min at rt before addition of 3-oxa-8-azabicyclo[3.2.1]octane hydrochloride (33 mg, 0.22 mmol). After 4 h 15 min the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with EtOAc/MeOH (1:0 to 19:1) afforded GB as light peach-coloured solids (40 mg, 66%).

LCMS (ES): Found 544.9 [M+H]⁺.

¹H NMR (300 MHz, DMSO-d₆) δ: 8.99 (d, J=1.7 Hz, 1H), 8.86 (d, J=1.3 Hz, 1H), 8.38 (dd, J=2.6, 1.5 Hz, 1H), 8.32 (d, J=2.6 Hz, 1H), 7.83 (d, J=1.7 Hz, 1H), 7.75 (d, J=3.8 Hz, 1H), 7.32 (d, J=3.8 Hz, 1H), 5.68-5.86 (m, 2H), 4.49-4.57 (m, 1H), 3.86-3.95 (m, 1H), 3.57-3.67 (m, 3H), 3.51 (d, J=10.7 Hz, 1H), 1.80-1.97 (m, 4H).

¹⁹F NMR (DMSO-d₆) δ: −64.80 (s, 3F).

### Example GC

### 5-[3-(Propan-2-yloxy)azetidine-1-carbonyl]-N-(pyrazin-2-yl)-N-({5-[5-(trifluoromethyl)-1,2,4-oxadiazol-3-yl]thiophen-2-yl}methyl)pyridazin-3-amine

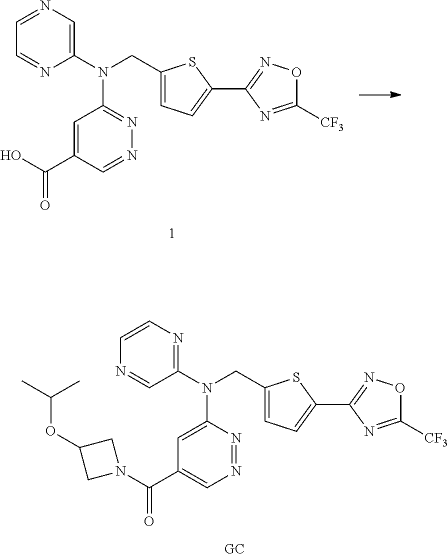 [SMILES: O=C(O)c1cnnc(N(Cc2ccc(-c3noc(C(F)(F)F)n3)s2)c2cnccn2)c1 ; CC(C)OC1CN(C(=O)c2cnnc(N(Cc3ccc(-c4noc(C(F)(F)F)n4)s3)c3cnccn3)c2)C1 ; "GC"]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 50 min at rt before addition of 3-(isopropoxy)azetidine hydrochloride (34 mg, 0.22 mmol). After 26 h the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with hexanes/EtOAc (1:0 to 0:1) afforded GC as pale yellow solids (38 mg, 62%).

LCMS (ES): Found 546.9 [M+H]⁺.

¹H NMR (300 MHz, DMSO-d₆) δ: 9.03 (d, J=1.7 Hz, 1H), 8.83 (d, J=1.3 Hz, 1H), 8.40 (dd, J=2.6, 1.5 Hz, 1H), 8.32 (d, J=2.6 Hz, 1H), 7.84 (d, J=1.7 Hz, 1H), 7.74 (d, J=3.8 Hz, 1H), 7.31 (d, J=3.8 Hz, 1H), 5.76 (d, J=1.9 Hz, 2H), 4.35-4.51 (m, 2H), 4.25-4.35 (m, 1H), 4.14-4.22 (m, 1H), 3.77-3.87 (m, 1H), 3.61 (spt, J=6.2 Hz, 1H), 1.08 (t, J=5.7 Hz, 6H).

¹⁹F NMR (282 MHZ, DMSO-d₆) δ: −64.80 (s, 3F).

### Example GD

### 1-{6-[(Pyrazin-2-yl)({5-[5-(trifluoromethyl)-1,2,4-oxadiazol-3-yl]thiophen-2-yl}methyl)amino]pyridazine-4-carbonyl}-3-(trifluoromethyl)azetidin-3-ol

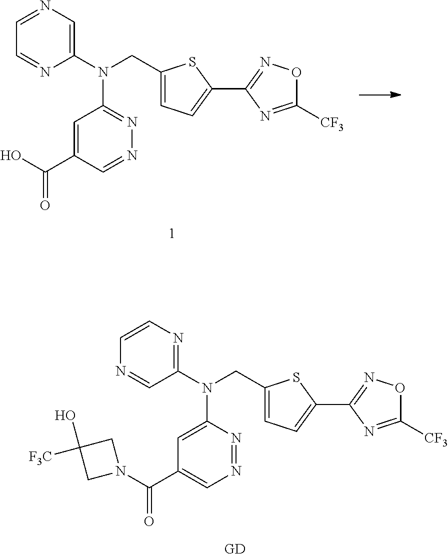 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CC(O)(C(F)(F)F)C1 ; "GD"]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 1 h at rt before addition of 3-(trifluoromethyl)azetidin-3-ol hydrochloride (40 mg, 0.22 mmol). After 26 h the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with hexanes/EtOAc (1:0 to 0:1) afforded GD as light peach-coloured solids (49 mg, 77%).

LCMS (ES): Found 572.8 [M+H]⁺.

¹H NMR (300 MHZ, DMSO-d₆) δ: 9.08 (d, J=1.7 Hz, 1H), 8.84 (d, J=1.3 Hz, 1H), 8.39 (dd, J=2.5, 1.4 Hz, 1H), 8.33 (d, J=2.6 Hz, 1H), 7.90 (d, J=1.7 Hz, 1H), 7.74 (d, J=3.6 Hz, 1H), 7.52 (s, 1H), 7.31 (d, J=3.8 Hz, 1H), 5.78 (s, 2H), 4.64 (d, J=10.2 Hz, 1H), 4.26-4.40 (m, 2H), 4.07 (br d, J=11.3 Hz, 1H).

¹⁹F NMR (282 MHz, DMSO-d₆) δ: −64.81 (s, 3F), −82.59 (s, 3F).

### Example GE

### N-Methanesulfonyl-N-methyl-6-[(pyrazin-2-yl)({5-[5-(trifluoromethyl)-1,2,4-oxadiazol-3-yl]thiophen-2-yl}methyl)amino]pyridazine-4-carboxamide

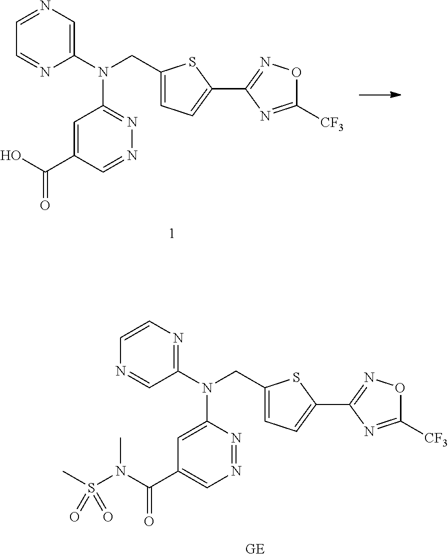 [SMILES: O=C(O)c1cnnc(N(Cc2ccc(-c3noc(C(F)(F)F)n3)s2)c2cnccn2)c1 ; CN(C(=O)c1cnnc(N(Cc2ccc(-c3noc(C(F)(F)F)n3)s2)c2cnccn2)c1)S(C)(=O)=O ; "GE"]

To a solution of acid 1 (50 mg, 0.11 mmol) in DMF (1 mL) was added N-methylmorpholine (25 μL, 0.22 mmol) followed by PyBOP (87 mg, 0.17 mmol).

The reaction mixture was stirred for 1 h at rt before addition of N-methyl methanesulfonamide (19 μL, 0.22 mmol). After 22 h the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with hexanes/EtOAc (1:0 to 0:1) afforded GE as a light yellow foam (35 mg, 58%).

LCMS (ES): Found 540.8 [M+H]⁺.

¹H NMR (300 MHz, DMSO-d₆) δ: 9.05 (d, J=1.7 Hz, 1H), 8.81 (d, J=1.5 Hz, 1H), 8.41 (dd, J=2.6, 1.3 Hz, 1H), 8.34 (d, J=2.4 Hz, 1H), 8.00 (d, J=1.9 Hz, 1H), 7.75 (d, J=3.8 Hz, 1H), 7.30 (d, J=3.8 Hz, 1H), 5.76 (s, 2H), 3.44 (s, 3H), 3.22 (s, 3H).

¹⁹F NMR (282 MHz, DMSO-d₆) δ: −64.79 (s, 3F).

### Example GF

### 5-{6-Oxa-1-azaspiro[3.3]heptane-1-carbonyl}-N-(pyrazin-2-yl)-N-({5-[5-(trifluoromethyl)-1,2,4-oxadiazol-3-yl]thiophen-2-yl}methyl)pyridazin-3-amine

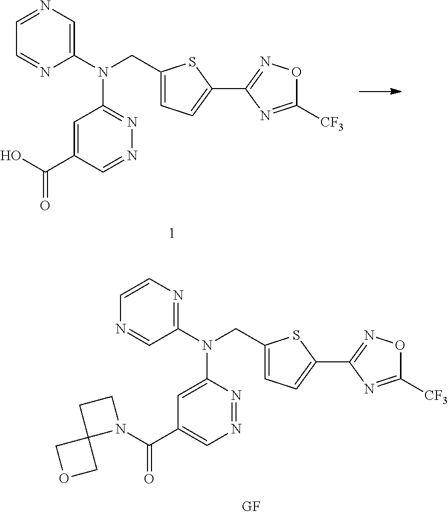 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CCC12COC2]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 40 min at rt before addition of 6-oxa-1-azaspiro[3.3]heptane hemioxalate (32 mg, 0.22 mmol). After 18 h the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with EtOAc/MeOH (1:0 to 9:1) afforded GF as light yellow semisolids (26 mg, 22%).

LCMS (ES): Found 530.9 [M+H]⁺.

¹H NMR (300 MHz, DMSO-d₆) δ: 9.04 (d, J=1.5 Hz, 1H), 8.82 (d, J=1.3 Hz, 1H), 8.40 (dd, J=2.5, 1.4 Hz, 1H), 8.33 (d, J=2.6 Hz, 1H), 7.86 (d, J=1.7 Hz, 1H), 7.74 (d, J=3.8 Hz, 1H), 7.31 (d, J=3.8 Hz, 1H), 5.76 (s, 2H), 5.29 (d, J=7.2 Hz, 2H), 4.57 (d, J=7.2 Hz, 2H), 4.09 (t, J=7.5 Hz, 2H), 2.52-2.59 (m, 2H).

¹⁹F NMR (282 MHz, DMSO-d₆) δ: −64.80 (s, 3F).

### Example GG

### N-{4-[(Pyrrolidin-1-yl)methyl]pyridin-2-yl}-N-({5-[5-(trifluoromethyl)-1,2,4-oxadiazol-3-yl]thiophen-2-yl}methyl)pyrazin-2-amine

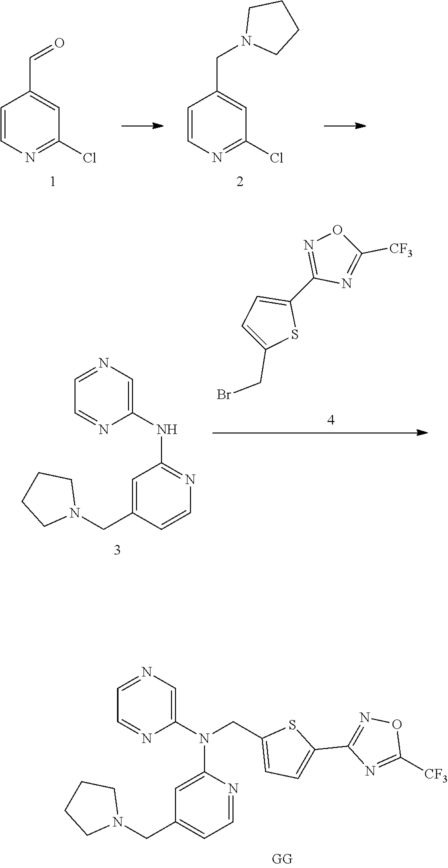 [SMILES: O=Cc1ccnc(Cl)c1 ; Clc1cc(CN2CCCC2)ccn1 ; c1cnc(Nc2cc(CN3CCCC3)ccn2)cn1 ; FC(F)(F)c1nc(-c2ccc(CBr)s2)no1 ; FC(F)(F)c1nc(-c2ccc(CN(c3cnccn3)c3cc(CN4CCCC4)ccn3)s2)no1]

To a solution of 2-chloroisonicotinaldehyde (1) (1.00 g, 7.07 mmol) and pyrrolidine (1.6 mL, 14.13 mmol) in 1,2-dichloroethane (20 mL) was added sodium triacetoxyborohydride (2.99 g, 14.13 mmol) and the mixture was stirred at rt for 2 hr. The reaction mixture was quenched with NaOH solution (5%, 20 mL) and phases separated. The aqueous phase was saturated with NaCl (ca 5 g required) and extracted with EtOAc (2×20 mL). The combined organics were concentrated in vacuo to afford 2 as a mobile yellow oil (1.18 g, 85%).

A suspension of 2 (462 mg, 2.35 mmol), 2-aminopyrazine (246 mg, 2.59 mmol), Cs₂CO₃ (1.53 g, 4.70 mmol) and Xantphos (136 mg, 0.24 mmol) in 1,4-dioxane (20.0 mL) was purged with Ar(g) for 30 min. Pd₂(dba)₃ (107 mg, 0.12 mmol) was added and the mixture was heated to 90° C. for 4 hr. The reaction was cooled to rt and partitioned between water (20 mL), brine (20 mL) and EtOAc (40 mL). The aqueous phase was re-extracted with EtOAc (2×20 mL). The combined organics were washed with brine (25 mL), dried over MgSO₄, filtered and concentrated in vacuo. Purification by flash column chromatography using DCM/MeOH (1:0 to 5:1) afforded 3 as a light orange-brown oil (270 mg, 45%). ^{t}BuOK (1.7 M/THF, 682 μL) was added to a solution of 3 (269 mg, 1.05 mmol) in DMF (11 mL) at 0° C. under Ar(g). The reaction mixture was stirred for 5 min then 4 (391 mg, 1.16 mmol) was added as solids in one portion. The reaction mixture was stirred for 50 min in the cooling bath and then partitioned between water (11 mL), brine (11 mL), and EtOAc (22 mL). The aqueous phase was separated and basified with NaOH solution (5%, 1 mL) followed by re-extraction with EtOAc (3×11 mL). The combined organics were washed with brine (10 mL), dried over MgSO₄, filtered, and concentrated in vacuo. Purification by flash column chromatography with EtOAc/MeOH (1:0 to 9:1) afforded a viscous light brown oil (215 mg). The residue was re-dissolved in DCM/MeOH (4:1, 25 mL) and treated with MP-TMT resin (1.3 meq/g, 500 mg) for 18 hr. The resin was removed by filtration and the filtrate concentrated in vacuo to afford a straw coloured residue, which was further purified by flash column chromatography with DCM/MeOH (1:0 to 9:1) to afford GG as a viscous light brown oil (88 mg, 17%).

LCMS (ES): Found 488.0 [M+H]⁺.

¹H NMR (300 MHz, DMSO-d₆) δ: 8.61 (d, J=1.5 Hz, 1H), 8.37 (dd, J=2.6, 1.5 Hz, 1H), 8.30 (d, J=5.5 Hz, 1H), 8.16 (d, J=2.6 Hz, 1H), 7.72 (d, J=3.8 Hz, 1H), 7.31 (s, 1H), 7.23 (d, J=3.8 Hz, 1H), 7.05 (dd, J=5.1, 1.1 Hz, 1H), 5.60 (s, 2H), 3.59 (s, 2H), 2.37-2.47 (m, 4H), 1.61-1.75 (m, 4H).

¹⁹F NMR (282 MHz, DMSO-d₆) δ: −64.80 (s, 3F).

### Example GH

### N-{4-[(3-Methoxyazetidin-1-yl)methyl]pyridin-2-yl}-N-({5-[5-(trifluoromethyl)-1,2,4-oxadiazol-3-yl]thiophen-2-yl}methyl)pyrazin-2-amine

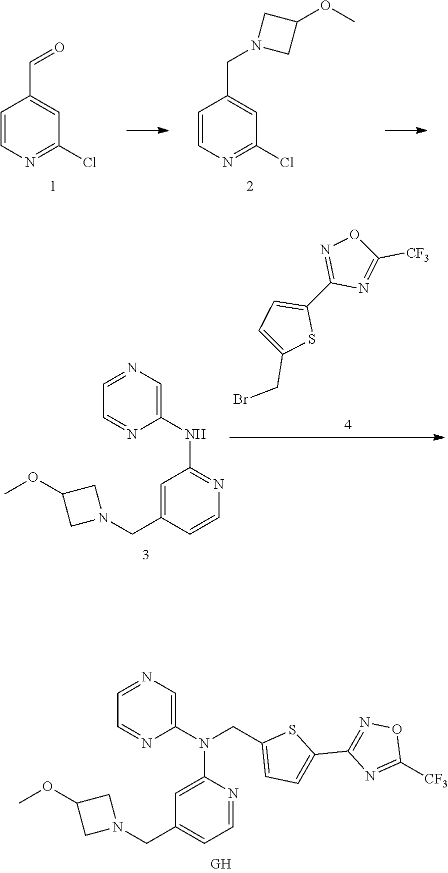 [SMILES: O=Cc1ccnc(Cl)c1 ; COC1CN(Cc2ccnc(Cl)c2)C1 ; COC1CN(Cc2ccnc(Nc3cnccn3)c2)C1 ; FC(F)(F)c1nc(-c2ccc(CBr)s2)no1 ; COC1CN(Cc2ccnc(N(Cc3ccc(-c4noc(C(F)(F)F)n4)s3)c3cnccn3)c2)C1]

To a suspension of 2-chloroisonicotinaldehyde (1) (1.00 g, 7.07 mmol), 3-methoxyazetidine hydrochloride (1.75 g, 14.13 mmol), and sodium acetate (1.74 g, 21.19 mmol) in 1,2-dichloroethane (20 mL) was added sodium triacetoxyborohydride (2.99 g, 14.13 mmol) and the mixture was stirred at rt for 2 hr. The reaction mixture was quenched with NaOH solution (5%, 20 mL) and phases separated. The aqueous phase was saturated with NaCl (ca 5 g required) and extracted with EtOAc (2×20 mL). The combined organics were concentrated in vacuo to afford 2 as a mobile yellow oil (1.53 g, quantitative).

A suspension of 2 (500 mg, 2.35 mmol), 2-aminopyrazine (246 mg, 2.59 mmol), Cs₂CO₃ (1.53 g, 4.70 mmol) and Xantphos (136 mg, 0.24 mmol) in 1,4-dioxane (20.0 mL) was purged with Ar(g) for 30 min. Pd₂(dba)₃ (107 mg, 0.12 mmol) was added and mixture was heated to 90° C. for 4 hr. The reaction was cooled to rt and partitioned between water (20 mL), brine (20 mL) and EtOAc (20 mL). The aqueous phase was re-extracted with EtOAc (2×20 mL). The combined organics were washed with brine (25 mL), dried over MgSO₄, filtered and concentrated in vacuo. Purification by flash column chromatography using EtOAc/MeOH (1:0 to 9:1) afforded 3 as a viscous dark straw-coloured oil (355 mg, 56%).

^{t}BuOK (1.7 M/THF, 849 μL) was added to a solution of 3 (355 mg, 1.31 mmol) in DMF (14 mL) at 0° C. under Ar(g). The reaction mixture was stirred for 10 min then 4 (487 mg, 1.44 mmol) was added as solids in one portion. The reaction mixture was stirred for 2 h 15 min in the cooling bath and then partitioned between water (14 mL), brine (14 mL), and EtOAc (28 mL). The aqueous phase was separated and basified with NaOH solution (5%, 1 mL) followed by re-extraction with EtOAc (3×14 mL). The combined organics were washed with brine (15 mL), dried over MgSO₄, filtered, and concentrated in vacuo. Purification by flash column chromatography with EtOAc/MeOH (1:0 to 7:1) afforded a yellow film (69 mg). The residue was re-dissolved in DCM/MeOH (4:1, 25 mL) and treated with MP-TMT resin (1.3 meq/g, 500 mg) for 6 h 30 min. The resin was removed by filtration and the filtrate concentrated in vacuo to afford a straw coloured residue, which was further purified by flash column chromatography with DCM/MeOH (1:0 to 9:1) to afford GH as a pale yellow film (12 mg, 2%).

LCMS (ES): Found 503.9 [M+H]⁺.

¹H NMR (300 MHz, DMSO-d₆) δ: 8.61 (d, J=1.3 Hz, 1H), 8.38 (dd, J=2.6, 1.5 Hz, 1H), 8.29 (d, J=5.1 Hz, 1H), 8.17 (d, J=2.6 Hz, 1H), 7.73 (d, J=3.8 Hz, 1H), 7.19-7.31 (m, 2H), 7.00 (dd, J=5.1, 0.9 Hz, 1H), 5.60 (s, 2H), 3.97 (quin, J=5.7 Hz, 1H), 3.60 (s, 2H), 3.43-3.53 (m, 2H), 3.13 (s, 3H), 2.81-2.92 (m, 2H).

¹⁹F NMR (282 MHz, DMSO-d₆) δ: −64.81 (s, 3F).

### Example GI

### N-{4-[(Morpholin-4-yl)methyl]pyridin-2-yl}-N-({5-[5-(trifluoromethyl)-1,2,4-oxadiazol-3-yl]thiophen-2-yl}methyl)pyrazin-2-amine

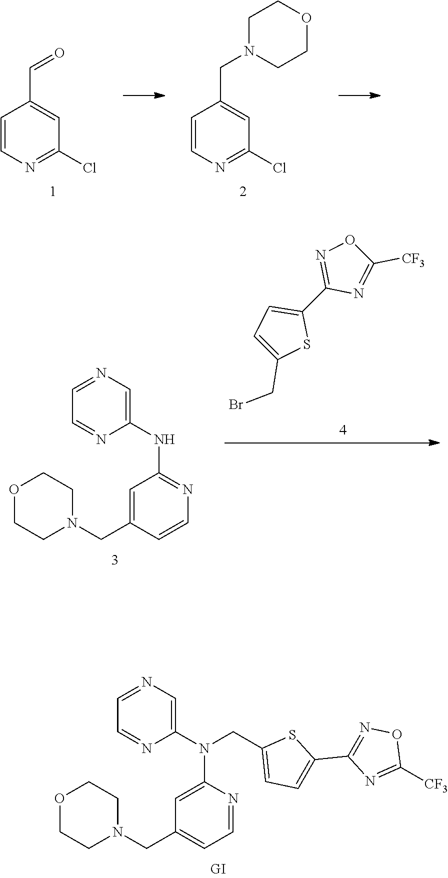 [SMILES: O=Cc1ccnc(Cl)c1 ; Clc1cc(CN2CCOCC2)ccn1 ; c1cnc(Nc2cc(CN3CCOCC3)ccn2)cn1 ; FC(F)(F)c1nc(-c2ccc(CBr)s2)no1 ; FC(F)(F)c1nc(-c2ccc(CN(c3cnccn3)c3cc(CN4CCOCC4)ccn3)s2)no1]

To a solution of 2-chloroisonicotinaldehyde (1) (1.00 g, 7.07 mmol) and morpholine (1.2 mL, 14.13 mmol) in 1,2-dichloroethane (20 mL) was added sodium triacetoxyborohydride (2.99 g, 14.13 mmol) and the mixture was stirred at rt for 18 hr. The reaction mixture was quenched with NaOH solution (5%, 20 mL) and phases separated. The aqueous phase was saturated with NaCl (ca 5 g required) and extracted with EtOAc (2×20 mL). The combined organics were concentrated in vacuo to afford 2 as a viscous yellow oil (1.51 g, quantitative). A suspension of 2 (500 mg, 2.35 mmol), 2-aminopyrazine (246 mg, 2.59 mmol), Cs₂CO₃ (1.53 g, 4.70 mmol) and Xantphos (136 mg, 0.24 mmol) in 1,4-dioxane (20.0 mL) was purged with Ar(g) for 30 min. Pd₂(dba)₃ (107 mg, 0.12 mmol) was added and mixture was heated to 90° C. for 18 hr. The reaction was cooled to rt and partitioned between water (20 mL), brine (20 mL) and EtOAc (50 mL). The aqueous phase was re-extracted with EtOAc (2×20 mL). The combined organics were extracted with HCl solution (5%, 3×20 mL), which were combined and basified with NaOH solution (5%, ca 85 mL). NaCl (25 g) was added and the aqueous mixture was extracted with EtOAc (3×20 mL), washed with brine (10 mL), dried over MgSO₄, filtered and concentrated in vacuo. Purification by flash column chromatography using EtOAc/MeOH (1:0 to 9:1) afforded 3 as a viscous pale straw-coloured oil (584 mg, 92%).

^{t}BuOK (1.7 M/THF, 1.1 mL) was added to a solution of 3 (450 mg, 1.66 mmol) in DMF (18 mL) at 0° C. under Ar(g). The reaction mixture was stirred for 10 min then 4 (616 mg, 1.82 mmol) was added as solids in one portion. The reaction mixture was stirred for 1 h 30 min in the cooling bath and then partitioned between water (18 mL), brine (18 mL), and EtOAc (3×36 mL). The combined organics were washed with brine (18 mL), dried over MgSO₄, filtered, and concentrated in vacuo. The residue was re-dissolved in DCM/MeOH (4:1, 50 mL) and treated with MP-TMT resin (1.3 meq/g, 640 mg) for 2 h 30 min. The resin was removed by filtration and the filtrate concentrated in vacuo to afford a viscous brown residue, which was purified by flash column chromatography with hexanes/EtOAc (1:0 to 0:1), followed by a second purification by flash column chromatography with DCM/MeOH (1:0 to 19:1) to afford GI as a viscous yellow oil (44 mg, 5%).

LCMS (ES): Found 504.0 [M+H]⁺.

¹H NMR (300 MHz, DMSO-d₆) δ: 8.63 (d, J=1.5 Hz, 1H), 8.37 (dd, J=2.6, 1.5 Hz, 1H), 8.31 (d, J=4.9 Hz, 1H), 8.17 (d, J=2.6 Hz, 1H), 7.73 (d, J=3.8 Hz, 1H), 7.33 (s, 1H), 7.23 (d, J=3.8 Hz, 1H), 7.06 (d, J=5.1 Hz, 1H), 5.60 (s, 2H), 3.52-3.62 (m, 4H), 3.49 (s, 2H), 2.29-2.43 (m, 4H).

¹⁹F NMR (282 MHz, DMSO-d₆) δ: −64.79 (s, 3F).

### Example GJ

### N-(Pyrazin-2-yl)-N-({5-[5-(trifluoromethyl)-1,2,4-oxadiazol-3-yl]thiophen-2-yl}methyl)-5-[3-(trifluoromethyl)azetidine-1-carbonyl]pyridazin-3-amine

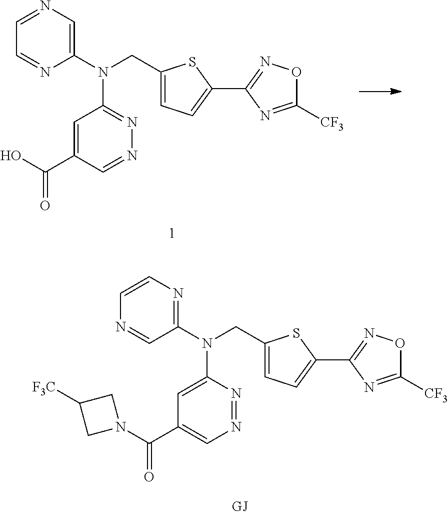 [SMILES: O=C(O)c1cnnc(N(Cc2ccc(-c3noc(C(F)(F)F)n3)s2)c2cnccn2)c1 ; O=C(c1cnnc(N(Cc2ccc(-c3noc(C(F)(F)F)n3)s2)c2cnccn2)c1)N1CC(C(F)(F)F)C1]

To a solution of acid 1 (50 mg, 0.11 mmol) in DMF (1 mL) was added N-methylmorpholine (25 μL, 0.22 mmol) followed by PyBOP (87 mg, 0.17 mmol). The reaction mixture was stirred for 30 min at rt before addition of 3-(trifluoromethyl)azetidine (28 mg, 0.22 mmol). After 25 h the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with hexanes/EtOAc (1:0 to 0:1) afforded GJ as pale beige solids (42 mg, 68%).

LCMS (ES): Found 556.9 [M+H]⁺.

¹H NMR (300 MHz, DMSO-d₆) δ: 9.06 (d, J=1.7 Hz, 1H), 8.84 (d, J=1.5 Hz, 1H), 8.39 (dd, J=2.6, 1.5 Hz, 1H), 8.33 (d, J=2.6 Hz, 1H), 7.88 (d, J=1.9 Hz, 1H), 7.74 (d, J=3.8 Hz, 1H), 7.31 (d, J=3.8 Hz, 1H), 5.77 (s, 2H), 4.55 (t, J=9.0 Hz, 1H), 4.43 (dd, J=9.4, 5.5 Hz, 1H), 4.31 (t, J=9.9 Hz, 1H), 4.06 (dd, J=10.8, 5.6 Hz, 1H), 3.62-3.77 (m, 1H).

¹⁹F NMR (282 MHz, DMSO-d₆) δ: −64.81 (s, 3F), −72.07 (d, J=9.3 Hz, 3F).

### Example GK

### 5-[3-(Difluoromethyl)azetidine-1-carbonyl]-N-(pyrazin-2-yl)-N-({5-[5-(trifluoromethyl)-1,2,4-oxadiazol-3-yl]thiophen-2-yl}methyl)pyridazin-3-amine

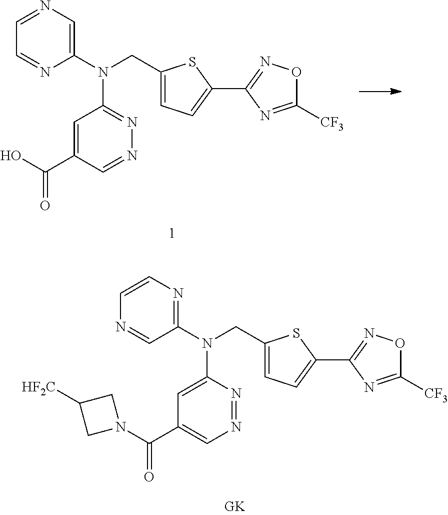

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 35 min at rt before addition of 3-(difluoromethyl)azetidine hydrochloride (32 mg, 0.22 mmol). After 3 h the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with hexanes/EtOAc (1:0 to 0:1) afforded GK as pale beige solids (37 mg, 63%).

LCMS (ES): Found 538.9 [M+H]⁺

¹H NMR (300 MHz, DMSO-d₆) δ: 9.04 (d, J=1.7 Hz, 1H), 8.84 (d, J=1.3 Hz, 1H), 8.39 (dd, J=2.6, 1.3 Hz, 1H), 8.33 (d, J=2.6 Hz, 1H), 7.85 (d, J=1.7 Hz, 1H), 7.74 (d, J=3.8 Hz, 1H), 7.32 (d, J=3.8 Hz, 1H), 6.34 (td, J=56.3, 4.5 Hz, 1H), 5.77 (s, 2H), 4.44 (t, J=8.9 Hz, 1H), 4.27 (dd, J=9.1, 5.7 Hz, 1H), 4.17 (t, J=10.0 Hz, 1H), 3.99 (dd, J=10.5, 5.8 Hz, 1H), 3.10-3.27 (m, 1H)

¹⁹F NMR (282 MHz, DMSO-d₆) δ: −64.81 (s, 3F), −124.48 (dd, J=56.4, 14.7 Hz, 2F)

### Example GL

### 5-[3-Methoxy-3-(trifluoromethyl)azetidine-1-carbonyl]-N-(pyrazin-2-yl)-N-({5-[5-(trifluoromethyl)-1,2,4-oxadiazol-3-yl]thiophen-2-yl}methyl)pyridazin-3-amine

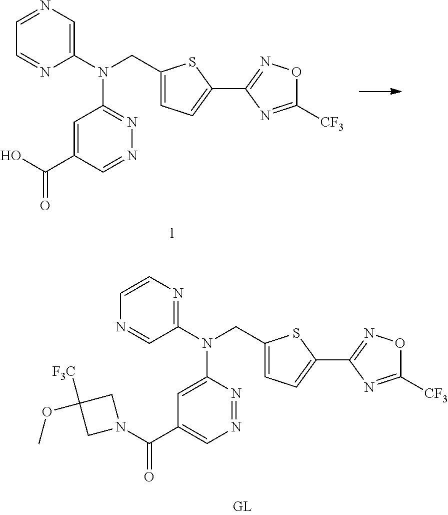 [SMILES: O=C(O)c1cnnc(N(Cc2ccc(-c3noc(C(F)(F)F)n3)s2)c2cnccn2)c1 ; COC1(C(F)(F)F)CN(C(=O)c2cnnc(N(Cc3ccc(-c4noc(C(F)(F)F)n4)s3)c3cnccn3)c2)C1]

To a solution of acid 1 (50 mg, 0.11 mmol) in DMF (1 mL) was added N-methylmorpholine (49 μL, 0.45 mmol) followed by PyBOP (87 mg, 0.17 mmol). The reaction mixture was stirred for 35 min at rt before addition of 3-methoxy-3-(trifluoromethyl)azetidine hydrochloride (43 mg, 0.22 mmol). After 3 h the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with hexanes/EtOAc (1:0 to 0:1) afforded GL as pale beige solids (46 mg, 70%).

LCMS (ES): Found 586.9 [M+H]⁺

¹H NMR (300 MHz, DMSO-d₆) δ: 9.10 (d, J=1.1 Hz, 1H), 8.84 (d, J=1.1 Hz, 1H), 8.40 (dd, J=2.3, 1.1 Hz, 1H), 8.33 (d, J=2.6 Hz, 1H), 7.93 (d, J=1.1 Hz, 1H), 7.74 (d, J=3.8 Hz, 1H), 7.31 (d, J=3.8 Hz, 1H), 5.78 (s, 2H), 0.00 (d, J=10.5 Hz, 1H), 4.56 (d, J=10.5 Hz, 1H), 4.36 (d, J=12.1 Hz, 1H), 4.24 (d, J=11.9 Hz, 1H), 3.48 (s, 3H).

¹⁹F NMR (282 MHz, DMSO-d₆) δ: −64.82 (s, 3F), −79.32 (s, 3F).

### Example GM

### N-(Pyrazin-2-yl)-5-(pyridin-2-yl)-N-({5-[5-(trifluoromethyl)-1,2,4-oxadiazol-3-yl]thiophen-2-yl}methyl)pyridazin-3-amine

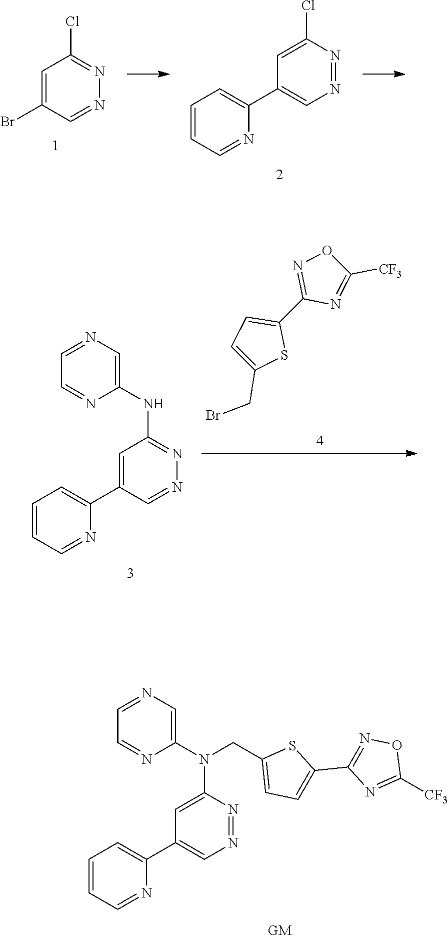 [SMILES: Clc1cc(Br)cnn1 ; Clc1cc(-c2ccccn2)cnn1 ; c1ccc(-c2cnnc(Nc3cnccn3)c2)nc1 ; FC(F)(F)c1nc(-c2ccc(CBr)s2)no1 ; FC(F)(F)c1nc(-c2ccc(CN(c3cnccn3)c3cc(-c4ccccn4)cnn3)s2)no1]

A suspension of 5-bromo-3-chloropyridazine (1) (500 mg, 2.58 mmol), pyridine-2-boronic acid pinacol ester (530 mg, 2.58 mmol), Cs₂CO₃ (3.37 g, 10.34 mmol), copper(I) chloride (256 mg, 2.58 mmol), and palladium(II)(dppf) chloride (189 mg, 0.26 mmol) in DMF (20 mL) was purged with Ar(g) for 10 min and then heated to 60° C. for 3 hr. The reaction was cooled to rt and partitioned between water (50 mL) and EtOAc (150 mL). The aqueous phase was re-extracted with EtOAc (50 mL). The combined organics were washed with water (50 mL), half saturated brine (3×40 mL), and brine (20 mL), dried over MgSO₄, filtered and concentrated in vacuo. Purification by flash column chromatography using hexanes/EtOAc (1:0 to 7:3) afforded 2 as off-white solids (129 mg, 26%). A suspension of 2 (100 mg, 0.52 mmol), 2-aminopyrazine (50 mg, 0.52 mmol), Cs₂CO₃ (340 mg, 1.04 mmol) and Xantphos (30 mg, 0.05 mmol) in 1,4-dioxane (2.0 mL) was purged with Ar(g) for 30 min. Pd₂(dba)₃ (24 mg, 0.03 mmol) was added and mixture was heated to 90° C. for 20 hr. The reaction was cooled to rt and partitioned between water (20 mL) and EtOAc (50 mL). The aqueous phase was re-extracted with EtOAc (20 mL). The combined EtOAc extracts were discarded, and the aqueous phase was re-extracted with DCM/MeOH (9:1, 4×50 mL), the combined organics filtered and concentrated in vacuo to afford 3 as yellow-beige solids (110 mg, 84%).

^{t}BuOK (1.7 M/THF, 209 μL) was added to a suspension of 3 (81 mg, 0.32 mmol) in DMF (3.2 mL) at 0° C. under Ar(g). The reaction mixture was stirred for 5 min then 4 (111 mg, 0.34 mmol) was added as solids in one portion. The reaction mixture was stirred for 45 min in the cooling bath and then poured into a mixture of water (5 mL) and brine (5 mL) followed by extraction with EtOAc (3×10 mL). The combined organics were washed with brine (10 mL), dried over MgSO₄, and treated with MP-TMT resin (1.3 meq./g, 250 mg) for 4 hr. The mixture was filtered and concentrated in vacuo. Purification by flash column chromatography with hexanes/EtOAc (1:0 to 0:1) afforded a brown gum which was re-purified by flash column chromatography with DCM/MeOH (1:0 to 19:1) to afford GM as tan solids (64 mg, 41%).

LCMS (ES): Found 483.1 [M+H]⁺

¹H NMR (300 MHz, DMSO-d₆) δ: 9.63 (d, J=1.9 Hz, 1H), 8.82 (d, J=1.5 Hz, 1H), 8.74-8.79 (m, 1H), 8.43 (dd, J=2.6, 1.5 Hz, 1H), 8.28-8.34 (m, 2H), 8.21-8.27 (m, 1H), 8.01 (td, J=7.8, 1.8 Hz, 1H), 7.74 (d, J=3.8 Hz, 1H), 7.55 (ddd, J=7.6, 4.8, 1.0 Hz, 1H), 7.33 (d, J=3.8 Hz, 1H), 5.80 (s, 2H).

¹⁹F NMR (282 MHz, DMSO-d₆) δ: −64.80 (s, 3F).

### Example GN

### 5-Methyl-N-(pyrazin-2-yl)-N-({5-[5-(trifluoromethyl)-1,2,4-oxadiazol-3-yl]thiophen-2-yl}methyl)pyridazin-3-amine

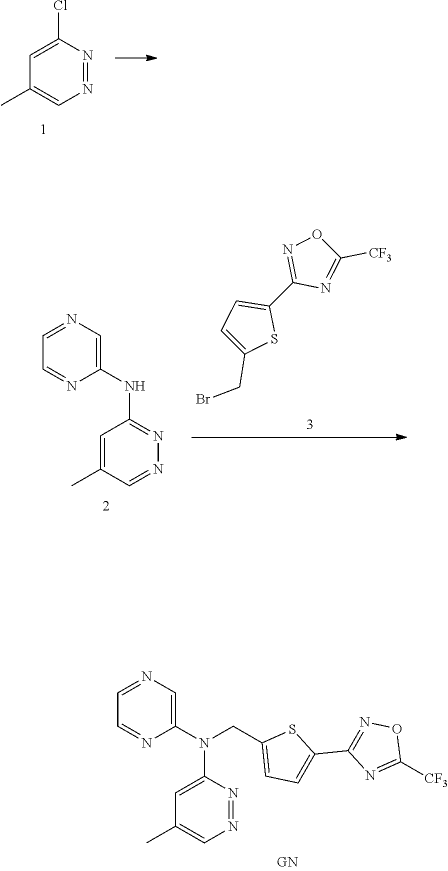

A suspension of 1 (500 mg, 2.80 mmol), 2-aminopyrazine (266 mg, 2.80 mmol), Cs₂CO₃ (1.82 g, 5.60 mmol) and Xantphos (162 mg, 0.14 mmol) in 1,4-dioxane (10.0 mL) was purged with Ar(g) for 20 min. Pd₂(dba)₃ (24 mg, 0.03 mmol) was added and mixture was heated to 90° C. for 20 hr. The reaction was cooled to rt, diluted with water (20 mL) and brine (20 mL), and extracted with EtOAc (50 mL, 2×25 mL). The combined organics were dried over MgSO₄, filtered and concentrated in vacuo. The residue was purified by flash column chromatography with hexanes/EtOAc (1:0 to 0:1) to afford light brown solids, which were triturated with DCM (2 mL, then 2×1 mL) and dried in vacuo to afford 2 as light beige solids (33 mg, 6%).

^{t}BuOK (1.7 M/THF, 79 μL) was added to a suspension of 2 (23 mg, 0.12 mmol) in DMF (1.0 mL) at 0° C. under Ar(g). The reaction mixture was stirred for 40 min then 3 (42 mg, 0.14 mmol) was added as solids in one portion. The reaction mixture was stirred for 1 h in the cooling bath and then poured into a mixture of water (5 mL) and brine (5 mL) followed by extraction with EtOAc (3×10 mL). The combined organics were washed with brine (10 mL), dried over MgSO₄, and concentrated in vacuo. Purification by flash column chromatography with hexanes/EtOAc (1:0 to 0:1) afforded GN as a viscous yellow-brown oil (24 mg, 47%).

LCMS (ES): Found 420.1 [M+H]⁺

¹H NMR (300 MHz, DMSO-d₆) δ: 8.83 (d, J=1.5 Hz, 1H), 8.70 (d, J=1.5 Hz, 1H), 8.41 (dd, J=2.6, 1.5 Hz, 1H), 8.26 (d, J=2.4 Hz, 1H), 7.73 (d, J=3.8 Hz, 1H), 7.61 (dd, J=1.7, 0.9 Hz, 1H), 7.28 (d, J=3.8 Hz, 1H), 5.69 (s, 2H), 2.30 (s, 3H)

¹⁹F NMR (282 MHz, DMSO-d₆) δ: −64.79 (s, 3F).

### Example GO

### 3-[(Pyrazin-2-yl)({5-[5-(trifluoromethyl)-1,2,4-oxadiazol-3-yl]thiophen-2-yl}methyl)amino]pyrazine-2-carboxamide

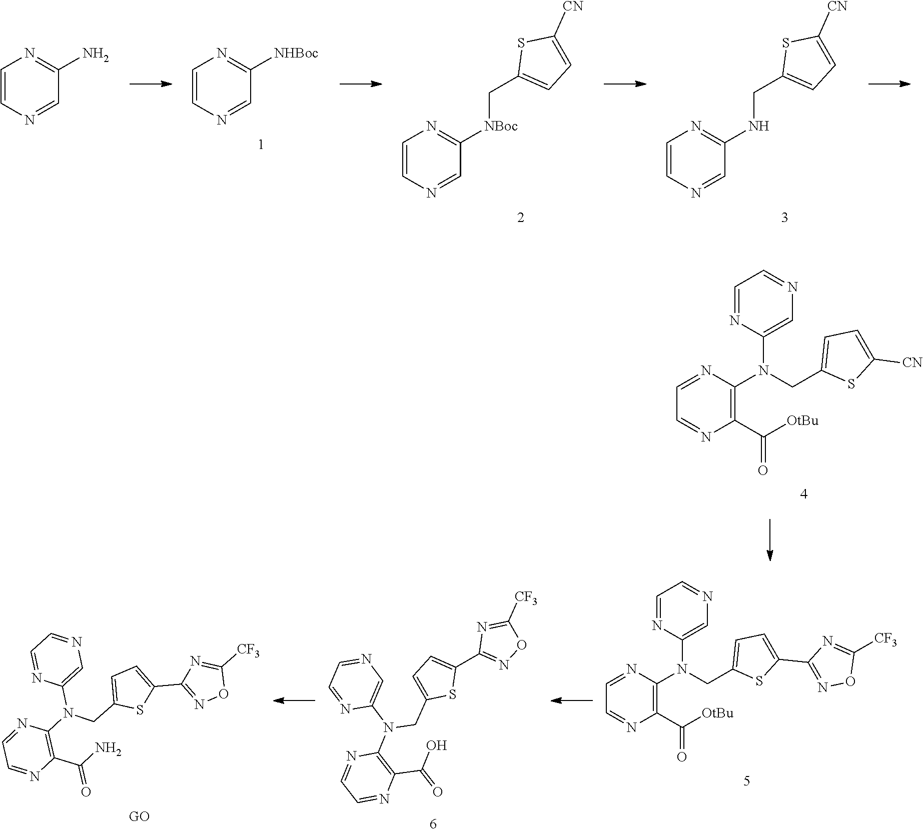

To a stirred solution of pyrazin-2-amine (1.902 g, 20 mmol) in THF (10 mL) at rt under an argon atmosphere, was added DMAP (catalytic amount) followed by (BOC)₂O (1M solution in THF, 22 mL, 22 mmol) dropwise. The reaction mixture was stirred for 2 h and then Et₃N (2.024 g, 20 mmol) was added. After 1 h (BOC)₂O (1M solution in THF, 5 mL, 5 mmol) was added dropwise and the reaction mixture stirred for a further 1 h. The reaction mixture was poured into 10% HCl solution (30 mL) and extracted with CH₂Cl₂ (30 mL) twice. The combined organic fractions were dried over MgSO₄, filtered and the solvent removed by evaporation in vacuo. The crude product was dissolved in MeOH (30 mL) and 1M NaOH solution (20 mL, 20 mmol) added. The reaction mixture was stirred for 2 h at rt and then neutralised using solid CO₂. Water (50 mL) was added and the product extracted with CH₂Cl₂ (50 mL) twice. The combined organic fractions were dried over MgSO₄, filtered and the solvent removed by evaporation in vacuo. The product was dissolved in hexane/CH₂Cl₂ treated with activated charcoal, filtered and the solvent removed by evaporation in vacuo to yield intermediate 1 as a white solid (3.18 g, 84%).

¹H NMR (300 MHz, DMSO-d6) δ: 10.20 (br s, 1H), 9.10 (d, J=1.5 Hz, 1H), 8.36 (dd, J=2.5, 1.6 Hz, 1H), 8.32 (d, J=2.6 Hz, 1H), 1.54 (s, 9H).

To a stirred solution of intermediate 1 (0.975 g, 5 mmol) in DMF (20 mL) under an argon atmosphere at rt was added NaH (60% dispersion in oil, 0.22 g, 5.5 mmol) in a single portion. The reaction mixture was stirred for 30 min and the cooled to 0° C. 5-(Bromomethyl)thiophene-2-carbonitrile (1.01 g, 5 mmol) was added as a solution in DMF (5 mL). The reaction mixture was allowed to warm to rt and then stirred for a further 1 h before being poured into Na₂CO₃ solution (20 mL) and extracted with CH₂Cl₂ (20 mL) twice. The combined organic fractions were dried over MgSO₄, filtered and the solvent removed by evaporation in vacuo. Purification by silica gel column chromatography, 5% EtOAc/CH₂Cl₂ elution, yielded intermediate 2 (1.236 g, 78%).

LCMS (ESI): Found 317.0 [M+H]⁺.

To a stirred solution of intermediate 2 (1.236 g, 3.9 mmol) in CH₂Cl₂ (10 mL) at rt was added TFA (2 mL). The reaction mixture was stirred overnight and then diluted with toluene (10 mL) and the solvent removed by evaporation in vacuo. Purification by silica gel column chromatography, 5% MeOH/CH₂Cl₂ elution, yielded intermediate 3, in quantitative yield.

¹H NMR (300 MHz, DMSO-d₆) δ: 8.00 (s, 1H), 7.99 (d, J=4.1 Hz, 1H), 7.85 (br t, J=6.1 Hz, 1H), 7.82 (d, J=3.8 Hz, 1H), 7.76 (d, J=2.6 Hz, 1H), 7.19 (d, J=4.0 Hz, 1H), 4.72 (d, J=5.7 Hz, 2H).

tert-Butyl 3-chloropyrazine-2-carboxylate (0.465 g, 2.17 mmol), Cs₂CO₃ (1.415 g, 3.34 mmol) and XantPhos (0.272 g, 0.47 mmol) were placed under an argon atmosphere. Intermediate 3 (0.469 g, 2.17 mmol) was added as a solution in 1,4-dioxane (7 mL). The reaction mixture was degassed and placed under an argon atmosphere. Pd₂(dba)₃ (0.197 g, 0.214 mmol) was added to the reaction mixture, which was then heated to 90° C. for 6 h. Upon cooling the reaction mixture was poured into water (10 mL) and extracted with CH₂Cl₂ (20 mL) twice. The combined organic fractions were dried over MgSO₄, filtered and the solvent removed by evaporation in vacuo. Purification by silica gel column chromatography, 20% EtOAc/CH₂Cl₂ elution, yielded intermediate 4 (0.746 g, 87%).

LCMS (ESI): Found 394.9 [M+H]⁺.

To a stirred solution of intermediate 4 (0.346 g, 0.88 mmol) in EtOH (20 mL) was added NH₂OH solution (50%, 0.2 mL). The reaction mixture was heated to 80° C. for 2 h. Upon cooling the solvent was removed by evaporation in vacuo. The crude product was redissolved in EtOH (20 mL) and the solvent removed by evaporation in vacuo. The process was repeated. The crude product was suspended in CH₂Cl₂ (10 mL) under an argon atmosphere at 0° C. Et₃N (0.37 mL, 2.64 mmol) was added followed by TFAA (0.244 mL, 1.76 mmol). The reaction mixture was allowed to warm to rt and stirred for 2 h and then poured into saturated NaHCO₃ solution (10 mL) and extracted with CH₂Cl₂ (10 mL) twice. The combined organic fractions were dried over MgSO₄, filtered and the solvent removed by evaporation in vacuo. Purification by silica gel column chromatography, 1:1 EtOAc/CH₂Cl₂ elution, yielded intermediate 5 (0.316 g, 71%).

LCMS (ESI): Found 505.9 [M+H]⁺.

To a stirred solution of intermediate 5 (0.316 g, 0.63 mmol) in CH₂Cl₂ (5 mL) at rt was added TFA (2 mL). The reaction mixture was stirred for 5 h, then diluted with toluene (5 mL) and the solvent removed by evaporation in vacuo. Purification by trituration with Et₂O yielded intermediate 6 (0.112 g, 40%).

LCMS (ESI): Found 449.2 [M+H]⁺.

¹H NMR (300 MHz, DMSO-d₆) δ: 13.29 (br s, 1H), 8.72 (d, J=2.4 Hz, 1H), 8.61 (d, J=1.1 Hz, 1H), 8.48 (d, J=2.4 Hz, 1H), 8.12-8.18 (m, 2H), 7.75 (d, J=3.8 Hz, 1H), 7.37 (d, J=3.8 Hz, 1H), 5.67 (s, 2H).

¹⁹F NMR (282 MHz, DMSO-d₆), δ: −64.79 (s, 3F).

To a stirred solution of intermediate 6 (0.045 g, 0.1 mmol) in DMF (5 mL) under an argon atmosphere at rt was added CDI (0.048 g, 0.3 mmol). The reaction mixture was stirred for 30 min and then NH₄Cl (0.154 g, 2.89 mmol) was added. The reaction mixture was stirred overnight and then poured into saturated Na₂CO₃ solution (10 mL) and extracted with CH₂Cl₂ (10 mL) twice. The combined organic fractions were dried over MgSO₄, filtered and the solvent removed by evaporation in vacuo. Purification by silica gel column chromatography, EtOAc elution, yielded GO (0.033 g, 73%).

LCMS (ESI): Found 448.9 [M+H]⁺.

¹H NMR (300 MHz, DMSO-d₆) δ: 8.67 (d, J=2.4 Hz, 1H), 8.43-8.48 (m, 2H), 8.08-8.15 (m, 2H), 7.99 (s, 1H), 7.73 (d, J=3.8 Hz, 1H), 7.45 (s, 1H), 7.35 (d, J=3.8 Hz, 1H), 5.62 (s, 2H).

¹⁹F NMR (282 MHz, DMSO-d₆), δ: −64.78 (s, 3F).

### Example GP

### 3-(Azetidine-1-carbonyl)-N-(pyrazin-2-yl)-N-({5-[5-(trifluoromethyl)-1,2,4-oxadiazol-3-yl]thiophen-2-yl}methyl)pyrazin-2-amine

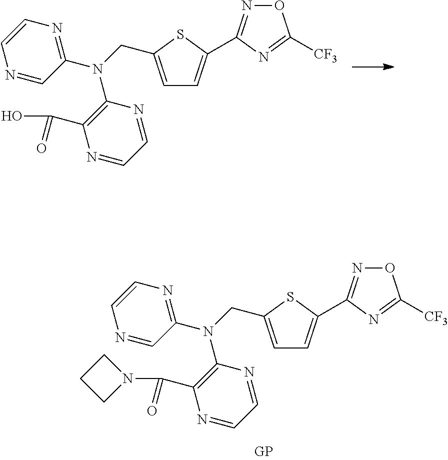 [SMILES: O=C(O)c1nccnc1N(Cc1ccc(-c2noc(C(F)(F)F)n2)s1)c1cnccn1 ; O=C(c1nccnc1N(Cc1ccc(-c2noc(C(F)(F)F)n2)s1)c1cnccn1)N1CCC1]

To a stirred solution of 3-[(pyrazin-2-yl)({5-[5-(trifluoromethyl)-1,2,4-oxadiazol-3-yl]thiophen-2-yl}methyl)amino]pyrazine-2-carboxylic acid (0.049 g, 0.1 mmol) in DMF (5 mL) under an argon atmosphere at rt was added CDI (0.019 g, 0.12 mmol). The reaction mixture was stirred for 30 min and then azetidine hydrochloride (0.021 g, 0.22 mmol) was added, followed by N,N-diisopropylethylamine (0.028 g, 0.22 mmol) The reaction mixture was stirred overnight and then poured into saturated Na₂CO₃ solution (10 mL) and extracted with CH₂Cl₂ (10 mL) twice. The combined organic fractions were dried over MgSO₄, filtered and the solvent removed by evaporation in vacuo. Purification by silica gel column chromatography, 10% MeOH/EtOAc elution, yielded GP (0.021 g, 43%).

LCMS (ESI): Found 488.9 [M+H]⁺.

¹H NMR (300 MHz, DMSO-d₆) δ: 8.68 (d, J=1.3 Hz, 1H), 8.61 (d, J=2.6 Hz, 1H), 8.24-8.37 (m, 3H), 7.74 (d, J=3.8 Hz, 1H), 7.41 (d, J=3.8 Hz, 1H), 5.69 (s, 2H), 4.23 (t, J=7.7 Hz, 2H), 3.56 (t, J=7.7 Hz, 2H), 2.18-2.45 (m, 2H).

¹⁹F NMR (282 MHz, DMSO-d₆), δ: −64.79 (s, 3F).

### Example GQ

### 1-{3-[(Pyrazin-2-yl)({5-[5-(trifluoromethyl)-1,2,4-oxadiazol-3-yl]thiophen-2-yl}methyl)amino]pyrazine-2-carbonyl}-3-(trifluoromethyl)azetidin-3-ol

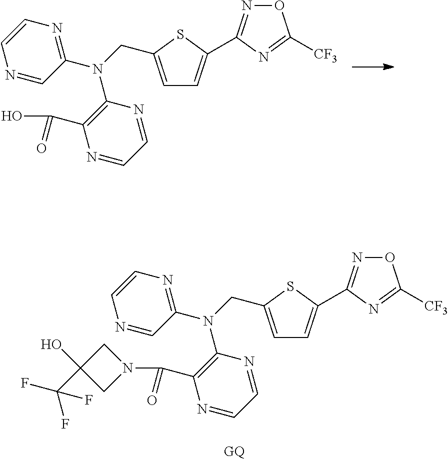 [SMILES: O=C(O)c1nccnc1N(Cc1ccc(-c2noc(C(F)(F)F)n2)s1)c1cnccn1 ; O=C(c1nccnc1N(Cc1ccc(-c2noc(C(F)(F)F)n2)s1)c1cnccn1)N1CC(O)(C(F)(F)F)C1]

To a stirred solution of 3-[(pyrazin-2-yl)({5-[5-(trifluoromethyl)-1,2,4-oxadiazol-3-yl]thiophen-2-yl}methyl)amino]pyrazine-2-carboxylic acid (0.049 g, 0.1 mmol) in DMF (5 mL) under an argon atmosphere at rt was added CDI (0.019 g, 0.12 mmol). The reaction mixture was stirred for 30 min and then 3-(trifluoromethyl)azetidin-3-ol hydrochloride (0.039 g, 0.22 mmol) was added, followed by N,N-diisopropylethylamine (0.028 g, 0.22 mmol) The reaction mixture was stirred overnight and then poured into saturated Na₂CO₃ solution (10 mL) and extracted with CH₂Cl₂ (10 mL) twice. The combined organic fractions were dried over MgSO₄, filtered and the solvent removed by evaporation in vacuo. Purification by silica gel column chromatography, EtOAc elution, yielded GQ (0.011 g, 19%).

LCMS (ESI): Found 572.8 [M+H]⁺.

¹H NMR (300 MHz, DMSO-d₆) δ: 8.71 (s, 1H), 8.68 (d, J=2.4 Hz, 1H), 8.42 (d, J=2.4 Hz, 1H), 8.23 (d, J=2.6 Hz, 1H), 8.17 (s, 1H), 7.75 (d, J=3.8 Hz, 1H), 7.56 (s, 1H), 7.42 (d, J=3.8 Hz, 1H), 5.77 (d, J=16.8 Hz, 1H), 5.65 (d, J=16.6 Hz, 1H), 4.54 (d, J=10.5 Hz, 1H), 4.28 (br d, J=10.4 Hz, 1H), 3.97 (d, J=11.1 Hz, 1H), 3.55 (br d, J=11.5 Hz, 1H).

¹⁹F NMR (282 MHz, DMSO-d₆), δ: −82.48 (s, 3F), −64.79 (s, 3F).

### Example GR

### N-[5-(Morpholine-4-carbonyl)pyridin-3-yl]-N-({5-[5-(trifluoromethyl)-1,2,4-oxadiazol-3-yl]thiophen-2-yl}methyl)pyrazin-2-amine

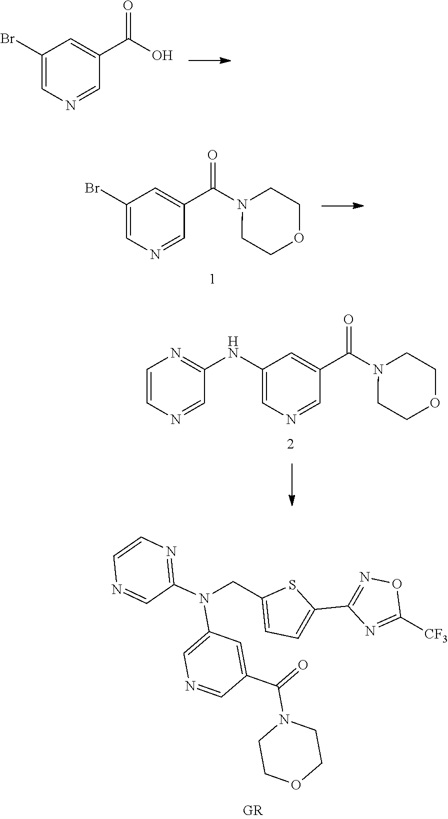 [SMILES: O=C(O)c1cncc(Br)c1 ; O=C(c1cncc(Br)c1)N1CCOCC1 ; O=C(c1cncc(Nc2cnccn2)c1)N1CCOCC1 ; O=C(c1cncc(N(Cc2ccc(-c3noc(C(F)(F)F)n3)s2)c2cnccn2)c1)N1CCOCC1]

To a stirred solution of 5-bromopyridine-3-carboxylic acid (1.01 g, 5 mmol) in THF (20 mL) under an argon atmosphere at rt was added CDI (0.973 g, 6 mmol). The reaction mixture was stirred for 2 h. Morpholine (0.96 mL, 11 mmol) was added and the reaction mixture was stirred for a further 1 h. The reaction mixture was poured into water (30 mL) and extracted with EtOAc (30 mL). The organic fraction was washed with saturated NaCl solution (30 mL), dried over MgSO₄, filtered and the solvent removed by evaporation in vacuo to yield intermediate 1 (1.059 g, 79%), which was used without further purification.

LCMS (ESI): Found 270.9 and 272.9[M+H]⁺.

Pyrazin-2-amine (1.05 g, 1.1 mmol), Cs₂CO₃ (0.652 g, 2 mmol), XantPhos (0.064 g, 0.11 mmol) and intermediate 1 (0.271 g, 1 mmol) were placed under an argon atmosphere, 1,4-dioxane (10 mL) was added and the reaction mixture degassed and placed under an argon atmosphere. Pd₂(dba)₃ (0.046 g, 0.05 mmol) was added and the reaction mixture heated to 90° C. overnight. Upon cooling the reaction mixture was poured into saturated NaCl solution (15 mL) and extracted with EtOAc (20 mL) twice. The combined organic fractions were dried over MgSO₄, filtered and the solvent removed by evaporation in vacuo. Purification by silica gel column chromatography, 10% MeOH/CH₂Cl₂ elution, yielded intermediate 2 (0.236 g, 83%).

LCMS (ESI): Found 286.0 [M+H]⁺.

To a stirred solution of intermediate 2 (0.086 g, 0.3 mmol) in DMF (5 mL) under an argon atmosphere at rt was added NaH (60% dispersion in oil, 0.013 g, 0.33 mmol) in a single portion. The reaction mixture was stirred for 30 min and then 3-[5-(bromomethyl)thiophen-2-yl]-5-(trifluoromethyl)-1,2,4-oxadiazole (0.094 g, 0.3 mmol) was added as a solution in DMF (2 mL). The reaction mixture was stirred for 1 h and then poured into saturated Na₂CO₃ solution (10 mL) and extracted with CH₂Cl₂ (15 ml) twice. The combined organic fractions were dried over MgSO₄, filtered and the solvent removed by evaporation in vacuo. Purification by silica gel column chromatography, 10% MeOH/CH₂Cl₂ elution, yielded GR (0.025 g, 16%).

LCMS (ESI): Found 517.9 [M+H]⁺.

¹H NMR (300 MHz, DMSO-d₆) δ: 8.68 (d, J=2.4 Hz, 1H), 8.49 (d, J=1.9 Hz, 1H), 8.29 (dd, J=2.7, 1.6 Hz, 1H), 8.16 (d, J=1.5 Hz, 1H), 8.09 (d, J=2.6 Hz, 1H), 7.85-7.88 (m, 1H), 7.75 (d, J=3.8 Hz, 1H), 7.19 (d, J=3.8 Hz, 1H), 5.48 (s, 2H), 3.46-3.71 (m, 8H).

¹⁹F NMR (282 MHz, DMSO-d₆), δ: −64.78 (s, 3F).

### Example GS

### 2-{6-[(Pyrazin-2-yl)({5-[5-(trifluoromethyl)-1,2,4-oxadiazol-3-yl]thiophen-2-yl}methyl)amino]pyridazin-4-yl}propan-2-ol

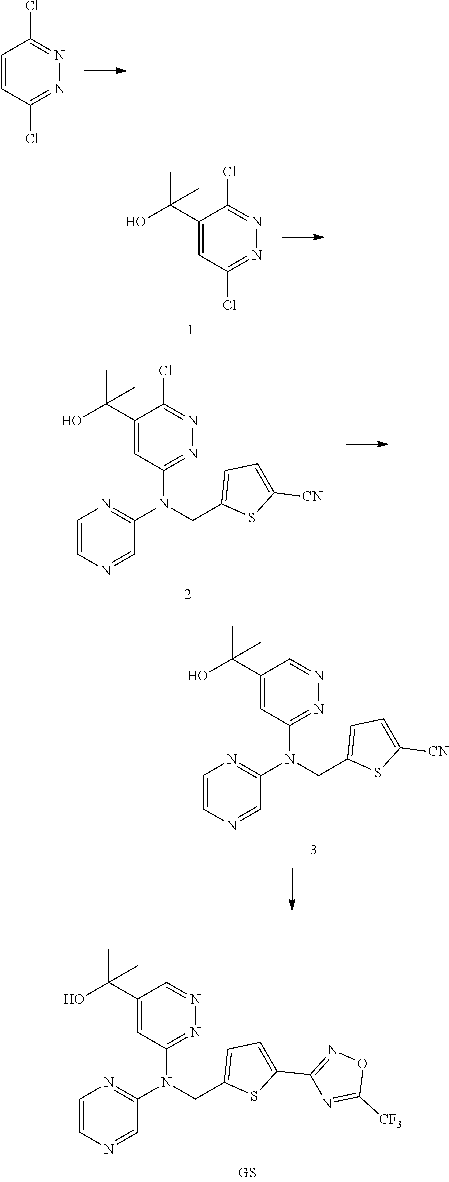 [SMILES: Clc1ccc(Cl)nn1 ; CC(C)(O)c1cc(Cl)nnc1Cl ; CC(C)(O)c1cc(N(Cc2ccc(C#N)s2)c2cnccn2)nnc1Cl ; CC(C)(O)c1cnnc(N(Cc2ccc(C#N)s2)c2cnccn2)c1 ; CC(C)(O)c1cnnc(N(Cc2ccc(-c3noc(C(F)(F)F)n3)s2)c2cnccn2)c1]

To a stirred solution of TMP (0.848 g, 6 mmol) in THF (50 mL) under an argon atmosphere at −78° C. was added nBuLi (1.6 M solution in hexanes, 7.44 mL, 5.5 mmol) dropwise. The reaction mixture was stirred at −78° C. for 15 min, warmed to 0° C. for 30 min and then cooled to −78° C. 3,6-Dichloropyridazine (0.745 g, 5 mmol) was added dropwise as a solution in THF (5 mL) and the reaction mixture stirred for 2 h. Acetone (0.435 g, 7.5 mmol) was added dropwise as a solution in THF (5 mL) and the reaction mixture stirred for 2 h at −78° C. and then allowed to warm to rt. The reaction mixture was poured into saturated NaHCO₃ solution (50 mL) and extracted with CH₂Cl₂ (50 mL) twice. The combined organic fractions were dried over MgSO₄, filtered and the solvent removed by evaporation in vacuo. Purification by silica gel column chromatography, 5% EtOAc/CH₂Cl₂ elution, yielded intermediate 1 (0.410 g, 40%).

LCMS (ESI): Found 206.9 [M+H]⁺.

Cs₂CO₃ (0.326 g, 1 mmol), XantPhos (0.064 g, 0.11 mmol) and intermediate 1 (0.104 g, 0.5 mmol) were placed under an argon atmosphere. 5-{[(Pyrazin-2-yl)amino]methyl}thiophene-2-carbonitrile (0.108 g, 0.5 mmol) was added as a solution in 1,4-dioxane (5 mL). The reaction mixture was degassed and placed under an argon atmosphere. Pd₂(dba)₃ (0.046 g, 0.05 mmol) was added and the reaction mixture heated to 101° C. for 5 h. After cooling the reaction mixture was poured into water (10 mL) and extracted with CH₂Cl₂ (10 mL) twice. The combined organic fractions were dried over MgSO₄, filtered and the solvent removed by evaporation in vacuo. Purification by silica gel column chromatography, EtOAc elution, yielded intermediate 2 (0.085 g, 44%).

LCMS (ESI): Found 386.9 [M+H]⁺.

To a stirred solution of intermediate 2 (0.085 g, 0.22 mmol) in EtOH (15 mL) was added ammonium formate (0.057 g, 0.9 mmol) followed by 10% Pd/C (0.01 g).

The reaction mixture was heated to 80° C. overnight. The reaction mixture was filtered and the solvent removed from the filtrate by evaporation in vacuo. The crude product was dissolved in CH₂Cl₂ (20 mL), washed with water (20 mL), dried over MgSO₄, filtered and the solvent removed by evaporation in vacuo. Purification by silica gel column chromatography, 5% MeOH/CH₂Cl₂ elution, yielded intermediate 3 (0.046 g, 59%).

LCMS (ESI): Found 353.0 [M+H]⁺.

To a stirred solution of intermediate 3 (0.046 g, 0.13 mmol) in EtOH (10 mL) was added NH₂OH solution (50%, 0.1 mL). The reaction mixture was heated to 80° C. for 1 h. Upon cooling the solvent was removed by evaporation in vacuo. The crude product was redissolved in EtOH (10 mL) and the solvent removed by evaporation in vacuo. The process was repeated. The crude product was dissolved in CH₂Cl₂ (10 mL) under an argon atmosphere at rt. Et₃N (0.054 mL, 0.39 mmol) was added followed by TFAA (0.036 mL, 0.26 mmol). The reaction mixture was stirred for 2 h and then poured into saturated NaHCO₃ solution (10 mL) and extracted with CH₂Cl₂ (10 mL) twice. The combined organic fractions were dried over MgSO₄, filtered and the solvent removed by evaporation in vacuo. Purification by silica gel column chromatography, 5% MeOH/CH₂Cl₂ elution, yielded GS (0.030 g, 50%).

LCMS (ESI): Found 463.9 [M+H]⁺.

¹H NMR (300 MHz, DMSO-d₆) δ: 9.06 (d, J=1.7 Hz, 1H), 8.71 (d, J=1.3 Hz, 1H), 8.42 (dd, J=2.6, 1.5 Hz, 1H), 8.27 (d, J=2.6 Hz, 1H), 7.74 (d, J=3.8 Hz, 1H), 7.67 (d, J=1.9 Hz, 1H), 7.29 (d, J=3.8 Hz, 1H), 5.72 (s, 2H), 5.45 (s, 1H), 1.44 (s, 6H).

¹⁹F NMR (282 MHz, DMSO-d₆), δ: −64.80 (s, 3F).

### Example GT

### 5-(3-Methyloxetan-3-yl)-N-(pyrazin-2-yl)-N-({5-[5-(trifluoromethyl)-1,2,4-oxadiazol-3-yl]thiophen-2-yl}methyl)pyridazin-3-amine

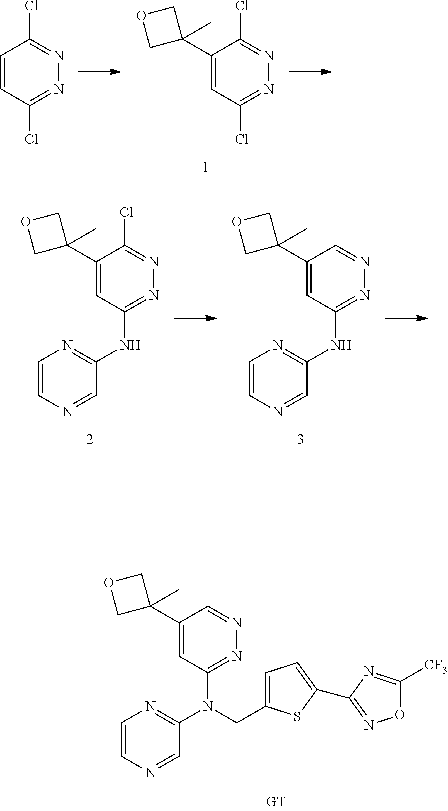 [SMILES: Clc1ccc(Cl)nn1 ; CC1(c2cc(Cl)nnc2Cl)COC1 ; CC1(c2cc(Nc3cnccn3)nnc2Cl)COC1 ; CC1(c2cnnc(Nc3cnccn3)c2)COC1 ; CC1(c2cnnc(N(Cc3ccc(-c4noc(C(F)(F)F)n4)s3)c3cnccn3)c2)COC1]

To a stirred suspension of 3-methyloxetane-3-carboxylic acid (0.327 g, 2.82 mmol) and 3,6-dichloropyridazine (0.299 g, 2 mmol) in water (2 mL) at 55° C. was added AgNO₃ (0.068 g, 0.4 mmol) as a solution in water (0.3 mL) followed by TFA (0.046 g, 0.4 mmol). (NH₄)₂S₂O₈ (0.778 g, 3.19 mmol) was added dropwise as a solution in water (1.5 mL). The reaction mixture was then heated to 75° C. for 1 h. Upon cooling the reaction mixture was poured into NaHCO₃ solution (15 mL) and extracted with CH₂Cl₂ (15 ml) twice. The combined organic fractions were dried over MgSO₄, filtered and the solvent removed by evaporation in vacuo. Purification by silica gel column chromatography, Et₂O elution, yielded intermediate 1 (0.316 g, 72%).

LCMS (ESI): Found 218.9 [M+H]⁺.

Cs₂CO₃ (0.932 g, 2.86 mmol), XantPhos (0.191 g, 0.33 mmol) and pyrazin-2-amine (0.136 g, 1.43 mmol) were placed under an argon atmosphere. Intermediate 1 (0.314 g, 1.43 mmol) was added as a solution in 1,4-dioxane (8 mL). The reaction mixture was degassed and placed under an argon atmosphere. Pd₂(dba)₃ (0.0137 g, 0.15 mmol) was added and the reaction mixture heated to 101° C. for 5 h. After cooling the reaction mixture was poured into water (10 mL) and extracted with CH₂Cl₂ (10 mL) twice. The combined organic fractions were dried over MgSO₄, filtered and the solvent removed by evaporation in vacuo. Purification by trituration with EtOAc yielded intermediate 2 (0.123 g, 31%).

LCMS (ESI): Found 278.0 [M+H]⁺.

To a stirred suspension of intermediate 2 (0.123 g, 0.44 mmol) in EtOH (20 mL) at rt was added ammonium formate (0.081 g, 1.3 mmol) followed by 10% Pd/C (0.03 g). The reaction mixture was heated to 80° C. for 1.5 h. A further portion of ammonium formate (0.063 g, 1 mmol) was added and the reaction mixture heated for a further 1.5 h. Upon cooling, the reaction mixture was filtered and the solvent removed by evaporation in vacuo. Purification by silica gel column chromatography, 10% MeOH/CH₂Cl₂ elution, yielded intermediate 3 (0.091 g, 85%).

LCMS (ESI): Found 244.0 [M+H]⁺.

To a stirred solution of intermediate 3 (0.91 g, 0.37 mmol) in DMF (5 mL) under an argon atmosphere at rt was added NaH (60% dispersion in oil, 0.016 g, 0.4 mmol) in a single portion. The reaction mixture was stirred for 30 min and then 3-[5-(bromomethyl)thiophen-2-yl]-5-(trifluoromethyl)-1,2,4-oxadiazole (0.125 g, 0.4 mmol) was added as a solution in DMF (2 mL). The reaction mixture was stirred for 2 h and then poured into saturated Na₂CO₃ solution (10 mL) and extracted with CH₂Cl₂ (15 ml) twice. The combined organic fractions were dried over MgSO₄, filtered and the solvent removed by evaporation in vacuo. Purification by silica gel column chromatography, EtOAc elution, yielded GT (0.072 g, 41%).

LCMS (ESI): Found 475.9 [M+H]⁺.

¹H NMR (300 MHz, DMSO-d₆) δ: 8.97 (d, J=1.9 Hz, 1H), 8.75 (d, J=1.3 Hz, 1H), 8.41 (dd, J=2.6, 1.5 Hz, 1H), 8.27 (d, J=2.6 Hz, 1H), 7.74 (d, J=3.6 Hz, 1H), 7.67 (d, J=1.9 Hz, 1H), 7.30 (d, J=3.8 Hz, 1H), 5.72 (s, 2H), 4.81 (d, J=6.0 Hz, 2H), 4.55 (d, J=6.0 Hz, 2H), 1.64 (s, 3H).

¹⁹F NMR (282 MHz, DMSO-d₆), δ: −64.80 (s, 3F).

### Example GU

### 5-(1,1-Difluoroethyl)-N-(pyrazin-2-yl)-N-({5-[5-(trifluoromethyl)-1,2,4-oxadiazol-3-yl]thiophen-2-yl}methyl)pyridazin-3-amine

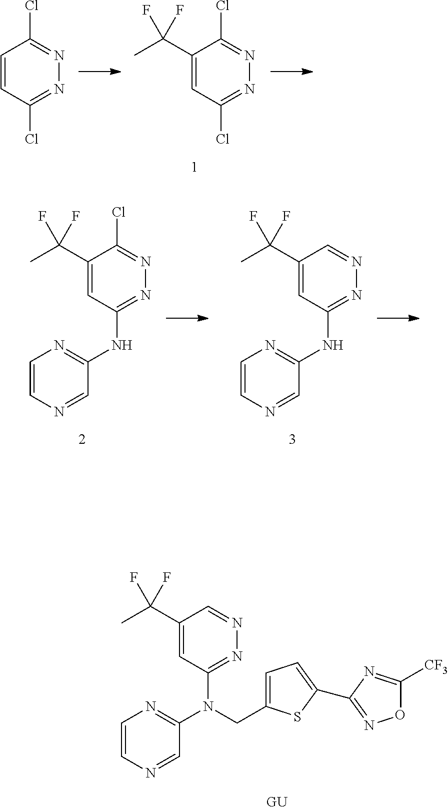 [SMILES: Clc1ccc(Cl)nn1 ; CC(F)(F)c1cc(Cl)nnc1Cl ; CC(F)(F)c1cc(Nc2cnccn2)nnc1Cl ; CC(F)(F)c1cnnc(Nc2cnccn2)c1 ; CC(F)(F)c1cnnc(N(Cc2ccc(-c3noc(C(F)(F)F)n3)s2)c2cnccn2)c1]

To a stirred suspension of 2,2-difluoropropanoic acid (0.465 g, 4.23 mmol) and 3,6-dichloropyridazine (0.447 g, 3 mmol) in water (3 mL) at 55° C. was added AgNO₃ (0.102 g, 0.6 mmol) as a solution in water (0.5 mL) followed by TFA (0.068 g, 0.6 mmol). (NH₄)₂S₂O₈ (1.092 g, 4.79 mmol) was added dropwise as a solution in water (2 mL). The reaction mixture was then heated to 75° C. for 1 h. Upon cooling the reaction mixture was poured into NaHCO₃ solution (15 mL) and extracted with CH₂Cl₂ (15 ml) twice. The combined organic fractions were dried over MgSO₄, filtered and the solvent removed by evaporation in vacuo. Purification by silica gel column chromatography, CH₂Cl₂ elution, yielded intermediate 1 (0.284 g, 44%).

LCMS (ESI): Found 213.0 [M+H]⁺.

Cs₂CO₃ (0.867 g, 2.66 mmol), XantPhos (0.191 g, 0.33 mmol) and pyrazin-2-amine (0.126 g, 1.33 mmol) were placed under an argon atmosphere. Intermediate 1 (0.283 g, 1.33 mmol) was added as a solution in 1,4-dioxane (8 mL). The reaction mixture was degassed and placed under an argon atmosphere. Pd₂(dba)₃ (0.0137 g, 0.15 mmol) was added and the reaction mixture heated to 101° C. for 5 h. After cooling the reaction mixture was poured into water (10 mL) and extracted with CH₂Cl₂ (10 mL) twice. The combined organic fractions were dried over MgSO₄, filtered and the solvent removed by evaporation in vacuo. The solid residue was washed with EtOAc, then dissolved in THF and treated with activated charcoal, filtered and the solvent removed by evaporation in vacuo to yield intermediate 2 (0.154 g, 43%).

LCMS (ESI): Found 272.1 [M+H]⁺.

To a stirred suspension of intermediate 2 (0.154 g, 0.57 mmol) in EtOH (15 mL) at rt was added ammonium formate (0.108 g, 1.71 mmol) followed by 10% Pd/C (0.04 g). The reaction mixture was heated to 80° C. for 2 h. Upon cooling, the reaction mixture was filtered and the solvent removed by evaporation in vacuo, yielded intermediate 3. Due to solubility issues, a proportion was purified by silica gel column chromatography, 10% MeOH/CH₂Cl₂.

LCMS (ESI): Found 228.1 [M+H]⁺.

To a stirred solution of intermediate 3 (0.043 g, 0.18 mmol) in DMF (5 mL) under an argon atmosphere at rt was added KOtBu (1.7M solution in THF, 0.118 mL, 0.2 mmol). The reaction mixture was stirred for 30 min and then 3-[5-(bromomethyl)thiophen-2-yl]-5-(trifluoromethyl)-1,2,4-oxadiazole (0.063 g, 0.2 mmol) was added as a solution in DMF (2 mL). The reaction mixture was stirred for 2 h and then poured into saturated Na₂CO₃ solution (15 mL) and extracted with CH₂Cl₂ (15 ml) twice. The combined organic fractions were dried over MgSO₄, filtered and the solvent removed by evaporation in vacuo. Purification by silica gel column chromatography, 2:1 EtOAc/hexane elution, yielded GU (0.038 g, 40%).

LCMS (ESI): Found 470.1[M+H]⁺.

¹H NMR (300 MHz, DMSO-d₆) δ: 9.15 (d, J=1.9 Hz, 1H), 8.85 (d, J=1.3 Hz, 1H), 8.43 (dd, J=2.6, 1.5 Hz, 1H), 8.34 (d, J=2.6 Hz, 1H), 7.87-7.91 (m, 1H), 7.75 (d, J=3.8 Hz, 1H), 7.32 (d, J=4.0 Hz, 1H), 5.77 (s, 2H), 2.02 (t, J=19.3 Hz, 3H).

¹⁹F NMR (282 MHz, DMSO-d₆), δ: −64.80 (s, 3F), −88.40 (q, J=19.7 Hz, 2F).

### Example GV

### 5-(Difluoromethyl)-N-(pyrazin-2-yl)-N-({5-[5-(trifluoromethyl)-1,2,4-oxadiazol-3-yl]thiophen-2-yl}methyl)pyridazin-3-amine

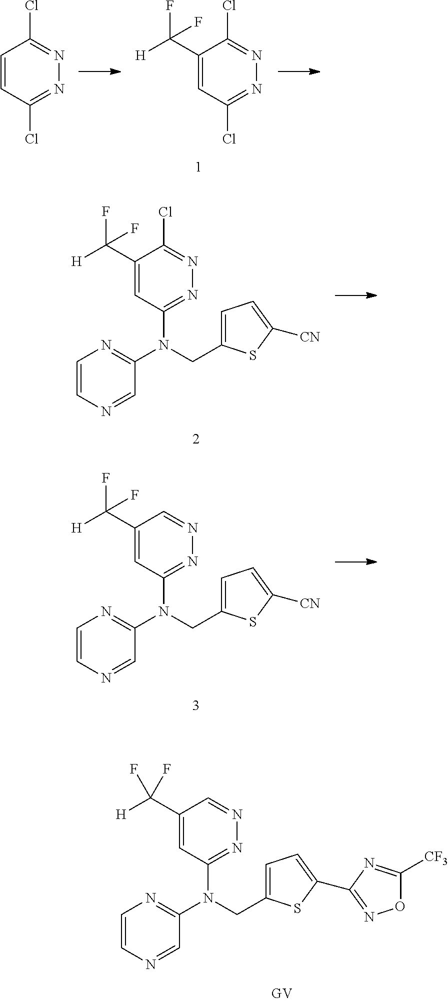 [SMILES: Clc1ccc(Cl)nn1 ; FC(F)c1cc(Cl)nnc1Cl ; N#Cc1ccc(CN(c2cnccn2)c2cc(C(F)F)c(Cl)nn2)s1 ; N#Cc1ccc(CN(c2cnccn2)c2cc(C(F)F)cnn2)s1 ; FC(F)c1cnnc(N(Cc2ccc(-c3noc(C(F)(F)F)n3)s2)c2cnccn2)c1]

To a stirred suspension of 2,2-difluoroacetic acid (1.218 g, 12.69 mmol) and 3,6-dichloropyridazine (1.341 g, 9 mmol) in water (6 mL) at 55° C. was added AgNO₃ (0.306 g, 1.8 mmol) as a solution in water (1.5 mL) followed by TFA (0.204 g, 1.8 mmol). (NH₄)₂S₂O₈ (3.276 g, 14.37 mmol) was added dropwise as a solution in water (6 mL). The reaction mixture was then heated to 75° C. for 1 h. Upon cooling the reaction mixture was poured into NaHCO₃ solution (50 mL) and extracted with CH₂Cl₂ (50 ml) twice. The combined organic fractions were dried over MgSO₄, filtered and the solvent removed by evaporation in vacuo. Purification by silica gel column chromatography, CH₂Cl₂ elution, yielded intermediate 1 (0.600 g, 34%).

MS (ESI): Found 199.0 [M+H]⁺.

Cs₂CO₃ (0.326 g, 1 mmol), XantPhos (0.064 g, 0.11 mmol) were placed under an argon atmosphere. Intermediate 1 (0.100 g, 0.5 mmol) was added as a solution in 1,4-dioxane (2 mL) followed by 5-{[(pyrazin-2-yl)amino]methyl}thiophene-2-carbonitrile (0.108 g, 0.5 mmol) as a solution in 1,4-dioxane (3 mL). The reaction mixture was degassed and placed under an argon atmosphere. Pd₂(dba)₃ (0.046 g, 0.05 mmol) was added and the reaction mixture heated to 101° C. for 6 h. After cooling the reaction mixture was poured into water (10 mL) and extracted with CH₂Cl₂ (10 mL) twice. The combined organic fractions were dried over MgSO₄, filtered and the solvent removed by evaporation in vacuo. Purification by silica gel column chromatography, 1:1 EtOAc/CH₂Cl₂ elution, followed by another silica gel column, 3:2 EtOAc/hexane elution, yielded intermediate 2 (0.090 g, 48%).

LCMS (ESI): Found 379.0 [M+H]⁺.

To a stirred solution of intermediate 2 (0.090 g, 0.24 mmol) in EtOH (20 mL) at rt was added ammonium formate (0.057 g, 0.9 mmol) followed by 10% Pd/C (0.03 g). The reaction mixture was heated to 80° C. for 1 h, then ammonium formate (0.057 g, 0.9 mmol) was added. The reaction mixture was heated for a further 1 h and then ammonium formate (0.057 g, 0.9 mmol) followed by 10% Pd/C (0.03 g) added. The reaction mixture was heated for a further 2 h, cooled and the catalyst removed by filtration, the solvent was removed by evaporation in vacuo. The crude product was dissolved in CH₂Cl₂ (15 mL), washed with NaCl solution, dried over MgSO₄, filtered and the solvent removed by evaporation in vacuo. Purification by silica gel column chromatography, EtOAc elution, yielded intermediate 3 (0.052 g, 63%).

LCMS (ESI): Found 345.1 [M+H]⁺.

To a stirred solution of intermediate 3 (0.052 g, 0.15 mmol) in EtOH (15 mL) was added NH₂OH solution (50%, 0.1 mL). The reaction mixture was heated to 80° C. for 1 h. Upon cooling the solvent was removed by evaporation in vacuo. The crude product was redissolved in EtOH (10 mL) and the solvent removed by evaporation in vacuo. The process was repeated. The crude product was dissolved in THF (5 mL) under an argon atmosphere at rt. Et₃N (0.063 mL, 0.45 mmol) was added followed by TFAA (0.042 mL, 0.3 mmol). The reaction mixture was stirred for 1 h at rt. Et₃N (0.063 mL, 0.45 mmol) was added followed by TFAA (0.042 mL, 0.3 mmol) and the reaction mixture stirred for a further 1 h. The reaction mixture was poured into saturated NaHCO₃ solution (10 mL) and extracted with CH₂Cl₂ (10 mL) twice. The combined organic fractions were dried over MgSO₄, filtered and the solvent removed by evaporation in vacuo. Purification by silica gel column chromatography, 2:1 EtOAc/hexane elution, yielded GV (0.042 g, 62%).

LCMS (ESI): Found 456.1 [M+H]⁺.

¹H NMR (300 MHz, DMSO-d₆) δ: 9.09-9.14 (m, 1H), 8.85 (d, J=1.5 Hz, 1H), 8.43 (dd, J=2.6, 1.5 Hz, 1H), 8.36 (d, J=2.6 Hz, 1H), 7.93 (d, J=1.3 Hz, 1H), 7.75 (d, J=3.8 Hz, 1H), 7.32 (s, 1H), 7.14 (t, J=55 Hz, 1H), 5.77 (s, 2H).

¹⁹F NMR (282 MHz, DMSO-d₆), δ: −64.80 (s, 3F), −116.30 (d, J=53.6 Hz, 2F).

### Example GW

### 3-{6-[(Pyrazin-2-yl)({5-[5-(trifluoromethyl)-1,2,4-oxadiazol-3-yl]thiophen-2-yl}methyl)amino]pyridazin-4-yl}oxetan-3-ol

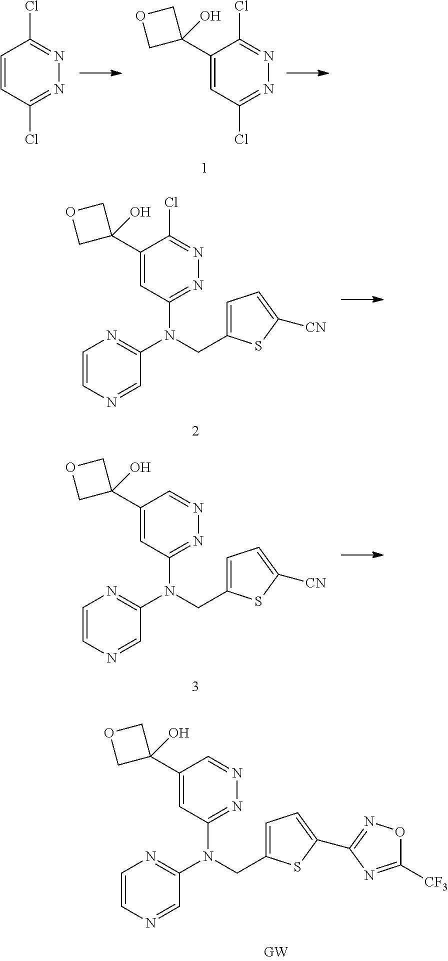 [SMILES: Clc1ccc(Cl)nn1 ; OC1(c2cc(Cl)nnc2Cl)COC1 ; N#Cc1ccc(CN(c2cnccn2)c2cc(C3(O)COC3)c(Cl)nn2)s1 ; N#Cc1ccc(CN(c2cnccn2)c2cc(C3(O)COC3)cnn2)s1 ; OC1(c2cnnc(N(Cc3ccc(-c4noc(C(F)(F)F)n4)s3)c3cnccn3)c2)COC1]

To a stirred solution of 3,6-dichloropyridazine (0.745 g, 5 mmol) in THF (30 mL) under an argon atmosphere at −78° C. was added 2,2,6,6-tetramethylpiperidinylmagnesium chloride lithium chloride complex (1M solution in THF/toluene, 5.5 mL, 5.5 mmol) dropwise. The reaction mixture was stirred for 1.5 h. Oxetan-3-one (0.396 g, 5.5 mmol) was added dropwise as a solution in THF (5 mL). The reaction mixture was stirred for 30 min at −78° C. and then allowed to warm to rt. The reaction mixture was poured into Na₂CO₃ solution (50 mL) and extracted twice with CH₂Cl₂ (50 mL). The combined organic fractions were dried over MgSO₄, filtered and the solvent removed by evaporation in vacuo. Purification by silica gel column chromatography, 2:1 EtOAc/hexane yielded intermediate 1 (0.551 g, 50%) as a white solid.

1H NMR (300 MHz, DMSO-d₆) δ: 8.14 (s, 1H), 6.80 (s, 1H), 5.11 (dd, J=7.4, 0.8 Hz, 2H), 4.67 (dd, J=7.5, 0.9 Hz, 2H).

Cs₂CO₃ (0.326 g, 1 mmol), XantPhos (0.064 g, 0.11 mmol), intermediate 1 (0.111 g, 0.5 mmol) and 5-{[(pyrazin-2-yl)amino]methyl}thiophene-2-carbonitrile (0.108 g, 0.5 mmol) were placed under an argon atmosphere and 1,4-dioxane (6 mL) added. The reaction mixture was degassed and placed under an argon atmosphere. Pd₂(dba)₃ (0.046 g, 0.05 mmol) was added and the reaction mixture heated to 101° C. for 6 h. After cooling the reaction mixture was poured into water (10 mL) and extracted with CH₂Cl₂ (10 mL) twice. The combined organic fractions were dried over MgSO₄, filtered and the solvent removed by evaporation in vacuo. Purification by silica gel column chromatography, EtOAc elution, yielded intermediate 2 (0.080 g, 40%).

LCMS (ESI): Found 401.1 [M+H]⁺.

To a stirred solution of intermediate 2 (0.080 g, 0.2 mmol) in EtOH (15 mL) at rt was added ammonium formate (0.057 g, 0.9 mmol) followed by 10% Pd/C (0.05 g). The reaction mixture was heated to 80° C. for 1 h, then ammonium formate (0.057 g, 0.9 mmol) was added. The reaction mixture was heated for a further 1 h and then ammonium formate (0.057 g, 0.9 mmol) followed by 10% Pd/C (0.05 g) added. The reaction mixture was heated for a further 2 h, cooled and the catalyst removed by filtration, the solvent was removed by evaporation in vacuo. The crude product was dissolved in CH₂Cl₂ (15 mL), washed with NaCl solution (15 mL), dried over MgSO₄, filtered and the solvent removed by evaporation in vacuo. Purification by silica gel column chromatography, 5% MeOH/CH₂Cl₂, yielded intermediate 3 (0.037 g, 51%).

LCMS (ESI): Found 367.1 [M+H]⁺.

To a stirred solution of intermediate 3 (0.037 g, 0.1 mmol) in EtOH (10 mL) was added NH₂OH solution (50%, 0.1 mL). The reaction mixture was heated to 80° C. for 1 h. Upon cooling the solvent was removed by evaporation in vacuo. The crude product was redissolved in EtOH (10 mL) and the solvent removed by evaporation in vacuo. The process was repeated. The crude product was dissolved in THF (5 mL) under an argon atmosphere at rt. Et₃N (0.042 mL, 0.3 mmol) was added followed by TFAA (0.028 mL, 0.2 mmol). The reaction mixture was stirred for 1 h at rt. Et₃N (0.042 mL, 0.3 mmol) was added followed by TFAA (0.028 mL, 0.2 mmol) and the reaction mixture stirred for a further 1 h. The reaction mixture was poured into saturated NaHCO₃ solution (10 mL) and extracted with CH₂Cl₂ (10 mL) twice. The combined organic fractions were dried over MgSO₄, filtered and the solvent removed by evaporation in vacuo. Purification by silica gel column chromatography, 5% MeOH/CH₂Cl₂ elution, gave a product that was further purified by silica gel column chromatography, 2.5% MeOH/EtOAc elution, to yield GW (0.018 g, 40%).

LCMS (ESI): Found 478.1 [M+H]⁺.

¹H NMR (300 MHz, DMSO-d₆) δ: 9.19 (d, J=1.9 Hz, 1H), 8.76 (d, J=1.3 Hz, 1H), 8.42 (dd, J=2.6, 1.5 Hz, 1H), 8.29 (d, J=2.4 Hz, 1H), 7.77 (d, J=1.9 Hz, 1H), 7.74 (d, J=3.8 Hz, 1H), 7.30 (d, J=3.8 Hz, 1H), 6.81 (s, 1H), 5.75 (s, 2H), 4.70-4.80 (m, 4H).

¹⁹F NMR (282 MHz, DMSO-d₆), δ: −64.79 (s, 3F).

### Example GX

### 5-tert-Butyl-N-(pyrazin-2-yl)-N-({5-[5-(trifluoromethyl)-1,2,4-oxadiazol-3-yl]thiophen-2-yl}methyl)pyridazin-3-amine

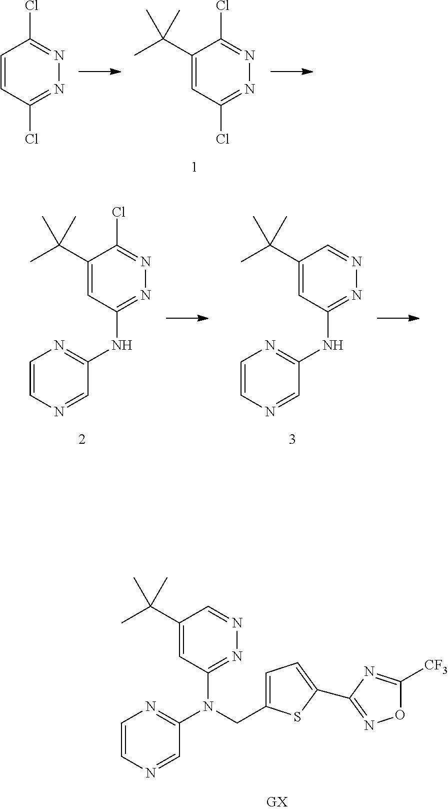 [SMILES: Clc1ccc(Cl)nn1 ; CC(C)(C)c1cc(Cl)nnc1Cl ; CC(C)(C)c1cc(Nc2cnccn2)nnc1Cl ; CC(C)(C)c1cnnc(Nc2cnccn2)c1 ; CC(C)(C)c1cnnc(N(Cc2ccc(-c3noc(C(F)(F)F)n3)s2)c2cnccn2)c1]

To a stirred suspension of 2,2-dimethylpropanoic acid (0.288 g, 2.82 mmol) and 3,6-dichloropyridazine (0.299 g, 2 mmol) in water (3 mL) at 55° C. was added AgNO₃ (0.068 g, 0.4 mmol) as a solution in water (0.3 mL) followed by TFA (0.046 g, 0.4 mmol). (NH₄)₂S₂O₈ (0.778 g, 3.19 mmol) was added dropwise as a solution in water (1.5 mL). The reaction mixture was then heated to 75° C. for 1 h. Upon cooling the reaction mixture was poured into NaHCO₃ solution (15 mL) and extracted with CH₂Cl₂ (15 ml) twice. The combined organic fractions were dried over MgSO₄, filtered and the solvent removed by evaporation in vacuo. Purification by silica gel column chromatography, 3:1 CH₂Cl₂/hexane elution, yielded intermediate 1 (0.33 g, 80%).

LCMS (ESI): Found 205.0 [M+H]⁺.

Cs₂CO₃ (1.023 g, 3.14 mmol), XantPhos (0.191 g, 0.33 mmol) and pyrazin-2-amine (0.149 g, 1.57 mmol) were placed under an argon atmosphere. Intermediate 1 (0.321 g, 1.57 mmol) was added as a solution in 1,4-dioxane (8 mL). The reaction mixture was degassed and placed under an argon atmosphere. Pd₂(dba)₃ (0.0137 g, 0.15 mmol) was added and the reaction mixture heated to 101° C. for 5.5 h. After cooling the reaction mixture was poured into water (15 mL) and extracted with CH₂Cl₂ (30 mL) twice. The combined organic fractions were dried over MgSO₄, filtered and the solvent removed by evaporation in vacuo. Purification by trituration with EtOAc yielded intermediate 2 (0.172 g, 42%).

LCMS (ESI): Found 264.1 [M+H]⁺.

To a stirred suspension of intermediate 2 (0.172 g, 0.65 mmol) in 1:1 MeOH/EtOH (40 mL) at rt was added ammonium formate (0.126 g, 2 mmol) followed by 10% Pd/C (0.06 g). The reaction mixture was heated to 80° C. for 2 h. Upon cooling the catalyst was removed by filtration, the solvent was removed by evaporation in vacuo. The crude product was dissolved in CH₂Cl₂ (15 mL), washed with NaCl solution (15 mL), dried over MgSO₄, filtered and the solvent removed by evaporation in vacuo to yielded intermediate 3 (0.121 g, 64%), which was used without further purification.

LCMS (ESI): Found 228.1 [M+H]⁺.

To a stirred solution of intermediate 3 (0.069 g, 0.3 mmol) in DMF (3 mL) under an argon atmosphere at rt was added NaH (60% dispersion in oil, 0.013 g, 0.33 mmol) in a single portion. The reaction mixture was stirred for 30 min and then 3-[5-(bromomethyl)thiophen-2-yl]-5-(trifluoromethyl)-1,2,4-oxadiazole (0.103 g, 0.33 mmol) was added as a solution in DMF (2 mL). The reaction mixture was stirred for 1 h and then poured into saturated Na₂CO₃ solution (10 mL) and extracted with CH₂Cl₂ (15 ml) twice. The combined organic fractions were dried over MgSO₄, filtered and the solvent removed by evaporation in vacuo. Purification by silica gel column chromatography, 2% MeOH/CH₂Cl₂ elution, yielded GX (0.06 g, 43%).

LCMS (ESI): Found 462.1 [M+H]⁺.

¹H NMR (DMSO-d₆) δ: 9.08 (d, J=1.9 Hz, 1H), 8.71 (d, J=1.3 Hz, 1H), 8.40 (dd, J=2.6, 1.5 Hz, 1H), 8.25 (d, J=2.6 Hz, 1H), 7.73 (d, J=3.8 Hz, 1H), 7.63 (d, J=2.1 Hz, 1H), 7.30 (d, J=3.8 Hz, 1H), 5.76 (s, 1H), 5.72 (s, 2H), 1.30 (s, 9H).

¹⁹F NMR (282 MHz, DMSO-d₆), δ: −64.80 (s, 3F).

### Example GY

### 5-[(2S)-2-Methylpyrrolidine-1-carbonyl]-N-(pyrazin-2-yl)-N-({5-[5-(trifluoromethyl)-1,2,4-oxadiazol-3-yl]thiophen-2-yl}methyl)pyridazin-3-amine

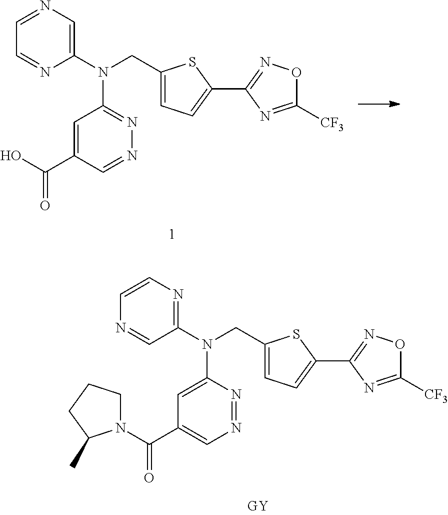 [SMILES: O=C(O)c1cnnc(N(Cc2ccc(-c3noc(C(F)(F)F)n3)s2)c2cnccn2)c1 ; C[C@H]1CCCN1C(=O)c1cnnc(N(Cc2ccc(-c3noc(C(F)(F)F)n3)s2)c2cnccn2)c1]

To a solution of acid 1 (50 mg, 0.11 mmol) in DMF (1 mL) was added N-methylmorpholine (25 μL, 0.22 mmol) followed by PyBOP (87 mg, 0.17 mmol). The reaction mixture was stirred for 25 min at rt before addition of 2-(S)-methylpyrrolidine (23 μL, 0.22 mmol). After 25 min the reaction mixture was diluted with EtOAc (30 mL), washed with HCl solution (5%, 3×10 mL), water (10 mL), sodium bicarbonate solution (5%, 3×5 mL), and brine (10 mL), before being dried over MgSO₄, filtered and concentrated in vacuo. Purification by flash column chromatography with EtOAc/MeOH (1:0 to 9:1) afforded a residue with was repurified by flash column chromatography with DCM/MeOH (1:0 to 97:3) to afford GY as off-white solids (36 mg, 63%).

LCMS (ES): Found 516.9 [M+H]⁺.

¹H NMR (300 MHz, DMSO-d₆) δ: 8.93-9.00 (m, 1H), 8.82 (d, J=1.3 Hz, 1H), 8.80 (d, J=1.3 Hz, 1H), 8.40 (dd, J=2.6, 1.5 Hz, 1H), 8.38 (dd, J=2.7, 1.5 Hz, 1H), 8.31 (d, J=2.6 Hz, 1H), 7.86 (d, J=1.7 Hz, 1H), 7.82 (d, J=1.7 Hz, 1H), 7.74 (d, J=3.8 Hz, 1H), 7.27-7.35 (m, 1H), 5.67-5.83 (m, 2H), 4.06-4.21 (m, 1H), 3.83-3.96 (m, 1H), 3.35-3.61 (m, 2H), 1.68-2.12 (m, 3H), 1.48-1.62 (m, 1H), 1.23 (d, J=6.4 Hz, 2H), 0.83 (d, J=6.4 Hz, 1H).

¹⁹F NMR (282 MHz, DMSO-d₆) δ: −64.80 (s, 3F).

### Example GZ

### 1,1,1,3,3,3-Hexafluoro-2-{6-[(pyrazin-2-yl)({5-[5-(trifluoromethyl)-1,2,4-oxadiazol-3-yl]thiophen-2-yl}methyl)amino]pyridazin-4-yl}propan-2-ol

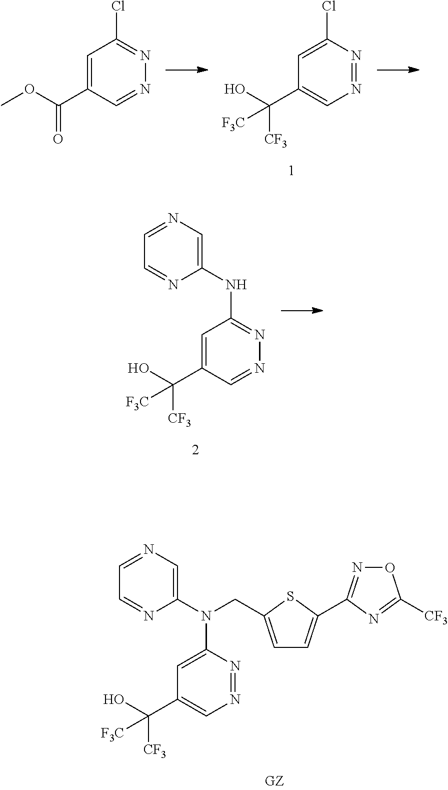 [SMILES: COC(=O)c1cnnc(Cl)c1 ; OC(c1cnnc(Cl)c1)(C(F)(F)F)C(F)(F)F ; OC(c1cnnc(Nc2cnccn2)c1)(C(F)(F)F)C(F)(F)F ; OC(c1cnnc(N(Cc2ccc(-c3noc(C(F)(F)F)n3)s2)c2cnccn2)c1)(C(F)(F)F)C(F)(F)F]

A solution of methyl 6-chloropyridazine-4-carboxylate (500 mg, 2.90 mmol) and (trifluoromethyl)trimethylsilane (520 μl, 3.48 mmol) in THF (10 mL) was treated with anhydrous cesium fluoride (22 mg, 0.14 mmol) and the reaction mixture stirred at rt for 24 hr. Hydrochloric acid (10%, 10 mL) was added and the mixture stirred for 2 hr 30 min before extraction with EtOAc (30 mL). The organics were washed with brine (10 mL), dried over MgSO₄, filtered and concentrated in vacuo. Purification by flash column chromatography using Hexanes/EtOAc (1:0 to 7:3) afforded 1 as pale yellow solids (75 mg, 9%).

A suspension of 1 (59 mg, 0.21 mmol), 2-aminopyrazine (20 mg, 0.21 mmol), Cs₂CO₃ (138 mg, 0.42 mmol) and Xantphos (12 mg, 0.02 mmol) in 1,4-dioxane (1.2 mL) was purged with Ar(g) for 15 min. Pd₂(dba)₃ (10 mg, 0.01 mmol) was added and mixture was heated to 90° C. for 3 hr. Reaction cooled to rt and partitioned between water (10 mL), brine (10 mL) and EtOAc (30 mL). The aqueous pH was adjusted to pH4 before being re-extracted with EtOAc (2×10 mL). The combined EtOAc extracts were washed with brine (10 mL), dried over MgSO₄, filtered and concentrated in vacuo. Purification by flash column chromatography using Hexanes/EtOAc (1:0 to 1:9) and subsequent trituration with DCM afforded 2 as light yellow solids (41 mg, 57%).

^{t}BuOK (1.7 M/THF, 135 μL) was added to a solution of (2) (37 mg, 0.11 mmol) in DMF (1.5 mL) at 0° C. under Ar(g). The reaction mixture was stirred for 10 min then (XX) (38 mg, 0.12 mmol) was added as solids in one portion. The reaction mixture was stirred for 45 min in the cooling bath and then poured into a mixture of water (10 mL), brine (10 mL), and hydrochloric acid (5%, 0.5 mL), followed by extraction with EtOAc (20 mL, 2×10 mL). The combined organics were washed with brine (10 mL), dried over MgSO₄, and treated with MP-TMT resin (1.3 meq./g, 100 mg) for 30 min. The mixture was filtered and concentrated in vacuo. Purification by flash column chromatography with Hexanes/EtOAc (1:0 to 1:3) afforded GZ as pale yellow solids (31 mg, 50%).

LCMS (ES): Found 572.1 [M+H]⁺.

¹H NMR (300 MHz, DMSO-d₆) δ: 9.51 (s, 1H), 9.13 (s, 1H), 8.87 (d, J=1.1 Hz, 1H), 8.40 (dd, J=2.4, 1.4 Hz, 1H), 8.37 (d, J=2.6 Hz, 1H), 7.89 (d, J=1.3 Hz, 1H), 7.75 (d, J=3.8 Hz, 1H), 7.33 (d, J=3.8 Hz, 1H), 5.79 (s, 2H).

¹⁹F NMR (282 MHz, DMSO-d₆) δ: −64.80 (s, 3F), −73.87 (s, 6F).

### ASSAY AND DATA

### Biochemical

### 1) Assay

i. Biochemical Assay Description

Activity against all zinc-dependent HDACs 1 to 11 was assessed by using an acetylated AMC-labeled peptide substrate. The substrate RHKK(Ac)AMC was used for HDAC1, HDAC2, HDAC3, HDAC6 and HDAC10; the substrate used for HDAC8 was RHK(Ac)K(Ac)AMC. Activity against the class IIa HDACs (HDAC4, 5, 7, 9) and HDAC11 was determined using a class IIa-specific substrate, BOC-Lys(trifluoroacetyl)-AMC (“Trifluoroacetyl Lysine”; see Lahm et al, 2007, PNAS, 104, 17335-17340). All assays were based on the AMC-labeled substrate and developer combination.

The protocol involved a two-step reaction: first, the substrate with the acetylated lysine side chain is incubated with a sample containing HDAC activity, to produce the deacetylated products, which are then digested in the second step by the addition of developer to produce the fluorescent signal proportional to the amount of deacetylated substrates.

ii. Enzymes

Human HDAC1 (GenBank Accession No. NM_004964), full length with C-terminal His-tag and C-terminal FLAG-tag, MW=56 kDa, expressed in baculovirus expression system.

Human HDAC2 (GenBank Accession No. NM_001527), full length with C-terminal GST-tag, MW=82.9 kDa, expressed inbaculovirus expression system.

Complex of human HDAC3 (GenBank Accession No. NM_003883), full length with C-terminal His tag, MW=49.7 kDa, and human NCOR2 (amino acid 395-489) (GenBank Accession No. NM_006312), N-terminal GST tag, MW=37.6 kDa, co-expressed in baculovirus expression system.

Human HDAC4 (GenBank Accession No. NM_006037), amino acids 627-1085 with N-terminal GST tag, MW=75.2 kDa, expressed in baculovirus expression system.

Human HDAC5 (GenBank Accession No. NM_005474), full length with C-terminal His tag, MW=51.1 kDa, expressed in baculovirus expression system.

Recombinant human HDAC6 (GenBank Accession No. BC069243), full length, MW=159 kDa, was expressed by baculovirus in Sf9 insect cells using an N-terminal GST tag.

Human HDAC7 (GenBank Accession No. AY302468), (a.a. 518-end) with N-terminal GST tag, MW=78 kDa, expressed in baculovirus expression system.

Human HDAC8 (GenBankAccession No. NM_018486), full length with C-terminal His tag, MW=42.6 kDa, expressed in a baculovirus expression system.

Human HDAC9 (GenBank Accession No. NM_178423), amino acids 604-1066 with C-terminal His tag, MW=50.7 kDa, expressed in baculovirus expression system.

Human HDAC10 (a.a. 1-481), GenBank Accession No. NM_032019 with N-terminal GST tag and C-terminal His tag, MW=78 kDa, expressed in baculovirus expression system.

Human HDAC11 (full length) (GenBank Accession No. NM_024827) with N-terminal His tag, MW=42.9 kDa, expressed in baculovirus expression system.

iii. Reaction Conditions

Assay Buffer: 50 mM Tris-HCl, pH8.0, 137 mM NaCl, 2.7 mM KCl, 1 mM MgC₂. Before use, 1 mg/mL BSA and DMSO are added.

HDAC1: 0.3 ng/ul HDAC1 and 50 μM HDAC substrate are in the reaction buffer with 1% DMSO final. Incubate for 1 hour at 30° C.

HDAC2: 0.07 ng/ul HDAC2 and 50 μM HDAC substrate are in the reaction buffer with 1% DMSO final. Incubate for 1 hour at 30° C.

HDAC3: 0.1 ng/ul HDAC3 and 50 μM HDAC substrate are in the reaction buffer with 1% DMSO final. Incubate for 1 hour at 30° C.

HDAC6: 0.3 ng/ul HDAC6 and 50 μM HDAC substrate are in the reaction buffer with 1% DMSO final. Incubate for 1 hours at 30° C.

HDAC8: 1 ng/ul HDAC8 and 100 M HDAC8 substrate are in the reaction buffer with 1% DMSO final. Incubate for 2 hours at 30° C.

HDAC10: 12 ng/ul HDAC10 and 50 μM HDAC substrate are in the reaction buffer with 1% DMSO final. Incubate for 2 hours at 30° C.

HDAC11: 5 ng/ul HDAC11 and 50 μMHDAC substrate are in the reaction buffer with 1% DMSO final. Incubate for 30 minutes at 30° C.

For class IIa HDACs, assay buffer is the same.

Other reaction conditions are as follows:

HDAC4: 0.004 ng/ul HDAC4 and 50 μM Class IIa HDAC substrate are in the reaction buffer with 1% DMSO final. Incubate for 30 minutes at room temperature.

HDAC5: 0.05 ng/ul HDAC5 and 50 μM Class IIa HDAC substrate are in the reaction buffer with 1% DMSO final. Incubate for 30 minutes at room temperature.

HDAC7: 0.001 ng/ul HDAC7 and 50 μM Class IIa HDAC substrate are in the reaction buffer with 1% DMSO final. Incubate for 30 minutes at room temperature.

HDAC9: 0.06 ng/ul HDAC9 and 50 μM Class IIa HDAC substrate are in the reaction buffer with 1% DMSO final. Incubate for 30 minutes at room temperature.

Control Inhibitor: Trichostatin A (TSA)

Fluorescent Deacetylated Standard: Biomol, Cat #KI-142;

For Standard Control, compound is added at assay concentration to 2.5 uM Fluorescent Deacetylated Standard; 10 doses in 6 uL.

For Fluorescence Background Control, compound is added at assay concentrations to 50 mM HDAC substrate; 10 doses in 6 uL.

Fluorescence background signal is then subtracted from compound data signal.

% Conversion must be between 5% and 15% to obtain optimum result.
iv. Assay Procedure

Stage 1: Deacetylation of substrate by incubation of HDAC enzymes with compounds

Stage 2: Development by addition of Developer to digest the deacetylated substrate, and generate the fluorescent colour; Detection: 360/460 Ex/Em

### 2) Inhibition of HDAC Enzymes

| | HDAC Isoform IC₅₀ (nM) | | | | | | | | | | |
|---|---|---|---|---|---|---|---|---|---|---|---|
| Example | 1 | 2 | 3 | 4 | 5 | 6 | 7 | 8 | 9 | 10 | 11 |
| S | **** | NT | NT | ** | *** | *** | *** | NT | *** | NT | NT |
| T | *** | NT | NT | ** | ** | *** | *** | NT | *** | NT | NT |
| U | **** | **** | **** | *** | *** | ** | *** | **** | *** | **** | **** |
| V | **** | **** | **** | *** | *** | * | *** | **** | *** | **** | **** |
| W | **** | NT | NT | *** | *** | *** | *** | NT | *** | NT | NT |
| X | **** | NT | NT | * | ** | ** | ** | NT | ** | NT | NT |
| Y | **** | NT | NT | * | ** | ** | ** | NT | ** | NT | NT |
| Z | **** | NT | NT | ** | ** | ** | ** | NT | *** | NT | NT |
| AA | **** | NT | NT | *** | *** | *** | *** | NT | *** | NT | NT |
| AB | **** | NT | NT | *** | *** | *** | **** | NT | *** | NT | NT |
| AC | **** | NT | NT | ** | ** | ** | *** | NT | *** | NT | NT |
| AD | *** | NT | NT | *** | *** | *** | *** | NT | *** | NT | NT |
| AE | **** | NT | NT | * | * | *** | ** | NT | *** | NT | NT |
| AF | **** | NT | NT | *** | ** | ** | *** | NT | *** | NT | NT |
| AG | **** | **** | **** | *** | *** | * | *** | *** | *** | **** | **** |
| AH | **** | NT | NT | **** | *** | *** | **** | NT | **** | NT | NT |
| AI | **** | NT | NT | **** | *** | *** | **** | NT | **** | NT | NT |
| AJ | **** | NT | NT | *** | *** | *** | *** | NT | *** | NT | NT |
| AK | **** | NT | NT | * | * | ** | * | NT | * | NT | NT |
| AL | **** | NT | NT | *** | *** | *** | *** | NT | *** | NT | NT |
| AM | **** | NT | NT | * | * | *** | ** | NT | *** | NT | NT |
| AN | **** | NT | NT | *** | *** | ** | *** | NT | *** | NT | NT |
| AO | **** | NT | NT | * | * | * | * | NT | ** | NT | NT |
| AP | **** | NT | NT | *** | *** | **** | *** | NT | *** | NT | NT |
| AQ | **** | NT | NT | *** | ** | *** | *** | NT | *** | NT | NT |
| AR | **** | NT | NT | * | * | * | * | NT | ** | NT | NT |
| AS | **** | **** | **** | *** | ** | ** | *** | *** | *** | **** | **** |
| AT | **** | NT | NT | *** | *** | *** | *** | NT | *** | NT | NT |
| AU | **** | NT | NT | *** | *** | *** | *** | NT | *** | NT | NT |
| AV | **** | NT | NT | *** | *** | *** | *** | NT | *** | NT | NT |
| AW | **** | NT | NT | *** | *** | *** | *** | NT | *** | NT | NT |
| AX | **** | NT | NT | *** | *** | *** | *** | NT | *** | NT | NT |
| AY | *** | NT | NT | *** | *** | ** | *** | NT | *** | NT | NT |
| AZ | **** | NT | NT | **** | *** | *** | **** | NT | **** | NT | NT |
| BA | **** | **** | **** | ** | ** | * | ** | *** | *** | **** | **** |
| BB | **** | NT | NT | * | * | *** | ** | NT | *** | NT | NT |
| BE | **** | **** | **** | * | * | * | * | *** | ** | **** | **** |
| BF | **** | **** | **** | *** | *** | *** | *** | *** | *** | **** | **** |
| BG | **** | NT | NT | *** | ** | ** | *** | NT | *** | NT | NT |
| BH | *** | **** | **** | ** | ** | * | ** | *** | *** | **** | **** |
| BI | **** | **** | **** | *** | *** | ** | * | *** | *** | **** | **** |
| BJ | *** | NT | NT | ** | *** | *** | *** | NT | *** | NT | NT |
| BK | **** | **** | **** | *** | *** | *** | *** | *** | *** | **** | **** |
| BL | **** | NT | NT | ** | ** | *** | ** | NT | *** | NT | NT |
| BM | *** | **** | *** | ** | ** | *** | *** | *** | *** | **** | **** |
| BN | **** | NT | NT | * | * | ** | * | NT | * | NT | NT |
| BO | **** | NT | NT | *** | *** | *** | *** | NT | *** | NT | NT |
| BP | **** | **** | **** | ** | * | * | *** | *** | *** | **** | **** |
| BQ | **** | **** | **** | *** | ** | * | *** | *** | *** | **** | **** |
| BR | **** | **** | **** | *** | *** | *** | *** | *** | *** | **** | **** |
| BS | **** | NT | NT | *** | *** | *** | *** | NT | *** | NT | NT |
| BT | **** | NT | NT | *** | *** | *** | *** | NT | *** | NT | NT |
| BU | **** | NT | NT | * | * | *** | ** | NT | ** | NT | NT |
| BV | **** | NT | NT | * | * | *** | ** | NT | ** | NT | NT |
| BW | **** | NT | NT | *** | *** | *** | *** | NT | *** | NT | NT |
| BX | **** | NT | NT | **** | *** | *** | *** | NT | *** | NT | NT |
| BY | **** | NT | NT | *** | *** | *** | *** | NT | *** | NT | NT |
| BZ | **** | **** | **** | * | * | * | * | *** | * | **** | **** |
| CA | **** | NT | NT | ** | ** | *** | ** | NT | ** | NT | NT |
| CB | **** | NT | NT | * | ** | ** | ** | NT | *** | NT | NT |
| CC | **** | NT | NT | *** | *** | *** | *** | NT | *** | NT | NT |
| CD | **** | NT | NT | ** | ** | *** | ** | NT | ** | NT | NT |
| CE | *** | NT | NT | *** | ** | * | ** | NT | *** | NT | NT |
| CF | **** | NT | NT | ** | *** | *** | *** | NT | *** | NT | NT |
| CG | **** | NT | NT | *** | ** | ** | *** | NT | *** | NT | NT |
| CH | **** | NT | NT | *** | *** | *** | *** | NT | *** | NT | NT |
| CI | *** | NT | NT | *** | *** | *** | *** | NT | *** | NT | NT |
| CJ | **** | NT | NT | *** | *** | *** | *** | NT | *** | NT | NT |
| CK | **** | NT | NT | ** | *** | *** | *** | NT | *** | NT | NT |
| CL | **** | NT | NT | *** | *** | *** | *** | NT | *** | NT | NT |
| CM | **** | NT | NT | *** | *** | *** | *** | NT | *** | NT | NT |
| CN | **** | NT | NT | *** | ** | ** | *** | NT | *** | NT | NT |
| CO | **** | NT | NT | * | ** | ** | ** | NT | *** | NT | NT |
| CP | **** | NT | NT | *** | *** | ** | *** | NT | *** | NT | NT |
| CQ | **** | NT | NT | *** | *** | *** | *** | NT | *** | NT | NT |
| CR | **** | **** | **** | * | * | * | * | *** | * | **** | **** |
| CS | *** | NT | NT | * | ** | ** | * | NT | *** | NT | NT |
| CT | **** | NT | NT | * | * | *** | * | NT | ** | NT | NT |
| CU | **** | NT | NT | * | ** | *** | ** | NT | *** | NT | NT |
| CV | **** | NT | NT | ** | ** | *** | ** | NT | *** | NT | NT |
| CW | **** | NT | NT | * | * | *** | * | NT | *** | NT | NT |
| CX | **** | NT | NT | * | * | *** | * | NT | ** | NT | NT |
| CY | **** | NT | NT | ** | * | *** | *** | NT | *** | NT | NT |
| CZ | **** | NT | NT | * | * | *** | * | NT | ** | NT | NT |
| DA | **** | NT | NT | ** | *** | *** | *** | NT | *** | NT | NT |
| DB | **** | NT | NT | * | * | *** | ** | NT | ** | NT | NT |
| DC | **** | NT | NT | * | * | *** | * | NT | ** | NT | NT |
| DD | **** | NT | NT | ** | *** | *** | *** | NT | *** | NT | NT |
| DE | **** | NT | NT | ** | ** | ** | ** | NT | *** | NT | NT |
| DF | **** | NT | NT | *** | *** | *** | *** | NT | *** | NT | NT |
| DG | **** | NT | NT | ** | ** | ** | *** | NT | *** | NT | NT |
| DH | *** | **** | *** | ** | ** | * | *** | *** | *** | **** | **** |
| DI | *** | **** | *** | ** | ** | * | ** | *** | *** | **** | **** |
| DJ | **** | NT | NT | *** | ** | * | *** | NT | *** | NT | NT |
| DK | *** | NT | NT | * | * | * | * | NT | *** | NT | NT |
| DL | *** | **** | **** | * | * | * | ** | *** | *** | **** | **** |
| DM | *** | NT | NT | * | ** | ** | ** | NT | ** | NT | NT |
| DN | *** | NT | NT | ** | * | * | * | NT | *** | NT | NT |
| DO | *** | NT | NT | ** | ** | * | ** | NT | *** | NT | NT |
| DP | **** | NT | NT | *** | *** | ** | *** | NT | *** | NT | NT |
| DQ | *** | **** | *** | * | * | * | * | *** | ** | **** | **** |
| DR | *** | **** | *** | * | * | * | * | *** | * | **** | **** |
| DS | **** | **** | *** | * | * | * | * | *** | ** | **** | **** |
| DT | **** | NT | NT | ** | * | ** | * | NT | ** | NT | NT |
| DU | **** | NT | NT | *** | *** | *** | ** | NT | *** | NT | NT |
| DV | **** | NT | NT | *** | ** | *** | *** | NT | *** | NT | NT |
| DW | **** | **** | **** | *** | *** | * | *** | *** | *** | **** | **** |
| DX | **** | **** | *** | ** | * | * | * | *** | *** | **** | **** |
| DY | **** | **** | **** | ** | ** | * | * | *** | *** | **** | **** |
| DZ | **** | **** | **** | *** | ** | * | ** | *** | *** | **** | **** |
| EA | **** | **** | *** | *** | ** | * | *** | *** | *** | **** | **** |
| EB | **** | **** | **** | *** | ** | * | ** | *** | *** | **** | **** |
| EC | **** | **** | **** | *** | *** | * | ** | *** | *** | **** | **** |
| ED | **** | **** | **** | *** | ** | * | *** | *** | *** | **** | **** |
| EE | **** | **** | **** | *** | ** | * | *** | *** | *** | **** | **** |
| EF | **** | **** | **** | *** | *** | * | *** | *** | *** | **** | **** |
| EG | **** | NT | NT | *** | *** | *** | **** | NT | **** | NT | NT |
| EH | **** | NT | NT | *** | *** | *** | *** | NT | **** | NT | NT |
| EI | **** | NT | NT | *** | ** | *** | *** | NT | *** | NT | NT |
| EJ | **** | NT | NT | *** | *** | *** | *** | NT | *** | NT | NT |
| EK | **** | NT | NT | **** | *** | *** | **** | NT | **** | NT | NT |
| EL | **** | NT | NT | *** | ** | ** | *** | NT | *** | NT | NT |
| EM | **** | NT | NT | *** | *** | ** | *** | NT | **** | NT | NT |
| EN | **** | NT | NT | *** | *** | * | *** | NT | **** | NT | NT |
| EO | **** | NT | NT | *** | *** | *** | *** | NT | **** | NT | NT |
| EP | **** | NT | NT | *** | *** | *** | *** | NT | **** | NT | NT |
| EQ | **** | NT | NT | *** | *** | ** | *** | NT | *** | NT | NT |
| ER | **** | NT | NT | *** | *** | * | *** | NT | *** | NT | NT |
| ES | **** | **** | **** | *** | *** | * | *** | *** | **** | **** | **** |
| ET | **** | NT | NT | *** | *** | * | *** | NT | **** | NT | NT |
| EU | **** | NT | NT | *** | *** | * | *** | NT | **** | NT | NT |
| EV | **** | NT | NT | *** | *** | *** | *** | NT | **** | NT | NT |
| EW | **** | NT | NT | *** | *** | *** | *** | NT | *** | NT | NT |
| EX | *** | NT | NT | *** | *** | ** | *** | NT | *** | NT | NT |
| EY | **** | NT | NT | *** | * | * | ** | NT | *** | NT | NT |
| EZ | **** | **** | **** | *** | *** | *** | *** | *** | *** | **** | **** |
| FA | **** | **** | *** | *** | ** | * | ** | *** | *** | **** | **** |
| FB | **** | **** | **** | ** | ** | *** | *** | **** | *** | **** | **** |
| FC | *** | **** | *** | ** | * | * | * | *** | *** | **** | **** |
| FD | *** | **** | *** | * | * | * | * | *** | *** | **** | **** |
| FE | **** | **** | *** | * | * | * | * | *** | *** | **** | **** |
| FF | *** | **** | *** | * | * | * | * | *** | ** | **** | **** |
| FG | **** | **** | **** | * | * | * | * | *** | ** | **** | **** |
| FH | *** | **** | *** | * | * | * | * | *** | ** | **** | **** |
| FI | *** | **** | *** | * | * | * | * | ** | ** | **** | **** |
| FJ | *** | **** | *** | * | * | * | * | *** | ** | **** | **** |
| FK | **** | **** | *** | * | * | * | * | ** | * | **** | **** |
| FL | **** | **** | **** | * | * | * | * | *** | ** | **** | **** |
| FM | *** | **** | **** | ** | * | * | ** | *** | *** | **** | **** |
| FN | **** | **** | **** | ** | ** | * | ** | *** | *** | **** | **** |
| FO | **** | **** | **** | ** | ** | * | ** | *** | *** | **** | **** |
| FP | **** | **** | **** | ** | * | * | *** | *** | *** | **** | **** |
| FQ | **** | **** | **** | *** | ** | ** | *** | *** | *** | **** | **** |
| FR | *** | **** | *** | * | * | * | * | *** | ** | **** | **** |
| FS | **** | **** | **** | *** | ** | * | ** | *** | *** | **** | **** |
| FT | **** | **** | **** | *** | ** | * | *** | *** | *** | **** | **** |
| FU | **** | **** | **** | *** | ** | * | *** | *** | *** | **** | **** |
| FV | **** | **** | **** | *** | ** | * | *** | *** | *** | **** | **** |
| FW | **** | **** | *** | *** | ** | * | *** | *** | *** | **** | **** |
| FX | **** | **** | **** | *** | *** | * | ** | *** | *** | **** | **** |
| FY | **** | **** | **** | *** | ** | * | *** | *** | *** | **** | **** |
| FZ | **** | **** | **** | *** | ** | * | ** | *** | *** | **** | **** |
| GA | **** | **** | **** | *** | ** | * | *** | *** | *** | **** | **** |
| GB | **** | **** | **** | *** | ** | * | *** | *** | *** | **** | **** |
| GC | **** | **** | **** | ** | * | * | * | *** | *** | **** | **** |
| GD | *** | **** | *** | *** | ** | * | ** | *** | *** | **** | **** |
| GE | **** | **** | **** | *** | ** | * | ** | *** | *** | **** | **** |
| GF | **** | **** | **** | ** | * | * | *** | *** | *** | **** | **** |
| GG | **** | **** | **** | ** | ** | ** | ** | *** | *** | **** | **** |
| GH | **** | **** | **** | *** | ** | * | *** | *** | *** | **** | **** |
| GI | **** | **** | **** | *** | *** | ** | *** | *** | *** | **** | **** |
| GJ | **** | **** | *** | ** | *** | * | ** | *** | *** | **** | **** |
| GK | *** | **** | *** | ** | ** | * | * | *** | *** | **** | **** |
| GL | *** | **** | *** | *** | ** | * | *** | *** | *** | **** | **** |
| GM | **** | **** | **** | *** | *** | * | * | *** | *** | **** | **** |
| GN | **** | **** | **** | ** | ** | * | ** | *** | *** | **** | **** |
| GO | **** | **** | **** | *** | *** | ** | *** | *** | *** | **** | **** |
| GP | **** | **** | **** | *** | *** | ** | *** | *** | *** | **** | **** |
| GQ | **** | **** | **** | *** | *** | ** | *** | *** | *** | **** | **** |
| GR | **** | **** | **** | ** | * | *** | * | *** | *** | **** | **** |
| GS | *** | **** | **** | ** | ** | * | ** | *** | *** | **** | **** |
| GT | **** | **** | **** | *** | ** | * | *** | *** | *** | **** | **** |
| GU | **** | **** | **** | *** | *** | * | *** | *** | *** | **** | **** |
| GV | **** | **** | **** | *** | ** | * | ** | *** | *** | **** | **** |
| GW | **** | **** | **** | *** | *** | * | ** | *** | *** | **** | **** |
| GY | **** | **** | **** | *** | ** | * | *** | *** | *** | **** | **** |

Key
**** >10 uM
*** ≤10 uM
** ≤1 uM
* ≤500 nM
NT Not tested

### Cellular

### 1) Assay

Acetylated α-Tubulin and Histone H3 in-Cell Western (ICW) Assays

A549 cells were maintained in DMEM (Life Technologies)+10% FBS+2 mM glutamine & penicillin (10 μg/mL) and streptomycin (100 mg/mL. 10,000 A549 cells per well were plated into 96 well tissue culture plates (Corning) and incubated at 37° C. overnight in a humidified atmosphere containing 5% CO₂ prior to addition of compounds. Compounds (dissolved in 100% DMSO) were diluted into cell culture media at 2× final assay concentration. 2× compound or DMSO (negative control) were added to cells (final DMSO concentration 0.4%). Untreated control wells received media. For acetylated α-tubulin assays, 40 μM scriptaid was used as a positive control. For acetylated histone H3 assays, 40 μM Trichostatin A (TSA) was used as a positive control. Cells were exposed to compound at 37° C. in a humidified atmosphere containing 5% CO₂ for 16 h.

Measurement of the impact of compounds on levels of acetylated α-tubulin and histone H3 was performed using specific antibodies and near-infrared immunofluorescent dyes. Briefly, media was removed from wells of assay plates. Cells were washed once with PBS and fixed in 3.7% formaldehyde PBS solution at room temperature for 20 min. Cells were then permeabilised by washing 5 times in 0.1% triton-X-100 PBS at room temperature with gentle agitation for 5 min per wash. Non-specific antibody staining was blocked by the addition of Odyssey blocking buffer (Li-Cor) for 1.5 h at room temperature with gentle agitation. Cells were then incubated with primary antibody (mouse monoclonal anti-acetylated α-tubulin (clone 6-11B-1), Sigma or rabbit polyclonal anti-acetylated histone H3, Millipore) diluted in Odyssey blocking buffer at 4° C. with gentle shaking overnight (no antibody control and secondary antibody only wells were incubated with Odyssey blocking buffer only). Cells were washed five times with 0.1% tween PBS and labelled with secondary antibody (IR dye 800CW-conjugated goat anti mouse IgG or -goat anti rabbit IgG, Li-Cor) and cell stains Sapphire700 (Li-Cor) and Draq5 (Biostatus) diluted in Odyssey blocking buffer at room temperature in the dark with gentle shaking for 1 h (no antibody control wells were incubated with Odyssey blocking buffer only and secondary antibody only with diluted secondary antibody but no cell stains). Cells were washed with 0.1% Tween PBS five times and scanned using a Li-Cor Odyssey SA plate reader in 700 and 800 nm channels. Acetylated α-tubulin or histone H3 signal was normalised to cell number using the cell stain signal. Percentage increase in acetylated α-tubulin or histone H3 after compound treatment over the mean of DMSO treated controls, relative to scriptaid and TSA positive controls, respectively was calculated. EC₅₀ values were calculated using GraphPad Prism software and nonlinear regression with 0% bottom and 100% top constraints.

### 2) EC₅₀ Data for Ac-α-Tubulin and Ac-Histone H3 in A549 Lung Adenocarcinoma Cells

| | EC₅₀ Ac-α-tubulin, | EC₅₀ Ac-histone H3, |
|---|---|---|
| Example | A549 cells | A549 cells |
| BA | * | *** |
| BH | ** | *** |
| DH | *** | *** |
| CE | *** | *** |
| DL | ** | *** |
| EF | ** | *** |
| DI | ** | *** |
| ER | *** | *** |
| DW | *** | *** |
| DZ | * | *** |
| EN | * | *** |
| EU | * | *** |
| EB | * | *** |
| ES | *** | *** |
| EA | * | *** |
| DX | * | *** |
| EC | * | *** |
| ED | * | *** |
| DQ | * | *** |
| DY | * | *** |
| EE | * | *** |
| DR | * | *** |
| DS | * | *** |
| FS | * | *** |
| FC | * | *** |
| FD | * | *** |
| FE | ** | *** |
| FF | * | *** |
| FG | * | *** |
| FH | * | *** |
| FU | * | *** |
| FV | * | *** |
| FW | * | *** |
| FI | * | *** |
| FJ | * | *** |
| FX | * | *** |
| GF | * | *** |
| FK | * | *** |
| FN | * | *** |
| FO | * | *** |
| GP | ** | *** |
| GH | * | *** |
| GI | * | *** |
| GJ | * | *** |
| GK | * | *** |
| GL | * | *** |

Key:
*** ≥10 uM
** <10uM > 1 uM
* ≤1 uM

### Comparative Data

The reported HDAC6 IC₅₀ and selectivity of a WO2017/222951 (Example 27) compound was compared to that obtained using the HDAC6 assay as described above, which differs from that described in WO2017/222951.

The differences in assay results, and a side-by-side comparison with a compound of the present invention using the assay described herein, are noted below:

| | Compound EB | WO2017/222951 (Example 27) | |
|---|---|---|---|
| | With assay | With assay | |
| | conditions in the | conditions in the | |
| | experimental | experimental | As reported |
| | section described | section described | in WO2017/ |
| | herein | herein | 222951 |
| HDAC6 IC₅₀ (nM) | 99.1 | 3577.5 | 160 |

### NUMBERED EMBODIMENTS

1. A compound of Formula I

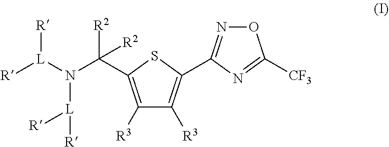

or a pharmaceutically acceptable salt thereof, wherein:

each R¹ is QR¹;

each Q is independently selected from a bond, —C₁-C₁₀ alkylene, —C₂-C₁₀ alkenylene, —C(O)—, —C(O)O—, —C(O)N(R¹)—, —N(R¹)C(O)—, —N(R¹)—, —N(SO₂(R¹)), —N(R¹)SO₂—, —C(O)NR⁴R⁵—, —N(R⁴R⁵)C(O)—, —N(R⁴R⁵)—, —S—, —SO—, —SO₂—, —S(O)O—, —SO₂N(R¹)— and —O—;

each R¹ is independently selected from H, C₁-C₁₀ alkyl, C₂-C₁₀ alkenyl, C₂-C₁₀ alkynyl, C₁-C₁₀ haloalkyl, C₁-C₁₀ heteroalkyl, aryl, heteroaryl, C₃-C₁₀ cycloalkyl, —(C₁-C₁₀ alkylene)-C₃-C₁₀ cycloalkyl, halogen, cyano, C₁-C₁₀ alkylene-aryl, C₁-C₁₀ alkylene heteroaryl, C₁-C₁₀ heterocycloalkyl and —(C₁-C₁₀ alkylene)-C₁-C₁₀ heterocycloalkyl;

each R² is independently selected from H, halogen and C₁-C₄ alkyl;

each R³ is independently selected from H, halogen, C₁-C₄ alkyl and C₁-C₁₀ haloalkyl;

each R⁴ and R⁵, when taken together with the nitrogen to which they are attached, form a 4- to 10-membered heteroarylene or heterocycloalkylene linker; and

each L is independently selected from a 5- to 10-membered nitrogen-containing heteroaryl;

wherein each cycloalkyl, heterocycloalkyl, aryl or heteroaryl may be substituted by up to three substituents selected from C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl) amino C₁-C₃ alkyl, bis(C₁-C₃ alkyl) amino C₁-C₃ alkyl, C₁-C₃ alkyl sulfonylamino, halo, nitro, cyano, trifluoromethyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl.

2. A compound according to numbered embodiment 1, wherein at least one L is a 6-membered nitrogen-containing heteroaryl.
3. A compound according to numbered embodiment 1 or numbered embodiment 2, wherein at least one L is a 5-membered nitrogen-containing heteroaryl.
4. A compound according to any preceding numbered embodiment, wherein each L is independently selected from a 5- or 6-membered nitrogen-containing heteroaryl.
5. A compound according to any preceding numbered embodiment wherein each L is independently selected from a 6-membered nitrogen-containing heteroaryl.
6. A compound according to any preceding numbered embodiment, wherein at least one L, preferably each L, contains two nitrogen atoms.
7. A compound according to any preceding numbered embodiment, wherein at least one L, preferably each L, is independently selected from the group consisting of pyridyl, pyrazolyl, pyrazinyl, pyrimidinyl, pyridazinyl, thiadiazolyl, oxadiazolyl and imidazolyl.
8. A compound according to any preceding numbered embodiment, wherein at least one L, preferably each L, is pyrazinyl or pyridazinyl.
9. A compound according to any preceding numbered embodiment, wherein at least one R², preferably each R², is H.
10. A compound according to any preceding numbered embodiment, wherein at least one R³, preferably each R³, is H or halogen.
11. A compound according to numbered embodiment 10, wherein R³ is H or F.
12. A compound according to any preceding numbered embodiment, wherein R′ is QR¹, wherein:

Q is independently selected from a bond, —C(O)N(R¹)—, —C(O)O— and —C(O)—,

R¹ is independently selected from H, C₁-C₁₀ alkyl, C₁-C₁₀ haloalkyl, C₁-C₁₀ heterocycloalkyl and heteroaryl.

13. A compound according to numbered embodiment 12, wherein each R¹ is independently selected from H, C₁-C₁₀ haloalkyl and C₁-C₁₀ heterocycloalkyl.
14. A compound according to any preceding numbered embodiment, wherein at least one R′ is H, preferably wherein at least one R′ on each L is H, preferably wherein each R′ on one L are H.
15. A compound according to any preceding numbered embodiment, wherein at least one R¹ is independently selected from H, C₁-C₁₀ haloalkyl, heterocycloalkyl, heteroaryl, cyano, —C(O)OR¹ and —C(O)R¹, wherein R¹ is heteroaryl or heterocycloalkyl.
16. A compound according to numbered embodiment 15, wherein C₁-C₁₀ haloalkyl is CF₃.
17. A compound according to any preceding numbered embodiment, as exemplified herein.
18. A compound according to any preceding numbered embodiment, for use in therapy.
19. A compound according to any preceding numbered embodiment, for use in the treatment or prevention of a condition mediated by histone deacetylase (HDAC).
20. A compound for use according to numbered embodiments 18 or 19, wherein the therapy is of cancer, cardiac hypertrophy, chronic heart failure, an inflammatory condition, a cardiovascular disease, a hemoglobinopathy, a thalassemia, a sickle cell disease, a CNS disorder, an autoimmune disease, diabetes, osteoporosis, MDS, benign prostatic hyperplasia, endometriosis, oral leukoplakia, a genetically related metabolic disorder, Charcot-Marie-Tooth disease, polycystic liver disease, rhabdomyolysis, an infection, Rubens-Taybi, fragile X syndrome, alpha-1 antitrypsin deficiency, peripheral neuropathy, organ transplantation, or respiratory disease.
21. A compound for use according to numbered embodiment 20, wherein the cancer is prostate cancer, endometrial cancer, ovarian cancer, cholangiocarcinoma, liver cancer, pancreatic cancer, lung cancer, leukemia, lymphoma, multiple myeloma, uterine cancer, bladder cancer, renal cancer, oesophageal cancer, breast cancer, gastric cancer, colorectal cancer, neuroblastoma, medulloblastoma, glioma or melanoma.
22. A compound for use according to numbered embodiment 20, wherein the inflammatory condition is a skin inflammatory condition, preferably selected from psoriasis, epidermolysis bullosa, epidermolysis bullosa acquisita, acne or eczema, a musculoskeletal inflammatory condition, preferably selected from rheumatoid arthritis, juvenile rheumatoid arthritis, ankylosing spondylitis or osteoarthritis, or an inflammatory condition of the gastrointestinal tract, preferably selected from an inflammatory bowel disease, preferably selected from Crohn's disease and ulcerative colitis.
23. A compound for use according to numbered embodiments 18 or 19, wherein the therapy is of a CNS disorder, preferably selected from stroke, a neuromuscular disorder (e.g. spinal muscular atrophy) or spinal cord injury.
24. A compound for use according to numbered embodiments 18 or 19, wherein the therapy is of a neurodegenerative disorder, preferably selected from Amyotrophic lateral sclerosis, Huntingdon's disease, Parkinson's disease or Alzheimer's disease.
25. A pharmaceutical composition comprising a compound according to any one of numbered embodiments 1 to 17, and a pharmaceutically acceptable carrier or diluent.
26. A product containing (a) a compound according to any one of numbered embodiments 1 to 17; and (b) another inhibitor of HDAC, for simultaneous, separate or sequential use in the treatment or prevention of a condition mediated by HDAC.
27. A product containing (a) a compound according to any one of numbered embodiments of 1 to 17, and (b) another chemotherapeutic or antineoplastic agent, for simultaneous, separate or sequential use in the treatment or prevention of cancer.
28. A method of treating a condition mediated by HDAC, comprising administering a pharmaceutically effective amount of a compound, composition or product according to any one of the preceding numbered embodiments.
29. Use of a compound, composition of product according to preceding numbered embodiment, for the manufacture of a medicament for use in the treatment or prevention of a condition mediate by histone deacetylase (HDAC).

1. A compound of Formula I 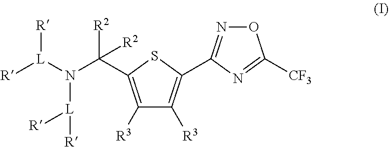
or a pharmaceutically acceptable salt thereof, wherein:
each R¹ is QR¹;
each Q is independently selected from a bond, —C₁-C₁₀ alkylene, —C₂-C₁₀ alkenylene, —C(O)—, —C(O)O—, —C(O)N(R¹)—, —C(O)N(R¹)SO₂—, —N(R¹)C(O)—, —N(R¹)—, —N(SO₂(R¹)), —N(R¹)SO₂—, —C(O)NR⁴R⁵—, —N(R⁴R⁵)C(O)—, —N(R⁴R⁵)—, —S—, —SO—, —SO₂—, —S(O)O—, —SO₂N(R¹)— and —O—;
each R¹ is independently selected from H, C₁-C₁₀ alkyl, C₂-C₁₀ alkenyl, C₂-C₁₀ alkynyl, C₁-C₁₀ haloalkyl, C₁-C₁₀ heteroalkyl, aryl, heteroaryl, C₃-C₁₀ cycloalkyl, —(C₁-C₁₀ alkylene)-C₃-C₁₀ cycloalkyl, halogen, cyano, C₁-C₁₀ alkylene-aryl, C₁-C₁₀ alkylene heteroaryl, C₁-C₁₀ heterocycloalkyl and —(C₁-C₁₀ alkylene)-C₁-C₁₀ heterocycloalkyl;
each R² is independently selected from H, halogen and C₁-C₄ alkyl;
each R³ is independently selected from H, halogen, C₁-C₄ alkyl and C₁-C₁₀ haloalkyl;
each R⁴ and R⁵, when taken together with the nitrogen to which they are attached, form a 4- to 10-membered heteroarylene or heterocycloalkylene linker; and
each L is independently selected from a 5- to 10-membered nitrogen-containing heteroaryl;
wherein each alkyl, heteroalkyl, cycloalkyl, heterocycloalkyl, aryl or heteroaryl may be substituted by up to three substituents selected from C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl) amino C₁-C₃ alkyl, bis(C₁-C₃ alkyl) amino C₁-C₃ alkyl, C₁-C₃ alkyl sulfonylamino, halo, nitro, cyano, C₁-C₃ haloalkyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl.

2. A compound according to claim 1, wherein at least one L is a 6-membered nitrogen-containing heteroaryl.

3. A compound according to claim 1 or 2, wherein at least one L is a 5-membered nitrogen-containing heteroaryl.

4. A compound according to any preceding claim, wherein each L is independently selected from a 5- or 6-membered nitrogen-containing heteroaryl.

5. A compound according to any preceding claim, wherein each L is independently selected from a 6-membered nitrogen-containing heteroaryl.

6. A compound according to any preceding claim, wherein at least one L, preferably each L, contains two nitrogen atoms.

7. A compound according to any preceding claim, wherein at least one L, preferably each L, is independently selected from the group consisting of pyridyl, pyrazolyl, pyrazinyl, pyrimidinyl, pyridazinyl, thiadiazolyl, oxadiazolyl, imidazolyl, pyrazolopyrimidinyl, imidazo(1,2-b)pyridazinyl, and (1,2,4)triazolo(4,3-b)pyridazinyl.

8. A compound according to any preceding claim, wherein at least one L, preferably each L, is pyrazinyl or pyridazinyl.

9. A compound according to any preceding claim, wherein at least one R² preferably each R², is H.

10. A compound according to any preceding claim, wherein at least one R³, preferably each R³, is H or halogen.

11. A compound according to claim 10, wherein R³ is H or F.

12. A compound according to any preceding claim, wherein R¹ is QR¹, wherein:
Q is independently selected from a bond, —C(O)N(R¹)—, —C(O)O— and —C(O)—,
R¹ is independently selected from H, C₁-C₁₀ alkyl, C₁-C₁₀ haloalkyl, C₁-C₁₀ heterocycloalkyl and heteroaryl.

13. A compound according to claim 12, wherein R¹ is independently selected from H, C₁-C₁₀ haloalkyl and C₁-C₁₀ heterocycloalkyl.

14. A compound according to any preceding claim, wherein at least one R′ is H, preferably wherein at least one R′ on each L is H, preferably wherein each R′ on one L are H.

15. A compound according to any preceding claim, wherein at least one R′ is independently selected from H, C₁-C₁₀ haloalkyl, heterocycloalkyl, heteroaryl, cyano, —C(O)OR¹ and —C(O)R¹, wherein R¹ is heteroaryl or heterocycloalkyl.

16. A compound according to claim 15, wherein C₁-C₁₀ haloalkyl is CF₃.

17. A compound according to any preceding claim, as exemplified herein.

18. A compound according to any preceding claim, for use in therapy.

19. A compound according to any preceding claim, for use in the treatment or prevention of a condition mediated by histone deacetylase (HDAC).

20. A compound for use according to claim 18 or 19, wherein the therapy is of cancer, cardiac hypertrophy, chronic heart failure, an inflammatory condition, a cardiovascular disease, a hemoglobinopathy, a thalassemia, a sickle cell disease, a CNS disorder, an autoimmune disease, diabetes, osteoporosis, MDS, benign prostatic hyperplasia, endometriosis, oral leukoplakia, a genetically related metabolic disorder, Charcot-Marie-Tooth disease, polycystic liver disease, rhabdomyolysis, an infection, Rubens-Taybi, fragile X syndrome, alpha-1 antitrypsin deficiency, peripheral neuropathy, organ transplantation, or respiratory disease.

21. A compound for use according to claim 20, wherein the cancer is prostate cancer, endometrial cancer, ovarian cancer, cholangiocarcinoma, liver cancer, pancreatic cancer, lung cancer, leukemia, lymphoma, multiple myeloma, uterine cancer, bladder cancer, renal cancer, oesophageal cancer, breast cancer, gastric cancer, colorectal cancer, neuroblastoma, medulloblastoma, glioma or melanoma.

22. A compound for use according to claim 20, wherein the inflammatory condition is a skin inflammatory condition, preferably selected from psoriasis, epidermolysis bullosa, epidermolysis bullosa acquisita, acne or eczema, a musculoskeletal inflammatory condition, preferably selected from rheumatoid arthritis, juvenile rheumatoid arthritis, ankylosing spondylitis or osteoarthritis, or an inflammatory condition of the gastrointestinal tract, preferably selected from an inflammatory bowel disease, preferably selected from Crohn's disease and ulcerative colitis.

23. A compound for use according to claim 18 or 19, wherein the therapy is of a CNS disorder, preferably selected from stroke, a neuromuscular disorder (e.g. spinal muscular atrophy) or spinal cord injury.

24. A compound for use according to claim 18 or 19, wherein the therapy is of a neurodegenerative disorder, preferably selected from Amyotrophic lateral sclerosis, Huntingdon's disease, Parkinson's disease or Alzheimer's disease.

25. A pharmaceutical composition comprising a compound according to any one of claims 1 to 17, and a pharmaceutically acceptable carrier or diluent.

26. A product containing (a) a compound according to any one of claims 1 to 17; and (b) another inhibitor of HDAC, for simultaneous, separate or sequential use in the treatment or prevention of a condition mediated by HDAC.

27. A product containing (a) a compound according to any one of claims 1 to 17, and (b) another chemotherapeutic or antineoplastic agent, for simultaneous, separate or sequential use in the treatment or prevention of cancer.

28. A method of treating a condition mediated by HDAC, comprising administering a pharmaceutically effective amount of a compound, composition or product according to any one of the preceding claims.

29. Use of a compound, composition of product according to preceding numbered embodiment, for the manufacture of a medicament for use in the treatment or prevention of a condition mediate by histone deacetylase (HDAC).
